(19) United States
(12) Patent Application Publication
(10) Pub. No.: US 2013/0174483 A1
(43) Pub. Date: Jul. 11, 2013

(54) PLANT ARTIFICIAL SEEDS AND METHODS FOR THE PRODUCTION THEREOF

(71) Applicant: E I DU PONT DE NEMOURS AND COMPANY, Wilmington, DE (US)

(72) Inventors: Timothy Caspar, Yorklyn, DE (US); Denise Gaspareto, Sao Joaquim Da Barra (BR); Lawrence Doka Gaultney, Elkton, MD (US); Ross Gilmour, Wilmington, DE (US); Beverly Hallahan, Wilmington, DE (US); David L. Hallahan, Wilmington, DE (US); Barry D. Johnson, Bridgeton, NJ (US); Brad H. Jones, Wilmington, DE (US); Katrina Kratz, Chadds Ford, PA (US); Prakash Lakshmanan, Indooroopilly (AU); Surbhi Mahajan, Grove City, OH (US); Brian D. Mather, Newark, DE (US); Barry Alan Morris, Wilmington, DE (US); Marcos Luciano Nunhez, Sao Paulo (S.P.) (BR); Jingjing Xu, Wilmington, DE (US)

(73) Assignee: E I DU PONT DE NEMOURS AND COMPANY, Wilmington, DE (US)

(21) Appl. No.: 13/721,105

(22) Filed: Dec. 20, 2012

Related U.S. Application Data
(60) Provisional application No. 61/578,410, filed on Dec. 21, 2011.

(51) Int. Cl. A01H 4/00; A01G 9/10

(52) U.S. Cl. CPC A01H 4/006; A01G 9/1026
USPC 47/65.8; 47/65.7; 47/66.7

(57) ABSTRACT

Composition and method for preparing artificial seeds of plantlets that can be developed into grown plants for propagation in the field are disclosed. In one embodiment, the artificial seeds are developed in degradable containers. The disclosed methods also allow for rapid propagation of in demand plants, such as sugarcane, to meet the ever increasing global demand for this plant.

### CROSS-REFERENCE TO RELATED APPLICATION

Benefit is claimed under 35 U.S.C. §119(e) to the filing date of U.S. Provisional Application No. 61/578,410, filed Dec. 21, 2011, the disclosure of which is herein incorporated by reference in its entirety.

### FIELD OF INVENTION

This invention relates to the production of plant artificial seeds. Specifically, it relates to the production of sugarcane artificial seeds.

### BACKGROUND

Some plants such as sugarcane, banana, pineapple, citrus, conifers and apple cannot be propagated via seeds due to: a) the loss of genetic identity during reproduction by seed; b) the long duration of growth for the plants before seed production; and c) the poor growth and survival rate of these plants' natural seeds under field growth conditions. Currently, these crops are propagated by either vegetative means or via seedlings. Thus attempts have been made to develop various economical alternatives for their propagation.

Artificial seeds have long been studied as an alternative means to propagate some plants (Kitto, S., Hort. Science, 20: 98-100, 1985). An artificial seed is an object that is man-made, and which includes components necessary to facilitate plant growth, and from which a plant may grow and be established from its own plant tissue, but wherein the plant tissue is typically not the same as the plant's natural seed. By contrast, a natural seed is produced by plants in a natural biological process without human intervention.

Traditional artificial seeds are alginate-encapsulated laboratory-cultured tissue that can be grown in vitro, but they suffer from very low survival rates in field environments due to both encapsulated material as well as biological challenges. Encapsulation is the process of adding the regenerable plant tissue to a container to provide an artificial seed. A regenerable plant tissue is a tissue capable of regenerating into a mature plant with the same features and genetic identity as the parent plant. A plantlet is one type of regenerable plant tissue. Plantlets can possess well-differentiated shoots and roots or they can be immature plantlets with only shoots that are capable of rooting when planted in soil or other growth media. Some of the challenges include the desiccation of exposed alginate-encapsulated tissue, attack by soil microorganisms, poor gas exchange of encapsulants, and immaturity and weakness of the laboratory-cultured tissue (Redenbaugh, K., Hort. Science, 22: 803-809, 1987 and Redenbaugh, K., Cell Cult and Somat Cell Genet Plants, 8: 35-74, 1991).

Artificial seeds have been used for the production of conifers using conifer embryos (Weyerhaeuser Corporation, WO1998033375). This method uses a complex, multi-compartment, individually-assembled design.

Sugarcane is commercially propagated vegetatively due to the loss of genetic identity during sexual reproduction by seed. Vegetative reproduction of this plant involves planting of stalk cuttings (multi-node stem sections called billets or whole stalks) horizontally in furrows. Each stalk has a bud or meristem, at each node. Meristems are undifferentiated cells found in zones of the plant where growth can take place. A node segment refers to a section of cane stalk containing a lateral bud, capable of regenerating a sugarcane plant. After planting, these buds produce shoots and roots, which become new sugarcane plants. The sugar and nutrients inside the stalk sections fuel the initial growth of the new plants.

The vegetative reproduction of sugarcane is a very laborious process and is fraught with issues. The main issues include the requirement of a large quantity of stalk material for planting (called “seed cane” in commercial cane production operations) that otherwise could be milled for sugar production, and the cost of dedicating a significant portion of the field and the labor involved to produce seed cane. Significant cost is involved in simply transporting multiple tons of sugarcane (10-15 ton/ha) needed to plant a field. Additionally, seed cane can contain diseases which are propagated by planting diseased sugarcane to the next generation. Hence, pathogen-free planting stocks need to be maintained, which involves large-scale stalk sterilization procedures, adding more cost to conventional propagation. For the introduction of new varieties of sugarcane, the vegetative propagation method is inefficient due to the long growing cycles and hence the relatively low multiplication factor (e.g., 5 to 15 kg of seed cane produced for each 1 kg of sugarcane planted) per growing cycle of 1 year duration.

Plene™ (Syngenta Co.), is a commercial product which consists of single node segments of the sugarcane stalk, trimmed of excess internode tissue to resemble miniaturized billets, and has been used as a vegetative propagule. A propagule is a plant material used for propagation.

Another process for culturing sugarcane meristems into bud masses from field-grown stalks of sugarcane has been disclosed (BSES, WO2011/085446 A1). This method allows for high multiplication factors, which can be used to accelerate variety release. However, the propagules from this process require hardening in a nursery before being transferred to the field, which limits their practicality for large scale sugarcane production.

Thus, there remains a need to develop novel and economical methods for improving the viability of the plant tissues incorporated into artificial seeds to enable direct planting of the plantlets into soil.

### SUMMARY OF INVENTION

The present invention provides artificial seeds to improve growth and viability of regenerable plant tissues and allow for a scaleable planting process of difficult to propagate plants such as sugarcane.

In one aspect, the invention is directed to an artificial seed comprising one or more regenerable plant tissues, a container comprising a degradable portion, an unobstructed airspace, and a nutrient source, and further comprising one or more features selected from the group consisting of: a penetrable or degradable region through which the regenerable plant tissue grows, a monolayer water soluble portion of the container, a region of the container that flows or creeps between about 1° C. and 50° C., a separable closure which is physically displaced during regenerable plant tissue growth, one or more openings in sides or bottom of the container, a conical or tapered region leading to an opening less than 2 cm wide at the apex and wherein the angle of the conical or tapered region is less than 135 degrees measured from opposite sides, and a plurality of flexible flaps through which the regenerable tissue grows.

In one embodiment of the invention, the container, region of the container, or a closure further comprises, or alternatively consists of, one or more of the following: polyesters, polyamides, polyolefins, cellulose, cellulose derivatives, polysaccharides, polyethers, polyurethanes, polycarbonates, poly(alkyl methacrylate)s, poly(alkyl acrylate)s, poly(acrylic acids), poly(meth)acrylic acids, polyphosphazenes, polyimides, polyanhydrides, polyamines, polydienes, polyacrylamides, poly(siloxanes), poly(vinyl alcohol), poly(vinyl esters), poly(vinyl ethers), natural polymers, block copolymers, crosslinked polymers, proteins, waxes, oils, plasticizers, antioxidants, nucleating agents, impact modifiers, processing aids, tougheners, colorants, fillers, stabilizers, flame retardants, natural rubber, polysulfones, or polysulfides; or blends thereof; or crosslinked versions thereof.

In another embodiment of the invention, the container further comprises a component selected from the group consisting of: a) amorphous poly(D,L-lactic acid), poly(lactic acid), poly(L-lactic acid), poly(D-lactic acid), poly(meso-lactic acid), poly(rac-lactic acid), or poly(D,L-lactic acid), (poly(hydroxyalkanoate), poly(hydroxybutyrate), poly(hydroxybutyrate-co-valerate), poly(caprolactone), poly(butylene succinate), poly(ethylene succinate), poly(ethylene carbonate), poly(propylene carbonate), starch, gelatin, thermoplastic starch, poly(butylene terephthalate adipate), poly(propylene terephthalate succinate), poly(propylene terephthalate adipate), poly(vinyl alcohol), poly(ethylene glycol), cellulose, chitosan, cellulose acetate, or cellulose butyrate acetate, b) a polyester with greater than 5 mol percent aliphatic monomer content, c) a crosslinked version of amorphous poly(D,L-lactic acid), poly(lactic acid), poly(L-lactic acid), poly(D-lactic acid), poly(meso-lactic acid), poly(rac-lactic acid), or poly(D,L-lactic acid), (poly(hydroxyalkanoate), poly(hydroxybutyrate), poly(hydroxybutyrate-co-valerate), poly(caprolactone), poly(butylene succinate), poly(ethylene succinate), poly(ethylene carbonate), poly(propylene carbonate), starch, gelatin, thermoplastic starch, poly(butylene terephthalate adipate), poly(propylene terephthalate succinate), poly(propylene terephthalate adipate), poly(vinyl alcohol), poly(ethylene glycol), cellulose, chitosan, cellulose acetate, cellulose butyrate acetate, or a polyester with greater than 5 mol percent aliphatic monomer content, d) a plasticizer, wherein the plasticizer is present at less than 30 wt % of the total composition, e) acetyl tributyl citrate, tributyl citrate, di-n-octyl sebacate, di-2-ethylhexylsebacate, di-2-ethylhexylsuccinate, diisooctyl adipate, di-2-ethylhexyl adipate, diisooctyl glutarate, di-2-ethylhexyl glutarate, poly(ethylene glycol), poly(ethylene glycol) monolaurate, sorbitol, glycerol, poly(propylene glycol), or water,

f) copolymers of two or more of caprolactone, lactic acid, D-lactide, L-lactide, meso-lactide, D,L-lactide, sebacic acid, succinic acid, adipic acid, glycolic acid, oxalic acid, ethylene glycol, 1,2-propanediol, 1,3-propanediol, 1,3-butanediol, 1,4-butanediol, 1,5-pentanediol, 2,2,4,4-tetramethyl-1,3-cyclobutanediol, 1,6-hexanediol, terephthalic acid, isophthalic acid, dimethyl siloxane, succinic anhydride, a diisocyanate, a crosslinker, or phthalic anhydride, g) an antioxidant, a nucleating agent, an impact modifier, a processing aid, a toughener, a colorant, a filler, a stabilizer, or a flame retardant, h) paper, water soluble paper, recycled paper, bond paper, kraft paper, waxed paper, or coated paper, i) a combination of two or more of components a) through h), and j) a blend comprising two or more of components a) through i).

In another embodiment, a region of the container or closure further comprises a component selected from the group consisting of: a) random, block or gradient copolymers of lactic acid with caprolactone, b) random, block or gradient copolymers of lactic acid with dimethylsiloxane, c) an alkyd resin, d) poly(vinyl alcohol), starch, cellulose, poly(ethylene glycol), agar, xanthan gum, alginate, hydroxypropylcellulose, methylcellulose, a water soluble protein, a water soluble carbohydrate, a water soluble synthetic polymer, or carboxymethylcellulose, e) blends of two or more of the following: poly(vinyl alcohol), starch, cellulose, glycerol, poly(ethylene glycol), citric acid, urea, water, sodium acetate, potassium nitrate, ammonium nitrate, fertilizers, agar, xanthan gum, alginate, hydroxypropylcellulose, methylcellulose, a water soluble protein, a water soluble carbohydrate, a water soluble synthetic polymer, a crosslinker, or carboxymethylcellulose, f) a gel comprising a block copolymer and an oil, g) sodium carboxymethylcellulose, h) wax-impregnated water soluble paper, i) amorphous poly(D,L-lactic acid), poly(lactic acid), poly(L-lactic acid), poly(D-lactic acid), poly(meso-lactic acid), poly(rac-lactic acid), or poly(D,L-lactic acid), poly(hydroxyalkanoate), poly(hydroxybutyrate), poly(hydroxybutyrate-co-valerate), poly(caprolactone), poly(butylene succinate), poly(ethylene succinate), poly(ethylene carbonate), poly(propylene carbonate), starch, thermoplastic starch, gelatin, poly(butylene terephthalate adipate), poly(propylene terephthalate succinate), poly(propylene terephthalate adipate), poly(vinyl alcohol), poly(ethylene glycol), cellulose, chitosan, cellulose acetate, cellulose butyrate acetate; or a crosslinked version thereof, j) a polyester with greater than 5 mol percent aliphatic monomer content, k) a crosslinked version of amorphous poly(D,L-lactic acid), poly(lactic acid), poly(L-lactic acid), poly(D-lactic acid), poly(meso-lactic acid), poly(rac-lactic acid), or poly(D,L-lactic acid), poly(hydroxyalkanoate), poly(hydroxybutyrate), poly(hydroxybutyrate-co-valerate), poly(caprolactone), poly(butylene succinate), poly(ethylene succinate), poly(ethylene carbonate), poly(propylene carbonate), starch, gelatin, thermoplastic starch, poly(butylene terephthalate adipate), poly(propylene terephthalate succinate), poly(propylene terephthalate adipate), poly(vinyl alcohol), poly(ethylene glycol), cellulose, chitosan, cellulose acetate, cellulose butyrate acetate, or a polyester with greater than 5 mol percent aliphatic monomer content, l) a plasticizer, wherein the plasticizer is present at less than 30 wt % of the total composition, m) acetyl tributyl citrate, tributyl citrate, di-n-octyl sebacate, di-2-ethylhexylsebacate, di-2-ethylhexylsuccinate, diisooctyl adipate, di-2-ethylhexyl adipate, diisooctyl glutarate, di-2-ethylhexyl glutarate, poly(ethylene glycol), poly(ethylene glycol) monolaurate, sorbitol, glycerol, poly(propylene glycol), or water, n) copolymers of two or more of caprolactone, lactic acid, D-lactide, L-lactide, meso-lactide, D,L-lactide, sebacic acid, succinic acid, adipic acid, glycolic acid, oxalic acid, ethylene glycol, 1,2-propanediol, 1,3-propanediol, 1,3-butanediol, 1,4-butanediol, 1,5-pentanediol, 2,2,4,4-tetramethyl-1,3-cyclobutanediol, 1,6-hexanediol, terephthalic acid, isophthalic acid, succinic anhydride, a diisocyanate, a crosslinker, or phthalic anhydride, o) an antioxidant, a nucleating agent, an impact modifier, a processing aid, a toughener, a colorant, a filler, a stabilizer, or a flame retardant, p) a wax, Parafilm® or Nescofilm®, q) paper, water soluble paper, recycled paper, bond paper, kraft paper, waxed paper, or coated paper; or r) a combination of two or more of components a) through q), and s) a blend comprising two or more of components a) through r).

In another embodiment, the container is expandable. Non-limiting examples of expandable methods include methods selected from the group consisting of: a) telescoping of two or more tubular members, b) unfolding, c) inflation, d) unraveling; and e) stretching.

In another embodiment of the invention, the nutrient source further comprises a component selected from the group consisting of: a) soil, b) coconut coir, c) vermiculite, d) an artificial growth medium, e) agar, f) a superabsorbent polymer, g) a plant growth regulator, h) a plant hormone, i) micronutrients, j) macronutrients, k) water, l) a fertilizer, m) peat, n) a combination of two or more of components a) through m), and o) a blend comprising two or more of components a) through n).

In another embodiment, the regenerable plant tissue is a regenerable tissue selected from the group consisting of: a) sugar cane, a graminaceous plant, saccharum spp, saccharum spp hybrids, miscanthus, switchgrass, energycane, sterile grasses, bamboo, cassava, corn, rice, banana, potato, sweet potato, yam, pineapple, trees, willow, poplar, mulberry, ficus spp, oil palm, date palm, poaceae, verbena, vanilla, tea, hops, Erianthus spp, intergeneric hybrids of Saccharum, Erianthus and Sorghum spp, African violet, apple, date, fig, guava, mango, maple, plum, pomegranate, papaya, avocado, blackberries, garden strawberry, grapes, canna, cannabis, citrus, lemon, orange, grapefruit, tangerine, or dayap, b) a genetically modified plant of a), c) a micropropagated version of a), and d) a genetically modified, micropropagated version of a).

In another embodiment, the container further comprises a component selected from the group consisting of: a) a cylindrical tube with a conical top, b) a two part tube with a porous bottom section and a nonporous top section, c) a flexible packet, d) a semi-flexible packet, e) a rolled tube structure, capable of unraveling, f) an anchoring device, g) a multi-part tube with a hinged edge, h) a multi-part tube held together with adhesive, i) a tubular shape, j) a container portion in contact with soil that degrades faster than the portion above soil, k) an airspace comprising multiple compartments, l) a closed bottom end that retains moisture, m) a cap attached by an adhesive joint, n) a cap attached by insertion into the container, and o) a weak region.

In another embodiment, the container or closure further comprises a material selected from the group consisting of: a) a transparent, translucent or semi-translucent material, b) an opaque material, c) a porous material, d) a nonporous material, e) a permeable material, f) an impermeable material; and g) any one of materials a) through f), wherein the material is biodegradable, hydrolytically degradable, or compostable.

In another embodiment, one or more of the openings are secured using a component selected from the group consisting of: a) a crimp, b) a fold, c) a porous material, d) mesh, e) screen, f) cotton, g) gauze; and h) a staple.

In another embodiment, the artificial seed further comprises an agent selected from the group consisting of: a) a fungicide, b) a nematicide, c) an insecticide, d) an antimicrobial compound, e) an antibiotic, f) a biocide, g) an herbicide, h) plant growth regulator or stimulator, i) microbes, j) a molluscicide, k) a miticide, l) an acaricide, m) a bird repellant, n) an insect repellant, o) a plant hormone; and p) a rodent repellant.

In another embodiment, a method for preparing the artificial seed comprising the steps of: a) preparing said container; b) preparing one or more regenerable plant tissues; and c) placing the tissue of step (b) inside the container prepared in step (a).

In another embodiment, a method of storing the artificial seed, comprising obtaining the artificial seed and storing said artificial seed before planting in one or more of the following conditions: a) ambient conditions, b) sub-ambient temperature, c) sub-ambient oxygen levels, or d) under sub-ambient illumination, and wherein the regenerable plant tissue remains viable.

In another embodiment, a method of planting the artificial seed, comprising obtaining the artificial seed and performing a step from the group consisting of: a) introducing one or more breaches in said artificial seed during planting, wherein the breaches facilitate the growth of the regenerable plant tissues, b) expanding the artificial seed, and c) the combination of a) and b).

### DESCRIPTION OF THE FIGURES

FIG. 1. FIG. 1 depicts the basic design of a container for use in preparation of artificial seeds. Numbers in this Figure are: (1) Parafilm® closures; (2) airspace; (3) plantlet; (4) paper cylinder; (5) agar medium; (6) optional cotton.

FIG. 2. FIG. 2 depicts a crenelated structure for a paper container.

FIG. 3. FIG. 3 is a graph showing sprouting fraction of sugarcane artificial seeds as a function of time after planting. The solid line depicts growth of plantlets in artificial seeds containing fungicide. The dashed line depicts growth of plantlets in artificial seeds without fungicide. Fraction of plantlets sprouting from the artificial seed is shown on the Y axis. Time (days) is shown on the X axis.

FIG. 4. FIG. 4 is a photograph of Petri plates containing plantlets that were cultured in the MS liquid medium for 10 days prior to their transfer to the MS agar medium in Petri plates for another 10 days. The plantlets were size-separated into smaller 1.0-1.5 cm plantlets (group 1) and the larger group trimmed to 1.6-2.0 cm (group 2) (Left two plates-1.6-2.0 cm (group 2); Right plate 1-1.5 cm long (group 1))

FIG. 5. FIG. 5 shows photographs of fully assembled artificial seeds. FIG. 5A is the side view of one smaller (4×0.8 cm) and one larger (6×1.1 cm) fully assembled artificial seed with soil inside. FIG. 5B shows the top view of assembled artificial seeds with plantlets in them seen through the Nescofilm® top closure.

FIG. 6. FIG. 6 shows photographs of fully assembled artificial seeds after 3 weeks in soil, successful artificial seeds show plantlets breaking past the Nescofilm® closure. FIG. 6A shows both small and large artificial seeds. FIG. 6B shows close up of top view of large artificial seed with plantlets breaking past or trying to break past the Nescofilm® closure; FIG. 6C shows the smaller artificial seeds.

FIG. 7. FIG. 7 is a graph depicting percentage of small (4 cm diameter) and large (6 cm diameter) artificial seeds with plantlets sprouted through Nescofilm® closures. Percentage of plants produced is shown on the Y axis and treatments on the X axis. Percentage survival of directly-planted plantlets (control) is shown in the right panel.

FIG. 8. FIG. 8 is a photograph of 17 days old plantlets (1.2-3.2 cm long), used for testing different transplanting substrates.

FIG. 9. FIG. 9 is a graph depicting survival/emergence (%) of the artificial seeds on the Y axis and number of days on the X axis (T1) shows percentage survival and (T2-T5) show shoot emergence of 17 days old plantlets. T1 shows the results with direct planting; T2 shows the results using a container with soil; T3 shows the results using a container with soil+water crystals; T4 shows the results using a container with perlite+peat moss+water crystals; and T5 shows the results using a container with water crystals from day 7 to day 63.

FIG. 10. FIG. 10 is a graph depicting shoot height and the number of plantlets emerging from artificial seeds after 63 days of growth in glasshouse (Y axis) and various treatments on the X axis. T1=direct planting; T2=container with soil; T3=container with soil+water crystal; T4=container with perlite+peat moss+water crystals; and T5=container with water crystals.

FIG. 11. FIG. 11 shows photographs of shoot and root-trimmed plantlets for encapsulation (FIG. 11A) and the artificial seeds ready for planting (FIG. 11B).

FIG. 12. FIG. 12 shows photographs of wells for manual planting artificial seeds were made in the middle of furrows by a metal rod device (FIG. 12A). Top view of seed constructs placed in the wells just before spraying with water (FIG. 12B).

FIG. 13. FIG. 13 is a graph depicting emergence and establishment of KQ228 plants (Y axis) from paper and plastic containers (X axis) and the survival and establishment of plants (without any container covering) planted directly in the soil.

FIG. 14. FIG. 14 shows photographs of plants produced from plastic artificial seeds (FIG. 14A) after 5 weeks of growth. Root system was well developed in plants in artificial seeds (FIG. 14B) and in direct planted ones (FIG. 14C).

FIG. 15. FIG. 15 shows diagrams of paper artificial seeds with additional windows on side for improved survival in horizontal planting. Numbers in this Figure are: (7) crenellation; (8) windows and (9) flat ends. FIG. 15A shows a diagram of paper artificial seeds with additional windows on side for improved survival in horizontal planting. Numbers in this Figure are: (7) crenellation and (8) windows. FIG. 15B shows a diagram of paper artificial seeds with additional windows on side for improved survival in horizontal planting. Numbers in this Figure are: (8) windows and (9) flat ends.

FIG. 16: FIG. 16 shows how crimping of the bottom ends of the wax paper tubes was performed. FIG. 16A shows how crimping of the bottom ends of the wax paper tubes was performed. FIG. 16B shows how crimping of the bottom ends of the wax paper tubes was further performed.

FIG. 17: FIG. 17 depicts a conical lidded wax paper tube artificial seed, wherein the conical lid is formed from a centrifuge tube with a hole cut in the end, and the base of the paper tube is crimped.

FIG. 18: FIG. 18 depicts a conical lidded wax paper tube artificial seed, wherein the conical lid is cut at an angle, and a flexible transparent film is glued adjacent to, with the free end covering the hole in the conical lid. This forms a flap reducing the moisture loss from the seed, while allowing the plant to push this aside. The base of the paper tube is crimped.

FIG. 19: FIG. 19 depicts conical lidded wax paper tube artificial seeds planted at various depths (8 or 12.5 cm) with superabsorbent beads at the base.

FIG. 20: FIG. 20 depicts an artificial seed structure consisting of two stacked conical plastic tubes with holes, with holes in the conical tips, and an open bottom end.

FIG. 21: FIG. 21 depicts an artificial seed structure consisting of a single conical tube fashioned from a 50 mL polypropylene centrifuge tube with a hole at the top end and a flexible transparent flap covering that hole and an open bottom end.

FIG. 22: FIG. 22 depicts an artificial seed structure constructed from two conical tubes fashioned from 15 mL and 50 mL polypropylene centrifuge tubes oriented in opposite directions and placed concentrically around a soil plug with the sugarcane plantlet. The annular cavity contains water swollen superabsorbent polymer. The inner tube has slots cut in the base to allow moisture to enter the cavity with the plant from the annular cavity. The wide end of the 50 mL tube is covered with unstretched Parafilm® M and the bottom end of the inner tube is open.

FIG. 23: FIG. 23 depicts an artificial seed structure constructed from two conical tubes fashioned from 15 mL and 50 mL polypropylene centrifuge tubes oriented in the same direction, placed concentrically, with the annular cavity left empty and the bottom ends left open.

FIG. 24: FIG. 24 depicts an artificial seed constructed from a tube with an expandable tent shaped film surrounding it. The film is expanded after removing a paper band that holds it in place prior to planting.

FIG. 25: FIG. 25 depicts a conical tube artificial seed possessing a slotted flexible film shaped into a conical end, on the end of a cylindrical tube. The “flaps” of the flexible film conical end can be pushed apart by a growing plantlet (not shown).

FIG. 26: FIG. 26 depicts a conical tube artificial seed constructed from a rolled plastic sheet with a sawtooth pattern on one side, resulting in “flaps” that can be pushed apart by a growing plantlet (not shown) and a “scroll” shape that can be expanded by a growing plantlet.

FIG. 27: FIG. 27 depicts a conical packet artificial seed constructed from poly(lactic acid) with a sugarcane plantlet and moist Metro-Mix® 360 inside, heat sealed along the bottom edge. The bottom end was cut and the top was cut with two perpendicular vertical lines as indicated by the dashed lines prior to planting.

FIG. 28: FIG. 28 depicts a conical tube artificial seed possessing a stake for anchoring purposes.

FIG. 29: FIG. 29 depicts a conical tube artificial seed possessing extendable flaps for archoring purposes.

FIG. 30: FIG. 30 depicts a tubular artificial seed with a plantlet inserted from a side opening.

FIG. 31: FIG. 31 depicts a packet type artificial seed with holes in the bottom half of each side and an open top.

FIG. 32: FIG. 32 depicts a packet type artificial seed with holes all along each side and a closed top.

FIG. 33: FIG. 33 depicts a conical tube artificial seed composed of two halves which are connected by a water soluble material along each edge. When the water soluble material dissolves, the two halves separate and can be pushed apart by the growing plantlet.

FIG. 34: FIG. 34 depicts a conical tube artificial seed composed of two halves with one edge glued with a flexible glue forming a hinged edge. This seed can be pivoted apart by the growing plantlet.

FIG. 35: FIG. 35A depicts a scroll-shaped artificial seed in which a band is used to hold it in a compressed state, and then removed to allow the seed to expand to its full size. This reduces the size of the seed during storage. FIG. 35B shows a schematic of a scroll-shaped artificial seed in which a band is used to hold it in a compressed state, and then removed to allow the seed to expand to its full size. This reduces the size of the seed during storage.

FIG. 36: FIG. 36 depicts a foldable artificial seed consisting of a flexible transparent tube surrounding a sugarcane plantlet and moist Metro-Mix® 360. A rubber band holds it in the folded state and is removed at planting. The purpose of this is to reduce the space occupied by the artificial seed prior to planting.

FIG. 37: FIG. 37A depicts a telescoping artificial seed fabricated from two sections of transparent plastic pipe. The smaller sections fit concentrically inside the larger section with a Parafilm® M band to create a snug fit. The two sections are in the collapsed state before planting and are expanded by telescoping them apart at planting. The purpose of this is to reduce the space occupied by the artificial seed prior to planting. Both ends of the artificial seed are open. FIG. 37B shows a schematic of a telescoping artificial seed fabricated from two sections of transparent plastic pipe. The smaller sections fit concentrically inside the larger section with a Parafilm® M band to create a snug fit. The two sections are in the collapsed state before planting and are expanded by telescoping them apart at planting. The purpose of this is to reduce the space occupied by the artificial seed prior to planting. Both ends of the artificial seed are open.

FIG. 38: FIG. 38 depicts an accordion-shaped expanding artificial seed fabricated from ribbed tubing with a more flexible top end that is collapsed and taped in place prior to planting. The tape is removed at planting to expand the seed structure. The purpose of this is to reduce the space occupied by the artificial seed prior to planting. The bottom end of the artificial seed is open.

FIG. 39: FIG. 39 depicts a tubular artificial seed with film ends that are slotted with two crossing cuts.

FIG. 40: FIG. 40 depicts a conical tube artificial seed with a separated compartment containing superabsorbent polymer, with plastic screens between this compartment and the compartment containing the plantlet, as well as a plastic screen attached to the bottom end.

FIG. 41: FIG. 41 depicts a conical tube artificial seed with a funnel shaped lid and an open bottom end.

FIG. 42: FIG. 42 depicts a conical tube artificial seed with a capped bottom end and two slots on opposite ends of the tube, thereby forming a cup to hold moisture in the seed.

FIG. 43: FIG. 43 depicts a telescoping conical tube artificial seed consisting of flexible sleeve bottom portion without a hole at the bottom fitting concentrically in a rigid tube with a conical hole at the top. The bottom sleeve is fabricated from poly(ε-caprolactone), allowing it to degrade in the soil.

FIG. 44: FIG. 44 depicts an ovoid-shaped synthetic seed structure.

FIG. 45: FIG. 45 depicts an expandable tube concept possessing a flexible top portion and a rigid bottom portion.

FIG. 46: FIG. 46 depicts a foldable flexible tube shaped artificial seed with heat sealed compartments along each edge. The top end is open and the bottom ends are either left open (FIG. 46A) or are heat sealed (FIG. 46B).

FIG. 47: FIG. 47 is a picture of films on top of optical targets. From left to right: Poly(lactic acid) (PLA4032D NatureWorks, Minnetonka, Minn.), 22 wt % poly(1,3-propanediol succinate) in PLA4032D, 50 wt % poly (1,3-propanediol succinate) in PLA4032D.

### DETAILED DESCRIPTION OF INVENTION

It is to be understood that this invention is not limited to the particular methodology, protocols, cell lines, genera, and reagents described, as such may vary. It is also to be understood that the terminology used herein is for the purpose of describing particular embodiments only, and is not intended to limit the scope of the present invention.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

One embodiment of the invention relates to the development of a plant artificial seed (FIG. 1) where a regenerable plant tissue (3) is placed in a container (4) and the container is planted in soil and the regenerable plant tissue is allowed to grow. An artificial seed of the present invention comprises a container and a regenerable plant tissue.

In another embodiment of the invention is provided an artificial seed comprising one or more regenerable plant tissues, a container comprising a degradable portion, an unobstructed airspace, and a nutrient source, and further comprising a feature selected from the group consisting of: a penetrable or degradable region through which the regenerable plant tissue grows, a monolayer water soluble portion of the container, a region of the container that flows between about 1° C. and 50° C., a separable closure which is physically displaced during regenerable plant tissue growth, one or more openings in sides or bottom of the container, a conical or tapered region leading to an opening less than 2 cm wide at the apex and wherein the angle of the conical or tapered region is less than 135 degrees measured from opposite sides, and a plurality of flexible flaps through which the regenerable tissue grows. The degradable region may be biodegradable, photodegradable, oxidatively degradable, hydrolytically degradable, or compostable. As used herein, “a region” means any component of the container or any associated closures.

A regenerable plant tissue is a tissue capable of regenerating into a mature plant with the same features and genetic identity as the parent plant. Regenerable plant tissues used for encapsulation in artificial seeds as described herein include, but are not limited to, apical or lateral meristematic tissue, callus, somatic embryos, natural embryos, plantlets, leaf whorls, stem and leaf cuttings, natural seeds, and buds. A plant of any age can be a source of these tissues. As used herein, “apical meristem” means the meristem at the apical end of the growing stalk. It is the tissue that generates new leaves as well as lateral meristems as the stalk elongates and grows in height.

Various meristematic tissues such as shoot apical meristem, lateral shoot meristem, root apical meristem, vascular meristem and young immature leaves are used in the practice of the present invention. In one embodiment, apical shoot meristem tissue can be used. In another embodiment, lateral shoot meristem tissue is used. In another embodiment leaf tissue is used. As used herein, “meristem” encompasses all kinds of meristems available from a plant.

As used herein, “container” means any hollow structure that can hold the regenerable plant tissue. The container can have a variety of shapes and forms, so long as the shape allows the container to hold the plant tissue. For example, the container can be spherical, tubular with circular, conical, cubic, ovoid or any other cross-sectional shape. In one embodiment of the invention, the regenerable plant tissue can have a volume of between 0.0001% and 90% of the container volume.

One class of regenerable plant tissues of interest is micropropagated plant tissue. Micropropagated tissue is typically grown in a highly hydrated environment, and thus typically lacks features such as full stomatal function and protective morphology such as a cuticle layer. These features are important for the regulation of moisture within the tissue and pose an issue for the survival of these tissues outside of the micropropagation environment. In particular, the field environment can be particularly harsh and challenging for the survival of micropropagated tissues. Micropropagated sugarcane plantlets lack desiccation tolerance and typically exhibit low survival in the field environment. The traditional solution for this is to condition the sugarcane plantlets in a greenhouse, however this is costly and time consuming and results in plants that are too large to plant economically in production fields. In order to support the survival of these tissues in a field environment, it is critical to offer protection from desiccation. This protection may involve protecting the tissue from wind, and creating a humid local environment around the tissue. This can be accomplished by creating a physical barrier or container around the tissue.

Another feature of micropropagated tissue is that it typically lacks robust, lignified structures such as woody stems. These are important to provide stiffness to a mature plant which prevents the plant from damage during winds. Due in part to the lack of such structures, and the sometimes decreased vigor of these tissues compared to natural seeds, it is challenging for micropropagated tissue to escape a container offering maximum protection against moisture loss and desiccation. Micropropagated sugarcane plantlets possess weak, grassy shoots, which are incapable of puncturing commonly-used packaging materials. Thus, it is important to develop mechanisms enabling the escape and proliferation of these tissues from packaging materials.

Ideally, containers reduce the rate of water loss the tissue experiences in the field environment, either through transpiration into the atmosphere or conduction and capillary action into the surrounding soil. The container must also allow sufficient gas permeability, to allow the tissue to obtain the gases it needs for photosynthesis and respiration. Additionally, it is beneficial that the container allow the passage of some light to the plant for photosynthesis. Assuming the container protects the tissue adequately to enable survival and growth, the tissue will grow to a size requiring it to escape and shed the container. This allows the roots to proliferate into the soil to reach additional nutrient and water sources, and allows the leaves and shoots to proliferate to increase photosynthesis.

In one embodiment, the invention provides novel packaging containers for the delivery and successful growth of micropropagated tissue, said novel packaging containers referred to hereinafter as artificial seed(s). In general, the artificial seed will have a top and bottom end, with the micropropagated tissue positioned such that the shoots grow toward the top end, and the roots grow toward the bottom end. In a non-limiting hypothesis of the invention, it is believed that the top region of the artificial seed is more important to protect from moisture loss than the bottom region, due to the fact that soil offers a buffer from evaporation and may also provide a source of moisture depending on the depth the artificial seed is planted.

Artificial seed of the invention may include one or more of the following mechanisms, including all seven, in order to balance the moisture retentive feature of the artificial seed while allowing the eventual escape and proliferation of the micropropagated tissue:

1) In one embodiment of the invention, weak regions of the artificial seed or lid(s) thereof are contemplated which block moisture loss while allowing shoots and roots of the developing plant to puncture them. It is not feasible for the entire container to be composed of such a weak material, as this would pose problems for handling, storage and planting;

2) In another embodiment of the invention, the artificial seed(s) comprise degradable regions or lids thereof which block moisture loss and degrade at a rate commensurate with the growth and development of protective structures within the plant itself, such that the container releases the plant at a developmentally favorable stage. The degradation mechanism includes, but is not limited to, one of the following: biodegradation, hydrolytic degradation, photo-degradation or oxidative degradation. In a particular embodiment, the artificial seed comprises, or alternatively consists of, two degradable materials having different degradation rates, wherein the degradation rate of the subsurface portion is more rapid than the degradation rate of the aerial portion. In a non-limiting example, once the subsurface portion has degraded, the aerial portion is displaced with the growth of the shoots;

3) In another embodiment of the invention, the artificial seed(s) comprise flap-like structures in which a plurality of flexible flaps converge to substantially enclose one or both ends of the structure, preferably the top end of the structure. The mechanical behavior of the flaps is designed through material choice and geometrical features (thickness, angle relative to emerging shoots) to enable weak plants to deflect and thereby escape the artificial seed;

4) In yet another embodiment of the invention, the artificial seed(s) comprise caps, lids or fastener structures that are displaced by the growing plant. In a particular embodiment, the caps, lids or fastener structures are displaced by a telescoping action or via the rupture of a weak adhesive joint;

5) In a further embodiment of the invention, the artificial seed(s) comprise tapered regions at the top, leading to openings which are small relative to the diameter or cross-section of the artificial seed. These tapered regions guide the shoots of the micropropagated tissue toward the opening(s) through which they can escape;

6) In a further embodiment of the invention, the artificial seed(s) comprise a water soluble top region or closure, wherein the closure is dissolved by irrigation or rainfall, thereby allowing the shoots of the micropropagated tissue to grow out of the artficial seed structure;

7) In a further embodiment of the invention, the aritificial seed(s) comprise a region or closure wherein the closure or region flows or creeps at a temperature between 1-50° C. This temperature range is commensurate with typical ambient temperatures experienced in field environments where this invention is directed.

In another embodiment, the container comprises a weak seam or slotted edge, allowing it to open and release the growing tissue. The weak seam may be created in the container by any means known in the art, including but not limited to perforation, thinning a region of the wall of the container, pre-stressing, creasing, or cracking a region of the container. In one embodiment, the container is an extruded cylindrical tube in which a weak seam is created along one or more edges by extruding a thinner region of material along the seam. In another embodiment, the container is a cylindrical tube with a slot cut along one edge. The material of the container is then flexible enough to allow the plantlet to push the container open. In one embodiment, the container can be constructed of two or more pieces or parts, which may be separable by the growth of the tissue or by dissolution or degradation of an adhesive connecting them. In one embodiment, the container consists of an extruded cylindrical tube with bands of soluble or degradable material along the length of the cylinder. This can be achieved through extrusion of a bi-component or multicomponent, or through the assembly of pieces using adhesive or heat sealing. In another embodiment, the container consists of two longitudinal halves of a tube, which are connected by adhesive. In another embodiment, two halves are connected along one edge through means including, but not limited to, heat sealing or adhesives, such that a hinged structure is created. In one embodiment, the adhesive consists of a water soluble polymer, including but not limited to poly(vinyl alcohol) or poly(vinyl pyrrolidone). The two halves may be connected using an adhesive or degradable material. The adhesive may be water soluble or flowable in a range of temperatures from about 1-50° C. The degradable material may be hydrolytically degradable, oxidatively degradable, biodegradable, compostable, or photodegradable. In another embodiment, the container consists of two connected sections of a tube. The connected sections may possess different porosity and/or degradability. The sections may be connected by means including, but not limited to, insertion, tape or an adhesive. In one embodiment the top section is composed of plastic and the bottom section is composed of paper.

The container may possess a conical or tapered feature. The angle of the conical feature, measured from one side of the conical section to the opposite side, may be varied, preferably less than 179 degrees, more preferably less than 135 degrees and most preferably less than 100 degrees. A conical tube is defined herein as a cylindrical tube with one or more conical features connected to it. The conical feature may be made of the same material as the cylindrical tube, or a different material. The conical or tapered feature may possess one or more holes, through which the plant can grow. Additionally, the holes provide rapid gas exchange. The size of the holes can vary from 0.1 to 30 mm, preferably from 1 to 20 mm and more preferably from 3 to 15 mm.

The container may be expandable or collapsible, such that prior to planting (for instance during storage) the seed occupies a smaller volume than it does after planting. The container may possess an expandable portion or component. As used herein, “expandable” means the capability of increasing in size. This is achieved for instance with concentric tubular or cylindrical containers that can be telescoped to form a longer tube.

As used herein, “telescoping” means the movement of two contacting objects in opposite directions without breaking contact. Also, the container may be partly or completely foldable, such that the folded container, prior to planting, occupies less space than the unfolded container after planting. The container may have pleated or ribbed sections, allowing collapsing while maintaining the same overall shape as the expanded version. The container may expand through the unfolding of an accordion-like structure. The container may possess rigidifying elements. As used herein, “a rigidifying element” means an element which increases the rigidity of an object. Rigidifying elements include, but are not limited to, creases, folds, inflated compartments, and thick or ribbed regions of the container. The container may be formed from a rolled sheet or tube, such that the structure can unroll or unravel, either at the time of planting or afterward through the growth of the tissue. As used herein, “unraveling” means unrolling of a rolled object without loss of the object's overall shape. The container may possess a collapsible film which can be expanded to form a protective tent around the artificial seed. In one embodiment, the container of the artificial seed may also be stretchable. As used herein, “stretching” means the act of elongation through deformation in one or more directions. In one embodiment, the container may be deflatable and inflatable. The deflation may be achieved through the application of external pressure or through vacuum sealing. Upon rupturing the seal, the container may spontaneously re-inflate. Alternatively, gas pressure may be applied to cause the inflation. In many cases, a restraint may be used to keep the container in a compact or collapsed form prior to planting. This restraint includes, but is not limited to, a band or tape, a glue or other fastener.

In one embodiment the artificial seed possesses a closed bottom end, which contains moisture. This closed end prevents the moisture from draining into the surrounding soil. Holes on the sides of the container are then situated to allow root growth, while maintaining the closed nature of the bottom end of the artificial seed.

The container may comprise a packet or a pouch. The packet may be completely sealed or may possess multiple openings. The packet may be made of biodegradable, photodegradable, oxidatively degradable or hydrolytically degradable material. The packet may be flexible or semi-flexible. Semi-flexible is defined as being capable of deformation through an external force, but returning to a shape similar to its original shape after removal of the external force. The packet may possess rigidifying elements. The packet may have shapes including, but not limited to, tubular, cylindrical, rectangular, square or round shapes.

The container may be transparent, translucent, semi-opaque or opaque. Transparent materials include but are not limited to polycarbonate and glass. Translucent materials include but are not limited to high density polyethylene and polypropylene. Semi-translucent materials include but are not limited to etched glass and coated plastics. Opaque materials include but are not limited to filled plastics, wood and paper.

The size of the container can vary. However, in one embodiment, the container possesses a cylindrical shape with a wall thicknesses ranging from 0.01-0.25 cm and dimensions of from 0.5-5 cm diameter and 1-30 cm length.

Various materials can be used to make the container, and in one embodiment of the invention the materials used to make the container comprise, or alternatively consist of: cellulosic material, such as, for example cellulose, ethyl cellulose, nitrocellulose, cellulose acetate, cellulose priopionate, cellulose acetate butyrate; with or without waxes and oils, synthetic and natural polymers and plastics such as, for example, gelatin, chitosan, zein, polyolefins, polypropylene, polyethylene, polyolefins, photodegradable polymers, oxidatively degradable polymers, polystyrene, acrylic copolymers, poly(alkyl (meth)acrylates), polyesters, polyethers, poly(vinyl acetate) copolymers, poly(acrylamide), poly(vinyl pyrrolidone), poly(vinyl pyridine), natural rubber, poly(ethylene oxide), polyamides, polysaccharides and polycarbonates, porous and non-woven materials, as well as crosslinked versions thereof, combinations thereof, copolymers thereof and plasticized versions thereof biodegradable plastics including poly(hydroxy alkanoate)s, poly(lactic acid), poly(L-lactide), poly(D-lactide), poly(D,L-lactide), stereocomplexes of poly(L-lactide) with poly(D-lactide), poly(1,2-propanediol succinate), and copolymers thereof and crosslinked versions thereof.

Porous materials include, but are not limited to, ceramics, nonwovens and textiles. The container may also be nonporous. Nonporous materials include but are not limited to plastic, glass and metal. The container may be fabricated from a permeable material. Permeability includes but is not limited to water permeability, gas permeability and oxygen permeability. Permeable materials include poly(vinyl alcohol), poly(dimethyl siloxane) and natural rubber. The container may be fabricated from impermeable materials. Impermeability includes but is not limited to moisture impermeable or barrier materials, gas impermeable or barrier materials and oxygen impermeable or barrier materials. Impermeable materials include but are not limited to glass, metal and polyethylene terephthalate. Waxes and/or oils can be used to coat the walls of the container. Waxes include but are not limited to paraffin wax, spermaceti wax, beeswax and carnauba wax.

It is preferred that the artificial seed described herein substantially or completely degrades in the field environment such that the planted containers do not accumulate in the field over years of repeated planting. In order to accomplish this, biodegradable materials may be used to construct the container and closures. Traditional biodegradable materials including poly(lactic acid), poly(1,3-propanediol succinate), polypropylene succinate), poly(hydroxybutyrate)s, poly(caprolactone) and cellulose derivatives are candidate biodegradable materials. In a preferred embodiment of poly(lactic acid), amorphous grades having a higher D-lactic acid content (typically >6 mol % D-lactic acid) are incorporated to provide higher degradation rates compared to more crystalline-containing poly(lactic acids) (<6 mol % D-lactic acid).

Another method of increasing degradability while reducing brittleness involves blending poly(lactic acid) or amorphous poly(lactic acid) with more rapidly degradable polymers, such as poly(caprolactone), poly(hydroxybutyrates) or thermoplastic starch (Rychter et al. Biomacromolecules 2006, 7, 3125). Blends can be formed by any method known in the art, including solution blending, melt blending, extrusion, compounding, reactive extrusion, etc. As used herein, “blends” means mixtures of two or more components. Blends may be miscible, immiscible, partially miscible and may consist of separate domains of each component. In one embodiment of the invention, the materials used to produce the container may comprise, or alternatively consist of, blends of poly(lactic acid), poly(hydroxybutyrate), poly(hydroxybutyrate-co-valerate), starch, cellulose, and chitosan, optionally with plasticizers including but not limited to sorbitol, glycerol, citrate esters, phthalate esters and water. Plasticizers are defined as substances which reduce the glass transition temperature of a material.

In another embodiment of the invention, the container comprises, or alternatively consists of, blends of poly(lactic acid) with poly(1,3-propanediol succinate). Such blends are optically translucent to translucent, which is advantageous to allow light to reach the tissue. Blends of crystalline poly(lactic acid) with poly(1,3-propanediol succinate) are partially miscible, as evidenced by the presence of two glass transition temperatures which change as a function of composition. Additionally, the optical clarity remains good even at high concentrations (even 50 wt %) of poly(1,3-propanediol succinate). Additionally, poly(1,3-propanediol succinate) is disclosed herein to exhibit rapid soil degradability, ideal for an artificial seed application.

Another method of increasing degradability while reducing brittleness involves plasticizing poly(lactic acid) with plasticizers including but not limited to citrate derivatives, citrate esters, acetyl butyl citrate, triethyl citrate, tributyl citrate, diethyl bishydroxymethyl malonate, phthalate esters, glycerol, poly(ethylene glycol), poly(ethylene glycol) monolaurate, oligomeric poly(lactic acid).

In another embodiment, the container is degradable at a rate that is commensurate with the growth of the tissue. In this embodiment, the container comprises, or alternatively consists of, poly(ε-caprolactone) or poly(hydroxyalkanoate). In one embodiment, the entire container is fabricated from poly(ε-caprolactone) or poly(hydroxyalkanoate) such that the portion in contact with the soil degrades at a rate sufficient to allow roots to escape and proliferate into the surrounding soil, and subsequently the top portion is then pushed off or shed by forces exerted by the growing shoots.

In another embodiment, the container and/or its closure(s) comprises, or alternatively consists of, dissolvable materials. In one such embodiment, the container and/or its closure(s) comprises, or alternatively consists of, blends of poly(vinyl alcohol) with starch, cellulose fibers and glycerol, optionally with crosslinking with a suitable agent, including but not limited to hexamethoxymethylmelamine or glutaraldehyde. This provides materials which are rapidly degradable in moist soil conditions, permitting rapid growth of the tissue inside. The starch may be from sources including but not limited to potato, corn, rice, wheat and cassava and may be modified or unmodified. Additional additives may include, but are not limited to, poly(ethylene glycol), citric acid, urea, water, salts including but not limited to sodium acetate, potassium nitrate and ammonium nitrate, fertilizers, agar, xanthan gum, alginate, and cellulose derivatives including but not limited to hydroxypropylcellulose, methylcellulose and carboxymethylcellulose.

The container may also comprise plasticizers, antioxidants, nucleating agents, tougheners, colorants, fillers, impact modifiers, processing aids, stabilizers, and flame retardants. Antioxidants include but are not limited to hydroquinone, Irganox® 1010, and vitamin E. Nucleating agents include but are not limited to calcium carbonate, cyclodextrin and phenylphosphonic acid zinc. Tougheners include but are not limited to styrenic block copolymers, Biomax® Strong, and oils. Colorants include but are not limited to pigments and dyes. Fillers include but are not limited to starch, mica and silica. Impact modifiers include but are not limited to Paraloid™ BPM-520, Biostrength® 280, and butadiene rubber. Processing aids include but are not limited to erucamide and stearyl erucamide. Stabilizers include but are not limited to UV stabilizers, hindered amine light stabilizers, antiozonants and organosulfur compounds. Flame retardants include but are not limited to aluminium trihydroxide (ATH), magnesium hydroxide (MDH), phosphonate esters, triphenyl phosphate, phosphate esters, ammonium pyrophosphate and melamine polyphosphate.

When the container is constructed of cellulosic material, it can optionally contain clay, alum, waxes, binders, glues, surfactants and barriers such as plastic or metallized layers. The cellulosic material may be porous and may possess multiple layers comprising, or alternatively consisting of, a variety of papers including but not limited to craft paper, bond paper, recycled paper, recycled newsprint, construction paper, chip board, cup stock, copier paper, wax paper, and coated papers.

In the presently disclosed invention, artificial seeds can be produced using a paper or a plastic container. The paper or plastic, to be used for container construction, has the following properties to be suitable for such application: it does not immediately overly soften by the aqueous nutrient source contained within it. The paper containers can be porous in nature, and can be degradable over the course of at least 5 years in soil. The plastic containers can be porous or non-porous, and may or may not be degradable in soil. The plastic material is either thermoplastic or thermoset materials.

In an embodiment, wax paper can be used to prepare the paper containers. In this case the size of the wax paper container can be around 1.19 cm in diameter and 4-6 cm in length.

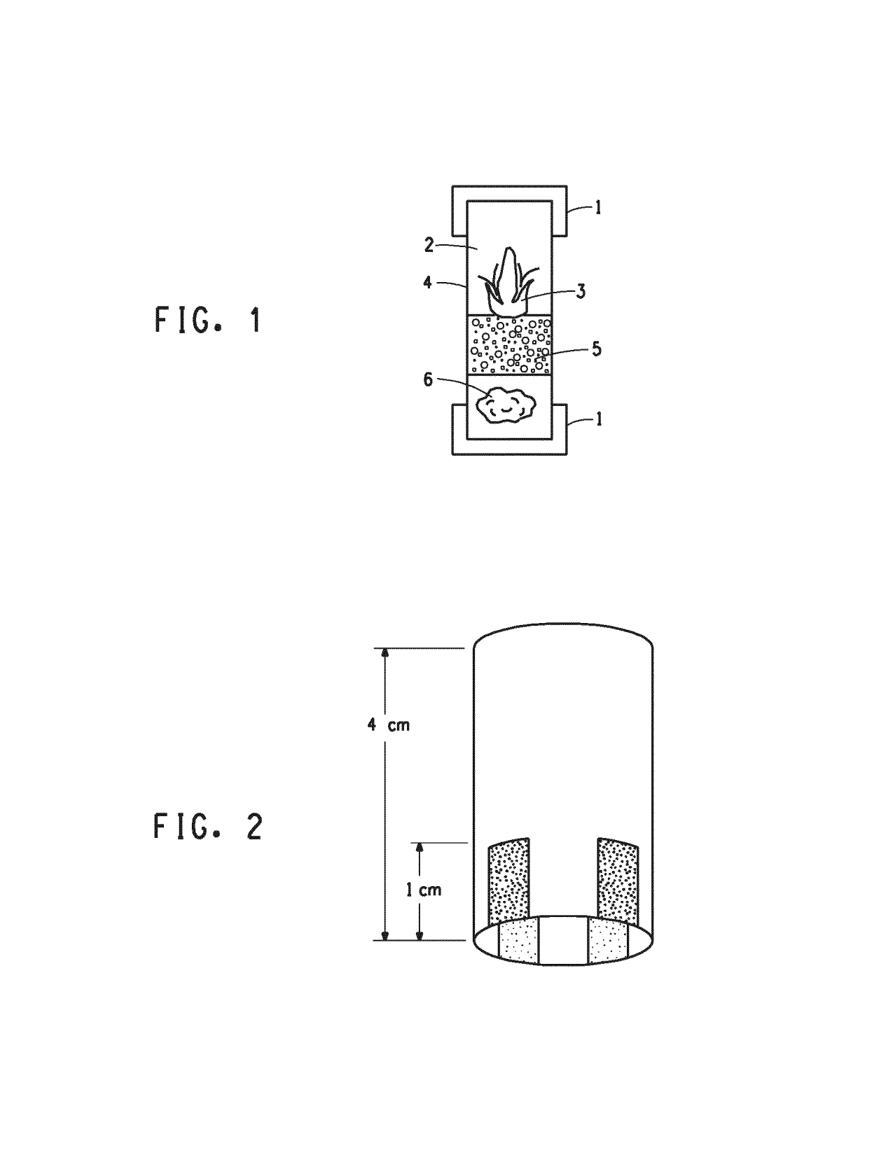

The cylindrical containers can have flat ends at the top and the bottom. In one embodiment, the bottom end of the container is crenellated (see FIG. 2). As used herein, “crenellation” means the creation of an irregular edge via the use of tabs of material extending from the edge and indentations into the edge. The size of crenellation can be from 0.65 cm to about 2 cm in length, with 2-6 tabs. In another embodiment, crenellation can be from 0.8 cm to about 1.2 cm in length, with 3-4 tabs.

Artificial seeds can also comprise one or more of a nutrient source (FIG. 1, (5)), solid objects such as pieces of cotton (FIG. 1, (6)), insecticides, fungicides, nematicides, antimicrobial compounds, antibiotics, biocides, herbicides, plant growth regulators or stimulators, microbes, molluscicides, miticides, acaricides, bird repellant(s), insect repellant(s), plant hormones, rodent repellant(s), fertilizers, hydrogels, superabsorbents, fillers, soil, soil amendments and water. Biocides include, but are not limited to, hypochlorite, sodium dichloro-s-triazinetrione, Plant Preservative Mixture™, obtained from Plant Cell Technology and trichloro-s-triazinetrione. Molluscicides include, but are not limited to, metaldehyde or methiocarb. Acaricides include, but are not limited to, ivermectin or permethrin. A bird repellent is defined as a substance that repels birds. Bird repellants include, but are not limited to, methyl anthranilate, methiocarb, chlorpyrifos and propiconazole. A rodent repellent is defined as a substance that repels rodents. Rodent repellents include, but are not limited to, thiram and methiocarb. Insect repellents include, but are not limited to, N,N-diethyl-m-toluamide, essential oils and citronella oil. Miticides include, but are not limited to, abamectin and chlorfenapyr. Plant hormones include, but are not limited to, abscisic acid, auxins, cytokinins, ethylene and gibberellins. Plant growth regulators include, but are not limited to, paclobutyrazol, ethephon, and ancymidol. As used herein, “superabsorbents” means absorbents which absorb water or aqueous solutions resulting in a hydrated gel such that the weight of the gel is 30 times or greater the weight of the dry superabsorbent. Superabsorbents include, but are not limited to, superabsorbent polymers, crosslinked poly(sodium acrylate), crosslinked poly(acrylic acid), crosslinked poly(acrylic acid) salts, acrylic acid modified starch, crosslinked copolymers of acrylic acid with poly(ethylene glycol) acrylate, poly(ethylene glycol) methacrylate, poly(ethylene glycol) diacrylate, acrylamide, vinyl acetate, acrylic acid salts, bisacrylamide, N-vinyl pyrrolidone, acrylate esters, methacryrlate esters, styrenic monomers, diene monomers and crosslinkers. The superabsorbent may be present in the artificial seed in a dry or swollen state. It may be swollen with water or aqueous solutions, including but not limited to nutrient solutions, fertilizer solutions and antimicrobial solutions. The superabsorbent may also be mixed with soil or other components of the nutrient media. In one embodiment, the superabsorbent may be present in a separate compartment of the seed. The compartment may be connected or not with the compartment containing the regenerable plant tissue. The compartment may be separated by a screen or mesh from the compartment containing the tissue. Microbes include but are not limited to beneficial microbes, nitrogen fixing bacteria, rhizobium, fungi, azotobacter, microrhyza, microbes that release cellulases, and microbes that participate in degradation of the artificial seed container.

The soil suitable for application inside the container where the regenerable plant tissue is to be inserted to grow should be able to provide aeration, water, nutrition, and anchorage to the growing regenerable plant tissue. Various kinds of soil that can be used in the container include synthetic soils like MetroMix® and vermiculite. It can also include natural soils such as sand, silt, loam, peat, and mixtures of these soils. The suitable soil can be present such that the container is at most 99% full.

The artificial seed of the disclosed invention comprises airspace (2) within the container. The artificial seed can also contain closures (FIG. 1, (1)). Closures are defined as lids, caps or objects that cover openings. In one embodiment the closure may be separable from the container. The regenerable plant tissue may be capable of lifting off or shedding the separable closure during its growth. Separable closures include but are not limited to caps, inserts, flat films, dome shaped caps and conical caps. The separable closure may be attached to the container using an adhesive or degradable material. The adhesive may be water soluble or flowable in a range of temperatures from about 1-50° C. The degradable material may be hydrolytically degradable, oxidatively degradable, biodegradable, compostable or photodegradable. The caps or lids may also be attached by simple physical means including but not limited to insertion or crimping.

As used herein, “nutrient source” means nutrients which can help sustain and provide for the growth of the plant from the regenerable tissue. Suitable nutrients include, but are not limited to, one or more of water, soil, coconut coir, vermiculite, an artificial growth medium, agar, a plant growth regulator, a plant hormone, a superabsorbent polymer, macronutrients, micronutrients, fertilizers, inorganic salts, (including but not limited to nitrate, ammonium, phosphate, potassium and calcium salts) vitamins, sugars and other carbohydrates, proteins, lipids, Murashige and Skoog (MS) nutrient formula, Hoagland's nutrient formula, Gamborg's B-5 medium, nutrient formula and native and synthetic soils, peat and vinasse, and combinations thereof. Macronutrients include but are not limited to nitrate, phosphate and potassium. Micronutrients include but are not limited to cobalt chloride, boric acid, ferrous sulfate and manganese sulfate. The nutrient source can also contain extracellular polysaccharides such as those described in Mager, D. M. and Thomas, A. D. Journal of Arid Environments, 2011, 75, 2, 91-7.

The nutrient source can also contain hormones and plant growth regulators including but not limited to, gibberellic acid, indole acetic acid, naphthalene acetic acid (NAA), ethephon, 6-benzylamino purine (6-ABP), 2,4-dichlorophenoxyacetic acid (2,4-D), paclobutrazole, ancymidol and abcissic acid.

The nutrients can be present in an aqueous solution or aqueous gel solution, such as those well known in the art of plant propagation, including but not limited to natural and synthetic gels including: agar, agarose, gellan gum, guar gum, gum arabic, Gelrite™, Phytagel™, superabsorbent polymers, carrageenan, amylose, carboxymethyl-cellulose, dextran, locust bean gum, alginate, xanthan gum, gelatin, pectin, starches, zein, polyacrylamide, polyacrylic acid, poly(ethylene glycol) and crosslinked versions thereof.

In one embodiment, the nutrients can be present in a silicate gel. Such a silicate gel can be formed by neutralizing a solution of sodium or potassium silicate with acid. In one embodiment, subsequent washing or soaking steps may be used to remove the excess salts. Optionally, the gel can then be infused with nutrients through soaking or other processes. Alternatively, the silicate gel can be formed from silicic acid, or from other precursors, including but not limited to alkoxysilanes, silyl halides, or silazanes.

When the container comprises a nutrient source, the regenerable plant tissue within the container is partially embedded or in contact with the nutrient source and can be partially exposed to the airspace within the container. The term “partially exposed to an airspace”, as used herein, refers to a regenerable plant tissue that is either in contact with or has been partially embedded (i.e., 0 to 90% of the tissue submerged) in the nutrient source present in the container, with the remainder exposed to the airspace within the container. The regenerable plant tissue can be partially or fully surrounded by the nutrient source. The regenerable plant tissue can also be placed on top of the nutrient source. As used herein, “airspace” means a void in the container that is empty of any solid or liquid material, and filled by atmospheric gasses such as air, for example. An airspace, as defined herein does not include the collective voids in a porous or particulate material.

It is advantageous for the function of the artificial seed that the airspace be free of obstructions that limit the growth of the regenerable plant tissue with exception of the limits of the container wall. As used herein, “an unobstructed airspace” means an airspace that is continuous and uninterrupted between any part of the regenerable plant tissue and any region of the container. As used herein, “tapered” means narrowing or becoming progressively narrower along a dimension.

For the purposes of the disclosed invention, regenerable plant tissues can be prepared using various methods well known in the relevant art, such as the method of tissue culture of meristematic tissue described in International Publication Number WO2011/085446, the disclosure of which is herein incorporated by reference. Other possible methods include using plant cuttings, embryos from natural seeds or somatic embryos obtained through somatic embryogenesis. In one embodiment meristems can be excised to form explants and cultured to increase the tissue mass. The term “explant” as used herein, refers to tissues which have been excised from a plant to be used in plant tissue culture.

The regenerable plant tissue of the invention may also be genetically modified. This genetic modification includes, but is not limited to, herbicide resistance, disease resistance, drought tolerance, and insect resistance. Genetically modified (also known as transgenic) plants may comprise a single transgenic trait or a stack of one or more transgene polynucleotides with one or more additional polynucleotides resulting in the production or suppression of multiple polypeptide sequences. Transgenic plants comprising stacks of polynucleotide sequences can be obtained by either or both of traditional breeding methods or through genetic engineering methods. These methods include, but are not limited to, breeding individual lines each comprising a polynucleotide of interest, transforming a transgenic plant comprising a gene with a subsequent gene and co-transformation of genes into a single plant cell.

As used herein, the term “stacked” includes having the multiple traits present in the same plant (i.e., both traits are incorporated into the nuclear genome, one trait is incorporated into the nuclear genome and one trait is incorporated into the genome of a plastid or both traits are incorporated into the genome of a plastid). In one non-limiting example, “stacked traits” comprise a molecular stack where the sequences are physically adjacent to each other. A trait, as used herein, refers to the phenotype derived from a particular sequence or groups of sequences. Co-transformation of genes can be carried out using single transformation vectors comprising multiple genes or genes carried separately on multiple vectors. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, International Publication Numbers WO 1999/25821, WO 1999/25854, WO 1999/25840, WO 1999/25855 and WO 1999/25853, the disclosures of each of which are herein incorporated by reference.

In some embodiments the polynucleotides encoding the polypeptides, alone or stacked with one or more additional insect resistance traits can be stacked with one or more additional input traits (e.g., herbicide resistance, fungal resistance, virus resistance, stress tolerance, disease resistance, male sterility, stalk strength, and the like) or output traits (e.g., increased yield, modified starches, improved oil profile, balanced amino acids, high lysine or methionine, increased digestibility, improved fiber quality, drought resistance, and the like). Thus, the polynucleotide embodiments can be used to provide a complete agronomic package of improved crop quality with the ability to flexibly and cost effectively control any number of agronomic pests.

Transgenes useful for preparing transgenic plants include, but are not limited to, the following:

1. Transgenes Conferring Resistance to Insects or Disease:

(A) Plant disease resistance genes. Plant defenses are often activated by specific interaction between the product of a disease resistance gene (R) in the plant and the product of a corresponding avirulence (Avr) gene in the pathogen. A plant variety can be transformed with cloned resistance gene to engineer plants that are resistant to specific pathogen strains. See, for example, Jones, et al., (1994) Science 266:789 (cloning of the tomato Cf-9 gene for resistance to Cladosporium fulvum); Martin, et al., (1993) Science 262:1432 (tomato Pto gene for resistance to Pseudomonas syringae pv. tomato encodes a protein kinase); Mindrinos, et al., (1994) Cell 78:1089 (Arabidopsis RSP2 gene for resistance to Pseudomonas syringae), McDowell and Woffenden, (2003) Trends Biotechnol. 21(4):178-83 and Toyoda, et al., (2002) Transgenic Res. 11(6):567-82. A plant resistant to a disease is one that is more resistant to a pathogen as compared to the wild type plant.

(B) Genes encoding a Bacillus thuringiensis protein, a derivative thereof or a synthetic polypeptide modeled thereon. See, for example, Geiser, et al., (1986) Gene 48:109, who disclose the cloning and nucleotide sequence of a Bt delta-endotoxin gene. Moreover, DNA molecules encoding delta-endotoxin genes can be purchased from American Type Culture Collection (Rockville, Md.), for example, under ATCC Accession Numbers 40098, 67136, 31995 and 31998. Other non-limiting examples of Bacillus thuringiensis transgenes being genetically engineered are given in the following patents and patent applications and hereby are incorporated by reference for this purpose: U.S. Pat. Nos. 5,188,960; 5,689,052; 5,880,275; 5,986,177; 6,023,013, 6,060,594, 6,063,597, 6,077,824, 6,620,988, 6,642,030, 6,713,259, 6,893,826, 7,105,332; 7,179,965, 7,208,474; 7,227,056, 7,288,643, 7,323,556, 7,329,736, 7,449,552, 7,468,278, 7,510,878, 7,521,235, 7,544,862, 7,605,304, 7,696,412, 7,629,504, 7,705,216, 7,772,465, 7,790,846, 7,858,849 and WO 1991/14778; WO 1999/31248; WO 2001/12731; WO 1999/24581 and WO 1997/40162, the disclosures of each of which are herein incorporated by reference.

(C) A polynucleotide encoding an insect-specific hormone or pheromone such as an ecdysteroid and juvenile hormone, a variant thereof, a mimetic based thereon or an antagonist or agonist thereof. See, for example, the disclosure by Hammock, et al., (1990) Nature 344:458, of baculovirus expression of cloned juvenile hormone esterase, an inactivator of juvenile hormone.

(D) A polynucleotide encoding an insect-specific peptide which, upon expression, disrupts the physiology of the affected pest. For example, see the disclosures of, Regan, (1994) J. Biol. Chem. 269:9 (expression cloning yields DNA coding for insect diuretic hormone receptor); Pratt, et al., (1989) Biochem. Biophys. Res. Comm. 163:1243 (an allostatin is identified in Diploptera puntata); Chattopadhyay, et al., (2004) Critical Reviews in Microbiology 30(1):33-54; Zjawiony, (2004) J Nat Prod 67(2):300-310; Carlini and Grossi-de-Sa, (2002) Toxicon 40(11):1515-1539; Ussuf, et al., (2001) Curr Sci. 80(7):847-853 and Vasconcelos and Oliveira, (2004) Toxicon 44(4):385-403. See also, U.S. Pat. No. 5,266,317 to Tomalski, et al., who disclose genes encoding insect-specific toxins.

(E) A polynucleotide encoding an enzyme responsible for a hyperaccumulation of a monoterpene, a sesquiterpene, a steroid, hydroxamic acid, a phenylpropanoid derivative or another non-protein molecule with insecticidal activity.

(F) A polynucleotide encoding an enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See, PCT Application WO 1993/02197 in the name of Scott, et al., which discloses the nucleotide sequence of a callase gene. DNA molecules which contain chitinase-encoding sequences can be obtained, for example, from the ATCC under Accession Numbers 39637 and 67152. See also, Kramer, et al., (1993) Insect Biochem. Molec. Biol. 23:691, who teach the nucleotide sequence of a cDNA encoding tobacco hookworm chitinase and Kawalleck, et al., (1993) Plant Molec. Biol. 21:673, who provide the nucleotide sequence of the parsley ubi4-2 polyubiquitin gene, and U.S. Pat. Nos. 6,563,020; 7,145,060 and 7,087,810.

(G) A polynucleotide encoding a molecule that stimulates signal transduction. For example, see the disclosure by Botella, et al., (1994) Plant Molec. Biol. 24:757, of nucleotide sequences for mung bean calmodulin cDNA clones, and Griess, et al., (1994) Plant Physiol. 104:1467, who provide the nucleotide sequence of a maize calmodulin cDNA clone.

(H) A polynucleotide encoding a hydrophobic moment peptide. See, PCT Application WO 1995/16776 and U.S. Pat. No. 5,580,852 disclosure of peptide derivatives of Tachyplesin which inhibit fungal plant pathogens) and PCT Application WO 1995/18855 and U.S. Pat. No. 5,607,914 (teaches synthetic antimicrobial peptides that confer disease resistance).

(I) A polynucleotide encoding a membrane permease, a channel former or a channel blocker. For example, see the disclosure by Jaynes, et al., (1993) Plant Sci. 89:43, of heterologous expression of a cecropin-beta lytic peptide analog to render transgenic tobacco plants resistant to Pseudomonas solanacearum.

(J) A gene encoding a viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See, Beachy, et al., (1990) Ann. Rev. Phytopathol. 28:451. Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

(K) A gene encoding an insect-specific antibody or an immunotoxin derived therefrom. Thus, an antibody targeted to a critical metabolic function in the insect gut would inactivate an affected enzyme, killing the insect. Cf. Taylor, et al., Abstract #497, SEVENTH INT'L SYMPOSIUM ON MOLECULAR PLANT-MICROBE INTERACTIONS (Edinburgh, Scotland, 1994) (enzymatic inactivation in transgenic tobacco via production of single-chain antibody fragments).

(L) A gene encoding a virus-specific antibody. See, for example, Tavladoraki, et al., (1993) Nature 366:469, who show that transgenic plants expressing recombinant antibody genes are protected from virus attack.

(M) A polynucleotide encoding a developmental-arrestive protein produced in nature by a pathogen or a parasite. Thus, fungal endo alpha-1,4-D-polygalacturonases facilitate fungal colonization and plant nutrient release by solubilizing plant cell wall homo-alpha-1,4-D-galacturonase. See, Lamb, et al., (1992) Bio/Technology 10:1436. The cloning and characterization of a gene which encodes a bean endopolygalacturonase-inhibiting protein is described by Toubart, et al., (1992) Plant J. 2:367.

(N) A polynucleotide encoding a developmental-arrestive protein produced in nature by a plant. For example, Logemann, et al., (1992) Bio/Technology 10:305, have shown that transgenic plants expressing the barley ribosome-inactivating gene have an increased resistance to fungal disease.

(O) Genes involved in the Systemic Acquired Resistance (SAR) Response and/or the pathogenesis related genes. Briggs, (1995) Current Biology 5(2), Pieterse and Van Loon, (2004) Curr. Opin. Plant Bio. 7(4):456-64 and Somssich, (2003) Cell 113(7):815-6.

(P) Antifungal genes (Cornelissen and Melchers, (1993) Pl. Physiol. 101:709-712 and Parijs, et al., (1991) Planta 183:258-264 and Bushnell, et al., (1998) Can. J. of Plant Path. 20(2):137-149. Also see, U.S. patent application Ser. Nos. 09/950,933; 11/619,645; 11/657,710; 11/748,994; 11/774,121 and U.S. Pat. Nos. 6,891,085 and 7,306,946. LysM Receptor-like kinases for the perception of chitin fragments as a first step in plant defense response against fungal pathogens (US 2012/0110696).

(O) Detoxification genes, such as for fumonisin, beauvericin, moniliformin and zearalenone and their structurally related derivatives. For example, see, U.S. Pat. Nos. 5,716,820; 5,792,931; 5,798,255; 5,846,812; 6,083,736; 6,538,177; 6,388,171 and 6,812,380.

(R) A polynucleotide encoding a Cystatin and cysteine proteinase inhibitors. See, U.S. Pat. No. 7,205,453.

(S) Defensin genes. See, WO 2003/000863 and U.S. Pat. Nos. 6,911,577; 6,855,865; 6,777,592 and 7,238,781.

(T) Genes conferring resistance to nematodes. See, e.g., PCT Application WO 1996/30517; PCT Application WO 1993/19181, WO 2003/033651 and Urwin, et al., (1998) Planta 204:472-479, Williamson, (1999) Curr Opin Plant Bio. 2(4):327-31; U.S. Pat. Nos. 6,284,948 and 7,301,069 and miR164 genes (WO 2012/058266).

(U) Genes that confer resistance to Phytophthora Root Rot, such as the Rps 1, Rps 1-a, Rps 1-b, Rps 1-c, Rps 1-d, Rps 1-e, Rps 1-k, Rps 2, Rps 3-a, Rps 3-b, Rps 3-c, Rps 4, Rps 5, Rps 6, Rps 7 and other Rps genes. See, for example, Shoemaker, et al., Phytophthora Root Rot Resistance Gene Mapping in Soybean, Plant Genome IV Conference, San Diego, Calif. (1995).

(V) Genes that confer resistance to Brown Stem Rot, such as described in U.S. Pat. No. 5,689,035 and incorporated by reference for this purpose.

(W) Genes that confer resistance to Colletotrichum, such as described in US Patent Application Publication US 2009/0035765 and incorporated by reference for this purpose. This includes the Rcg locus that may be utilized as a single locus conversion.

2. Transgenes that Confer Resistance to a Herbicide:

(A) A polynucleotide encoding resistance to a herbicide that inhibits the growing point or meristem, such as an imidazolinone or a sulfonylurea. Exemplary genes in this category code for mutant ALS and AHAS enzyme as described, for example, by Lee, et al., (1988) EMBO J. 7:1241 and Miki, et al., (1990) Theor. Appl. Genet. 80:449, respectively. See also, U.S. Pat. Nos. 5,605,011; 5,013,659; 5,141,870; 5,767,361; 5,731,180; 5,304,732; 4,761,373; 5,331,107; 5,928,937 and 5,378,824; U.S. patent application Ser. No. 11/683,737 and International Publication WO 1996/33270.

(B) A polynucleotide encoding a protein for resistance to Glyphosate (resistance imparted by mutant 5-enolpyruvl-3-phosphikimate synthase (EPSP) and aroA genes, respectively) and other phosphono compounds such as glufosinate (phosphinothricin acetyl transferase (PAT) and Streptomyces hygroscopicus phosphinothricin acetyl transferase (bar) genes), and pyridinoxy or phenoxy proprionic acids and cyclohexones (ACCase inhibitor-encoding genes). See, for example, U.S. Pat. No. 4,940,835 to Shah, et al., which discloses the nucleotide sequence of a form of EPSPS which can confer glyphosate resistance. U.S. Pat. No. 5,627,061 to Barry, et al., also describes genes encoding EPSPS enzymes. See also, U.S. Pat. Nos. 6,566,587; 6,338,961; 6,248,876 B1; 6,040,497; 5,804,425; 5,633,435; 5,145,783; 4,971,908; 5,312,910; 5,188,642; 5,094,945, 4,940,835; 5,866,775; 6,225,114 B1; 6,130,366; 5,310,667; 4,535,060; 4,769,061; 5,633,448; 5,510,471; Re. 36,449; RE 37,287 E and 5,491,288 and International Publications EP 1173580; WO 2001/66704; EP 1173581 and EP 1173582, which are incorporated herein by reference for this purpose. Glyphosate resistance is also imparted to plants that express a gene encoding a glyphosate oxido-reductase enzyme as described more fully in U.S. Pat. Nos. 5,776,760 and 5,463,175, which are incorporated herein by reference for this purpose. In addition glyphosate resistance can be imparted to plants by the over expression of genes encoding glyphosate N-acetyltransferase. See, for example, U.S. Pat. Nos. 7,462,481; 7,405,074 and US Patent Application Publication Number US 2008/0234130. A DNA molecule encoding a mutant aroA gene can be obtained under ATCC Accession Number 39256, and the nucleotide sequence of the mutant gene is disclosed in U.S. Pat. No. 4,769,061 to Comai. EP Application Number 0 333 033 to Kumada, et al., and U.S. Pat. No. 4,975,374 to Goodman, et al., disclose nucleotide sequences of glutamine synthetase genes which confer resistance to herbicides such as L-phosphinothricin. The nucleotide sequence of a phosphinothricin-acetyl-transferase gene is provided in EP Application Numbers 0 242 246 and 0 242 236 to Leemans, et al., De Greef, et al., (1989) Bio/Technology 7:61, describe the production of transgenic plants that express chimeric bar genes coding for phosphinothricin acetyl transferase activity. See also, U.S. Pat. Nos. 5,969,213; 5,489,520; 5,550,318; 5,874,265; 5,919,675; 5,561,236; 5,648,477; 5,646,024; 6,177,616 B1 and 5,879,903, which are incorporated herein by reference for this purpose. Exemplary genes conferring resistance to phenoxy proprionic acids and cyclohexones, such as sethoxydim and haloxyfop, are the Acc1-S1, Acc1-S2 and Acc1-S3 genes described by Marshall, et al., (1992) Theor. Appl. Genet. 83:435.

(C) A polynucleotide encoding a protein for resistance to herbicide that inhibits photosynthesis, such as a triazine (psbA and gs+ genes) and a benzonitrile (nitrilase gene). Przibilla, et al., (1991) Plant Cell 3:169, describe the transformation of Chlamydomonas with plasmids encoding mutant psbA genes. Nucleotide sequences for nitrilase genes are disclosed in U.S. Pat. No. 4,810,648 to Stalker and DNA molecules containing these genes are available under ATCC Accession Numbers 53435, 67441 and 67442. Cloning and expression of DNA coding for a glutathione S-transferase is described by Hayes, et al., (1992) Biochem. J. 285:173.

(D) A polynucleotide encoding a protein for resistance to Acetohydroxy acid synthase, which has been found to make plants that express this enzyme resistant to multiple types of herbicides, has been introduced into a variety of plants (see, e.g., Hattori, et al., (1995) Mol Gen Genet. 246:419). Other genes that confer resistance to herbicides include: a gene encoding a chimeric protein of rat cytochrome P4507A1 and yeast NADPH-cytochrome P450 oxidoreductase (Shiota, et al., (1994) Plant Physiol 106:17), genes for glutathione reductase and superoxide dismutase (Aono, et al., (1995) Plant Cell Physiol 36:1687) and genes for various phosphotransferases (Datta, et al., (1992) Plant Mol Biol 20:619).

(E) A polynucleotide encoding resistance to a herbicide targeting Protoporphyrinogen oxidase (protox) which is necessary for the production of chlorophyll. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306 B1; 6,282,837 B1 and 5,767,373 and International Publication WO 2001/12825.

(F) The aad-1 gene (originally from Sphingobium herbicidovorans) encodes the aryloxyalkanoate dioxygenase (AAD-1) protein. The trait confers tolerance to 2,4-dichlorophenoxyacetic acid and aryloxyphenoxypropionate (commonly referred to as “fop” herbicides such as quizalofop) herbicides. The aad-1 gene, itself, for herbicide tolerance in plants was first disclosed in WO 2005/107437 (see also, US 2009/0093366). The aad-12 gene, derived from Delftia acidovorans, which encodes the aryloxyalkanoate dioxygenase (AAD-12) protein that confers tolerance to 2,4-dichlorophenoxyacetic acid and pyridyloxyacetate herbicides by deactivating several herbicides with an aryloxyalkanoate moiety, including phenoxy auxin (e.g., 2,4-D, MCPA), as well as pyridyloxy auxins (e.g., fluoroxypyr, triclopyr).

(G) A polynucleotide encoding a herbicide resistant dicamba monooxygenase disclosed in US Patent Application Publication 2003/0135879 for imparting dicamba tolerance;

(H) A polynucleotide molecule encoding bromoxynil nitrilase (Bxn) disclosed in U.S. Pat. No. 4,810,648 for imparting bromoxynil tolerance;

(I) A polynucleotide molecule encoding phytoene (crtl) described in Misawa, et al., (1993) Plant J. 4:833-840 and in Misawa, et al., (1994) Plant J. 6:481-489 for norflurazon tolerance.

3. Transgenes conferring or Contributing to an Altered Grain Characteristic

(A) Altered fatty acids, for example, by

(1) Down-regulation of stearoyl-ACP to increase stearic acid content of the plant. See, Knultzon, et al., (1992) Proc. Natl. Acad. Sci. USA 89:2624 and WO 1999/64579 (Genes to Alter Lipid Profiles in Corn).

(2) Elevating oleic acid via FAD-2 gene modification and/or decreasing linolenic acid via FAD-3 gene modification (see, U.S. Pat. Nos. 6,063,947; 6,323,392; 6,372,965 and WO 1993/11245).

(3) Altering conjugated linolenic or linoleic acid content, such as in WO 2001/12800.

(4) Altering LEC1, AGP, Dek1, Superal1, mi1 ps, various Ipa genes such as Ipa1, Ipa3, hpt or hggt. For example, see, WO 2002/42424, WO 1998/22604, WO 2003/011015, WO 2002/057439, WO 2003/011015, U.S. Pat. Nos. 6,423,886, 6,197,561, 6,825,397 and US Patent Application Publication Numbers US 2003/0079247, US 2003/0204870 and Rivera-Madrid, et al., (1995) Proc. Natl. Acad. Sci. 92:5620-5624.

(5) Genes encoding delta-8 desaturase for making long-chain polyunsaturated fatty acids (U.S. Pat. No. 8,058,571), delta-9 desaturase for lowering saturated fats (U.S. Pat. No. 8,063,269), Primula Δ6-desaturase for improving omega-3 fatty acid profiles.

(6) Isolated nucleic acids and proteins associated with lipid and sugar metabolism regulation, in particular, lipid metabolism protein (LMP) used in methods of producing transgenic plants and modulating levels of seed storage compounds including lipids, fatty acids, starches or seed storage proteins and use in methods of modulating the seed size, seed number, seed weights, root length and leaf size of plants (EP 2404499).

(7) Altering expression of a High-Level Expression of Sugar-Inducible 2 (HSI2) protein in the plant to increase or decrease expression of HSI2 in the plant. Increasing expression of HSI2 increases oil content while decreasing expression of HSI2 decreases abscisic acid sensitivity and/or increases drought resistance (US Patent Application Publication Number 2012/0066794).

(B) Altered phosphorus content, for example, by the

(1) Introduction of a phytase-encoding gene would enhance breakdown of phytate, adding more free phosphate to the transformed plant. For example, see, Van Hartingsveldt, et al., (1993) Gene 127:87, for a disclosure of the nucleotide sequence of an Aspergillus niger phytase gene.

(2) Modulating a gene that reduces phytate content. In maize, this, for example, could be accomplished, by cloning and then re-introducing DNA associated with one or more of the alleles, such as the LPA alleles, identified in maize mutants characterized by low levels of phytic acid, such as in WO 2005/113778 and/or by altering inositol kinase activity as in WO 2002/059324, US Patent Application Publication Number 2003/0009011, WO 2003/027243, US Patent Application Publication Number 2003/0079247, WO 1999/05298, U.S. Pat. No. 6,197,561, U.S. Pat. No. 6,291,224, U.S. Pat. No. 6,391,348, WO 2002/059324, US Patent Application Publication Number 2003/0079247, WO 1998/45448, WO 1999/55882, WO 2001/04147.

(C) Altered carbohydrates affected, for example, by altering a gene for an enzyme that affects the branching pattern of starch or, a gene altering thioredoxin such as NTR and/or TRX (see, U.S. Pat. No. 6,531,648. which is incorporated by reference for this purpose) and/or a gamma zein knock out or mutant such as cs27 or TUSC27 or en27 (see, U.S. Pat. No. 6,858,778 and US Patent Application Publication Number 2005/0160488, US Patent Application Publication Number 2005/0204418, which are incorporated by reference for this purpose). See, Shiroza, et al., (1988) J. Bacteriol. 170:810 (nucleotide sequence of Streptococcus mutant fructosyltransferase gene), Steinmetz, et al., (1985) Mol. Gen. Genet. 200:220 (nucleotide sequence of Bacillus subtilis levansucrase gene), Pen, et al., (1992) Bio/Technology 10:292 (production of transgenic plants that express Bacillus licheniformis alpha-amylase), Elliot, et al., (1993) Plant Molec. Biol. 21:515 (nucleotide sequences of tomato invertase genes), Søgaard, et al., (1993) J. Biol. Chem. 268:22480 (site-directed mutagenesis of barley alpha-amylase gene) and Fisher, et al., (1993) Plant Physiol. 102:1045 (maize endosperm starch branching enzyme II), WO 1999/10498 (improved digestibility and/or starch extraction through modification of UDP-D-xylose 4-epimerase, Fragile 1 and 2, Ref1, HCHL, C4H), U.S. Pat. No. 6,232,529 (method of producing high oil seed by modification of starch levels (AGP)). The fatty acid modification genes mentioned herein may also be used to affect starch content and/or composition through the interrelationship of the starch and oil pathways.

(D) Altered antioxidant content or composition, such as alteration of tocopherol or tocotrienols. For example, see, U.S. Pat. No. 6,787,683, US Patent Application Publication Number 2004/0034886 and WO 2000/68393 involving the manipulation of antioxidant levels and WO 2003/082899 through alteration of a homogentisate geranyl geranyl transferase (hggt).

(E) Altered essential seed amino acids. For example, see, U.S. Pat. No. 6,127,600 (method of increasing accumulation of essential amino acids in seeds), U.S. Pat. No. 6,080,913 (binary methods of increasing accumulation of essential amino acids in seeds), U.S. Pat. No. 5,990,389 (high lysine), WO 1999/40209 (alteration of amino acid compositions in seeds), WO 1999/29882 (methods for altering amino acid content of proteins), U.S. Pat. No. 5,850,016 (alteration of amino acid compositions in seeds), WO 1998/20133 (proteins with enhanced levels of essential amino acids), U.S. Pat. No. 5,885,802 (high methionine), U.S. Pat. No. 5,885,801 (high threonine), U.S. Pat. No. 6,664,445 (plant amino acid biosynthetic enzymes), U.S. Pat. No. 6,459,019 (increased lysine and threonine), U.S. Pat. No. 6,441,274 (plant tryptophan synthase beta subunit), U.S. Pat. No. 6,346,403 (methionine metabolic enzymes), U.S. Pat. No. 5,939,599 (high sulfur), U.S. Pat. No. 5,912,414 (increased methionine), WO 1998/56935 (plant amino acid biosynthetic enzymes), WO 1998/45458 (engineered seed protein having higher percentage of essential amino acids), WO 1998/42831 (increased lysine), U.S. Pat. No. 5,633,436 (increasing sulfur amino acid content), U.S. Pat. No. 5,559,223 (synthetic storage proteins with defined structure containing programmable levels of essential amino acids for improvement of the nutritional value of plants), WO 1996/01905 (increased threonine), WO 1995/15392 (increased lysine), US Patent Application Publication Number 2003/0163838, US Patent Application Publication Number 2003/0150014, US Patent Application Publication Number 2004/0068767, U.S. Pat. No. 6,803,498, WO 2001/79516.

4. Genes Creating a Site for Site-Specific DNA Integration.

This includes the introduction of FRT sites that may be used in the FLP/FRT system and/or Lox sites that may be used in the Cre/Loxp system. For example, see, Lyznik, et al., (2003) Plant Cell Rep 21:925-932 and WO 1999/25821, which are hereby incorporated by reference. Other systems that may be used include the Gin recombinase of phage Mu (Maeser, et al., (1991) Vicki Chandler, The Maize Handbook ch. 118 (Springer-Verlag 1994), the Pin recombinase of E. coli (Enomoto, et al., 1983) and the R/RS system of the pSRi plasmid (Araki, et al., 1992).

5. Genes Affecting Abiotic Stress Resistance

Including but not limited to flowering, ear and seed development, enhancement of nitrogen utilization efficiency, altered nitrogen responsiveness, drought resistance or tolerance, cold resistance or tolerance and salt resistance or tolerance and increased yield under stress.

(A) For example, see: WO 2000/73475 where water use efficiency is altered through alteration of malate; U.S. Pat. Nos. 5,892,009, 5,965,705, 5,929,305, 5,891,859, 6,417,428, 6,664,446, 6,706,866, 6,717,034, 6,801,104, WO 2000/060089, WO 2001/026459, WO 2001/035725, WO 2001/034726, WO 2001/035727, WO 2001/036444, WO 2001/036597, WO 2001/036598, WO 2002/015675, WO 2002/017430, WO 2002/077185, WO 2002/079403, WO 2003/013227, WO 2003/013228, WO 2003/014327, WO 2004/031349, WO 2004/076638, WO 199809521.

(B) WO 199938977 describing genes, including CBF genes and transcription factors effective in mitigating the negative effects of freezing, high salinity and drought on plants, as well as conferring other positive effects on plant phenotype.

(C) US Patent Application Publication Number 2004/0148654 and WO 2001/36596 where abscisic acid is altered in plants resulting in improved plant phenotype such as increased yield and/or increased tolerance to abiotic stress.

(D) WO 2000/006341, WO 2004/090143, U.S. Pat. Nos. 7,531,723 and 6,992,237 where cytokinin expression is modified resulting in plants with increased stress tolerance, such as drought tolerance, and/or increased yield. Also see, WO 2002/02776, WO 2003/052063, JP 2002/281975, U.S. Pat. No. 6,084,153, WO 2001/64898, U.S. Pat. No. 6,177,275 and U.S. Pat. No. 6,107,547 (enhancement of nitrogen utilization and altered nitrogen responsiveness).

(E) For ethylene alteration, see, US Patent Application Publicaiton Number 2004/0128719, US Patent Application Publication Number 2003/0166197 and WO 2000/32761.

(F) For plant transcription factors or transcriptional regulators of abiotic stress, see, e.g., US Patent Application Publication Number 2004/0098764 or US Patent Application Publication Number 2004/0078852.

(G) Genes that increase expression of vacuolar pyrophosphatase such as AVP1 (U.S. Pat. No. 8,058,515) for increased yield; nucleic acid encoding a HSFA4 or a HSFA5 (Heat Shock Factor of the class A4 or A5) polypeptides, an oligopeptide transporter protein (OPT4-like) polypeptide; a plastochron2-like (PLA2-like) polypeptide or a Wuschel related homeobox 1-like (WOX1-like) polypeptide (U. Patent Application Publication Number US 2011/0283420).

(H) Down regulation of polynucleotides encoding poly (ADP-ribose) polymerase (PARP) proteins to modulate programmed cell death (U.S. Pat. No. 8,058,510) for increased vigor.

(I) Polynucleotide encoding DTP21 polypeptides for conferring drought resistance (US Patent Application Publication Number US 2011/0277181).

(J) Nucleotide sequences encoding ACC Synthase 3 (ACS3) proteins for modulating development, modulating response to stress, and modulating stress tolerance (US Patent Application Publication Number US 2010/0287669).

(K) Polynucleotides that encode proteins that confer a drought tolerance phenotype (DTP) for conferring drought resistance (WO 2012/058528).

Other genes and transcription factors that affect plant growth and agronomic traits such as yield, flowering, plant growth and/or plant structure, can be introduced or introgressed into plants, see e.g., WO 1997/49811 (LHY), WO 1998/56918 (ESD4), WO 1997/10339 and U.S. Pat. No. 6,573,430 (TFL), U.S. Pat. No. 6,713,663 (FT), WO 1996/14414 (CON), WO 1996/38560, WO 2001/21822 (VRN1), WO 2000/44918 (VRN2), WO 1999/49064 (GI), WO 2000/46358 (FR1), WO 1997/29123, U.S. Pat. No. 6,794,560, U.S. Pat. No. 6,307,126 (GAI), WO 1999/09174 (D8 and Rht) and WO 2004/076638 and WO 2004/031349 (transcription factors).

6. Genes Conferring Increased Yield

(A) A transgenic crop plant transformed by a 1-AminoCyclopropane-1-Carboxylate Deaminase-like Polypeptide (ACCDP) coding nucleic acid, wherein expression of the nucleic acid sequence in the crop plant results in the plant's increased root growth, and/or increased yield, and/or increased tolerance to environmental stress as compared to a wild type variety of the plant (U.S. Pat. No. 8,097,769).

(B) Over-expression of maize zinc finger protein gene (Zm-ZFP1) using a seed preferred promoter has been shown to enhance plant growth, increase kernel number and total kernel weight per plant (US Patent Application Publication Number 2012/0079623).

(C) Constitutive over-expression of maize lateral organ boundaries (LOB) domain protein (Zm-LOBDP1) has been shown to increase kernel number and total kernel weight per plant (US Patent Application Publication Number 2012/0079622).

(D) Enhancing yield-related traits in plants by modulating expression in a plant of a nucleic acid encoding a VIM1 (Variant in Methylation 1)-like polypeptide or a VTC2-like (GDP-L-galactose phosphorylase) polypeptide or a DUF1685 polypeptide or an ARF6-like (Auxin Responsive Factor) polypeptide (WO 2012/038893).

(E) Modulating expression in a plant of a nucleic acid encoding a Step 20-like polypeptide or a homologue thereof gives plants having increased yield relative to control plants (EP 2431472).

7. Gene Silencing

In some embodiments the stacked trait may be in the form of silencing of one or more polynucleotides of interest resulting in suppression of one or more target pest polypeptides. In some embodiments the silencing is achieved through the use of a suppression DNA construct.

In some embodiments one or more polynucleotides encoding the polypeptides or fragments or variants thereof may be stacked with one or more polynucleotides encoding one or more polypeptides having insecticidal activity or agronomic traits as set forth supra and optionally may further include one or more polynucleotides providing for gene silencing of one or more target polynucleotides as discussed infra.

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The term “suppression” includes lower, reduce, decline, decrease, inhibit, eliminate and prevent. “Silencing” or “gene silencing” does not specify mechanism and is inclusive, and not limited to, anti-sense, cosuppression, viral-suppression, hairpin suppression, stem-loop suppression, RNAi-based approaches and small RNA-based approaches.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50% or any integer between 51% and 100% identical) to all or part of the sense strand (or antisense strand) of the gene of interest.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as siRNA (short interfering RNA) constructs and miRNA (microRNA) constructs.

“Antisense inhibition” refers to the production of antisense RNA transcripts capable of suppressing the expression of the target protein.

“Antisense RNA” refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns or the coding sequence.

“Cosuppression” refers to the production of sense RNA transcripts capable of suppressing the expression of the target protein. “Sense” RNA refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see, Vaucheret, et al., (1998) Plant J. 16:651-659 and Gura, (2000) Nature 404:804-808).

Another variation describes the use of plant viral sequences to direct the suppression of proximal mRNA encoding sequences (PCT Publication WO 1998/36083).

Recent work has described the use of “hairpin” structures that incorporate all or part, of an mRNA encoding sequence in a complementary orientation that results in a potential “stem-loop” structure for the expressed RNA (PCT Publication WO 1999/53050). In this case the stem is formed by polynucleotides corresponding to the gene of interest inserted in either sense or anti-sense orientation with respect to the promoter and the loop is formed by some polynucleotides of the gene of interest, which do not have a complement in the construct. This increases the frequency of cosuppression or silencing in the recovered transgenic plants. For review of hairpin suppression, see, Wesley, et al., (2003) Methods in Molecular Biology, Plant Functional Genomics: Methods and Protocols 236:273-286.

A construct where the stem is formed by at least 30 nucleotides from a gene to be suppressed and the loop is formed by a random nucleotide sequence has also effectively been used for suppression (PCT Publication WO 1999/61632).

The use of poly-T and poly-A sequences to generate the stem in the stem-loop structure has also been described (PCT Publication WO 2002/00894).

Yet another variation includes using synthetic repeats to promote formation of a stem in the stem-loop structure. Transgenic organisms prepared with such recombinant DNA fragments have been shown to have reduced levels of the protein encoded by the nucleotide fragment forming the loop as described in PCT Publication WO 2002/00904.

RNA interference refers to the process of sequence-specific post-transcriptional gene silencing in animals mediated by short interfering RNAs (siRNAs) (Fire, et al., (1998) Nature 391:806). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire, et al., (1999) Trends Genet. 15:358). Such protection from foreign gene expression may have evolved in response to the production of double-stranded RNAs (dsRNAs) derived from viral infection or from the random integration of transposon elements into a host genome via a cellular response that specifically destroys homologous single-stranded RNA of viral genomic RNA. The presence of dsRNA in cells triggers the RNAi response through a mechanism that has yet to be fully characterized.

The presence of long dsRNAs in cells stimulates the activity of a ribonuclease III enzyme referred to as dicer. Dicer is involved in the processing of the dsRNA into short pieces of dsRNA known as short interfering RNAs (siRNAs) (Berstein, et al., (2001) Nature 409:363). Short interfering RNAs derived from dicer activity are typically about 21 to about 23 nucleotides in length and comprise about 19 base pair duplexes (Elbashir, et al., (2001) Genes Dev. 15:188). Dicer has also been implicated in the excision of 21- and 22-nucleotide small temporal RNAs (stRNAs) from precursor RNA of conserved structure that are implicated in translational control (Hutvagner, et al., (2001) Science 293:834). The RNAi response also features an endonuclease complex, commonly referred to as an RNA-induced silencing complex (RISC), which mediates cleavage of single-stranded RNA having sequence complementarity to the antisense strand of the siRNA duplex. Cleavage of the target RNA takes place in the middle of the region complementary to the antisense strand of the siRNA duplex (Elbashir, et al., (2001) Genes Dev. 15:188). In addition, RNA interference can also involve small RNA (e.g., miRNA) mediated gene silencing, presumably through cellular mechanisms that regulate chromatin structure and thereby prevent transcription of target gene sequences (see, e.g., Allshire, (2002) Science 297:1818-1819; Volpe, et al., (2002) Science 297:1833-1837; Jenuwein, (2002) Science 297:2215-2218 and Hall, et al., (2002) Science 297:2232-2237). As such, miRNA molecules of the invention can be used to mediate gene silencing via interaction with RNA transcripts or alternately by interaction with particular gene sequences, wherein such interaction results in gene silencing either at the transcriptional or post-transcriptional level.

Methods and compositions are further provided which allow for an increase in RNAi produced from the silencing element. In such embodiments, the methods and compositions employ a first polynucleotide comprising a silencing element for a target pest sequence operably linked to a promoter active in the plant cell; and, a second polynucleotide comprising a suppressor enhancer element comprising the target pest sequence or an active variant or fragment thereof operably linked to a promoter active in the plant cell. The combined expression of the silencing element with suppressor enhancer element leads to an increased amplification of the inhibitory RNA produced from the silencing element over that achievable with only the expression of the silencing element alone. In addition to the increased amplification of the specific RNAi species itself, the methods and compositions further allow for the production of a diverse population of RNAi species that can enhance the effectiveness of disrupting target gene expression. As such, when the suppressor enhancer element is expressed in a plant cell in combination with the silencing element, the methods and composition can allow for the systemic production of RNAi throughout the plant; the production of greater amounts of RNAi than would be observed with just the silencing element construct alone; and, the improved loading of RNAi into the phloem of the plant, thus providing better control of phloem feeding insects by an RNAi approach. Thus, the various methods and compositions provide improved methods for the delivery of inhibitory RNA to the target organism. See, for example, US Patent Application Publication 2009/0188008.

As used herein, a “suppressor enhancer element” comprises a polynucleotide comprising the target sequence to be suppressed or an active fragment or variant thereof. It is recognize that the suppressor enhancer element need not be identical to the target sequence, but rather, the suppressor enhancer element can comprise a variant of the target sequence, so long as the suppressor enhancer element has sufficient sequence identity to the target sequence to allow for an increased level of the RNAi produced by the silencing element over that achievable with only the expression of the silencing element. Similarly, the suppressor enhancer element can comprise a fragment of the target sequence, wherein the fragment is of sufficient length to allow for an increased level of the RNAi produced by the silencing element over that achievable with only the expression of the silencing element.

It is recognized that multiple suppressor enhancer elements from the same target sequence or from different target sequences or from different regions of the same target sequence can be employed. For example, the suppressor enhancer elements employed can comprise fragments of the target sequence derived from different region of the target sequence (i.e., from the 3′UTR, coding sequence, intron, and/or 5′UTR). Further, the suppressor enhancer element can be contained in an expression cassette, as described elsewhere herein, and in specific embodiments, the suppressor enhancer element is on the same or on a different DNA vector or construct as the silencing element. The suppressor enhancer element can be operably linked to a promoter. It is recognized that the suppressor enhancer element can be expressed constitutively or alternatively, it may be produced in a stage-specific manner employing the various inducible or tissue-preferred or developmentally regulated promoters that are discussed elsewhere herein.

In specific embodiments, employing both a silencing element and the suppressor enhancer element the systemic production of RNAi occurs throughout the entire plant. In further embodiments, the plant or plant parts of the invention have an improved loading of RNAi into the phloem of the plant than would be observed with the expression of the silencing element construct alone and, thus provide better control of phloem feeding insects by an RNAi approach. In specific embodiments, the plants, plant parts and plant cells of the invention can further be characterized as allowing for the production of a diversity of RNAi species that can enhance the effectiveness of disrupting target gene expression.

In specific embodiments, the combined expression of the silencing element and the suppressor enhancer element increases the concentration of the inhibitory RNA in the plant cell, plant, plant part, plant tissue or phloem over the level that is achieved when the silencing element is expressed alone.

As used herein, an “increased level of inhibitory RNA” comprises any statistically significant increase in the level of RNAi produced in a plant having the combined expression when compared to an appropriate control plant. For example, an increase in the level of RNAi in the plant, plant part or the plant cell can comprise at least about a 1%, about a 1%-5%, about a 5%-10%, about a 10%-20%, about a 20%-30%, about a 30%-40%, about a 40%-50%, about a 50%-60%, about 60-70%, about 70%-80%, about a 80%-90%, about a 90%-100% or greater increase in the level of RNAi in the plant, plant part, plant cell or phloem when compared to an appropriate control. In other embodiments, the increase in the level of RNAi in the plant, plant part, plant cell or phloem can comprise at least about a 1 fold, about a 1 fold-5 fold, about a 5 fold-10 fold, about a 10 fold-20 fold, about a 20 fold-30 fold, about a 30 fold-40 fold, about a 40 fold-50 fold, about a 50 fold-60 fold, about 60 fold-70 fold, about 70 fold-80 fold, about a 80 fold-90 fold, about a 90 fold-100 fold or greater increase in the level of RNAi in the plant, plant part, plant cell or phloem when compared to an appropriate control. Examples of combined expression of the silencing element with suppressor enhancer element for the control of Stinkbugs and Lygus can be found in US Patent Application Publication 2011/0301223 and US Patent Application Publication 2009/0192117.

Some embodiments relate to down-regulation of expression of target genes in insect pest species by interfering ribonucleic acid (RNA) molecules. PCT Publication WO 2007/074405 describes methods of inhibiting expression of target genes in invertebrate pests including Colorado potato beetle. PCT Publication WO 2005/110068 describes methods of inhibiting expression of target genes in invertebrate pests including in particular Western corn rootworm as a means to control insect infestation. Furthermore, PCT Publication WO 2009/091864 describes compositions and methods for the suppression of target genes from insect pest species including pests from the Lygus genus. PCT Publication WO 2012/055982 describes ribonucleic acid (RNA or double stranded RNA) that inhibits or down regulates the expression of a target gene that encodes: an insect ribosomal protein such as the ribosomal protein L19, the ribosomal protein L40 or the ribosomal protein S27A; an insect proteasome subunit such as the Rpn6 protein, the Pros 25, the Rpn2 protein, the proteasome beta 1 subunit protein or the Pros beta 2 protein; an insect β-coatomer of the COP1 vesicle, the γ-coatomer of the COP1 vesicle, the β′-coatomer protein or the ζ-coatomer of the COP1 vesicle; an insect Tetraspanine 2 A protein which is a putative transmembrane domain protein; an insect protein belonging to the actin family such as Actin 5C; an insect ubiquitin-5E protein; an insect Sec23 protein which is a GTPase activator involved in intracellular protein transport; an insect crinkled protein which is an unconventional myosin which is involved in motor activity; an insect crooked neck protein which is involved in the regulation of nuclear alternative mRNA splicing; an insect vacuolar H+-ATPase G-subunit protein and an insect Tbp-1 such as Tat-binding protein.

“Drought” refers to a decrease in water availability to a plant that, especially when prolonged, can cause damage to the plant or prevent its successful growth (e.g., limiting plant growth or seed yield). “Drought tolerance” is a trait of a plant to survive under drought conditions over prolonged periods of time without exhibiting substantial physiological or physical deterioration. “Increased drought tolerance” of a plant is measured relative to a reference or control plant, and is a trait of the plant to survive under drought conditions over prolonged periods of time, without exhibiting the same degree of physiological or physical deterioration relative to the reference or control plant grown under similar drought conditions. Typically, when a transgenic plant comprising a recombinant DNA construct or suppression DNA construct in its genome exhibits increased drought tolerance relative to a reference or control plant, the reference or control plant does not comprise in its genome the recombinant DNA construct or suppression DNA construct.

One of ordinary skill in the art is familiar with protocols for simulating drought conditions and for evaluating drought tolerance of plants that have been subjected to simulated or naturally-occurring drought conditions. For example, one can simulate drought conditions by giving plants less water than normally required or no water over a period of time, and one can evaluate drought tolerance by looking for differences in physiological and/or physical condition, including (but not limited to) vigor, growth, size, or root length, or in particular, leaf color or leaf area size. Other techniques for evaluating drought tolerance include measuring chlorophyll fluorescence, photosynthetic rates and gas exchange rates.

A drought stress experiment may involve a chronic stress (i.e., slow dry down) and/or may involve two acute stresses (i.e., abrupt removal of water) separated by a day or two of recovery.

The regenerable plant tissue can be obtained from any plant species, including crops such as, but not limited to: a graminaceous plant, saccharum spp., saccharum spp. hybrids, sugarcane, miscanthus, switchgrass, energycane, sterile grasses, bamboo, cassava, rice, potato, sweet potato, yam, banana, pineapple, citrus, trees, willow, poplar, mulberry, ficus spp., oil palm, date palm, poaceae, verbena, vanilla, tea, hops, Erianthus spp., intergenic hybrids of Saccharum, Erianthus and Sorghum spp., African violet, date, fig, conifers, apple, guava, mango, maple, plum, pomegranate, papaya, avocado, blackberries, garden strawberry, grapes, canna, cannabis, lemon, orange, grapefruit, tangerine, dayap, maize, wheat, sorghum and cotton.

In one embodiment, the regenerable plant tissue used in the artificial seed can be from sugarcane. The regenerable plant tissue can be prepared using several methods including excision of meristems from the top of the sugarcane stalks, followed by tissue culture on solid or liquid media, or temporarily immersed in liquid nutrients and combinations thereof. In one embodiment, the regenerable sugarcane tissue can be prepared using tissue culture on a solid medium, followed by temporary immersion in liquid nutrient media.

The meristem tissue can be allowed to grow and proliferate using a proliferation medium. The proliferation medium can include, but is not limited to, culturing in various liquid nutrient media, culturing on solid media, temporary immersion in liquid nutrient media, and any variations thereof. In one embodiment, the proliferation medium used in the current method comprises MS nutrients and can additionally comprise ingredients not limited to: 30 g/L sucrose, one or more cytokinins, including 6-BAP, auxins, or combinations of cytokinin and auxin, with or without inhibitors of the plant hormone, gibberellin. However, other nutrient formulations such as the well known in the art Gamborg's B-5 medium, other carbon sources such as glucose and mannitol, other cytokinins, such as kinetin and zeatin can also be used.

The meristem tissues can be allowed to proliferate from about 3 weeks to about 52 weeks. The temperature used for proliferation can vary from about 15° C. to about 45° C. Temperature control for growth of the regenerable plant tissues can be achieved using constant temperature incubators as is well known in the relevant art.

Following growth of the meristem tissue, proliferated buds are formed which contain independent meristem structures capable of differentiating into shoots, and subsequently into well-formed plantlets at later stages. As used herein, “proliferated bud tissue” means a meristematic tissue with the capacity to multiply and self-regenerate into similar meristem structures. Over time, the base of this tissue, which was the original plant tissue, can blacken due to polyphenol production and can be removed by mechanical trimming methods well known in the relevant art.

During the steps described above, the meristem tissue can be subjected to light to allow for growth. The light intensity suitable for the current invention can be from 1 micro (μ) Einstein per square meter per second (μE/m²/s) to about 1500 (μE/m²/s). The light can be produced by various devices suitable for this purpose such as fluorescent bulbs, incandescent bulbs, the sun, plant growth bulbs and light emitting diodes (LEDs). The amount of light required for growth of the meristem tissue can vary from 1 hour photoperiod to 24 hours photoperiod. In an embodiment, a 16 hours photoperiod using 30 μE/m²/s can be used.

After the meristem tissue forms the proliferated bud tissue, it can then be cut into small pieces (fragmented) to form tissue fragments. These tissue fragments can be 0.5-10 mm in size. Alternatively, they can be 1-5 mm in size. These tissue fragments can then be cultured for 0-5 weeks further to form plantlets, which are suitable for encapsulation in the artificial seeds. The culturing processes to form the plantlets can include, but is not limited to, culturing in various liquid nutrient media, culturing on solid media, temporary immersion in liquid culture, and any variations thereof. The plantlets that are formed in these processes possess shoots, with or without roots.

Artificial seeds of the type described in the present invention comprise a container assembly. The container assembly may be prepared using any variety of materials disclosed above. In the present method, the regenerable plant tissue, which has been further cultivated to produce a plantlet may be used. The plantlet may be partially embedded into a nutrient-containing agar plug at the bottom of the container of the artificial seed such that part of the tissue (e.g., approximately 80%) is optionally exposed to the airspace above the nutrient source. Alternatively the plantlet can be placed such that between about 1% and 99.9% is exposed to the airspace. The plantlet can be oriented or not, and can be trimmed to fit inside the container. Alternately, the plantlet can be placed in a soil layer in the container, such that airspace is present above it.

In the present method it is desirable to create an airspace within the container. The purpose of the airspace is to allow rapid gas exchange with the plantlet, helping to sustain the tissue and allow it to grow. The container can possess porosity which can allow a rate of gas transport such that equilibrium can be maintained between the airspace and the outside environment. Thus, as the plantlet consumes or releases oxygen or carbon dioxide, due to either respiration or photosynthesis, these gases are rapidly equilibrated with the outside atmosphere. In addition, the exposure of the plantlet to the airspace fosters the development of tissue that is better adapted to the harsher conditions the plantlet can be exposed to once it emerges from the seed (for example reduced humidity, wind, higher light). In the artificial seed, the plantlet is exposed to less harsh conditions due to the protection of the container. In the present invention, the airspace is also transparent to visible light, which allows the plantlet to perform photosynthesis. The airspace can also provide some thermal insulation for the plantlet. The airspace may consist of multiple compartments. These compartments may be connected or adjoined and may be in communication with each other. The airspace inside the container artificial seed is at least 1% of the total volume of the container.

To prevent fungal contamination of the artificial seed, the container can be treated with a solution of a fungicide prior to its assembly. Many fungicides can be used for this purpose. Examples include, but are not limited to: Maxim® XL, Maxim® 4FS, Ridomil Gold®, Uniform®, Quilt®, amphotericin B, cycloheximide, nystatin, griseofulvin, pentachloronitrobenzene, thiabendazole, benomyl, 2-(thiocyanatomethylthio)-1,3-benzothiazole, carbendazim, fuberidazole, thiophanate, thiophanate-methyl, chlozolinate, iprodione, procymidone, vinclozolin, imazalil, oxpoconazole, pefurazoate, prochloraz, triflumizole, triforine, pyrifenox, fenarimol, nuarimol, azaconazole, bitertanol, bromuconazole, cyproconazole, difenoconazole, diniconazole, epoxiconazole, fenbuconazole, fluquinconazole, flusilazole, flutriafol, hexaconazole, imibenconazole, ipconazole, metconazole, myclobutanil, penconazole, propiconazole, prothioconazole, simeconazole, tebuconazole, tetraconazole, triadimefon, triadimenol, triticonazole, benalaxyl, furalaxyl, metalaxyl, metalaxyl-M (mefenoxam), oxadixyl, ofurace, aldimorph, dodemorph, fenpropimorph, tridemorph, fenpropidin, piperalin, spiroxamine, edifenphos, iprobenfos, (IBP), pyrazophos, isoprothiolane, benodanil, flutolanil, mepronil, fenfuram, carboxin, oxycarboxin, thifluzamide, furametpyr, penthiopyrad, boscalid, bupirimate, dimethirimol, ethirimol, cyprodinil, mepanipyrim, pyrimethanil, diethofencarb, azoxystrobin, strobilurins, enestrobin, picoxystrobin, pyraclostrobin, kresoxim-methyl, trifloxystrobin, dimoxystrobin, metominostrobin, orysastrobin, famoxadone, fluoxastrobin, fenamidone, pyribencarb, fenpiclonil, fludioxonil, quinoxyfen, biphenyl, chloroneb, dicloran, quintozene (PCNB), tecnazene (TCNB), tolclofos-methyl, etridiazole, ethazole, fthalide, pyroquilon, tricyclazole, carpropamid, diclocymet, fenoxanil, fenhexamid, pyributicarbi, naftifine, terbinafine, polyoxin, pencycuron, cyazofamid, amisulbrom, zoxamide, blasticidin-S, kasugamycin, streptomycin, streptomycin sulfate, validamycin, cymoxanil, iodocarb, propamocarb, prothiocarb, binapacryl, dinocap, ferimzone, fluazinam, fentin acetate, fentin chloride, fentin hydroxide, oxolinic acid, hymexazole, octhilinone, fosetyl-Al, phosphorous acid and salts, teclofthalam, triazoxide, flusulfamide, diclomezine, silthiofam, diflumetorim, dimethomorph, flumorph, benthiavalicarb, iprovalicarb, valiphenal, mandipropamid, oxytetracycline, methasulfocarb, fluopicolide, acibenzolar-S-methyl, probenazole, tiadinil, isotianil, ethaboxam, cyflufenamid, proquinazid, metrafenone, copper (different, salts), sulphur, ferbam, mancozeb, maneb, metiram, propineb, thiram, zineb, ziram, captan, captafol, folpet, chlorothalonil, dichlofluanid, tolylfluanid, dodine, guazatine, iminoctadine, anilazine, dithianon, mineral oils, organic oils, potassium bicarbonate, tridemorph anilinopyrimidines, antibiotics, cycloheximid, griseofulvin, dinitroconazole, etridazole, perfurazoate, 5-Chloro-7-(4-methyl-piperidin-1-yl)-6-(2,4,6-trifluoro-phenyl)-[1,2,4]tr-iazolo[1,5-a]pyrimidine, 2-Butoxy-6-iodo-3-propyl-chromen-4-one, 3-(3-Bromo-6-fluoro-2-methyl-indole-1-sulfonyl)-[1,2,4]triazole-1-sulfoni-c acid dimethylamide, nabam, metam, polycarbamate, dazomet, 3-[5-(4-Chloro-phenyl)-2,3-dimethyl-isoxazolidin-3-yl]-pyridine, Bordeaux mixture, copper acetate, copper hydroxide, copper oxychloride, basic copper sulfate, nitrophenyl derivatives, dinobuton, nitrophthalisopropyl phenylpyrroles, sulfur, sulfur organometallic compounds, phthalide, toloclofos-methyl, N-(2-{4-[3-(4-Chloro-phenyl)-prop-2-ynyloxy]-3-methoxy-phenyl}-ethyl)-2-m-ethanesulfonylamino-3-methyl-butyramide, N-(2-{4-[3-(4-Chloro-phenyl)-prop-2-ynyloxy]-3-methoxy-phenyl}-ethyl)-2-e-thanesulfonylamino-3-methyl-butyramide; 3,4-Dichloro-isothiazole-5-carboxylic acid(2-cyano-phenyl)-amide, Flubenthiavalicarb, 3-(4-Chloro-phenyl)-3-(2-isopropoxycarbonylamino-3-methyl-butyrylamino)-p-ropionic acid methyl ester, {2-Chloro-5-[1-(6-methyl-pyridin-2-ylmethoxyimino)-ethyl]-benzyl}-carbami-c acid methyl ester, {2-Chloro-5-[1-(3-methyl-benzyloxyimino)-ethyl]-benzyl}-carbamic acid methyl ester, hexachlorbenzene amides of following formula in which X is CHF2 or CH3; and R1, R2 are independently from each other halogen, methyl or halomethyl; enestroburin, sulfenic acid derivatives, cinnemamides and analogs such as, flumetover amide fungicides such as cyclofenamid or (Z)-N-[a-(cyclopropylmethoxyimino)-2,3-difluoro-6-(difluoromethoxy)benzyl]-2-phenylacetamide, thiabendozole, and triffumizole.

Additionally, the container may comprise one or more antimicrobials, including but not limited to: bleach, Plant Preservative Mixture™, quaternary ammonium or pyridinium salts, the copper salt of cyanoethylated sorbitol (as described in U.S. Pat. No. 6,978,724), silver salts and silver nanoparticles can be used. Additionally, the container may comprise one or more antibiotics, including but not limited to: cefotaxime, carbenicillin, chloramphenicols, tetracycline, erythromycin, kanamycin, neomycin sulfate, streptomycin sulfate, gentamicin sulfate, ampicillin, penicillin, ticarcillin, polymyxin-B and rifampicin chlorhexidine, chlorhexidine acetate, chlorhexidine gluconate, chlorhexidine hydrochloride, chlorhexidine sulfate, hexamethylene biguanides, oligo-hexamethyl biguanides, silver acetate, silver benzoate, silver carbonate, silver chloride, silver iodate, silver iodide, silver lactate, silver laurate, silver nitrate, silver oxide, silver palmitate, silver protein, silver sulfadiazine, polymyxin, tetracycline, tobramycin, gentamicin, rifampician, bacitracin, neomycin, chloramphenical, miconazole, tolnaftate, oxolinic acid, norfloxacin, nalidix acid, pefloxacin, enoxacin, ciprofloxacin, ampicillin, amoxicillin, piracil, vancomycin, polyhexamethylene biguanide, polyhexamethylene biguanide hydrochloride, polyhexamethylene biguanide hydrobromide, polyhexamethylene biguanide borate, polyhexamethylene biguanide acetate, polyhexamethylene biguanide gluconate, polyhexamethylene biguanide sulfonate, polyhexamethylene biguanide maleate, polyhexamethylene biguanide ascorbate, polyhexamethylene biguanide stearate, polyhexamethylene biguanide tartrate, polyhexamethylene biguanide citrate and combinations thereof.

In order to prevent insect damage, the artificial seed may also comprise one or more insecticides. Examples of suitable pesticidal compounds include, but are not limited to, abamectin, cyanoimine, acetamiprid, nitromethylene, nitenpyram, clothianidin, dimethoate, dinotefuran, fipronil, lufenuron, flubendamide, pyripfoxyfen, thiacloprid, fluxofenime, imidacloprid, thiamethoxam, beta cyfluthrin, fenoxycarb, lamda cyhalothrin, diafenthiuron, pymetrozine, diazinon, disulphoton; profenofos, furathiocarb, cyromazin, cypermethrin, tau-fluvalinate, tefluthrin, chlorantraniliprole, flonicamid, metaflumizone, spirotetramat, Bacillus thuringiensis products, azoxystrobin, acibenzolor s-methyl, bitertanol, carboxin, Cu₂O, cymoxanil, cyproconazole, cyprodinil, dichlofluamid, difenoconazole, diniconazole, epoxiconazole, fenpiclonil, fludioxonil, fluoxastrobin, fluquiconazole, flusilazole, flutriafol, furalaxyl, guazatin, hexaconazole, hymexazol, imazalil, imibenconazole, ipconazole, kresoxim-methyl, mancozeb, metalaxyl, R-metalaxyl, mefenoxam, metconazole, myclobutanil, oxadixyl, pefurazoate, paclobutrazole, penconazole, pencycuron, picoxystrobin, prochloraz, propiconazole, pyroquilone, SSF-109, spiroxamin, tebuconazole, thiabendazole, thiram, tolifluamide, triazoxide, triadimefon, triadimenol, trifloxystrobin, triflumizole, triticonazole, uniconazole.

The artificial seed may comprise other crop protection chemicals, including but not limited to nematicides, termiticides, molluscicides, miticides and acaricides.

In the process of artificial seed preparation and following addition of the plantlet, and in some cases, the nutrients, the opening in the container can be secured. A container can have more than one opening. Alternatively, a container can have a top opening and a bottom opening. Depending on the design and method of planting, optionally one or both openings can be secured. Identical materials can be used as closures for the top opening and the bottom opening of the container. Alternatively, different materials can be used as closures for securing the opening(s). Suitable materials to be used as closures in the disclosed invention include, but are not limited to: various types of paper, wax, Parafilm®, pre-stretched Parafilm®, biodegradable polymers including poly(lactide), poly(L-lactide), poly(D-lactide), poly(D,L-lactide), stereocomplexes of poly(L-lactide) with poly(D-lactide) and poly(hydroxyl alkanoate)s, natural and synthetic polymers including but not limited to poly(ethylene glycol), poly(acrylic acid) and its salts, poly(vinyl alcohol), poly(styrene), poly(alkyl (meth)acrylates), poly(vinyl acetate), poly(vinyl pyrollidinone), poly(vinyl pyridine), polyacrylamide, polycarbonate, epoxy resins, alkyd resins, polyolefins, photodegradable polymers, polyesters, polyamides, starch, gelatin, natural rubber, polysachharides including but not limited to alginate, carrageenan, cellulose, carboxymethylcellulose and its salts, xanthan gum, guar gum, zein, chitosan, locust bean gum, gum arabic, pectin, agar, agarose, crosslinked versions thereof, plasticized versions thereof, copolymers thereof and combinations thereof. In one embodiment, the closure possesses a wax coating. Waxes include but are not limited to paraffin wax, spermaceti wax, beeswax and carnauba wax.

In one embodiment of the invention, the closure is made of biodegradable plastic materials such as poly(lactic acid), poly(hydroxybutyrate), poly(hydroxybutyrate-co-valerate), or blends thereof, optionally with starch, cellulose, chitosan and plasticizers, including but not limited to sorbitol, glycerol, citrate esters, phthalate esters and water. These blends may be formed by solution blending or melt blending.

In another embodiment, the closure comprises, or alternatively consists of, rapidly dissolvable blends of poly(vinyl alcohol) with starch, cellulose fibers and glycerol, optionally crosslinked, with a suitable agent, including but not limited to hexamethoxymethylmelamine or glutaraldehyde. This provides materials which are rapidly degradable in moist soil conditions, permitting rapid growth of the tissue inside. The starch may be from sources including but not limited to potato, corn, rice, wheat and cassava, and may be modified or unmodified. Additional additives may include, but are not limited to poly(ethylene glycol), citric acid, urea, water, salts including but not limited to sodium acetate, potassium nitrate and ammonium nitrate, fertilizers, agar, xanthan gum, alginate, cellulose derivatives including but not limited to hydroxypropylcellulose, methylcellulose and carboxymethylcellulose.

In the disclosed invention, the container may have a top and a bottom opening which can be secured. In an embodiment of the disclosed invention, pre-stretched Parafilm® F can be used to secure both the top opening and the bottom opening of the container. In another embodiment, the closure for the bottom opening can be pre-stretched Parafilm® M and the closure for the top opening can be a water-soluble plastic film, possibly composed of poly(vinyl alcohol), poly(vinyl pyrollidone), poly((meth)acrylic acid) and its salts or poly(ethylene glycol). In yet another embodiment, the closure for the top opening can be pre-stretched Parafilm® M and the closure for the bottom opening can be a wax-impregnated water-soluble paper. As used herein, wax-impregnated water-soluble paper means water soluble paper wherein wax has been introduced to the pores and/or surface of the material.

In another embodiment, the closure for the openings comprise, or alternatively consist of, alkyd resin films. Such alkyd resins are well known in the art, and can be formed through the reaction of unsaturated vegetable oils with polyols and cured with metal catalysts. Suitable alkyd resins include, but are not limited to Beckosol® 11-035 and Amberlac® 1074 (Reichhold Corp, Durham, N.C.).

In another embodiment, the closure for the openings comprises, or alternatively consists of, block copolymers. These polymers include two or more segments of chemically distinct constitutional repeating units, linked covalently. These block copolymers may be biodegradable. In one embodiment, polyester block copolymers are used. Such polymers may be elastomeric, allowing the plantlets to puncture them easily. The block copolymers contain blocks including but not limited to: poly(lactic acid), poly(lactide), poly(L-lactic acid), poly(D-lactic acid), poly(D,L-lactic acid), poly(caprolactone), poly(caprolactone-co-lactic acid), poly(dimethylsiloxane), poly(vinyl alcohol), poly(vinyl acetate), poly(ethylene glycol), polypropylene glycol), poly(carbonate)s, polyethers, polyesters. In one embodiment, the block copolymers can consist of poly(L-lactic acid-b-caprolactone-co-D,L-lactic acid-b-L-lactic acid). In another embodiment, the block copolymer consists of poly(D,L-lactic acid-b-dimethyl siloxane-b-D,L-lactic acid).

The closure useful in the current invention may comprise oil. The oil suitable for application in the current invention has the following characteristics: it should melt between about 30° C. to 38° C. and be solid at room temperature (from about 20° C. to about 25° C.). Various types of oil and triglycerides (fat) can be used. Non-limiting examples include butter, cocoa butter, palm oil, palm stearine and lard. In one embodiment, vegetable oil shortening, e.g., Crisco®, can be used. In another embodiment, the closure may be composed of an oil-gel. An oil-gel is defined as an oil that, through combination with one or more additives, does not flow over a finite range of temperature suitable for the application. In one embodiment, the oil-gel is formed by dissolving a compound in an oil at elevated temperature, and then cooling that solution to form a gel. Suitable oils include, but are not limited to, vegetable oil, castor oil, soybean oil, isopropyl myristate, rapeseed oil, and mineral oil. Suitable compounds include, but are not limited to block polymers and associative, low molecular weight substances. Block polymers include, but are not limited to, styrenic block copolymers such as those sold under the trade name Kraton® (Kraton Polymers, Houston, Tex.), block copolymers of ethylene oxide and propylene oxide, such as those sold under the name Pluronic® (BASF, Ludwigshafen, Germany). Styrenic block copolymers include but are not limited to poly(styrene-b-isoprene-b-styrene), poly(styrene-b-butadiene-b-styrene) and hydrogenated versions thereof. Oil-gels suitable for this application will have mechanical properties weak enough to permit penetration by the growing regenerable plant tissue.

In another embodiment the openings can be secured using porous materials, including but not limited to, screens, meshes, gauze, cotton, clay, cheesecloth, and rockwool.

Alternatively, the top and bottom openings can be secured by folding, crimping, pinching, stapling, or fastening the opposing sides of the container together. In one embodiment, the bottom opening can be secured by stapling its sides together using a common, galvanized steel staple.

In another embodiment, the openings can be secured by the flap-like structures, wherein one or more flexible flaps protrude over the opening. The flaps are flexible enough to allow the plantlet to push them apart as it grows. In one embodiment, the flaps form a slotted lid or “flower” or “blossom”-shaped lid.

In another embodiment, the container can have one or more openings on the side of the container. These side openings can be in addition to the top and bottom openings. Alternatively, the container can have only side openings without top or bottom openings. These openings can also be secured using methods and materials described above.

In another embodiment, the container can possess anchoring devices. Such devices include, but are not limited to flaps, barbs, stakes and ribs. The anchoring devices can be foldable or collapsed, to reduce space prior to planting. In such cases, a restraint may be used to hold the anchoring device in a folded or collapsed state. Such restraints may include, but are not limited to tapes, bands, and adhesives.

Following methods of assembly of the container, adding the plantlet or the regenerable plant tissue, the nutrient medium, if required, and securing the top opening and the bottom opening, the artificial seeds thus created, can be planted in soil. Any kind of soil such as field soil, sandy soil, silty soil, clay soil, organic rich soil, organic poor soil, high pH soil, low pH soil, loam, synthetic soil, vermiculite, potting soil, nursery soil, topsoil, mushroom soil and sterilized versions thereof can be used for this purpose. In an embodiment, Metro-Mix® 360 (and field soil—such as that from farms or other natural sources around the world) can be used for planting the plantlets or the regenerable plant tissue in the containers. The artificial seeds will then sprout or germinate at some frequency thereafter. As used herein, “sprouting” and “germination” mean the protrusion of the regenerable tissue from the boundaries of the container of the artificial seed due to growth of the regenerable tissue.

The artificial seeds described herein are suited for storage prior to planting. Storage conditions may include, but are not limited to ambient temperature, refrigerated temperature, sub-ambient temperature, sub-ambient oxygen concentration, sub-ambient illumination, in light or in darkness, in external packaging, under air or in an inert atmosphere. Sub-ambient temperature is defined as temperature below the ambient temperature. Sub-ambient illumination is defined as illumination levels below the ambient illumination. Sub-ambient oxygen is defined as levels of oxygen below that present in the natural atmosphere. The storage duration may be as long as one year, or a few months, but may also be on the order of weeks or days.

In one embodiment, holes, cuts, breaches or slits may be made in the artificial seed at the time of planting in order to facilitate the growth of the regenerable plant tissue. This can enable the shoots or the roots to grow out of and escape the container.

The present invention provides for production of artificial seeds of plants that can develop into fully grown crops for propagation in the field. For example, the disclosed invention can provide for an economical method of propagating hard-to-scale up plants such as sugarcane that can allow their rapid propagation to meet the growing global demand for sugarcane production. Also, the present invention can provide for a simpler, safer and more economical planting method compared to the traditional planting of sugarcane stalks and billets via either mechanical or manual means. Simply reducing the weight and volume of planting material, from sugarcane stalks and billets to artificial seeds, can save the energy and time required to transport planting materials to the field for planting.

The above description of various illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The teachings provided herein of the invention can be applied to other purposes, other than the examples described above. The invention may be practiced in ways other than those particularly described in the foregoing description and examples. Numerous modifications and variations of the invention are possible in light of the above teachings and, therefore, are within the scope of the appended claims.

These and other changes may be made to the invention in light of the above detailed description. In general, in the following claims, the terms used should not be construed to limit the invention to the specific embodiments disclosed in the specification and the claims.

Certain teachings related to viable plant artificial seeds were disclosed in U.S. Provisional patent application No. 61/578,410, filed Dec. 21, 2011, the disclosure of which is herein incorporated by reference in its entirety.

The entire disclosure of each document cited (including patents, patent applications, journal articles, abstracts, manuals, books, or other disclosures) in the Background of the Invention, Detailed Description, and Examples is herein incorporated by reference in their entireties.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight; temperature is in degrees centigrade; and pressure is at or near atmospheric.

### EXAMPLES

### Materials

Wax paper containers (1.19 cm OD, Aardvark, “Colossal” size) were obtained from Precision Products Group, Inc, 245 Falley Dr, Westfield, Mass.

Vermiculite (part number 65-3120, Whittemore, grade D3, fine) was obtained from Griffin Greenhouse and Nursery Supplies in Morgantown, Pa.

Conviron model BDW-120 and Conviron CGR-962 were purchased from Conviron, Manitoba Canada.

Porous filter tape was from Carolina Biological Supply Company, Burlington, N.C.

Decagon EC-5 probe was from Decagon Devices, Inc., Pullman, Wash.

Metro-Mix®-360 soil was from Sun Gro Horticulture, Vancouver, Canada.

Osmocote™ was from the Scotts Company, Marysville, Ohio.

Fungicide (Maxim 4FS) was from Syngenta, Wilmington, Del.

Thrive® was from Yates (Padstow, NSW, Australia)

Water Crystals were from (Searles®, Kilcoy, QLD, Australia)

1.1 cm and 0.8 cm diameter plastic drinking straws composed of polypropylene were obtained from a local store in Brisbane, Australia.

Cold-water soluble plastic bags were obtained from Extra Packaging Corp, Boca Raton, Fla.

Hot water soluble plastic bags were obtained from Extra Packaging Corp. 736 Glouchester St. Boca Raton, Fla.).

Poly(1,3-propanediol succinate) (177-330 um thick melt-pressed film) was prepared from monomers using the method described in Chrissafis, K. et al. Polymer Degradation and Stabilization 2006, 91, 60-68.

Parafilm® F and Parafilm® M were obtained from Pechiney Plastic Packaging, Chicago, Ill.

Water soluble paper (Aquasol® ASW-60) was obtained from Aquasol Corporation, North Tonawanda, N.Y.

Poly (3-hydroxybutyrate-co-3-hydroxyvalerate) containing 12% valerate comonomer was obtained from Sigma Aldrich, St. Louis, Mo.

Poly(lactic acid) (Ingeo™ 4032D) was obtained from NatureWorks, LLC (Minnetonka, Minn.).

Macozeb was obtained from Searles®.

Crisco™ oil was obtained from J. M. Smucker Co. Orrville, Ohio.

1-naphthaleneacetic acid (NAA, >95% purity) was obtained from Sigma Aldrich.

Perlite and peat moss were obtained from Centenary Landscaping supplies (Darra, QLD)

Poly(ε-caprolactone) was obtained from Sigma Aldrich (St. Louis, Mo.).

Beckosol® 11-035 alkyd resin was obtained from Reichhold Inc (Durham, N.C.).

ε-caprolactone, 3,6-Dimethyl-1,4-dioxane-2,5-dione, and tin (II) 2-ethylhexanoate were obtained from Sigma Aldrich (St. Louis, Mo.).

Kraton® A1535 poly(styrene-b-ethylene-co-butylene-co-styrene-b-styrene) block copolymer was obtained from Kraton Polymers (Houston, Tex.).

Cellulose acetate butyrate (CAB) rigid tubing of 0.625 inch outer diameter and 0.5 inch inner diameter was purchased from McMaster-Carr.

Porous polyethylene (PPE) rigid tubing of 0.75 inch outer diameter, 0.5 inch inner diameter, and 20 μm pore size was purchased from Interstate Specialty Products and cut into 6 inch lengths.

Aminopropyl-terminated PDMS of 900-1100 cSt viscosity was purchased from Gelest (Morrisville, Pa.).

Soybean oil was obtained from MP Biomedicals, (Solon, Ohio).

BD Difco Agar was obtained from VWR.

Phytatray™ II, was obtained from Sigma Aldrich, St. Louis Mo.

Murashige & Skoog (MS) Basal Medium w/ Vitamins was obtained from PhytoTechnology Laboratories (Shawnee Mission, Kans.).

Plant Preservative Mixture™ (PPM) was obtained from Plant Cell Technology, Washington, D.C.

Cobalt (II) napthenate (55 wt % in mineral spirits) was obtained from Electron Microscopy Sciences, Hatfield Pa.

15 mL and 50 mL centrifuge tubes were obtained from VWR, Radnor Pa.

Autoclave tape was obtained from VWR, Radnor Pa.

10 uL disposable loops were obtained from Becton Dickinson and Co., Sparks, Md.

Tetrahydrofuran (THF), hexanes and chloroform solvents were obtained from EMD Chemicals, a branch of Merck KGaA, Darmstadt, Germany.

Poly(acrylic acid), partial sodium salt-graft-poly(ethylene oxide) was obtained from Sigma Aldrich, St Louis, Mo.

Rite in the Rain copier paper was obtained from J.L. Darling Corp, Tacoma, Wash.

Special Mix Coco, Gold Label Special Mix® Substrates was obtained from Gold Label Americas, Olivehurst, Calif.

Tropstrato HT®—potting soil was obtained from Vida Verde, Mogi Mirim, SP, Brazil.

Glycerol and Urea were purchased from Synth, Diadema, SP, Brazil.

Corn Starch (unmodified, 73% amylopectin and 27% amylose), was obtained from Sigma Aldrich.

Antifoaming agent, Hypermaster 602 was supplied from Montenegro Quimicai, Piracaia, SP, Brazil.

Citric acid can be obtained from Sigma Aldrich (St. Louis, Mo.).

Hexamethoxymethylmelamine (HMMM) (Cymel® 303 LF resin) cross-linking agent with an average degree of methylation of 97% was obtained from Cytec, Barcelona, Spain.

Poly(vinyl alcohol) (Elvanol® 52-22) was obtained from E.I. DuPont de Nemours and Company, Wilmington, Del.

Long cellulose fibers were supplied from MD Papeis, Formitex, Caieiras, SP, Brazil.

### Growth Media

Proliferation agar medium contained Murashige and Skoog (MS) basal medium with vitamins (Phytotechnology Laboratories, Shawnee Mission, Kans.) plus 30 g/L sucrose (Grade 1 sucrose, Sigma, St. Louis, Mo.), 8 g/L Difco™ Agar, and 6-benzylaminopurine 0.9 milligram per liter (mg/L) (Phytotechnology Laboratories, Shawnee Mission, Kans.), at pH 5.7).

Regeneration medium, contained MS basal medium with vitamins (Phytotechnology Laboratories, Shawnee Mission, Kans.) plus 30 g/L sucrose and 0.2% Plant Preservative Mixture™ (PPM, Plant Cell Technology, Washington, D.C.), at pH 5.7)

Hoagland's growth medium was prepared as follows: First, individual stock solutions were prepared: 2M KNO₃ (202 grams per liter, g/L); 2M Ca(NO₃)₂×2 H₂O (236 g/L); Iron (Sprint 300 Fe chelate, 38.5 g/L); 2M MgSO₄×7H₂O (493 g/L); 1 M NH₄NO₃ (80 g/L). The micronutrients with phosphate were prepared using: H₃BO₃ (2.86 g/L); MnCl₂×4H₂O (1.81 g/L); ZnSO₄×7H₂O (0.22 g/L); CuSO₄ (0.051 g/L); H₃MoO₄×H₂O (0.09 g/L); 1M KH₂PO₄ (pH to 6.0 with 3M KOH (136 g/L). To prepare Hoagland's growth medium, the stock solutions were combined with about 0.5 L water as follows: 2M KNO₃ (2.5 milliliters, mL); 2M Ca(NO₃)₂ (2.5 mL); Iron (1.5 mL); 2M MgSO₄ (1.0 mL); 1M NH₄NO₃ (1.0 mL); Micronutrient Solution (1.0 mL). Finally, the mixture was diluted to a total volume of 1 L with water.

### Example 1

### Production of Sugarcane Regenerable Plant Tissue, Subsequent Fragmentation and Preparation of Plantlets

The Example below was designed to prepare plantlets that can be used for encapsulation in the paper and plastic containers for production of artificial seeds of sugarcane.

### Week 1—Culture Initiation

1. Sugarcane stalks from 2 to 12-month-old plants of varieties CP01-1372 or KQ228 were cut the day of or one day before the excision of meristematic tissue (hereafter termed explant) for culturing. Leaf blades were trimmed closely, leaving the leaf sheaths intact. The stalks were stored in plastic bags overnight at room temperature if necessary.
2. The stalks were trimmed to get closer to the meristem and then two to three outer leaf sheaths were removed. The stalks were sprayed with 70% ethanol to saturate the outer surface. Ethanol was sprayed to maintain sterility on the surface of each leaf sheath. The stalks were then transferred into laminar flow hoods.
3. Leaf sheaths were then removed to establish the position of the meristem, the stalk was cut 2-3 centimeter (cm) below this point and 2-3 cm above this point and was placed on the sterile surface of a petri dish.
4. Finally, the meristem was split in half longitudinally and the two trimmed halves placed directly onto the proliferation medium. The cut surface was embedded into the medium and petri dishes were sealed with porous filter tape to allow gas exchange and maintain sterility.
5. The explant was grown at 26° C., with light intensity of 30 microEinsteins/m²/s from Philips F32T8/ADV841/XEN 25 watt fluorescent tubes.

### Weeks 2-3: Culture Establishment and Initial Stage of Explant Growth and Proliferation

1. The explants became brown at the cut surfaces due to polyphenols exuding into the medium.
2. Under sterile conditions, the cut ends of explant were trimmed with care taken not to shear off the regenerable tissues from which buds arise. The blackened outer tissue of the explant was removed as needed with minimum tissue excision.
3. The shoots from any lateral buds that arose from the upper side of the section were trimmed.
4. Leaves and shoots were trimmed as necessary.
5. The growing explants were transferred to fresh medium once per week.

### Weeks 4-5: Proliferated Bud Development

1. Once the explants began to proliferate, they were divided into smaller pieces of proliferating buds.
2. The blackened tissue of proliferating buds was removed and each bud piece was given a fresh cut surface for good contact with the fresh proliferation agar medium.
3. The leaves and shoots growing from the buds were trimmed to <1 cm with sterile scissors or scalpels.
4. As much of the original stalk tissue as possible was removed leaving behind only the proliferating buds.
5. The buds were transferred to fresh medium.

### Weeks 5-6: Fragmentation and Plantlet Regeneration

1. Proliferated bud tissue was typically ready for fragmentation and regeneration of plantlets after 7 weeks of growth. However, proliferated buds were occasionally used as young as 6 weeks or as old as 9 weeks after initiation.
2. Fragmentation was done by trimming the proliferated bud masses with scissors to shorten the shoots to 2-3 millimeters (mm).
3. The ‘trimmed’ proliferated buds were then fragmented using sterile scalpels to cut the bud mass into 2-3 mm pieces using a 2 mm grid pattern as a guide.
4. The 2 mm roughly cubic fragments were put directly into the plantlet regeneration medium (MS with 30 g/L sucrose without plant growth regulators).
5. Fragments were cultured in 50-100 mL of liquid regeneration medium in sterile 250 milliliters (mL) polycarbonate flasks with air filters with 15-20 fragments per flask on a rotary shaker at 75 revolution per minute (rpm) to form plantlets.
6. Cultures were incubated at 26° C. with 60 microEinsteins/m²/s light from Philips F32T8/ADV841/XEN 25 watt cool white fluorescent tube in the containers for a period of 2-3 weeks to provide plantlets for use in the artificial seed.

### Example 2

### Encapsulation of Sugarcane Plantlets in Wax Paper Containers to Provide Artificial Seeds

Artificial seeds were constructed as shown in FIG. 1. A cylindrical wax paper container (4) (Aardvark colossal drinking straw, 1.19 cm outer diameter) was cut into 6 cm lengths. A small piece of cotton (6) was inserted at one opening of the wax paper container and the container was autoclaved. In a sterile laminar flow hood, the other opening of the wax paper container, that did not contain the cotton, was stabbed into a Petri dish containing a approximately 1 cm layer of 0.8 weight percent (wt %) Difco™ agar containing MS nutrients, 0.2 wt % Plant Preservative Mixture™ (PPM) and 30 g/L sucrose, twice to get a approximately 2 cm plug of agar (5) that was pushed down, using a thinner wax paper container, onto the cotton layer in the wax paper container. Sugarcane plantlets, which had been regenerated (3) from proliferated bud tissue fragments in plantlet regeneration medium for 14 days post-fragmentation (as described in Example 1) were placed on top of the agar and then both openings of the wax paper container were secured (1) with manually pre-stretched Parafilm® M to provide artificial seeds.

The artificial seeds were planted in autoclaved vermiculite in 10 cm plastic pots with plastic trays underneath to collect water, and oriented vertically, so that the top openings of the artificial seeds were about 0.5 cm above the vermiculite surface. The artificial seeds were left in a walk-in growth chamber (Conviron model BDW-120) at 22° C. (day) and 20° C. (night), with 16 hours photoperiod at 220 uE/m²/s. The vermiculite was watered daily with filtered distilled water and the pots were covered with a clear plastic dome.

After 6 days in the growth chamber, one sugarcane plantlet began to sprout (leaf protruding) through the Parafilm® M top closure. After 13 days, 3 of the artificial seeds had plantlets sprouting through the top closure and one plantlet had sprouted roots through the bottom closure. The clear plastic dome was removed from the pots containing the sprouted artificial seeds and they were watered with half-strength Hoagland's nutrient medium. After 17 days, a fourth artificial seed had a plantlet sprouting through the top. When the experiment was stopped at 38 days, 4 of the 6 artificial seeds without sprouted plantlets contained live plants inside the container. In another un-sprouted artificial seed fungal growth was observed, although the tissue was still green and alive. The 4 plantlets that had sprouted continued to grow and appeared healthy

### Example 3

### Comparison of the Effect of Flat Openings Versus Crenellated Openings in Artificial Seeds on the Growth of Plantlets

This Example was designed to study the effect of crenellation at the bottom opening of the artificial seeds on improving root penetration. Wax paper containers were cut to 4 cm lengths with flat top and bottom openings, and compared to 5 cm paper containers with crenellated openings. The crenellation was the result of cutting three, 1 cm long and 3-4 mm wide tabs out of one opening of the container (FIG. 2) Crenellation was only used at the bottom opening of the wax paper container. A comparison was also made between the presence and absence of agar in artificial seeds on the growth of the tissue in this Example. Artificial seeds (with or without crenellation and with or without agar) were constructed in a laminar flow hood. In this experiment, agar had the same composition as described in Example 2, except that 20 g/L sucrose was used instead of 30 g/L sucrose. Manually pre-stretched Parafilm® M was used to enclose the top and bottom openings of the containers after sugarcane plantlets, which had been regenerated from meristem tissue fragments (variety CP01-1372) in regeneration medium for 15 days post-fragmentation were added to the wax paper containers. No cotton was used in this experiment.

The artificial seeds were planted in a growth chamber (Conviron CGR-962) at 31° C. day, 22° C. night, 14 hours photoperiod, 220 uE/m²) in Metro-Mix®-360 soil, in 10 cm plastic pots with a tray on the bottom and clear plastic closures on top. Initially, at day 0, the soil was watered at 100 mL per 10 cm pot, and was watered the same amount weekly thereafter. Osmocote™ fertilizer granules were applied to the soil as recommended by the manufacturer. Table 1 summarizes the results of this experiment.

TABLE 1
Effect of crenellated openings and the presence or absence of agar
on the sprouting of sugarcane from the artificial seeds

| | | % | % Sprouts 10 |
|---|---|---|---|
| | Initial # of | Sprouting | cm or taller at |
| | containers | by day 23 | day 23 |
| Flat opening - agar | 10 | 90 | 50 |
| Crenellated opening - | 10 | 100 | 50 |
| agar | | | |
| Flat opening - no agar | 6 | 67 | 33 |
| Crenellated opening - | 4 | 50 | 50 |
| no agar | | | |

As shown in Table 1, crenellation at the opening of the container had no substantial effect on sprouting or growth of the plant tissue in artificial seeds. There appeared to be a slight detrimental effect of omitting agar on the sprouting of the plant tissue.

### Example 4

### Effect of Planting Artificial Seeds at the Surface or Slightly Deeper in the Vermiculite

Cylindrical wax paper containers (5 cm long, 1.19 cm diameter) were prepared with flat openings, autoclaved, and stabbed onto agar as described in Example 2. Sugarcane plantlets, which had been regenerated from meristem tissue fragments (variety KQ228) in plantlet regeneration medium for 14 days post-fragmentation were placed into the containers with the top opening secured by pre-stretched Parafilm® M. The artificial seeds thus prepared were planted in vermiculite, either with slight protrusion above the surface (<0.5 cm), or with slight burial below the surface (<0.5 cm). The artificial seeds were incubated in 10 cm plastic pots in a walk-in growth chamber at 31° C. during the day, 22° C. during the night, and 14 hours photoperiod, 220 uE/m²). The results of this experiment are shown in Table 2.

TABLE 2
Effect of burial of the artificial seeds in vermiculite on sprouting
and plant growth. The presence of shoots or roots protruding out
through the Parafilm ® M closure is indicated at each time point.

| | Slight burial below the | | Slight protrusion above the | |
|---|---|---|---|---|
| | surface (<0.5 cm) 5 artificial | | surface (<0.5 cm) 5 artificial | |
| | seeds planted initially | | seeds planted initially | |
| days after | # | # with roots | # | # with roots |
| planting | sprouting | emerging | sprouting | emerging |
| 5 | 3 | ND | 1 | ND |
| 7 | 4 | ND | 3 | ND |
| 9 | 4 | ND | 3 | ND |
| 12 | 4 | 4 | 4 | 0 |
| 22 | 4 | 4 | 4 | 1 |

ND = not determined.

Overall, no substantial differences in sprouting and growth of the plantlets were observed among the artificial seeds that had been buried versus those that had been set on the surface of the vermiculite.

### Example 5

### Effect of Fungicides on Viability of Plantlets in Artificial Seeds

This experiment was performed to study the effect of fungicides on the prevention of fungal attack and on the viability of the plantlets in the artificial seeds. Crenellated cylindrical wax paper containers (5 cm total length, as in Example 3) were utilized. A solution of fungicide, Maxim 4FS, was created by dispersing 80 mg Maxim 4FS in 30 mL deionized (DI) water, followed by the addition of 70 mL of 70% ethanol. The resulting solution was clear. Finally, twelve containers were immersed in this solution in a laminar flow hood for approximately 1 minute (min), followed by drying on a sterile paper towel for 30 min. A control set of 17 containers were immersed in 70% ethanol and allowed to dry in the laminar flow hood. The containers were assembled with the standard agar medium as described in Example 2 and plantlets (cultivar CP01-1372), which were cultured for 14-days from proliferated bud tissue fragments, were inserted into the growth medium. Both openings of the artificial seeds thus prepared were secured with pre-stretched Parafilm® M.

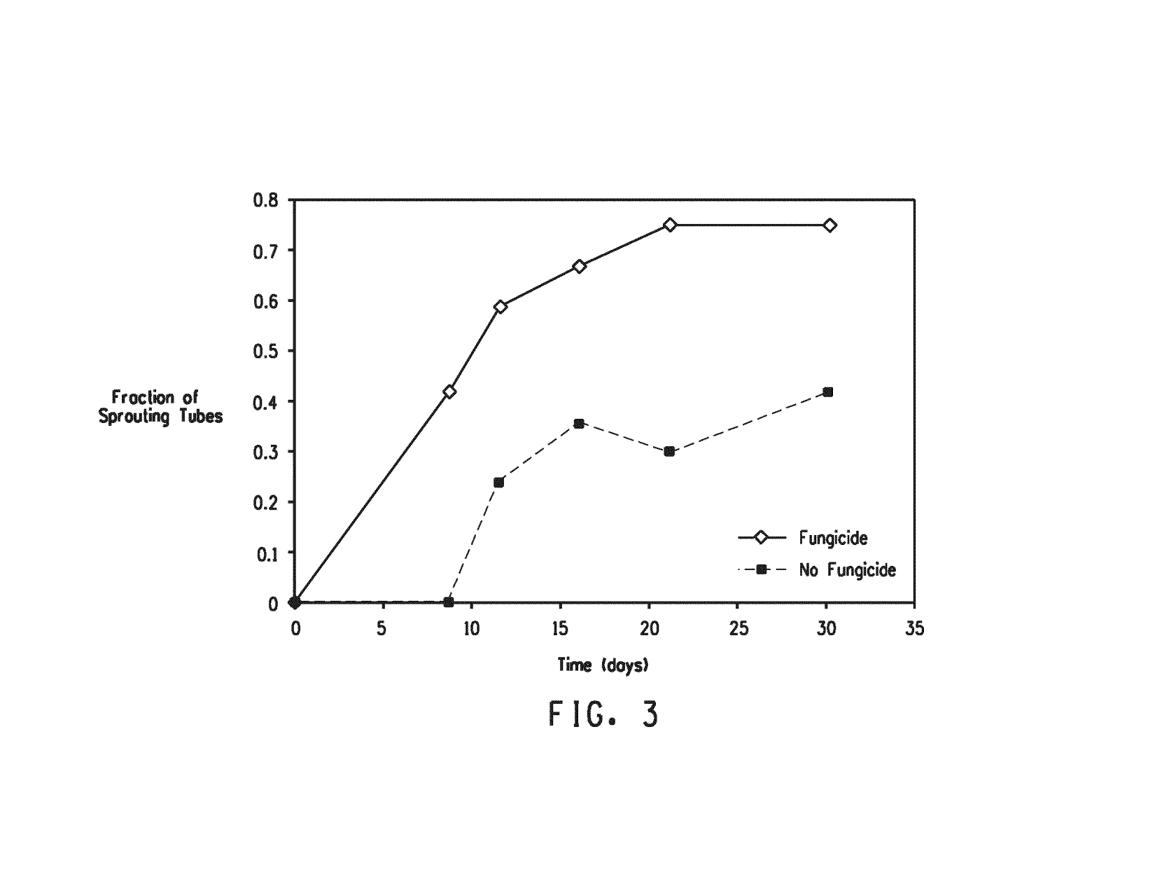

The artificial seeds were planted in Metro-Mix® 360 in 10 cm plastic pots with clear plastic dome and trays in a growth chamber at 31° C. during the day, 22° C. during the night, and a 13 hr photoperiod (220 uE/m²). Watering was performed from the bottom, at approximately 100 mL/pot/week. Algal growth was observed on the surface of the soil after 9 days, indicating the high moisture content of the soil. Soil moisture (measured with a Decagon EC-5 probe) ranged from ˜0.3-0.7 cubic meters per cubic meters (m³/m³) volumetric water content over the course of the experiment. The number of plantlets sprouting out of the top opening of the artificial seeds was monitored periodically by visual inspection. As can be seen in FIG. 3, the presence of the fungicide had a substantial effect on improving sprouting from the artificial seeds (represented by the curve with white squares in FIG. 3) compared to those artificial seeds that did not contain the fungicide (represented by black squares in FIG. 3). Furthermore, plant vigor appeared to be lower in the fungicide-free set. Upon completion of the experiment, the artificial seeds were removed from the soil and their contents were examined. A substantially larger amount of fungal growth was observed on the inner surface of the artificial seeds that had not been treated with fungicide compared to the fungicide-treated samples.

### Example 6

### Suitablility of Various Top Closure Materials for Application in Artificial Seeds

This experiment was conducted to screen a series of alternate materials to be used as top closure for the artificial seeds using the wax paper containers. Cylindrical wax paper containers (4 cm total length) with crenellation at the bottom opening were used and were assembled as described in Example 5, with the exception of the material used for the top opening closure. The bottom opening closures in this experiment were composed of pre-stretched Parafilm® M. The containers were also soaked in the fungicide Maxim 4FS solution prior to assembly as described in Example 5. Top closure materials used in this test included cold-water soluble plastic bags based on poly(vinyl alcohol) (Extra packaging), hot water soluble plastic bags also based on poly(vinyl alcohol) (Extra packaging), poly(1,3-propanediol succinate) (177-330 micron thickness melt-pressed film), pre-stretched Parafilm® F and pre-stretched Parafilm® M. The cold-water soluble bag film was attached to the top opening of the paper container using household silicone caulk while the poly(1,3-propanediol succinate) was attached using a hot glue gun. The containers were assembled with the standard agar medium described in Example 2 and 15-day-old liquid culture-derived plantlets were used (cultivar CP01-1372). The artificial seeds were planted in Metro-Mix® 360 in 10 cm plastic pots with trays without clear plastic domes in a growth chamber (Conviron model BDW-120) at 31° C. during the day, at 22° C. during the night, and a 13 hr photoperiod (220 uE/m²). Watering was performed from the bottom, at approximately 100 mL/pot/week. Soil moisture (measured with a Decagon EC-5 probe) ranged from ˜0.3-0.6 m³/m³ volumetric water content over the course of the experiment. Sprouting of the plantlets from the top of the artificial seeds was monitored by visual inspection over the course of the experiment and the results are shown in Table 3.

TABLE 3
Effect of various materials used to secure the top opening
of the artificial seeds on sprouting of plants

| | Number of | | |
|---|---|---|---|
| Top Closure | artificial seeds | Number sprouting | Percent sprouting |
| Material | planted | at day 47 | at day 47 |
| cold-water soluble | 10 | 10 | 100% |
| plastic bag film | | | |
| hot water soluble | 10 | 3 | 30% |
| plastic bag film | | | |
| poly(1,3- | 10 | 1 | 10% |
| propanediol | | | |
| succinate) | | | |
| Parafilm ® F | 10 | 9 | 90% |
| Parafilm ® M | 10 | 9 | 90% |

As can be seen from the above table, the cold-water soluble plastic top closures provided the best sprouting performance, and were comparable to Parafilm® M and Parafilm® F. Closures that were stronger, or less sensitive to moisture (hot water soluble plastic bags and poly(1,3-propanediol succinate)) produced a lower percentage of sprouting plantlets.

### Example 7

### Screening the Bottom Closure Materials for Artificial Seeds

This experiment was performed to screen various materials for the bottom opening closure for the wax paper container for preparation of artificial seeds. In this case, flat-ended 4 cm long wax paper cylindrical containers were prepared. The containers were also soaked in Maxim 4FS solution prior to assembly as described in Example 5. Parafilm® M was used as the top closure in all tests. Bottom closure materials included the same materials described in Example 6, with the addition of a water soluble paper (Aquasol® ASW-60), composed of sodium carboxymethyl cellulose, which had been wax-impregnated. The wax impregnation was performed by soaking the water soluble paper sheet in a 12 weight percent (wt %) solution of paraffin wax (mp 53-57° C.) in cyclohexane, and allowing the solvent to evaporate in a fume hood for 18 hr at 20-25° C. The wax impregnation was intended to slow the dissolution rate of the water soluble paper. Additionally, poly (3-hydroxybutyrate-co-3-hydroxyvalerate including 2% valerate co-monomer) was investigated as one of the bottom opening closure materials. This closure was prepared by melt pressing pellets of polymer into a 125-177 micrometer (nm) thick film. These new materials were attached to the bottom of the container using a hot glue gun. On top of the moisture sensitive closure materials (e.g., cold and hot water soluble bags and wax-impregnated water soluble paper) a few drops of molten Crisco™ oil (T approximately 60° C.) was applied to prevent the moisture from the agar plug, inside the wax paper container, from dissolving or softening the bottom opening closure prior to adding the regenerable tissue. The Crisco™ oil was allowed to cool and harden before agar plug was added. The artificial seeds were assembled with the standard agar medium as described in Example 2 and 20-day old liquid cultured regenerable sugarcane tissues were used (cultivar KQ228). The artificial seeds were manually placed into depressions made in Metro-Mix® 360Metro-Mixt 360 in 10 cm plastic pots with trays (without clear plastic domes) in a growth chamber (Conviron model BDW-120) at 31° C. during the day, 22° C. during the night, and a 13 hr photoperiod, 220 uE/m²). Watering was performed from the bottom, at approximately 100 mL/pot/week. Soil moisture was measured with a Decagon EC-5 probe and it ranged from ˜0.3-0.6 m³/m³ volumetric water content over the course of the experiment. Sprouting of the plantlets from artificial seeds was monitored by visual inspection over the course of the experiment and the results are shown in Table 4.

TABLE 4
Examination of various materials for application as the
bottom opening closure in artificial seeds

| | Number of | | |
|---|---|---|---|
| Bottom opening | artificial seeds | Number sprouting | Percent sprouting |
| Closure Material | planted | at day 45 | at day 45 |
| Cold-water soluble | 10 | 3 | 30% |
| plastic bags | | | |
| Hot water soluble | 11 | 1 | 9% |
| plastic bags | | | |
| poly(1,3- | 6 | 3 | 50% |
| propanediol | | | |
| succinate) | | | |
| Wax-impregnated | 12 | 9 | 75% |
| ASW60 | | | |
| Parafilm ® M | 18 | 4 | 22% |
| poly(3-hydroxy | 10 | 2 | 20% |
| butyrate-co-3- | | | |
| hydroxy valerate) | | | |

Results summarized in Table 4 indicate that the wax-impregnated water-soluble paper ASW 60 and poly(1,3-propanediol succinate) outperformed Parafilm® M as the bottom opening closure material, under the test conditions. Cold-water soluble plastic bags and poly(3-hydroxy butyrate-co-3-hydroxy valerate) closures performed comparably to Parafilm® M.

### Example 8

### Screening Materials to be Used for the Assembly of the Container of Artificial Seeds

This experiment was performed to screen various materials to be used for the assembly of the cylindrical container of the artificial seed. When wax paper was used as the material, 4 cm long wax paper containers with flat-openings were prepared. These paper containers were soaked in Maxim 4FS solution prior to assembly. The other material tested was poly(3-hydroxy butyrate-co-3-hydroxyvalerate). The container with this material was prepared by melt pressing pellets of polymer into a 125-177 μm thick film. Another material tested was poly(lactic acid) (Ingeo™ 4032D, NatureWorks, Minnetonka, Minn.), which was melt pressed into a film with thickness ranging from 245-490 μm. These two plastic film materials were manually wrapped into single-walled containers of similar length and diameter to the wax paper, and attached using a hot glue gun. For all containers in this experiment, pre-stretched Parafilm® M was used for both the top opening and the bottom opening closures. The plastic film materials were not treated with fungicide.

The artificial seeds were assembled with the standard agar medium described in Example 2 and 20-day-old regenerable sugarcane tissues were used (cultivar KQ228). The artificial seeds were planted in Metro-Mix® 360 in 10 cm plastic pots with trays (no clear plastic domes) in a growth chamber (Conviron model BDW-120), at 31° C. during the day, at 22° C. during the night, and a 13 hr photoperiod, (220 uE/m²). Watering was performed from the bottom of the artificial seeds, at approximately 100 mL/pot/week. Soil moisture was measured with a Decagon EC-5 probe and it ranged from ˜0.3-0.6 m³/m³ volumetric water content over the course of the experiment. Sprouting of tissues from artificial seeds was monitored by visual inspection over the course of the experiment as shown in Table 5.

TABLE 5
Comparison of the effect of various materials used for the assembly
of artificial seeds on the sprouting of sugarcane plantlets

| | Number of | | |
|---|---|---|---|
| | artificial seeds | Number sprouting | Percent sprouting |
| Body Material | planted | at day 45 | at day 45 |
| Poly(lactic acid) | 10 | 0 | 0% |
| Wax paper | 18 | 4 | 22% |
| poly(3-hydroxy | 7 | 1 | 14% |
| butyrate-co-3- | | | |
| hydroxy valerate) | | | |

Results summarized in Table 5 indicate that a higher percentage of the tissues sprouted from wax paper container artificial seeds compared to those made of either poly(lactic acid) or poly(3-hydroxy butyrate-co-3-hydroxy valerate). After the experiment was completed, the containers were removed from the soil for inspection (day 55). The poly(lactic acid) artificial seeds showed no signs of degradation, whereas only slight decomposition was observed in the case of the poly(3-hydroxy butyrate-co-3-hydroxy valerate). On the other hand, containers from the artificial seeds prepared with wax paper showed high levels of degradation.

### Example 9

### Effect of Eliminating Airspace in the Containers of the Artificial Seed

The purpose of the experiment was to study the effect of completely filling the cylindrical wax paper container with the agar medium described above, thereby eliminating the airspace within the artificial seed. This was compared to the standard design in which airspace was left above the plantlet. Wax paper containers (Aardvark “Colossal” wax paper straws) were cut to 4 cm lengths with flat openings. In this experiment, agar had the same composition as in Example 2, except 0.6% Difco agar was used instead of 8 g/L agar. For the wax paper straws which were completely filled, a ˜3 cm layer of agar was added to the straw, followed by pushing in the plantlet and finally adding more agar to the top of the wax paper straws. The control containers were made with a ˜2 cm agar plug. Manually pre-stretched Parafilm® M was used to secure both openings of the container after the 14 day old sugarcane plantlets (CP01-1372) were introduced to complete the artificial seed.

The artificial seeds were planted in a growth chamber (Conviron CGR-962, 31° C. day, 22° C. night, 14 hr photoperiod (220 μE/m²) in Metro-Mix®-360 soil with 0.5 wt % Osmocote™ 17 in 10 cm plastic pots with a tray on bottom and clear plastic lid on top (removed at 10 days). Initially, the soil was watered at 100 mL per 10 cm pot, and was watered the same amount weekly thereafter. Results are given below in Table 6.

TABLE 6
Effect of filling containers on sprouting and growth.

| | | | | Approximate |
|---|---|---|---|---|
| | | | | height of |
| | Artificial seed | # Sprouted | # Sprouted | plants |
| | planted | by day 10 | by day 17 | day 35 (cm) |
| Full—no airspace | 10 | 5 | 9 | 5-12.5 |
| Control—with | 10 | 9 | 10 | 20-45 |
| airspace | | | | |

Results summarized in Table 6 indicate that filling the containers with agar had a detrimental effect on the sprouting rate, growth rate and final size of the sugarcane plantlets of the artificial seeds.

### Example 10

### The Age of Regenerated Plantlet Determines Sprouting Frequency of Plantlets in Artificial Seeds

### Preparation of Sugarcane Plantlets and Artificial Seeds

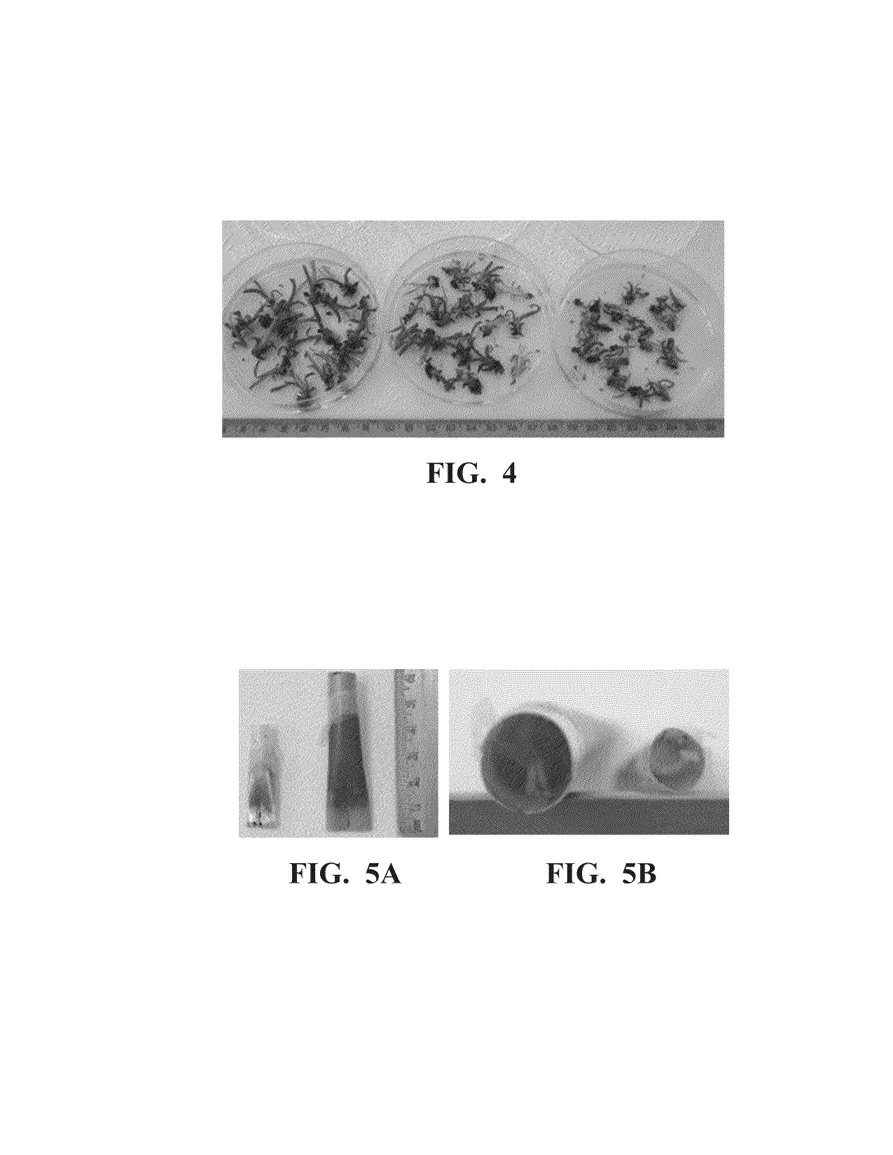

In this experiment 3 mm meristem tissue fragments of sugarcane variety KQ228 were produced under the same experimental conditions as described in Example 1 except at noted below. They were grown in plantlet regeneration medium (liquid culture) for 10, 15 and 20 days to generate plantlets of different ages for encapsulation in artificial seeds. Since the 10-day-old plantlets were very small and not well developed, all cultures were transferred to an MS medium solidified with agar (6 g/L) and grown for another 10 days. Cultures were maintained in a growth chamber set at 26° C. with 16 h photoperiod (30 microEinsteins/m²/s from Philips 25 watt fluorescent tubes). From each of the three ages, plantlets of 1-1.5 cm length were separated and designated Group 1, whereas the larger plantlets were trimmed to 1.6-2.0 cm length and designated as Group 2 (FIG. 4). Artificial seeds were constructed with cylindrical plastic containers of two different sizes (4.0 cm length×0.8 cm diameter and 6.0 cm length×1.1 cm diameter; open at both ends), produced from commercially available plastic cylinders (polypropylene drinking straws, obtained from a local store in Brisbane, Australia). The smaller containers received 1-1.5 cm long plantlets (Group 1) while the larger ones had 1.5-2 cm long plantlets (Group 2). With the plantlet age and container size combinations a total of six different combinations [2 cylindrical containers sizes (4 and 6 cm long)×3 plantlet ages (10, 15, 20 days in liquid regeneration medium and followed by 10 days on agar medium)] were tested. The bottom opening of the plastic container was kept partially closed by stapling. This assembly facilitated unimpeded root growth. Other components and the steps involved in constructing the artificial seeds according to this Example are detailed below.

The plastic container was packed ¾th of the volume with garden soil (commercially sold as top dressing for plant nurseries) with one 2 mm long water crystal (Searles® Water Crystals), a highly hygroscopic synthetic compound capable of holding large amounts of water and nutrients, placed in the middle of the soil column. Sugarcane plantlets as prepared above were then inserted into plastic containers in such a way that at least half the plantlet length was buried in the soil, leaving the remaining portion exposed to the airspace. Next, liquid MS nutrients containing 1 g/L media Mancozeb (Searles®), a commercial general purpose fungicide was added to the soil to full water holding capacity. The construction of the artificial seed was completed by securing the top opening with fully stretched Nescofilm® (Bando Chemical Industries, Japan), a flexible, moisture proof, thermoplastic sealing film (FIG. 5).

### Planting Artificial Seeds in Greenhouse

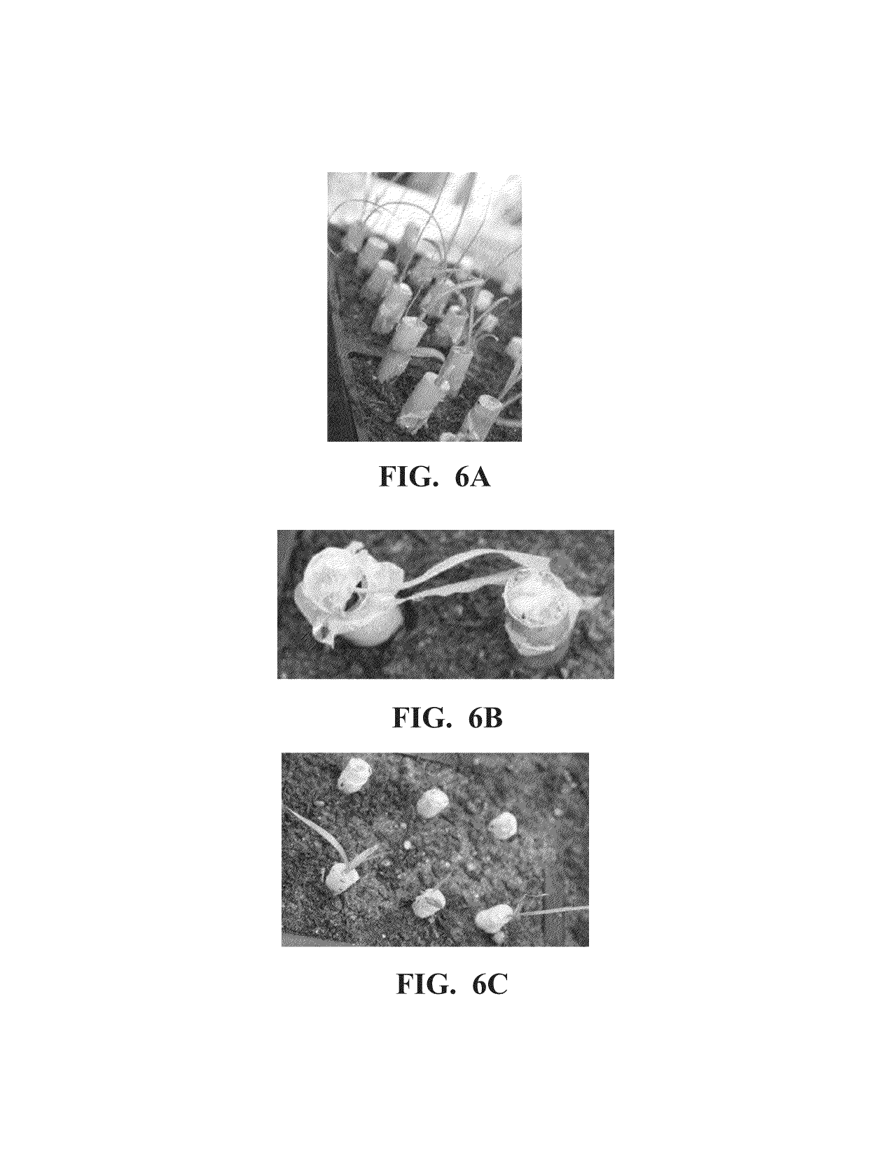

Artificial seeds prepared as described above were planted in plastic seedling trays (35×29 cm; 64 wells, 4 cm deep) containing garden soil (commercially available top dressing) filled to the top. Each artificial seed was planted in such a way that at least 1 cm of small artificial seeds (4 cm long) and 2 cm of larger ones (6 cm long) were kept above the soil (FIG. 6). At least 20 artificial seeds were planted for each type of container construct. As a control treatment, 20 plantlets from group 1 of each culture age (10, 15, 20 days in liquid regeneration medium and 10 days on agar medium thereafter), were planted directly in soil. All trays were covered with transparent plastic flat sheets for 12 days. The trays were irrigated to full field capacity twice weekly alternately with tap water and Thrive® (4 g/9 L), a commercially available general purpose nutrient preparation.

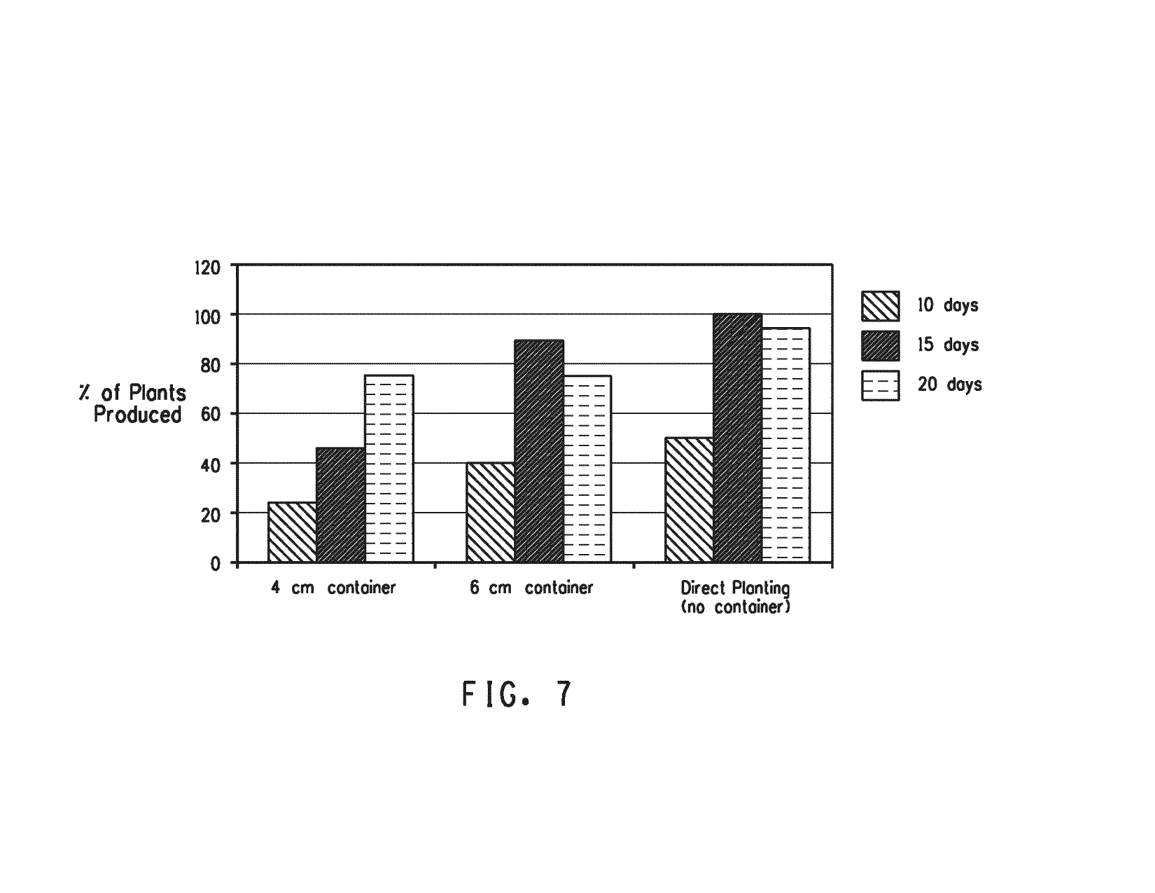

The percentage of sprouted plants from artificial seeds (defined as those with shoots which emerged through the Nescofilm®) was recorded three weeks after planting (FIG. 7). The data show that the age of the plantlet in the artificial seeds played a significant role in its survival and sprouting ability. In this test, after 3 weeks, the sprouting percentage of the plantlets that had been initially grown in liquid cultures for 20 days (FIG. 7, light grey column), was substantially higher (at least 70% growth) irrespective of whether they had been encapsulated in an artificial seed (FIG. 7, medium grey and dark grey columns) or whether they had been planted directly in the soil. The percentage survival of plantlets obtained from a 10-day liquid culture ranged from 25-40% in artificial seeds and slightly higher when planted directly in the soil. The high rate of mortality in artificial seeds was due to the high incidence of fungal contamination.

### Example 11

### Comparison of Different Planting Substrates in Plantlet Survival and Emergenece from Artificial Seeds

### Preparation of Sugarcane Plantlets and Artificial Seeds

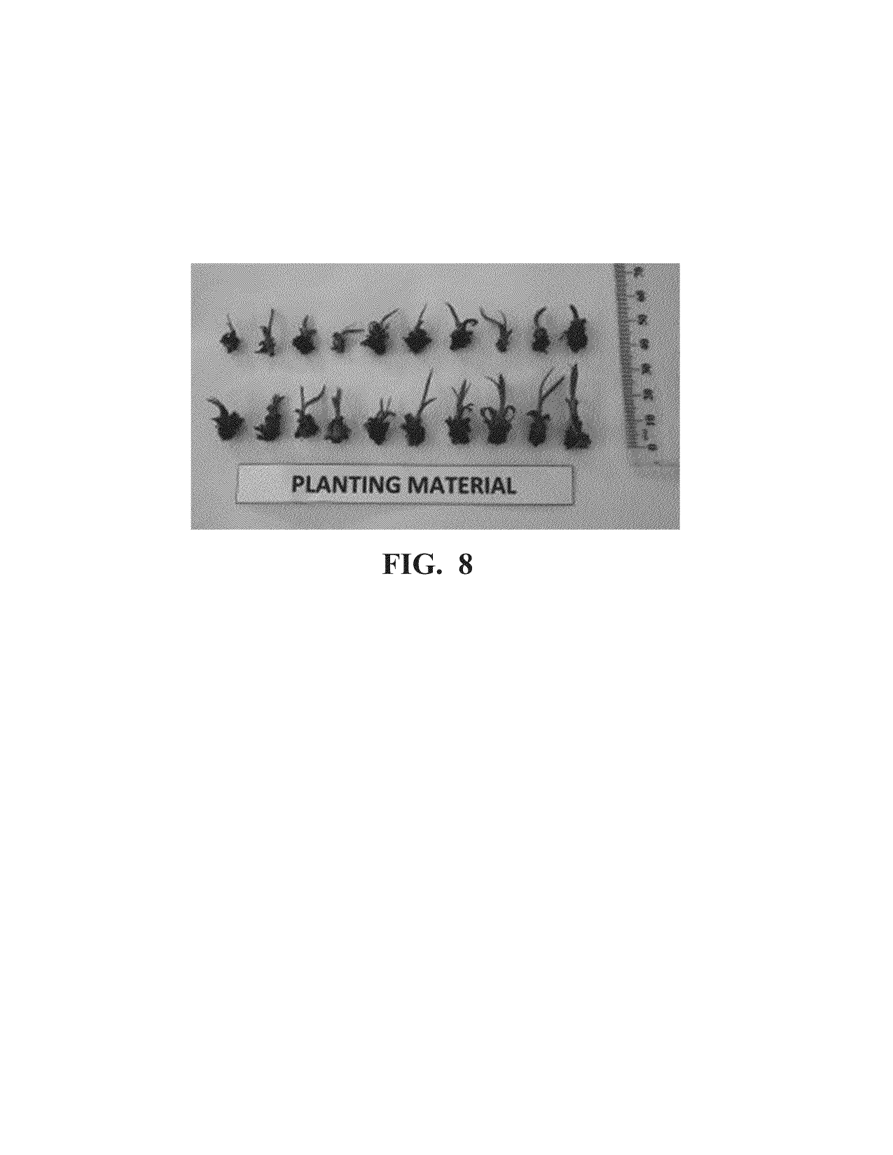

This experiment was performed to determine whether soil-less medium (e.g. Perlite, peat moss and Searles® water crystals) improved survival and growth of plantlets in artificial seeds. Sugarcane variety KQ228 was used. Preparation of tissue fragments was similar to that described in Example 1. Proliferated bud fragments (3 mm) were then grown in the plantlet regeneration medium with the addition of 2 μM NAA for 17 days to prime them for rooting. At the end of this stage the size of the plantlets formed ranged from 1.2-3.2 cm. These plantlets were used for encapsulation in the artificial seeds. For most of them root development was not visible (FIG. 8).

Plastic cylindrical containers (6 cm long, 1.1 cm diameter, polypropylene drinking straws) were used in this experiment and they were prepared following the procedure described in Example 10 except that 5 different compositions (T), as listed below, were used in this experiment. The composition of the treatments were: in T1 the plantlet was planted directly in the soil without use of a seed container; in T2 the artificial seed contained garden soil (similar to that used in Example 10); in T3 the artificial seed contained garden soil, and water crystals (1 g dry crystals per L of soil; Searles®); in T4 the artificial seed contained peat moss and perlite mix in equal volume plus water crystal (1 g dry crystals per L of soil); in T5 the artificial seed contained water crystal only. Ridomil (1 g/L soil) fungicide and Thrive® (Yates) nutrients, supplied as liquid (0.44 g/L solution), had been added to all treatments.

### Experimental Details

At least 30 artificial seeds were prepared for each treatments and a similar number of plantlets were also used for direct planting (control). About 75% of the volume of each container was filled with soil-less medium and the containers were irrigated to full field capacity with Thrive® solution. Experiments were performed in plastic trays (500 mm×380 mm×80 mm) perforated with 20 holes (1 cm in diameter) in a glasshouse with no environmental control. All trays were lined with 2 layers of paper towels and then filled with garden soil, and moistened with water to full field capacity. Artificial seeds were planted with their top openings kept at least 1 cm above the soil. All treatments were irrigated with water on day 0 and once every week thereafter. All treatments were fertilized on day 0 and then fortnightly with 2.0 L per tray of Thrive® (4 g/9 L). The number of plantlets surviving in the control (T1) and shoot emergence in treatments (T2-T5) were recorded on day 7, 14, 21, 28, 31, 40 and 63.

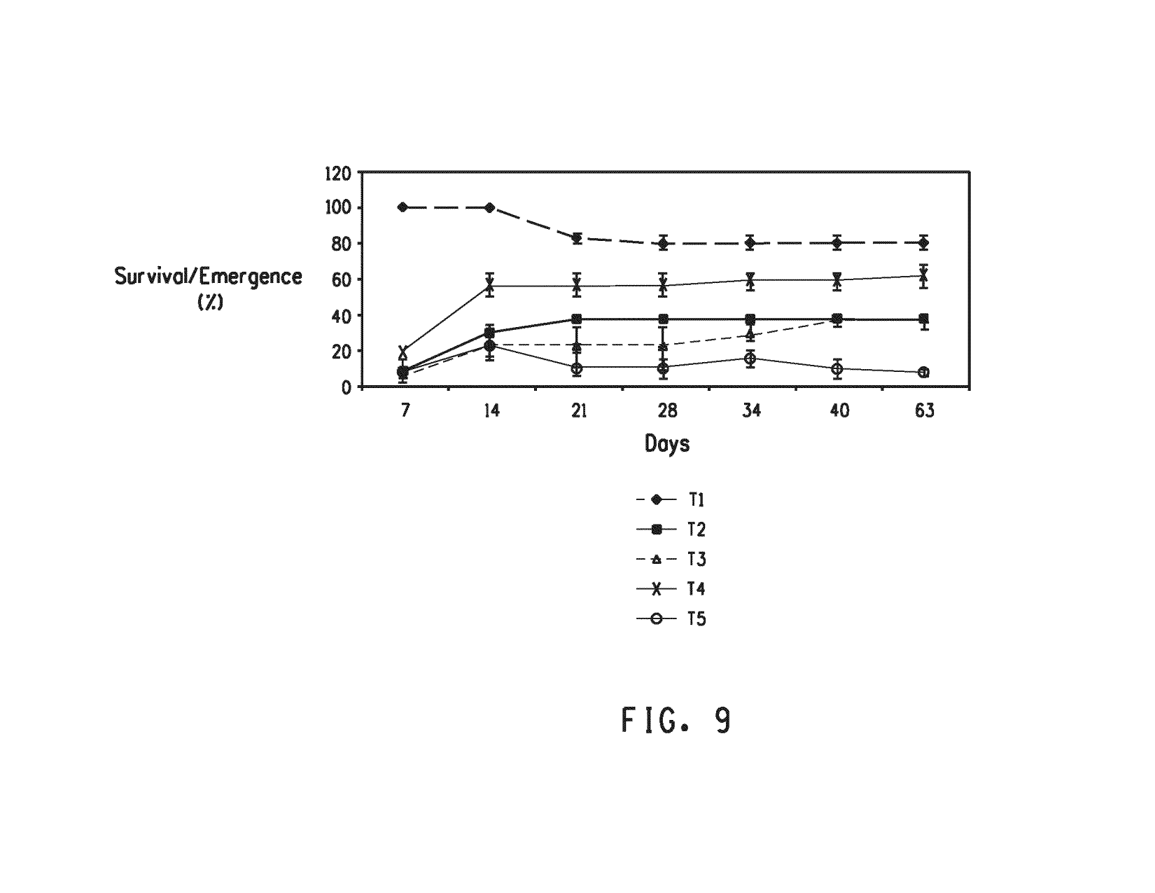

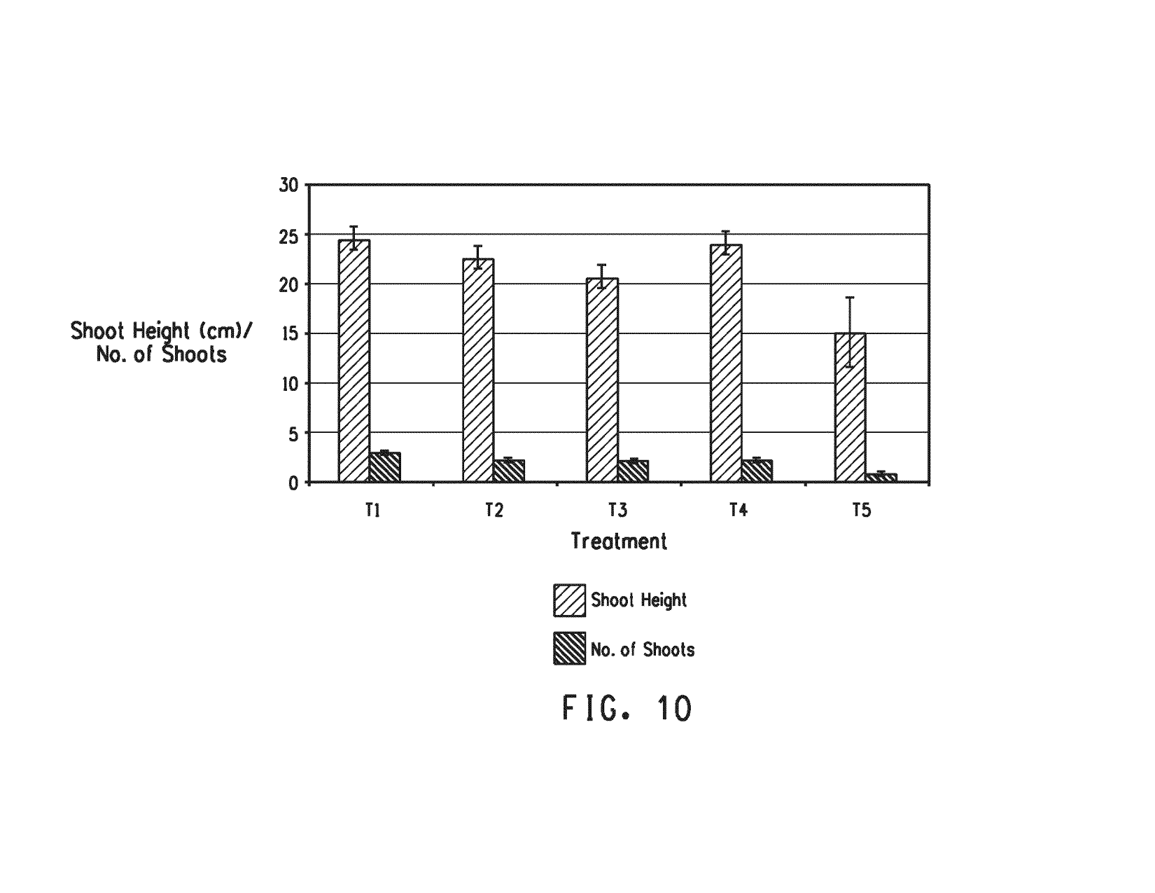

Results summarized in FIG. 9 indicate that the highest survival (80%) was observed with the control T1 in which the plantlet was planted directly in the soil without a container. The T4 container, with peat moss and perlite mix in equal volume plus water crystal, had the highest sprouting percentage of plantlets (63%) amongst the tests with containers followed by T2, containing only garden soil (37%), and T3, containing garden soil and water crystals (33%). T5, containing only water crystals, demonstrated the lowest sprouting (7%).

Results summarized in FIG. 10 indicate that shoot height and number of shoots was similar in T1-T4, but were significantly lower for T5 treatment.

The results of this test demonstrate that the combined use of water crystals with other substrates such as peat moss and perlite is a good choice for establishing plantlets without well-developed roots.

### Example 12

### Performance of Artificial Seeds in the Field

### Preparation of Artificial Seed Constructs

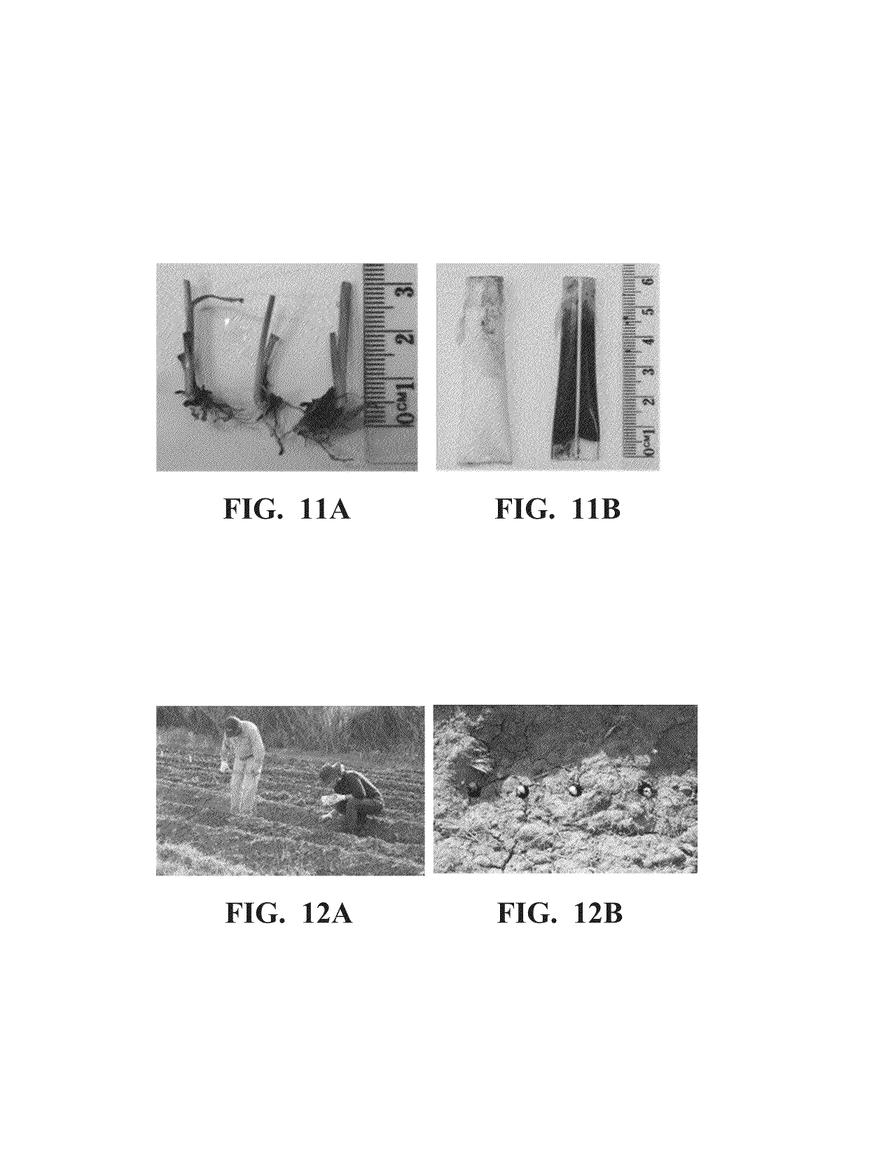

This experiment was designed to demonstrate sprouting and successful establishment of plantlets derived from sugarcane artificial seeds. Sugarcane variety KQ228 was used in this experiment. Preparation of the artificial seeds was similar to that described in Example 10, except that both plastic and wax paper (Aardvark colossal drinking straw, 1.19 cm diameter) cylindrical containers were employed for comparison. The plastic containers were 6 cm long 1.1 cm diameter with bottom opening stapled for partial closure. In addition, the garden soil mix used was supplemented with the fungicide Ridomil (1 g per L of soil) and Searles® Water Crystals (1 g dry crystals per L of soil), and saturated with half-strength liquid Thrive® (Yates). Water crystals were pre-hydrated with half-strength Thrive® (2 g/9 L) and then mixed with the soil prior to preparing the construct. Sugarcane plantlets were cultured for 2-3 weeks in liquid culture and then cultured for an additional 4-6 weeks old (plantlets were grown on agar with 30 g/L sucrose and MS nutrients; FIG. 11), and were trimmed to 3-4 cm height prior to insertion into the containers. Both the shoots and roots of the plantlets were trimmed. Plantlets were placed about 1.5 cm deep in soil in the container.

### Planting in the Field

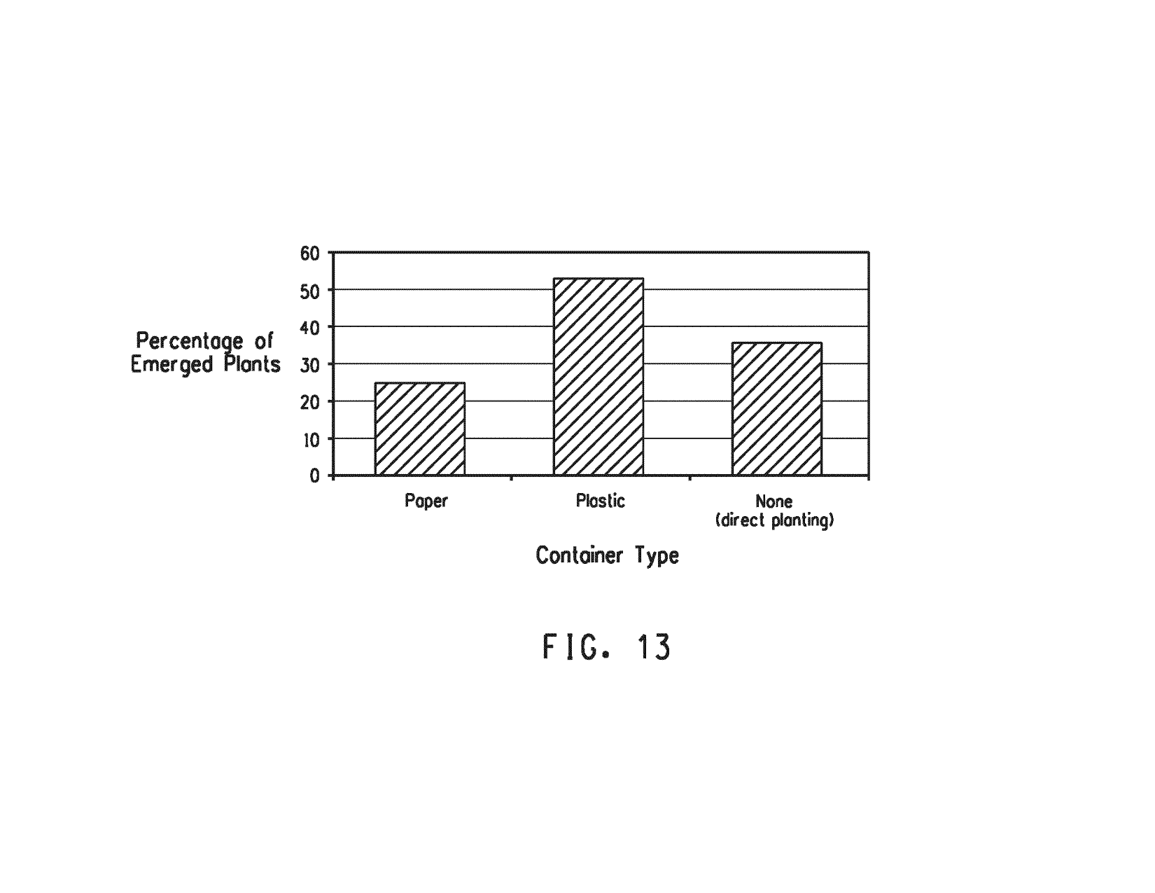

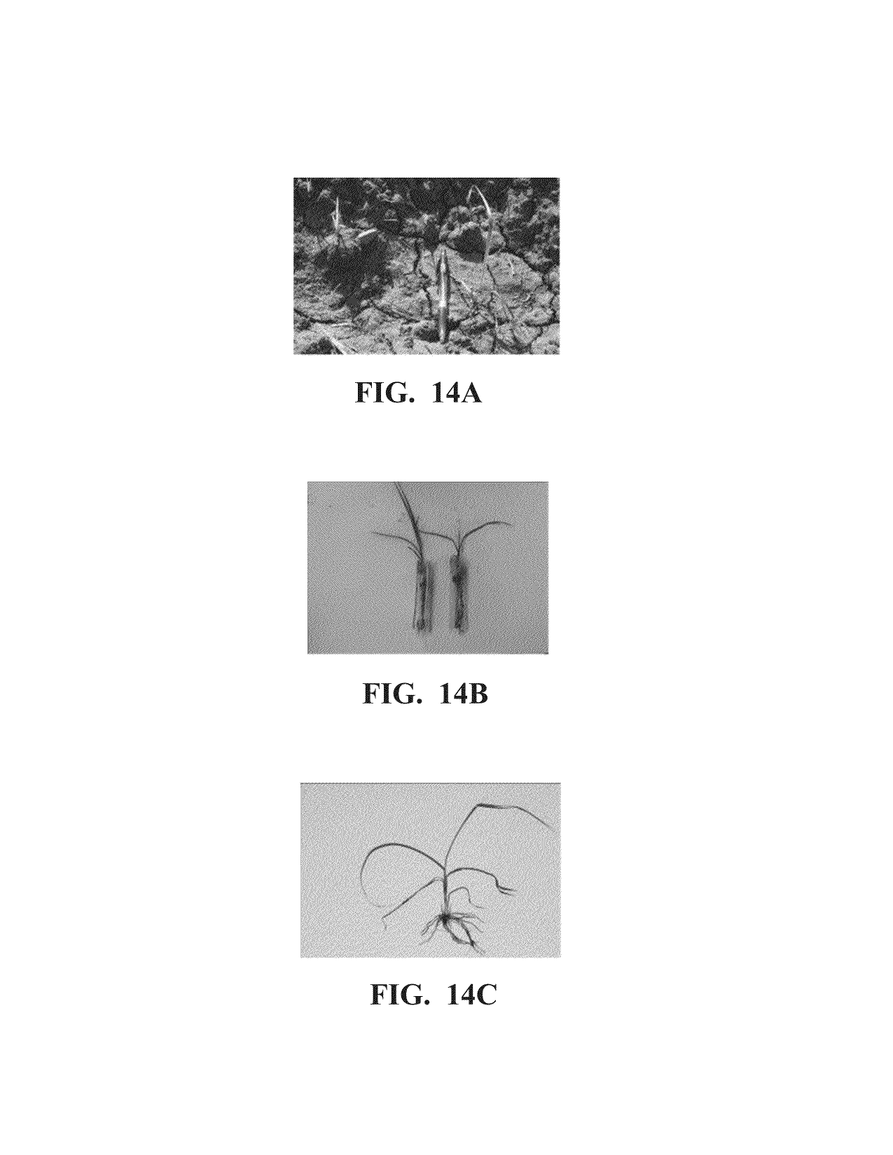

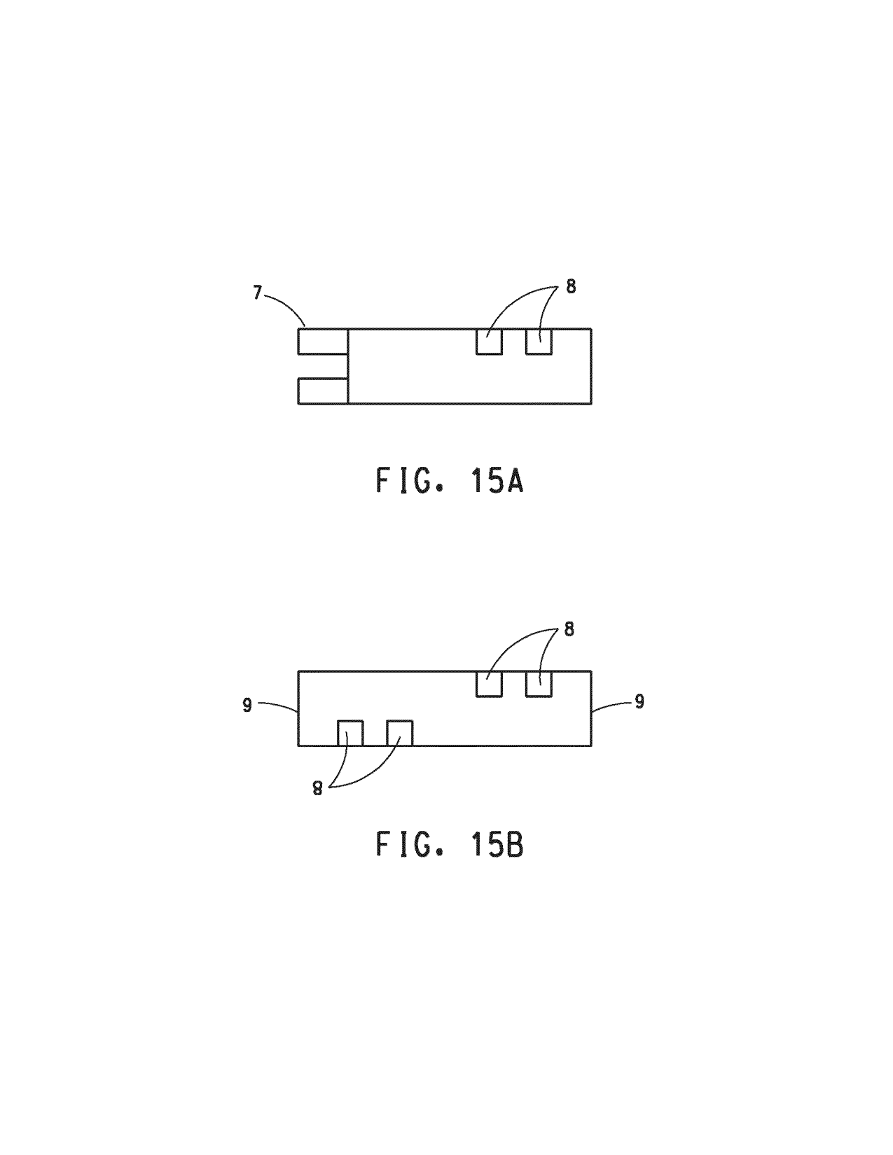

Field trial was conducted in an experimental farm in BSES Burdekin Research Station, Ayr, Australia. The field was prepared similar to commercial practice (i.e., 1.5 m row spacing) used for conventional sugarcane billet planting as is well known in the relevant art. About 100 meter long furrows were prepared with 1.5 m gap between furrows and irrigated to full field capacity 2-3 days prior to planting. Artificial seeds were planted within the furrows in wells that were 5-6 cm deep and 1.2 cm diameter, and sprayed with water immediately after planting to establish good connections between the soil and the artificial seed. The planted furrows were irrigated every third day for the first 10 days and then the irrigation continued once a week. Nearly 100 artificial seeds each of paper and plastic were planted (FIG. 12). As control, planlets of similar age, and produced similarly, were planted directly in the field and received similar field treatments.

Five weeks after planting, the number of plants emerged from the artificial seeds were recorded. FIG. 13 shows that nearly 55% of plantlets in artificial seeds with plastic containers grew and emerged through the Nescofilm® closures and survived. These results show a much higher rate of plantlet emergence when plastic containers are used for construction of the artificial seeds compared to the artificial seeds constructed with paper containers or direct planting. FIG. 14 shows photographs of plants produced from artificial seeds made from plastic containers (top panel) and in plantlets directly planted in soil (Bottom panel-right) after 5 weeks of growth. The root system was well developed in the plants in artificial seeds (Bottom panel left)

Results obtained from this field experiment indicate that using artificial seeds in paper or plastic containers allowed establishing of plantlets in the field under conditions similar to commercial planting practices.

### Example 13

### Effect of Side Openings on Survival and Sprouting of Plantlets in Artificial Seeds 1N Horizontally Planted Artificial Seeds

The purpose of the Example was to compare growth and survival of plantlets in artificial seeds planted in soil in horizontal orientation that had additional openings at the side of the container (FIG. 15, 8), with containers that had openings only on the top and the bottom ends of the artificial seed.

Wax paper containers (5 cm long) were sterilized by autoclaving. The containers were either crennelated (FIG. 15, 7) at one opening or flat on both ends (FIG. 15, 9). Circular openings (5 mm diameter) were punched in the walls of the containers near either the flat end in the case of the crennelated containers, or near both ends in the case of the flat ended containers. The containers were assembled with agar plugs containing nutrients as described in Example 2 and sugarcane plantlets, which had been regenerated from meristem tissue fragments (variety KQ228) in the plantlet regeneration medium for 15 days post-fragmentation were placed into the containers. All openings were secured with pre-stretched Parafilm® M. The artificial seeds thus prepared were planted in Metro-Mix® 360 with either the side openings exposed, or buried slightly under the soil. A control set was created without side openings and were planted horizontally, but left partially exposed to the surface in that the side of the artificial seed was visible through the soil, but the openings of the artificial seed did not extend above the soil surface. Artificial seeds were grown in 10 cm plastic pots in a (Conviron CGR-962, 31° C. day, 22° C. night, 14 hr photoperiod, 220 uE/m²) growth chamber, initially with plastic domes covering the pots. The plastic covers were removed after 16 days. Results of the experiment are given below in Table 7.

TABLE 7
Effect of windows on the survival and sprouting of plantlets in the artificial seeds when
planted horizontally

| | | # artificial | # Sprouted | # Sprouted | # Alive but | |
|---|---|---|---|---|---|---|
| | | seeds | through side | through top | not | |
| | Planting | planted | openings by | opening by | sprouting | # Dead by |
| Container | orientation | initially | day 19 | day 19 | by day 19 | day 19 |
| Crennelated | Horizontal, | 10 | 3 | 0 | 2 | 5 |
| with 2 side | partially | | | | | |
| openings at flat | covered with | | | | | |
| opening | soil | | | | | |
| Flat openings, | Horizontal, | 5 | 1 | 1 | 1 | 2 |
| with 2 side | partially | | | | | |
| openings at | covered with | | | | | |
| each end | soil | | | | | |
| Flat openings, | Horizontal, | 5 | 0 | 0 | 0 | 5 |
| with 2 side | lightly and | | | | | |
| openings at | completely | | | | | |
| each end | covered with | | | | | |
| | soil | | | | | |
| Crennelated | Horizontal, | 5 | 0 | 0 | 0 | 5 |
| without side | partially | | | | | |
| openings | covered with | | | | | |
| | soil | | | | | |
| Crennelated | Vertical, top | 5 | N/A | 2 | 0 | 3 |
| without side | exposed to | | | | | |
| openings | surface | | | | | |

The results in Table 7 indicate that the presence of side openings improved survival of the plantlets when artificial seeds are planted horizontally.

### Example 14

### Effect of Planting Artificial Seeds Upside-Down

The purpose of the experiment was to study the effect of planting artificial seeds in an upside-down, vertical orientation (with plantlet shoots pointing downward). Wax paper containers (5 cm long) were prepared with crenellation, but without side openings. The containers were assembled with agar plugs containing nutrients as described in Example 2 and sugarcane plantlets, which had been regenerated from fragmented meristem tissue (variety CP01-1372) in plantlet regeneration medium for 14 days post-fragmentation were placed into the containers. All openings were secured with pre-stretched Parafilm® M. The artificial seeds thus prepared were planted in Metro-Mix® 360 vertically in either an upside down or normal (right side up) orientation. Artificial seeds were incubated in 10 cm plastic pots in a (Conviron CGR-962, 31° C. day, 22° C. night, 14 hr photoperiod, 220 uE/m²) growth chamber, initially with plastic domes covering the pots. The plastic covers were removed after approximately 14 days. Results are given below in Table 8.

TABLE 8
Effect of planting artificial seeds in an upside-down configuration

| | | | # Alive but | |
|---|---|---|---|---|
| | # artificial seeds | | not | |
| Planting | planted | # Sprouted by | sprouting by | # Dead by |
| orientation | initially | day 18 | day 18 | day 18 |
| Vertical, upside | 8 | 5 | 1 | 2 |
| down | | | | |
| Vertical, right | 9 | 8 | 0 | 1 |
| side up control | | | | |

The results summarized in Table 8, indicate that the artificial seeds that were planted in the upside down configuration resulted in lower sprouting compared to the control artificial seeds planted in the right side up orientation.

### Example 15

### Synthesis of Polyester Block Copolymers for Use in Artificial Seeds

The synthesis of a series of polyester block copolymer was undertaken in order to create a biodegradable material suitable for use as synthetic seed lids, which had mechanical properties suitable to allow penetration by the emerging plant shoots. First, 3.00 g 3,6-Dimethyl-1,4-dioxane-2,5-dione was weighed into a 50 mL round bottom flask containing a magnetic stirbar in a nitrogen atmosphere in a glove box. Next, 0.020 g tin (II) 2-ethylhexanoate was weighed into the flask. 3.00 g ε-caprolactone was added to the flask, along with 0.025 g 1,4-benzenedimethanol. A condenser was attached to the flask and it was removed from the glove box and promptly purged with nitrogen gas. The flask was then heated to 140° C. under a nitrogen atmosphere with an oil bath and stirred magnetically for 24 hours. After 24 hours, a small amount of polymer was sampled out for analysis, and an additional 3.00 g 3,6-Dimethyl-1,4-dioxane-2,5-dione was added. Heating and stirring were resumed for 3 hours. The final product was cooled to room temperature, dissolved in chloroform and dripped into an excess of hexane/methanol (90/10 v/v) in order to precipitate the polymer. The final product was dried in a vacuum oven at 60° C. for 3 days.

A series of polymers were synthesized using this methodology, with variations described in Table 9, including the use of mechanical stirring, and chiral monomers, in order to achieve various properties. The polymer molecular weights were characterized using size exclusion chromatography in tetrahydrofuran (THF) solvent, with a multiangle laser light scattering detector. The middle block molecular weight was determined from the sample taken at the end of the first step, and this was subtracted from the molecular weight of the final product to determine the first and last block molecular weights (they were assumed to be equally distributed due to the difunctionality of the initiator).

TABLE 9
Composition and synthetic procedure parameters for polyester block
copolymers.

| | | | | | Measured block | |
|---|---|---|---|---|---|---|
| | | | | Targeted block | molecular | Measured |
| | | | | molecular | weights, using | polydispersity |
| | | | 2^{nd} Step | weights (first, | size exclusion | (PDI) by size |
| | | Stirring | heating | middle, last, | chromatography, | exclusion |
| Sample | Composition | method | duration | kg/mol) | number average | chromatography |
| PLA-7 | Poly(D,L-lactide-b- | Magnetic | 3 hrs | 8.2, 33.1, 8.2 | 2.0, 19.4, 2.0 | 1.83 |
| | D,L-lactide-co-ε- | | | | | |
| | caprolactone-b-D,L- | | | | | |
| | lactide) | | | | | |
| PLA-8 | Poly(L-lactide-b-D,L- | Mechanical | 8 hrs | 8.2, 33.1, 8.2 | 1.5, 18.0, 1.5 | 1.43 |
| | lactide-co-ε- | | | | | |
| | caprolactone-b-L- | | | | | |
| | lactide) | | | | | |
| PLA-9 | Poly(L-lactide-b-D,L- | Mechanical | 8 hrs | 8.2, 65.4, 8.2 | ND | 1.40 |
| | lactide-co-ε- | | | | | |
| | caprolactone-b-L- | | | | | |
| | lactide) | | | | | |

### Example 16

### Comparison of Additional Film Lid Compositions in Artificial Seeds

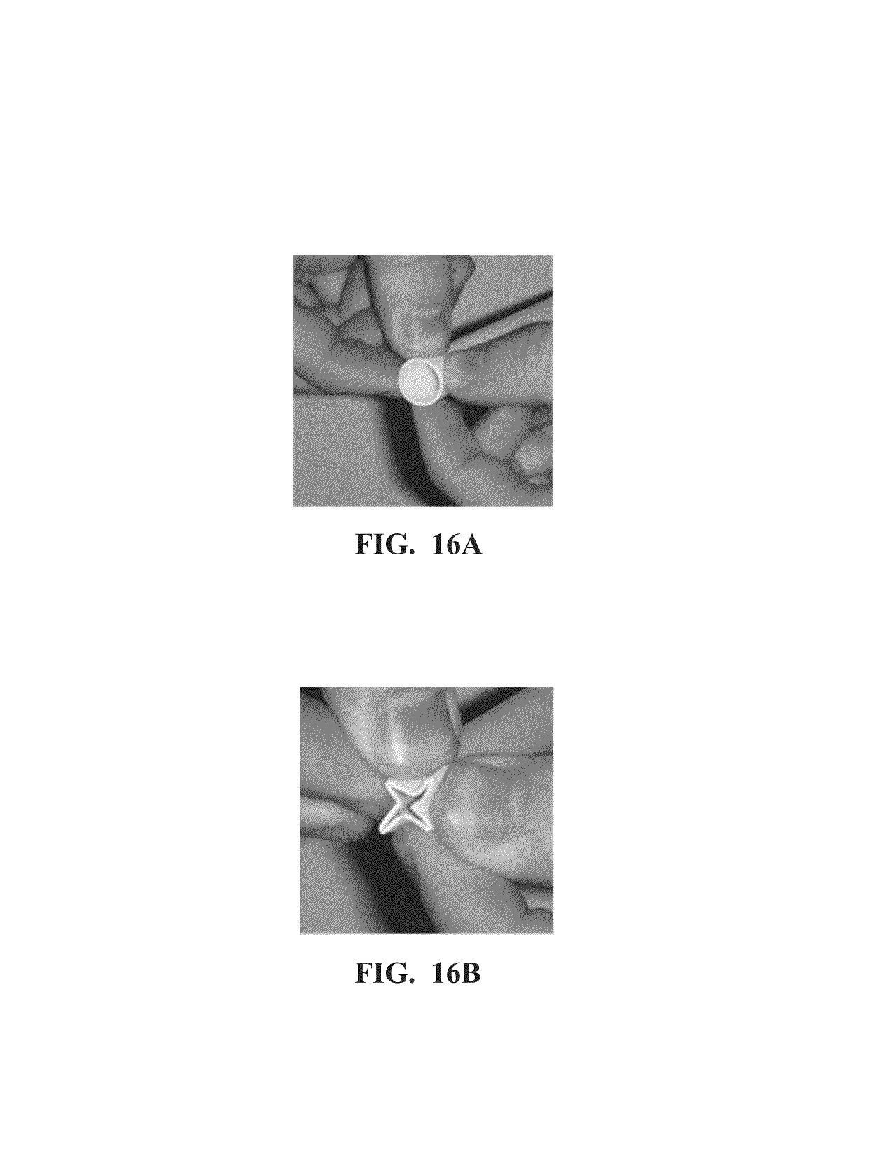

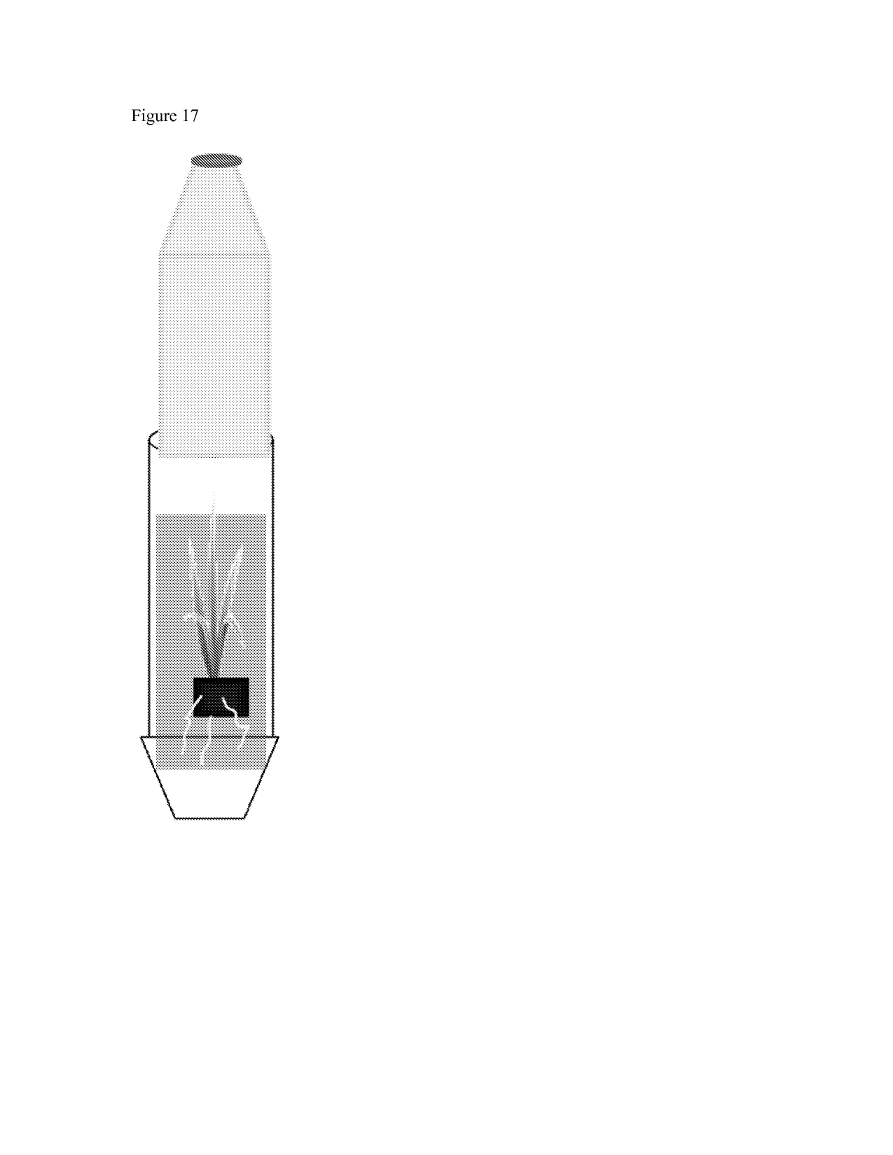

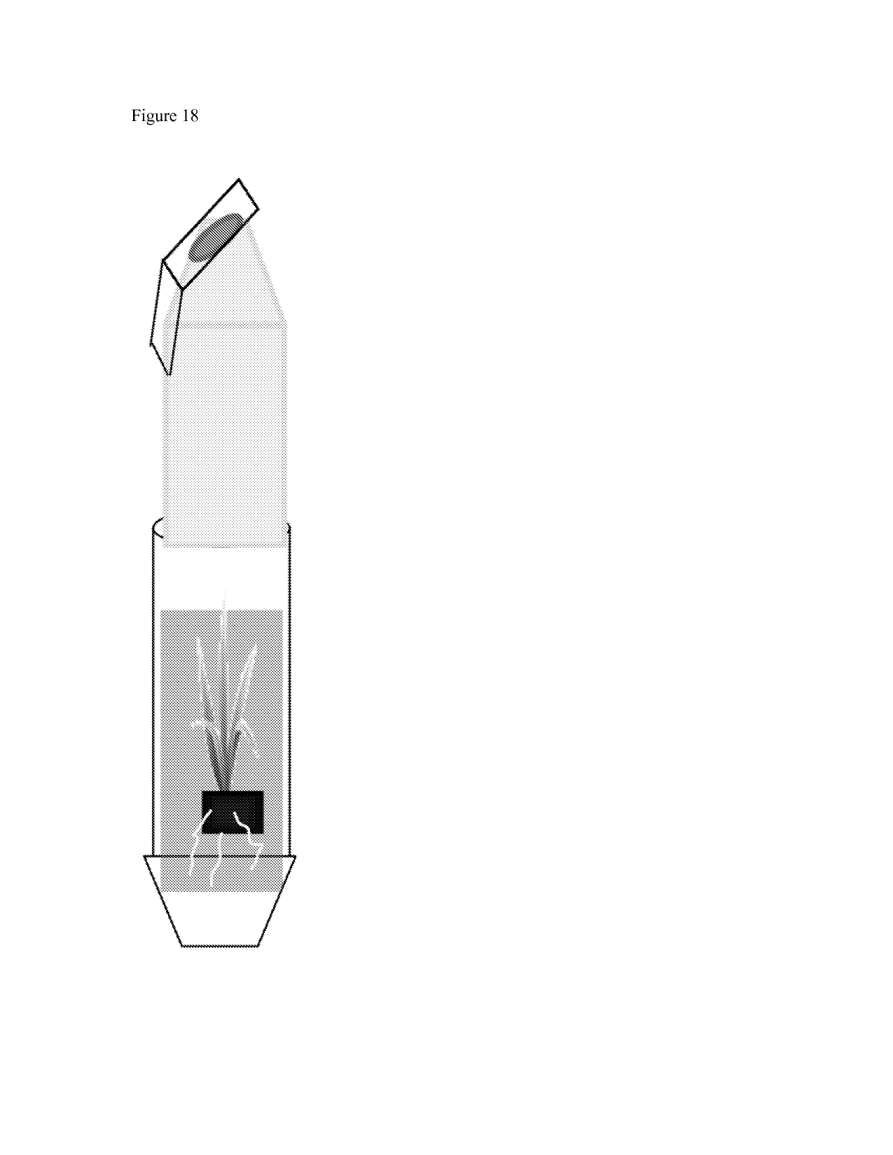

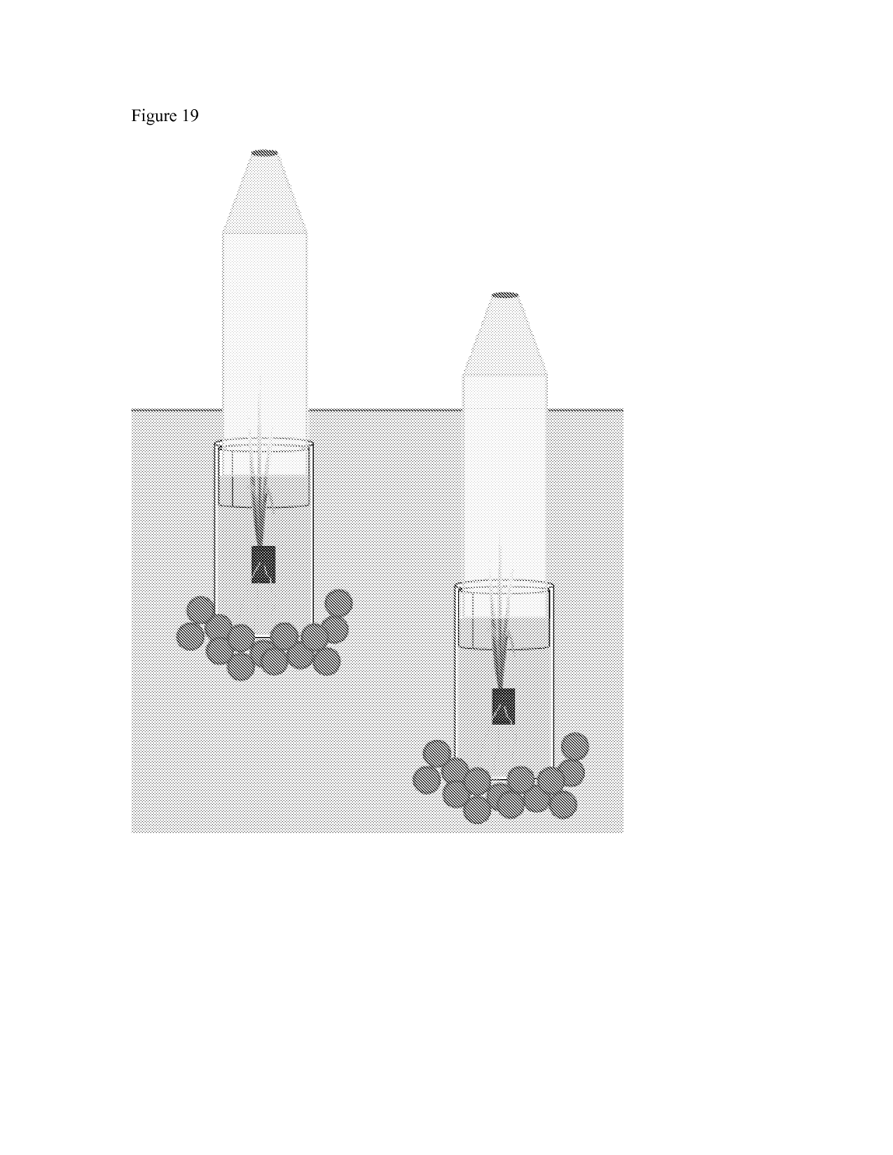

Wax paper containers (5 cm long, 1.19 cm diameter) were cut from longer sections. The bottom ends were manually crimped (FIG. 16). Then, Metro-Mix® 360 was added to the tube, to create an approximately 1 cm thick layer in the bottom of the tube. A sugarcane plantlet, which had been trimmed to approximately 3 cm length was then placed on top of the soil plug, and additional soil was added to the tube so that the tube was approximately 75% full, and 1 mL water was added. The top of the tube was secured with one of several methods described below. In one case, pre-stretched Parafilm® M was used to cover the top of the artificial seed. In another case, a 38 um thick film was formed by casting PLA-8 (Example 15) from a 25 wt % solution in THF onto a poly(tetrafluoroethylene) (PTFE) sheet using a 10 mil doctor blade. The film was then dried at room temperature for 5 hours, followed by drying in a vacuum oven at approximately 60° C. for 18 hours. Finally the film was attached to the end of the tube by heating the film until it softened (˜80° C.) on a poly(tetrafluoroethylene) (PTFE) coated foil on a hot plate, followed by manually pressing against the top of the tube. In another case, an alkyd film was formed by mixing 2.20 g Beckosol® 11-035 (Reichhold Inc, Durham, N.C.) with 0.545 g palm oil (Sigma Aldrich, St. Louis, Mo.), and 0.020 g cobalt (II) napthenate (55 wt % in mineral spirits, Electron Microscopy Sciences, Hatfield Pa.) using a magnetic stirbar, then coating that mixture on a poly(tetrafluoroethylene) (PTFE) sheet using a 245 um doctor blade and allowing to cure at room temperature for 24 hours at room temperature. The final thickness of the film was 75 um, and it was adhered to the top end of the paper tube using masking tape. In another case, translucent ⅜″ diameter cylindrical plastic caps (Alliance Express, Erie, Pa.) were inserted into the top of the tube. In another case, a conical lid was created by cutting the lid off a 1.7 mL microcentrifuge tube (SafeSeal Microcentrifuge Tubes, Sorenson BioScience Inc, Salt Lake City, Utah), and then cutting the tip off the tapered end, producing a ˜3-5 mm hole in the tapered end of the tube, and then inserting the wide end this tube in the top end of the paper tube (FIG. 17). In another case, a microcentrifuge tube was prepared similarly, except that the cut on the bottom end was at a ˜45 degree angle to the axis of the tube, and a 100 um thick Mylar® film, cut in a ˜1 cm×˜2 cm rectangle was bent at an angle such that it could be glued to the side of the plastic tube and cover the slanted hole (FIG. 18). This created a flap over the hole that could be pushed aside by the growing shoots of the plantlet. In another case, the lid was cut off a 1.7 mL microcentrifuge tube, but no hole was created in the bottom end, and this was adhered to the top of the paper tube using molten PLA-7 (Example 15), by first dipping the microcentrifuge tube in the molten PLA-7 which was maintained at 140° C. on a hot plate, and then pressing it on top of the paper section. In another case, a ˜1.5 cm square piece of 100 um thick Mylar® film was adhered to the ends of the paper tube using molten PLA-7. In a final embodiment, a rectangular piece of Mylar® film (˜1.5×2.5 cm) was bent at a 90 degree angle in the middle of the longest dimension, and then hot glued to the side of the paper tube such that the bent portion covered the open end of the tube, forming a flap. The artificial seeds thus prepared were planted in Metro-Mix® 360 such that the top of the paper sections were approximately 0.3-0.5 cm above the soil surface, in 10 cm plastic pots and grown in a (Conviron model BDW-120) at 31° C. during the day, 22° C. during the night, and a 13 hr photoperiod, 220 uE/m²).

TABLE 10
Effects of various lid types on sprouting of artificial seeds.

| | # artificial seeds | # Sprouted by |
|---|---|---|
| Top closure | planted initially | day 30 |
| Pre-stretched | 15 | 11 |
| Parafilm ® M | | |
| PLA 8 film | 15 | 13 |
| Alkyd film | 15 | 14 |
| ⅜″ Cylindrical | 16 | 7 |
| plastic cap | | |
| Conical lid with | 15 | 10 |
| hole | | |
| Conical lid with | 15 | 8 |
| hole and flap | | |
| Conical lid | 9 | 7 |
| without hole, | | |
| glued to tube | | |
| Mylar ® film | 10 | 6 |
| glued over tube | | |
| end | | |
| Mylar ® flap at | 10 | 4 |
| 90 degree angle | | |
| Bare plants | 42 | 42 |

As can be seen in Table 10, the PLA 8 film and Alkyd films outperformed the pre-stretched Parafilm® M. In several cases, the plantlets were able to extend their shoots through the conical tube with flap designs, as well as the paper tube with 90 degree Mylar® flap. Also, the plantlets were able to push off several of the ⅜″ cylindrical plastic caps.

### Example 17

### Measurement of Mechanical Properties of Polyester Block Copolymers

Mechanical properties of selected lid materials were measured in puncture mode, in order to assess the ease of penetration by plantlet shoots. This was performed on a TA-XT2i Texture Analyser (Texture Technologies, Scarsdale, N.Y.). A metal cylindrical probe 2 mm in diameter and 38 mm in length, tapered on the end with a 1 mm rounded tip was mounted on the load cell arm of the texture analyzer. The films were mounted on the open ends of 1.19 cm diameter paper tubes. The probe was set up to move downward such that it impinged on the film in the center of the paper tube at a 90 degree angle to the film surface. Studies were conducted in both modes of constant deformation and constant load (creep).

TABLE 11
Mechanical properties of lid film materials. Values separated by
commas represent replicates of the test.

| | | Strain at break | Force at break | Time to |
|---|---|---|---|---|
| | | (mm), constant | (g), constant | failure (sec) |
| | Thickness | strain rate (0.2 | strain rate (0.2 | (constant |
| Film | (um) | mm/s) | mm/s) | load, 20 g) |
| Pre-stretched | 50 | 3.0, 11.1 | 24.5, 44.7 | 914, >1500 |
| parafilm ® M | | | | |
| PLA-8 | 43 | 5.3 | 48.3 | 170, 255 |
| (Example 15) | | | | |
| Alkyd film | 100 | 4.5, 6.6 | 34.4, 32.2 | 63, 86 |
| (Example 16) | | | | |

In Table 11, the PLA-8 and the Alkyd film samples had comparable force at break when tested at constant deformation rate and faster time to failure under constant load, suggesting that they would be more easily punctured than the pre-stretched Parafilm® M.

### Example 18

### Comparison of Conical Lid to Film Lid in Artificial Seed

Wax paper containers (5 cm long, 1.19 cm diameter) were prepared by cutting sections from a longer tube. The bottom ends were either manually crimped (FIG. 16), or a small (˜1 cm thick) plug of rockwool was inserted in the bottom. Then, Metro-Mix® 360 was added to the tube, to create an approximately 1 cm thick layer. A trimmed sugarcane plantlet was then placed on top of the soil plug, and additional soil was added to the tube so that the tube was approximately 75% full. Then, 1 mL water was added to the soil in each tube. The top of the tube was either secured with pre-stretched Parafilm® M, a 150-225 um thick PLA-7 (Example 15) film or a conical tube, which was created as in Example 16 by cutting the lid and bottom tip off a 1.7 mL microcentrifuge tube (SafeSeal Microcentrifuge Tubes, Sorenson BioScience Inc, Salt Lake City, Utah), producing a ˜3-5 mm hole in the narrow end of the tube, and inserting the wide end of this tube in the top end of the paper tube (FIG. 17). The artificial seeds thus prepared were planted in Metro-Mix® 360 such that the top of the paper sections were approximately 0.3-0.5 cm above the soil surface, in 10 cm plastic pots and grown in a (Conviron model BDW-120) at 31° C. during the day, 22° C. during the night, and a 13 hr photoperiod, 220 uE/m²).

TABLE 12
Effect of various lid types on sprouting.

| | | # artificial seeds | | # Visibly |
|---|---|---|---|---|
| | Bottom | planted | # Sprouted by | Trapped at |
| Top closure | closure | initially | day 25 | day 14 |
| Conical lid with | Manually | 20 | 19 | 1 |
| hole | Crimped | | | |
| Pre-stretched | Manually | 20 | 13 | 4 |
| Parafilm ® M | Crimped | | | |
| PLA-7 | Manually | 15 | 10 | 0 |
| | Crimped | | | |
| Pre-stretched | Rockwool | 16 | 11 | 3 |
| Parafilm M | plug | | | |

In Table 12, the results of the experiment show that a higher fraction of plantlets successfully sprouted through the conical lid, compared to film type lids, including pre-stretched Parafilm® and PLA-7. Furthermore, it was observed that pre-stretched Parafilm® lids had a higher number of trapped plantlets (where the shoots were visibly impinging on the inner lid surface) compared to conical lids.

### Example 19

### Field Testing of Conical Lidded Artificial Seed

Wax paper containers (6 cm long, 2 cm diameter) were prepared by cutting from longer sections. The paper tube sections were flat on both ends. The caps were removed from 15 mL polypropylene centrifuge tubes (VWR International, LLC, Radnor, Pa.), and the conical tip was cut at a 90 degree angle to the tube axis, revealing a ˜5-8 mm diameter hole. Short (˜2 cm long) sections of 2 cm diameter paper tube were cut and then slitted along their length to act as a wedge or shim to hold the plastic tubes snugly inside the paper tubes. Sugarcane plantlets from tissue culture were trimmed to ˜8 cm lengths. The root ends of the sugarcane plantlets were rolled in Metro-Mix® 360 to create a soil covered root ball. The plantlet was then inserted in the 6 cm long paper tube section such that the root ball was roughly 1 cm above the bottom, and the shoot end was protruding out of the top end. Then, Metro-Mix® 360 was added from top and bottom around the plant such that the bottom was filled to the opening, and about 1 cm was left unfilled at the top. It was gently compacted with a pen and more soil was added until the tube was filled approximately 1 cm from the top. Then the paper insert was pressed into the top of the paper tube, around the plantlet shoots. The 15 mL centrifuge tube was then inserted, wide end down, over the shoots of the plantlet, into the paper tube, so that it was inserted about 2 cm into the paper tube. Then, 4 mL water was added to the soil in each tube through the hole in the plastic section. The artificial seeds were planted in a field environment at DuPont Stine Haskell Research Center in Newark, Del. The soil had been tilled and prepared in a flat fashion and had been fertilized using urea. The artificial seeds were planted in rows with 30 cm spacing in a vertical orientation in several different conditions. In one condition, they were planted 8 cm deep in the soil. In another condition they were planted 8 cm deep with approximately 30 mL of superabsorbent beads (Magic Water Beads, magicwaterbeads.com) pre-swollen in water, placed around the base of each tube (FIG. 19). In another condition, they were planted 12.5 cm deep, with approximately 30 mL of superabsorbent beads (Magic water beads, magicwaterbeads.com) pre-swollen in water, placed around the base of each tube (FIG. 19). In another condition, they were planted 12.5 cm deep, with approximately 30 mL of superabsorbent beads (Magic water beads, magicwaterbeads.com) pre-swollen in Murashige and Skoog (MS) nutrient media, placed around the base of each tube. In another condition, a 20 cm deep and 20 cm diameter hole was excavated, and the field soil was replaced with Metro-Mix® 360, and the artificial seeds were planted 8 cm deep with approximately 30 mL of superabsorbent beads (Magic water beads, magicwaterbeads.com) pre-swollen in water, placed around the base of each tube. As a comparison, bare plantlets were also planted directly into the field, such that the roots were approximately 1 cm deep. The field was irrigated immediately after planting and generally 3 times per week thereafter.

TABLE 13
Results of field experiment with 15 mL conical lid artificial seeds

| | | # artificial seeds | |
|---|---|---|---|
| Planting | | planted | % Sprouted by |
| condition | Superabsorbent | initially | day 33 |
| 8 cm deep | none | 20 | 75% |
| 8 cm deep | Water swollen | 50 | 80% |
| 12.5 cm deep | Water swollen | 30 | 23% |
| 8 cm deep in | Water swollen | 33 | 60% |
| Metro Mix ® 360 | | | |
| 8 cm deep | MS swollen | 20 | 85% |
| Bare plantlets | none | 72 | 6% |

In Table 13, 15 mL artificial seeds provided increased survival compared to bare plantlets. The use of shallower planting resulted in increased survival. Additionally, it was observed that the plastic conical tube served to collect dew at certain times during the experiment. Additionally, it was observed that as the plants grew large enough for the shoots to impinge on the hole that was made in the conical tube, that several of the plants (68% by day 90) grew tillers adjacent to the plastic tube body. This appeared to occur by growing through the region of the seed composed of the paper section which had degraded in the soil.

### Example 20

### Effect of Size and Dimension of Conical Lid in Field Survival of Artificial Seeds

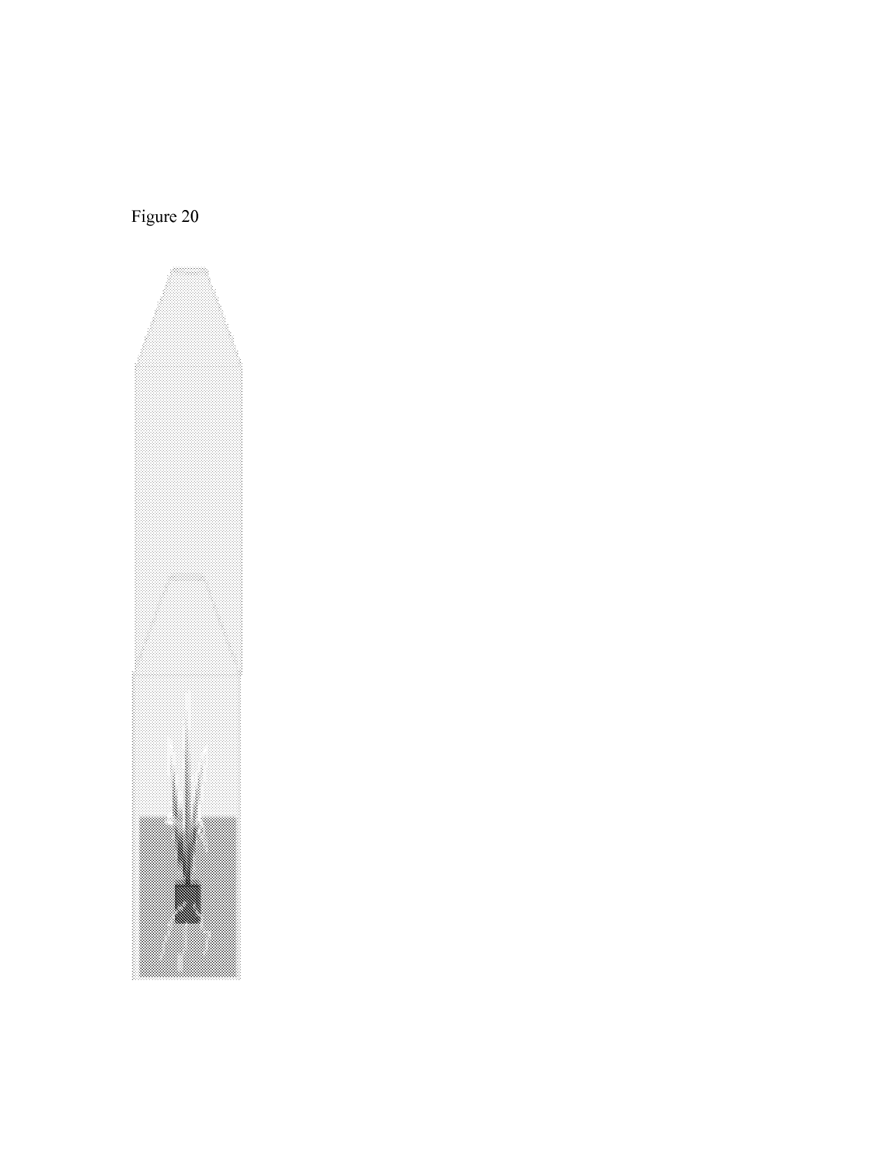

Wax paper containers with 15 mL conical lids were fabricated as described in Example 19. In addition, sugarcane plantlets were planted in 2″ pots in Metro-Mix® 360 which had been saturated with water and were trimmed to approximately 6-8 cm length. The caps were removed from 15 mL conical centrifuge tubes (VWR International, LLC, Radnor, Pa.) and the bottom tapered tips were cut such that a ˜5-8 mm hole was created. The caps were removed from 50 mL conical centrifuge tubes (VWR International, LLC, Radnor, Pa.) and the bottom tapered tips were cut such that a ˜1.5 cm hole was created. The 15 mL and 50 mL conical tubes were positioned over the shoots of the plantled sugarcane plantlets and then forcibly pressed down with a twisting motion, such that the plantlet as well as the soil surrounding it were taken up in the conical tube. This resulted in a soil plug approximately 3-6 cm tall inside the base of the tube. For one treatment, a second 50 mL tube was stacked on top of the first 50 mL tube containing the plantlet (FIG. 20). This created a second “chamber” above the plantlet. The tubes were then lifted out of the pots, stored overnight in plastic bags and transported to the field for planting in the morning. The artificial seeds were planted in a field environment at DuPont Stine Haskell Research Center in Newark, Del. The soil had been tilled and prepared in a flat fashion and had been fertilized using urea. The artificial seeds were planted in rows with 30 cm spacing, 8 cm deep in the soil, in a vertical orientation. As a comparison, bare plantlets were also planted directly into the field, such that the roots were approximately 1 cm deep. The field was irrigated immediately after planting and generally 3 times per week thereafter.

TABLE 14
Results of field experiment with various conical lid artificial seed designs.

| | | | Average height |
|---|---|---|---|
| | | | from soil |
| | # Artificial | | surface to |
| | seeds planted | % Sprouted | dewlap (cm) |
| Design | initially | by day 37 | day 60 |
| 15 mL conical tube | 30 | 53% | 5.82 |
| with paper section | | | |
| 15 mL conical tube | 30 | 50% | 3.60 |
| without paper | | | |
| 50 mL conical tube | 31 | 77% | 7.17 |
| without paper | | | |
| Two 50 mL conical | 30 | 93% | 12.74 |
| tubes stacked on top | | | |
| of each other | | | |
| Bare plantlet controls | 72 | 55% | 6.44 |

In Table 14, it is noted that the 50 mL tubes had higher survival than the 15 mL tubes and that the stacked 50 mL tubes had higher survival than the single 50 mL tubes in both sprouting as well as plant height. Furthermore, the 50 mL and stacked 50 mL conical tubes provided increased survival compared to the bare plantlet controls.

### Example 21

### Effect of Conical Tube End and Protective Flaps on Field Survival of Artificial Seeds

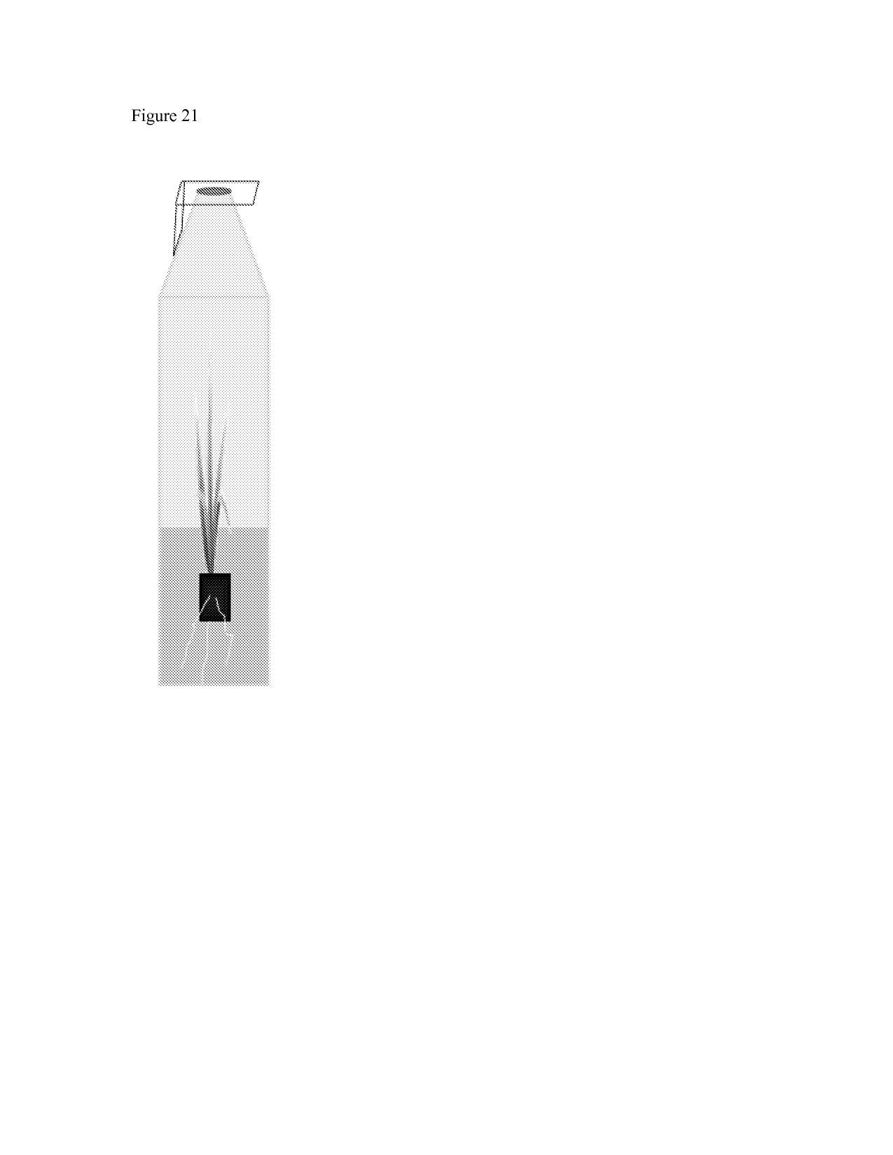

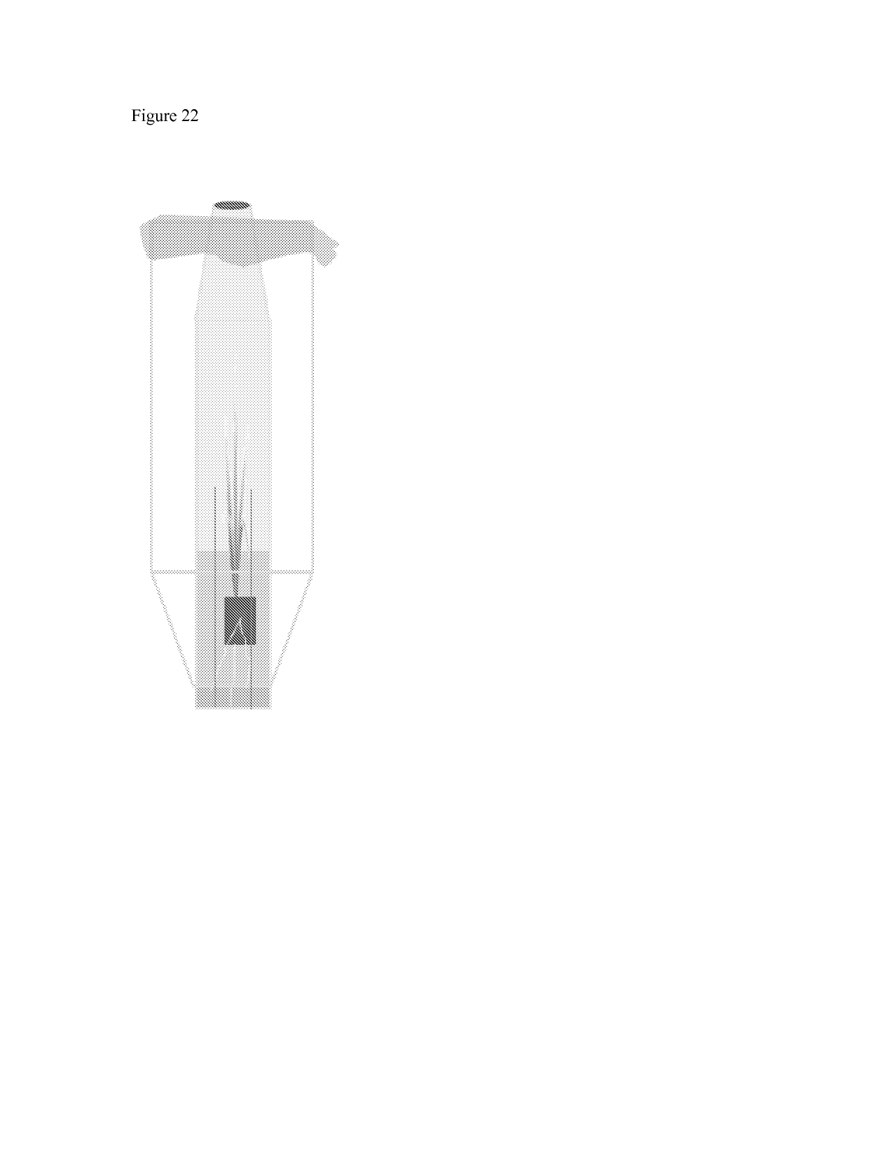

15 mL and 50 mL conical tube artificial seeds were fabricated as described in Example 20. In addition, sugarcane plantlets were planted in 2″ pots in Metro-Mix® 360 which had been saturated with water and were trimmed to approximately 6-8 cm length. The caps were removed from 50 mL conical centrifuge tubes (VWR International, LLC, Radnor, Pa.) and the entire conical tips were cut off, resulting in a cylindrical tube open on both ends. In another treatment, the caps were removed from 50 mL conical centrifuge tubes (VWR International, LLC, Radnor, Pa.) and the tip of the conical ends were cut, revealing a ˜5-8 mm hole. A 100 um thick Mylar® rectangular film (˜2 cm×˜1 cm) was bent at such an angle that when hot glued to the side tip of the conical tube, the free end loosely covered the ˜5-8 mm hole (FIG. 21). In another treatment, the caps were removed from 15 mL centrifuge tubes (VWR International, LLC, Radnor, Pa.) and the tips of the conical end were cut leaving a 5-8 mm hole. Four 4.5 cm slots were cut along the axis of the tube from end with the larger opening toward the tapered tip. The caps were removed from 50 mL conical centrifuge tubes (VWR International, LLC, Radnor, Pa.) and the tips were cut to create a hole large enough for the 15 mL conical tube to be inserted upside down. The structure was inverted so that the wide end of the 50 mL tube was pointed upward. A superabsorbent powder, poly(acrylic acid), partial sodium salt-graft-poly(ethylene oxide) (Sigma Aldrich, St Louis, Mo.) was swollen in deionized water at a ratio of 1:223 (weight of powder:weight of water). This gel was then inserted into the annular cavity between the two tubes. Parafilm® M was then stretched over the wide end of the 50 mL tube, with a hole in the middle where the 15 mL tube protruded (FIG. 22). The opening in the 15 mL tube was left open. All types of conical tubes were positioned over the shoots of the planted sugarcane plantlets and then forcibly pressed down with a twisting motion, such that the plant as well as the soil surrounding it were taken up in the conical tube. This resulted in a soil plug approximately 3-6 cm tall inside the base of the structure. The tubes were then lifted out of the pots and transported to the field for planting. The artificial seeds were planted Aug. 24, 2012 in a field environment at DuPont Stine Haskell Research Center in Newark, Del. The soil had been tilled and prepared in a flat fashion and had been fertilized using urea. The artificial seeds were planted in rows with 30 cm spacing, 8 cm deep in the soil, in a vertical orientation. As a comparison, bare plantlets were also planted directly into the field, such that the roots were approximately 1 cm deep. The field was irrigated immediately after planting and generally 3 times per week thereafter.

TABLE 15
Results of field experiments with various conical lid artificial seed designs.

| | | | Average height |
|---|---|---|---|
| | | | from soil |
| | # artificial | | surface to |
| | seeds planted | % Sprouted | dewlap (cm) |
| Design | initially | by day 35 | day 47 |
| 15 mL conical tube | 55 | 73% | 7.81 |
| (no paper) | | | |
| 50 mL conical tube | 85 | 89% | 9.03 |
| (no paper) | | | |
| 50 mL cylindrical tube | 46 | 78% | 6.94 |
| (no paper) with open end | | | |
| 50 mL conical tube | 44 | 66% | 8.44 |
| (no paper) with Mylar flap | | | |
| covering hole in top | | | |
| 50 mL tube with 15 mL tube | 40 | 73% | 7.87 |
| inside, superabsorbent gel | | | |
| in annular space | | | |
| Bare plantlet controls | 58 | 67% | 8.19 |

In Table 15, the results of the experiment showed that the 50 mL conical tube with a hole in the top without paper performed the best in terms of survival. This result was better than the same sized cylindrical tube lacking the conical tip.

### Example 22

### Effect of Various Conical Tube Designs and Storage on Field Survival of Artificial Seeds

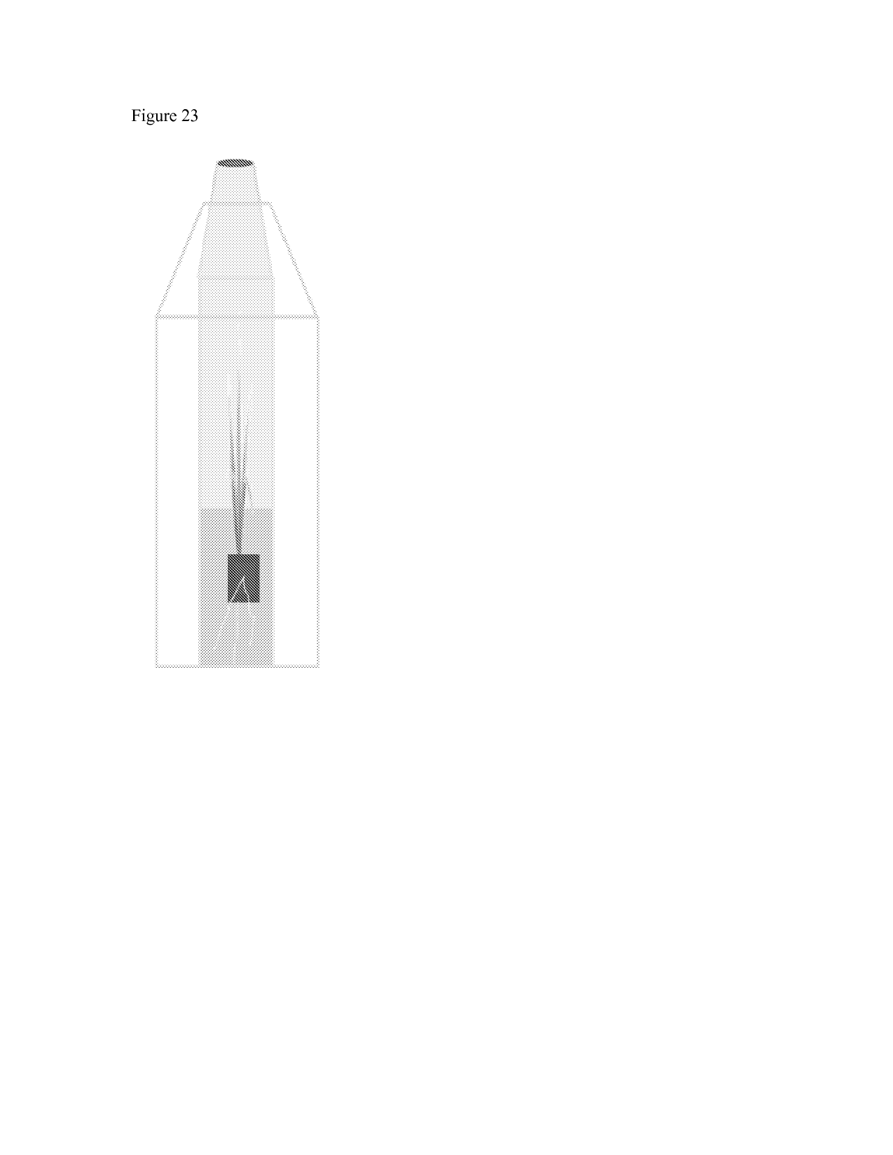

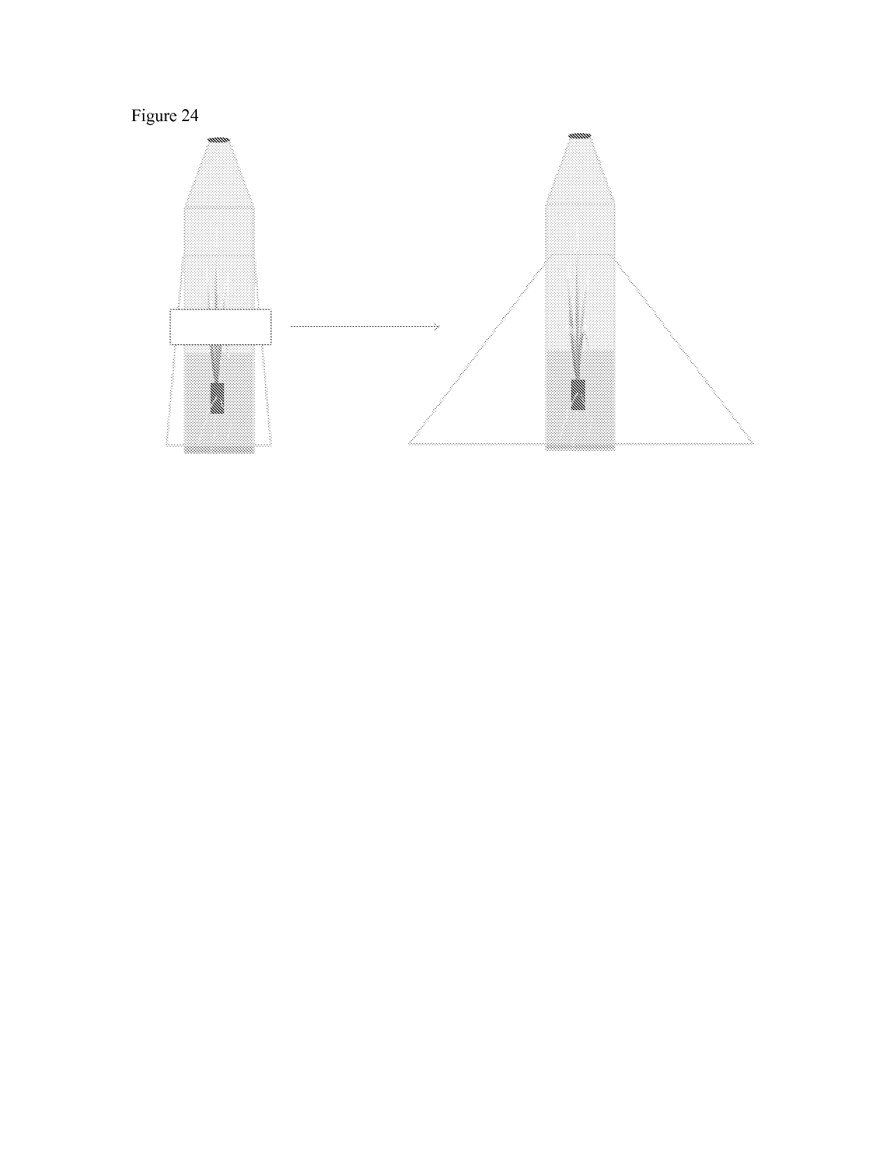

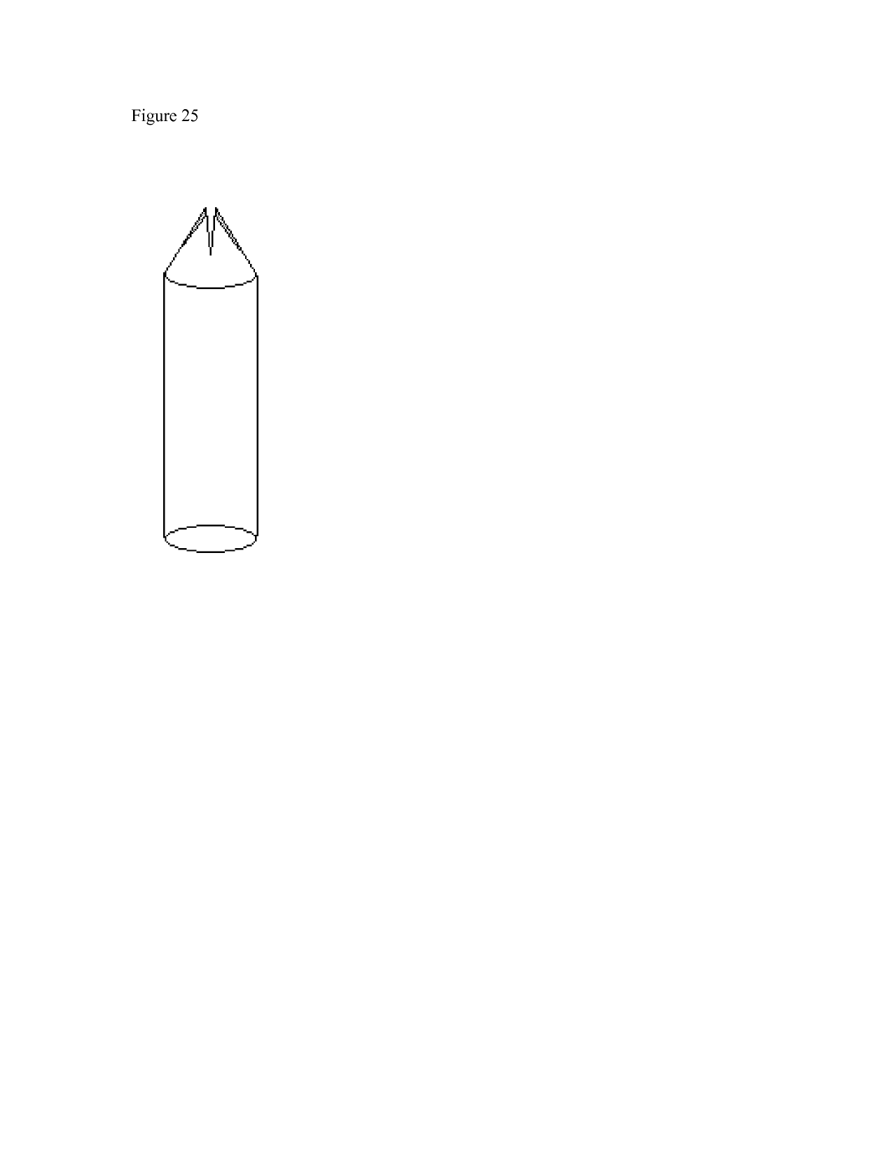

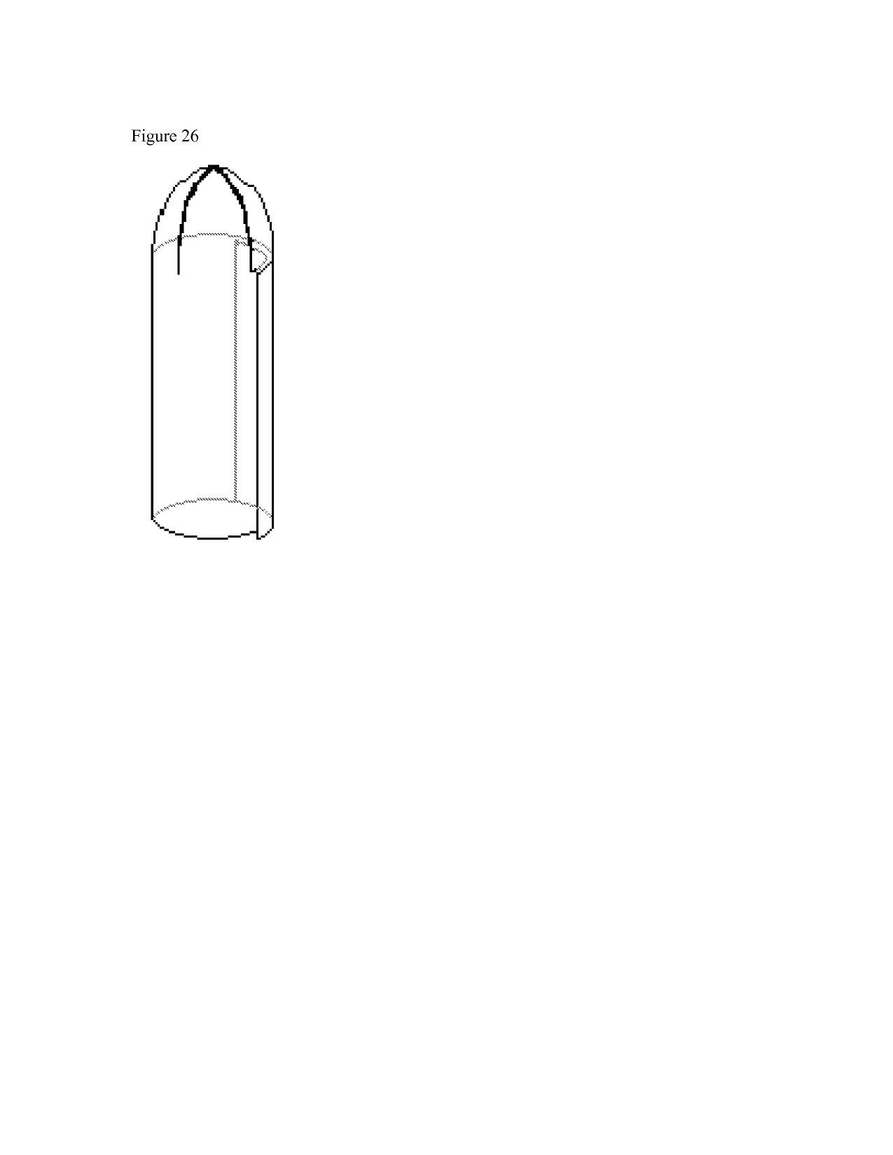

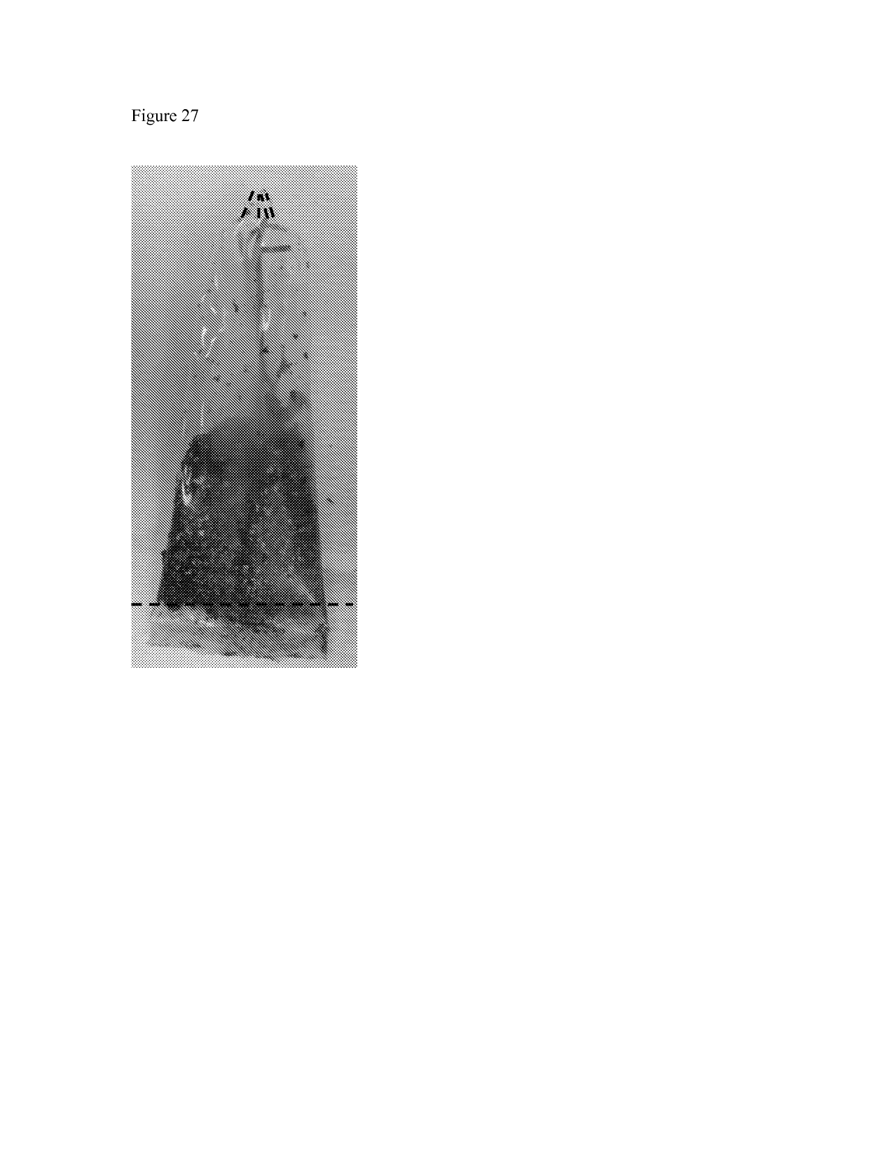

15 mL and 50 mL conical tube artificial seeds were fabricated as described in Example 20. In addition, sugarcane plantlets were planted in 2″ pots in Metro-Mix® 360 which had been saturated with water and were trimmed to approximately 6-8 cm length. The caps were removed from 15 mL conical centrifuge tubes (VWR International, LLC, Radnor, Pa.) and the tips were cut, resulting in a 5-8 mm hole in the tip. The caps were removed from 50 mL conical centrifuge tubes (VWR International, LLC, Radnor, Pa.) and the tips were cut in order to make a hole large enough for the 15 mL conical tube to fit. The 15 mL conical tube was then inserted into the 50 mL conical tube in an orientation such that both conical ends were pointed upward and the 15 mL tube fit snugly inside the 50 mL tube (FIG. 23). In another treatment, the caps were removed from 15 mL conical centrifuge tubes (VWR International, LLC, Radnor, Pa.) and the tip of the conical ends were cut, resulting in a ˜5-8 mm hole. A polyethylene sample bag corner was cut to form a triangular shaped tent, with the height from the large open end to the corner approximately equal to the length of the 15 mL conical tube. A small approximately 1 cm hole was made by cutting off the corner of this triangle, in order to allow the tip of the 15 mL conical tube to be inserted. This resulted in a plastic film tent surrounding the 15 mL conical tube. Autoclave tape was folded on itself to form a band large enough to hold the tent section around the tube. This band was removed prior to planting, and the tent was expanded to its maximum coverage (FIG. 24). This structure was planted such that the edges of the tent were covered by the soil. In another treatment, the caps were removed from 50 mL conical centrifuge tubes (VWR International, LLC, Radnor, Pa.) and the tip of the conical ends were cut, resulting in a ˜2 cm hole. A polyethylene sample bag corner was cut to form a triangular shaped tent, with the height from the large open end to the corner approximately equal to the height of the original conical section of the 50 mL conical tube. The sample bag corner was hot glued to the opening in the conical tube in order to form a tent-like covering over the hole. Then, scissors were used to cut two approximately 1 cm slots in this tent like covering at 90 degree angles to each other, with the cut direction oriented along the axis of the tube. This created an opening through which the plantlet's shoots could grow (FIG. 25). In another treatment, poly(lactic acid) pellets (PLA2002D, NatureWorks, Minnetonka, Minn.) were hot pressed at 190° C. into films that were 200-380 um thick. These films were cut into rectangular pieces approximately 12 cm×10 cm. A sawtooth pattern with approximately 2 cm deep and 3 cm wide triangular features was cut along one of the 10 cm edges. Next, the films were rolled into overlapping tube shapes, and inserted into 50 mL conical centrifuge tubes (VWR International, LLC, Radnor, Pa.) with the sawtooth pattern pointing into the cone. The conical tubes were then placed in an oven at 120° C. with a conical dowel made of poly(acetal) (22 mm diameter, 15 cm length) inserted in the middle of the tube for 2-5 minutes in order to soften the film to conform to the tube shape. This was then removed and cooled to room temperature on a laboratory bench top, resulting in the triangular features from the sawtooth pattern pointing toward each other in a cone-like shape (FIG. 26). The rolled poly(lactic acid) film and dowel were removed from the 50 mL centrifuge tube. The above described tubes were positioned over the shoots of the planted sugarcane plantlets and then forcibly pressed down with a twisting motion, such that the plant as well as the soil surrounding it were taken up in the conical tube. This resulted in a soil plug approximately 4-6 cm tall inside the base of the structure. The tubes were then lifted out of the pots and transported to the field for planting. The artificial seeds were planted Sep. 13, 2012 in a field environment at DuPont Stine Haskell Research Center in Newark, Del. As a comparison, bare plantlets were also planted directly into the field, such that the roots were approximately 1 cm deep. The field was irrigated immediately after planting and no irrigation was provided thereafter. The soil had been tilled and prepared in a flat fashion and had been fertilized using urea. The artificial seeds were planted in rows with 30 cm spacing, 8 cm deep in the soil, in a vertical orientation. In another treatment, poly(lactic acid) pellets (PLA2002D, NatureWorks, Minnetonka, Minn.) were dissolved in chloroform at 12.5 wt %. This solution was poured into 50 mL centrifuge tubes (VWR International, LLC, Radnor, Pa.). The excess solution was poured out of the tubes and the residue on the inner walls was allowed to dry at ambient conditions inside a fume hood for 24 h. Then, the tubes were placed in a vacuum oven and dried at 50° C. for 3 days with a steady flow of air through the chamber. The poly(lactic acid) castings were pulled out of the 50 mL centrifuge tubes. These solution cast tubes were positioned over the shoots of the planted sugarcane plantlets and then forcibly pressed down with a twisting motion, such that the plant as well as the soil surrounding it were taken up in the conical tube. This resulted in a soil plug approximately 4-6 cm tall inside the base of the structure. The tubes were then lifted out of the pots and heat sealed along the bottom edge using a Quick Seal impulse sealer (National Instrument Co, Baltimore, Md.). These seeds were separated into two groups and stored at either ambient temperature or 15° C. for 9 days before being planted in the field. Planting was accomplished by cutting the bottom edge that had been heat sealed off, and cutting the tip of conical section with two perpendicular cuts directed along the axis about 1 cm long (FIG. 27). The tube was then planted approximately 5 cm deep in the soil.

TABLE 16
Results of field experiments with various conical lid artificial seed designs.

| | # Artificial | | |
|---|---|---|---|
| | seeds planted | % Sprouted | % Sprouted |
| Design | initially | by day 22 | by day 29 |
| 15 mL conical tube | 31 | 42% | 58% |
| 50 mL conical tube | 31 | 32% | 42% |
| 15 mL conical tube nested | 30 | 67% | 80% |
| inside 50 mL conical tube | | | |
| 15 mL conical tube with PE | 33 | 48% | 54% |
| bag tent | | | |
| 50 mL conical tube with PE | 30 | 37% | 50% |
| bag corner top | | | |
| Rolled poly(lactic acid) tubes | 24 | 29% | 46% |
| Ambient temperature/1 wk | 13 | 38% | |
| Stored poly(lactic acid) tubes | | | |
| 15° C./1 wk Stored poly(lactic | 12 | 25% | |
| acid) tubes | | | |
| Bare plantlets | 168 | 8% | |
| | | (day 21) | |

As shown in Table 16, the best performing artificial seed was the 15 mL tube nested inside of the 50 mL conical tube. The tented structures showed similar survival compared to the non-tented structures. All seed structures provided improved survival compared to bare plantlets.

### Example 23

### Variations on Conical Tube Artificial Seed

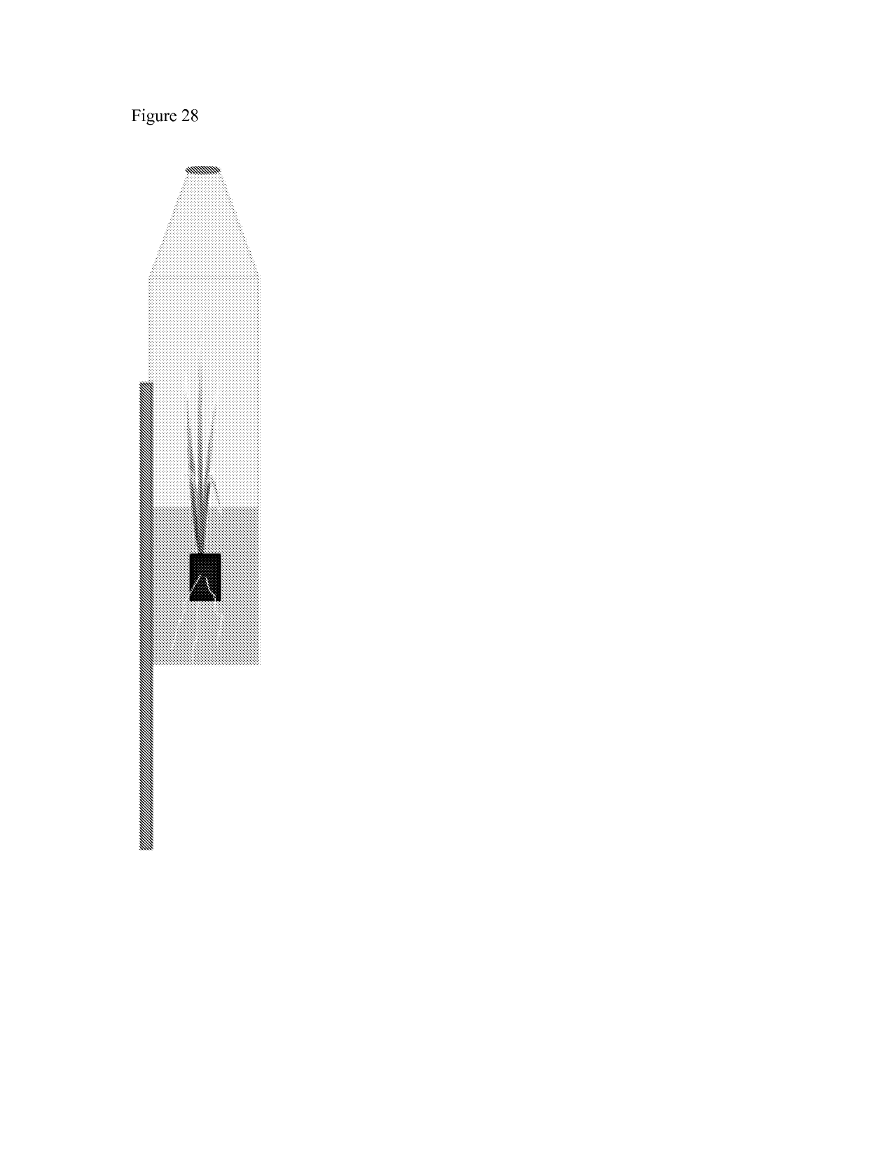

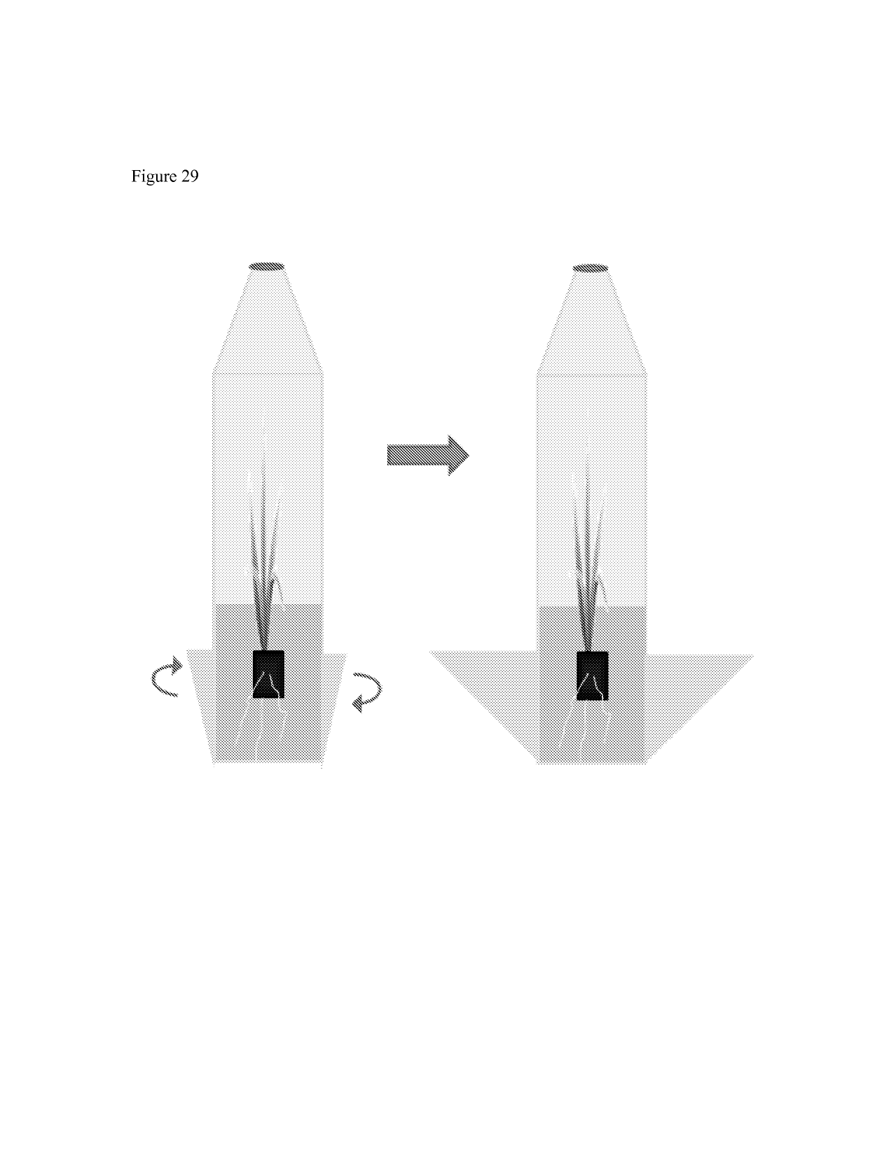

This study was performed in order to explore various practical implementations of conical tube artificial seeds, and to address potential issues such as soil and moisture retention during handling and storage. 15 mL conical tube artificial seeds without paper sections were fabricated as described in Example 20. In addition to this design, the following modifications were included as treatments. In one case, a cold-water soluble poly(vinyl alcohol) film (Extra Packaging, Boca Raton, Fla.) was hot glued to cover the opening at the top of the 15 mL conical tube artificial seed. This was intended to improve moisture retention in the seed. After planting, these seeds were watered from the top, simulating rain, in order to dissolve the film. In one treatment, a thin plastic rod was made by cutting the loop end off 10 uL disposable loops (Becton Dickinson and Co., Sparks, Md.), producing a plastic rod approximately 11 cm long. These were then hot glued to the side of the 15 mL conical tube artificial seed such that they extended approximately 5 cm below the bottom of the tube, with the sharp end pointing downward. This was intended to anchor the tube in the soil (FIG. 28). In another case, autoclave tape (VWR International, LLC, Radnor, Pa.) was used to cover both the top and bottom of the 15 mL conical tube artificial seed. This seed was stored for 1 week at room temperature before planting, and the tape was removed at the time of planting. In another treatment, a small circle of plastic window screen (Lowe's Home Improvement, Newark Del.) was hot glued to the bottom of the 15 mL conical tube artificial seed. This was intended to facilitate retention of soil during storage and handling. In another treatment, 15 mL conical tube artificial seeds were created using a potting soil containing coco coir (Special Mix Coco, Gold Label Special Mix® Substrates, Gold Label Americas, Olivehurst, Calif.) instead of Metro-Mix® 360. In another treatment, triangular pieces of 100 um thick Mylar® film were hot glued to the base of the 15 mL conical tube artificial seeds in order to serve as a foldable anchor (FIG. 29). In another treatment, paper tubes were fabricated from Rite in the Rain® all weather copier paper (J.L. Darling Corp, Tacoma, Wash.), which has improved moisture resistance compared to kraft or bond paper. The tubes were formed by wrapping this paper around a 15 mL centrifuge tube and hot gluing along the edge. The tubes were cut into 5 cm sections and were covered by pre-stretched Parafilm® M. The paper tube artificial seeds were planted in Metro-Mix® 360 such that the top of the paper sections were approximately 0.3 cm above the soil surface, in 10 cm plastic pots and grown in a (Conviron model BDW-120) at 31° C. during the day, 22° C. during the night, 80% relative humidity and a 13 hr photoperiod, 220 uE/m²). The 15 mL conical plastic tube artificial seeds, were planted in 10 cm plastic pots in Metro-Mix® 360 at a depth of 4-5 cm.

TABLE 17
Results of comparing various tube seed structures in the growth chamber.

| | #Artificial seeds | Number sprouting |
|---|---|---|
| Design | planted initially | at day 28 |
| 15 mL conical tube | 20 | 19 |
| (reproduced from Example 20) | | |
| 15 mL conical tube with | 18 | 10 |
| water soluble film lid | | |
| 15 mL conical tube with plastic | 24 | 21 |
| window screen covering base | | |
| 15 mL conical tube with | 20 | 19 |
| plastic stake anchor | | |
| 15 mL conical tube with | 20 | 20 |
| Mylar ® film anchor | | |
| 15 mL conical tube with taped ends, | 24 | 21 |
| stored for 1 week prior to planting | | |
| 15 mL conical tube with coco | 20 | 20 |
| coir containing potting soil | | |
| Rite in the Rain ® paper tube with | 24 | 15 |
| pre-stretched Parafilm ® M lids | | |

As can be determined from Table 17, various practical modifications of conical tube synthetic seed, including the use of screened bottoms, stakes and anchors did not have significant deleterious effects on survival. Roots were also observed penetrating the window screen when the artificial seeds were exhumed at the end of the experiment, at day 40.

### Example 24

### Effect of Cone Angle of Conical Tube Artificial Seed on Sprouting

The purpose of this study was to determine the optimum cone angle for the conical tube artificial seed design. 50 mL conical tube artificial seeds without paper sections were fabricated as described in Example 20. In addition to this design, the following modifications were included as treatments. In one treatment, the conical tips were entirely cut off the ends of the 50 mL conical centrifuge tubes (VWR International, LLC, Radnor, Pa.), creating cylindrical tubes. 100 um thick Mylar® film was cut into circles of the same diameter as the 50 mL tubes. A hole punch was used to make a 5 mm hole in the center of the circle. The circle was then hot glued to the top of the tube. In another treatment, the conical tips were entirely cut off the ends of the 50 mL conical centrifuge tubes (VWR International, LLC, Radnor, Pa.), creating cylindrical tubes. 100 um thick Mylar® film was cut into circles of the slightly larger diameter than the 50 mL tubes. A hole was punched in the middle, and a single slit was cut radially out from the center hole to the outside edge. The resultant closed arc was forcibly overlapped to create a cone with a wider angle (approximately 135 degrees) than the standard 50 mL conical tubes (65 degrees). The tubes with various cone angles were assembled with plantlets and soil inside, as described in Example 20. They were planted in 10 cm plastic pots at a depth of 4-5 cm and grown in a (Conviron model BDW-120) at 31° C. during the day, 25° C. during the night, and a 13 hr photoperiod, 220 uE/m²).

TABLE 18
Results of comparing various tube seed structures in the growth
chamber.

| | | | | Number | | |
|---|---|---|---|---|---|---|
| | | | | of tubes | | |
| | | | | in which | | Number |
| | | | | tops | Number | visibly |
| | | | | were | sprouting | trapped |
| | | | Number | pushed | through | but |
| | Cone | Number | sprouting at | off at | hole at | alive at |
| Seed structure | angle | planted | day 21 | day 21 | day 21 | day 21 |
| 50 mL conical | 65 | 20 | 20 | 0 | 20 | 0 |
| tube (reproduced | | | | | | |
| from Example | | | | | | |
| 20) | | | | | | |
| 50 mL | ~135 | 20 | 5 | 0 | 5 | 15 |
| cylindrical | | | | | | |
| tube with | | | | | | |
| slight conical | | | | | | |
| Mylar ® top | | | | | | |
| with hole | | | | | | |
| 50 mL | 180 (no | 20 | 5 | 3 | 2 | 15 |
| cylindrical | cone) | | | | | |
| tube with flat | | | | | | |
| Mylar ® top | | | | | | |
| with hole | | | | | | |

Table 18 shows that the tubes with the smaller cone angle produced better sprouting results. In the case with the flat top or shallow cone, most of the plants were trapped although alive.

### Example 25

### Production of Polyester-Polysiloxane Block Polymer Film for Artificial Seed Closures

The synthetic procedure described below was carried out to provide an alternative material with enhanced biodegradability for use as an artificial seed closure. The material is a block polymer comprised of poly(lactide) (PLA)—a rigid, glassy polymer at room temperature—and poly(dimethylsiloxane) (PDMS)—a liquid at room temperature. The relative contents of PLA and PDMS in the material are selected to yield an overall mechanical response that is similar to manually pre-stretched Parafilm® M.

Aminopropyl-terminated PDMS of 900-1100 cSt viscosity was purchased from Gelest (DMS-A31) and used as a difunctional macroinitiator for the polymerization of lactide. Under oxygen- and water-free conditions, 40 g of the PDMS was added to a 1 L round bottom flask. To the flask, 60 g of lactide (Sigma-Aldrich), 40 μL of tin(II) 2-ethylhexanoate (Sigma-Aldrich), and 461 mL of toluene (EMD Chemicals) were added. The reaction mixture was heated under stirring to 100° C. for 24 hrs. The resultant solution of poly(lactide-b-dimethylsiloxane-b-lactide) (LDL) triblock polymer was dried using a rotary evaporator. The solid LDL polymer was re-dissolved in 435 g methylene chloride (EMD Chemicals), precipitated in a 10-fold volumetric excess of methanol (EMD Chemicals), filtered and washed with methanol, and then dried under vacuum at 45° C. Approximately 87 g of LDL were obtained.

The total number-averaged molecular weight Mₙ and composition f_{PLA} (weight fraction of PLA) of the LDL, determined by nuclear resonance spectroscopy, and the polydispersity index PDI, determined by size exclusion chromatography, are provided in Table A. A film of LDL was prepared by first dissolving the polymer in chloroform (EMD Chemicals) at 20 wt. %. This solution was cast on a Teflon® substrate using a doctor blade with a 5 cm wide and 254 um thick gap. After drying under ambient conditions for 5 days, a film of approximately 75 um thickness was obtained. The elastic modulus E, tensile strength σ_{f}, and strain at break ε_{f} of the LDL was measured under uniaxial tension, as shown in Table 19. For comparison, the corresponding values of pre-stretched Parafilm® M are also provided. In this case, prior to measurement, the Parafilm® M sample, having equal initial length and width, was subjected to 200% uniaxial strain along its length, followed by 200% uniaxial strain along its width.

TABLE 19
Molecular and mechanical properties of LDL and Parafilm ® M

| Material | Mₙ (kg/mol) | f_{PLA} | PDI | E (MPa) | σ_{f} (MPa) | ε_{f} (%) |
|---|---|---|---|---|---|---|
| LDL | 50 | 0.57 | 1.37 | 52 | 5.2 | 210 |
| Parafilm ® | — | — | — | 19 | 3.6 | 130 |
| M | | | | | | |

### Example 26

### Effect of Container Length and Closure Type on Viability of Artificial Seeds

Wax paper containers were cut into 4 cm and 7 cm lengths. One open end of each container was secured with either a 38 um thick LDL film, prepared as described in Example 25, or a 254 um thick soybean oil gel film. The latter was prepared by dissolving Kraton® A1535 poly(styrene-b-ethylene-co-butylene-co-styrene-b-styrene) triblock polymer in soybean oil (MP Biomedicals, Solon, Ohio) at 9 wt. % and 155° C., and casting the hot solution on a glass substrate using a doctor blade with a 5 cm wide and 254 um thick gap, preheated to 155° C. Upon cooling to room temperature, the physical gelation of the triblock polymer in the oil yielded a solid, but highly deformable film. LDL film was affixed to the wax paper container using a thin layer of cyanoacrylate adhesive (Sigma-Aldrich, St. Louis, Mo.). Soybean oil gel film was affixed by heating the film, still adhered to the glass substrate, to near its sol-gel transition (approximately 80° C.), pressing the end of the wax-paper container into the softened film, and cooling to room temperature to re-solidify the film.

The 4 and 7 cm wax paper containers, having their bottom ends secured with LDL or soybean oil gel film, were then loaded approximately one-third full with dry Metro-Mix® 360 growing media. One regenerated sugarcane plantlet was then added to each container. The regenerated plantlets were prepared from cultivar CPO-1372 according to a procedure similar to that described in Example 1 The regenerated plantlets varied in length from several centimeters to over 10 cm. After adding a plantlet to a 4 cm container, the shoots of the plantlet were trimmed to fit within the 4 cm length. For the 7 cm containers, the shoots of the plantlets were still trimmed to fit within a 4 cm container, i.e., all plantlets were trimmed to the same length, regardless of container size. The 4 cm containers were then filled to the top with additional Metro-Mix® 360 and 1 mL of deionized water was added to the container via pipette. After the addition of water, the soil level in the 4 cm tube compacted to fill approximately two thirds of the container. The 7 cm containers were then filled with a 4 cm thick layer of Metro-Mix® 360 and 1 mL of deionized water was added to the container via pipette. The top end of the container was secured with LDL or soybean oil gel film as described previously. Identical materials were used for the top and bottom closure of each container, that is, each container was closed exclusively by LDL film or exclusively by soybean oil gel film.

The artificial seeds were planted in 10 cm plastic pots with slits cut along the bottom surface and filled with Metro-Mix® 360. The pots were further placed in a plastic tray to collect water. All artificial seeds were planted in a vertical orientation; 4 cm containers were planted with the top closure flush with the soil level and 7 cm containers were planted with the top closure 3 cm above the soil level. The pots were maintained in an environmental chamber with a 16 hr photoperiod of 3000 lum/ft² luminosity and a 31/20° C. day/night cycle. The pots were watered, generally, at frequencies of several days.

The number of artificial seeds planted of each combination of container length and closure type is provided in Table 20, as well as the percentage of artificial seeds that sprouted and survived the 4 week duration of observation and their average height. The artificial seeds exhibited high sprouting and survival rates, a minimum of 60%. For comparison, bare plantlets transplanted directly from regeneration to Metro-Mix® 360 in the same environmental chamber exhibited 46% survival, respectively, after 4 weeks. Therefore, enclosure of the regenerated plantlets in the wax-paper containers provided a marked increase in viability. It is further evident that LDL closures provided enhanced viability—a minimum of 90% —in comparison to soybean oil gel closures. While the latter are more deformable, and hence more readily punctured by the shoots of the encapsulated plantlet, discoloration of the plantlet shoots was observed when in contact with the top closure. This suggests a certain degree of phytotoxicity of the soybean oil gel to the plantlets, which likely explains the lower degree of success of the corresponding artificial seeds. In contrast, no discoloration of plantlet shoots in contact with LDL closures was observed.

TABLE 20
Viability of sugarcane plants from artificial seeds of varying
container length, closure type, and plantlet type

| | | Number | Survival of | Mean Height of |
|---|---|---|---|---|
| Container | | of Seeds | Plants After 4 | Plants After 4 |
| Length (cm) | Closure Type | Planted | Weeks (%) | Weeks (cm) |
| 4 | LDL | 58 | 90 | 19 |
| 4 | soybean oil gel | 59 | 80 | 18 |
| 7 | LDL | 29 | 97 | 27 |
| 7 | soybean oil gel | 30 | 60 | 17 |

### Example 27

### Viability of Artificial Seeds from Cylindrical Containers in Field Testing

This experiment compared the growth of sugarcane plantlets in a field environment from three different artificial seeds, primarily distinguished by the material comprising the body of the seed container. Wax paper containers were cut into 21.6 cm lengths. Cellulose acetate butyrate (CAB) rigid tubing of 1.59 cm outer diameter and 1.25 cm inner diameter was purchased from McMaster-Carr (Santa Fe Springs, Calif.) and cut into 21.6 cm lengths. Porous polyethylene (PPE) rigid tubing of 1.90 cm outer diameter, 1.25 cm inner diameter, and 20 μm pore size was purchased from Interstate Specialty Products (Sutton, Mass.) and cut into 15.24 cm lengths. One open end of each wax paper, CAB, and PPE container was secured with a 38.1 um thick LDL film, as described in Example 26. The containers were then loaded with 1 g of dry Metro-Mix® 360 growing media. One regenerated sugarcane plantlet of cultivar CPO-1372, prepared by a procedure similar to that described in Example 1, was added to each container. No plantlets were trimmed prior to or after addition to a container. After plantlet addition, all containers were loaded with an additional 1 g of dry Metro-Mix®-360 and 2 mL of deionized water, then the top end of the container was secured with LDL film using cyanoacrylate adhesive (Sigma-Aldrich, St. Louis, Mo.).

The artificial seeds were planted in a field at the DuPont Stine-Haskell Research Center located in Newark, Del. The field was prepared to give a flat planting surface. The artificial seeds were planted in rows, with 1.5 m between rows and 15 cm between adjacent seeds within a row. The artificial seeds were planted in a vertical orientation such that the encapsulated plantlet's shoots were facing upwards and approximately 4 cm of the container was beneath the soil level. The field was irrigated immediately after planting and generally 3 times per week thereafter. The number of artificial seeds of each container type planted, as well as the percentage of seeds that sprouted and survived the 4 week duration of observation are listed in Table 21. The CAB and PPE containers led to a substantially higher survival rate, in comparison to wax paper containers.

TABLE 21
Viability of sugarcane plants from artificial seeds of
varying container type in field

| | Number of | Survival of Plants |
|---|---|---|
| Container Type | Seeds Planted | After 4 Weeks (%) |
| Wax Paper | 33 | 15 |
| CAB | 32 | 69 |
| PPE | 18 | 67 |

### Example 29

### Encapsulation of Sugarcane Plantlets in Rapidly Biodegradable Containers to Provide Artificial Seeds

The aliphatic polyester poly(ε-caprolactone) (PCL) was used to construct rapidly biodegradable containers. PCL was purchased from Sigma-Aldrich (St. Louis, Mo.) and dissolved in chloroform at 10 wt %. This solution was cast on a glass substrate using a stainless steel doctor blade with a 5 cm wide and 254 um thick gap. The resultant PCL film was dried, yielding a final thickness of 0.001-0.002 inches. After removal from the glass substrate, two pieces of film, each measuring 5 cm in width and 10.2 cm in length, were overlaid and heat-sealed along the two longer edges and one of the shorter edges to create an open pouch. The pouch was loaded with 1 g of dry Metro-Mix® 360. A regenerated sugarcane plantlet was then added to the pouch, followed by an additional 1.2 g of dry Metro-Mix® 360 and 2.1 g of deionized water. The plantlets were prepared from cultivar CPO-1372 according to a procedure similar to that described in Example 1. The plantlet's shoots were trimmed, if necessary, to fit within the pouch and the remaining open edge was sealed, forming a closed, air-tight PCL container around the plantlet.

The as-prepared artificial seeds were planted in 10 cm plastic pots with slits cut along the bottom surface and filled with Metro-Mix® 360. The pots were further placed in a plastic tray to collect water. All artificial seeds were planted roughly 2-3 inches deep in a vertical orientation such that the encapsulated plantlet's shoots were facing upwards. The pots were maintained in an environmental chamber with a 13 hr photoperiod of 1900 lum/ft² luminosity and a 31/22° C. day/night cycle. The relative humidity was controlled at a constant value of 80%. The pots were watered at a frequency of 1-2 times per week. For comparison, plantlets from the same batch used to prepare the artificial seeds were planted bare in identically prepared and maintained pots.

On account of the rapid biodegradability of PCL and the relatively thin form in which it is utilized in the above-described artificial seeds, the sugarcane plants resulting from said artificial seeds exhibit an establishment process that is distinct from the previously described tubular artificial seeds. Periodic sampling of the artificial seeds indicated that macroscopic breakdown and fragmentation—a direct result of biodegradation—of the buried portion of the PCL container occurred over a time scale of roughly one to two weeks after planting. This phenomenon enabled establishment of the plant roots in the soil surrounding the original PCL container. Over the same time period and, in fact, over the six week duration of the experiment, no visual evidence of degradation of the above-surface portion of the PCL was observed. However, the plant shoots clearly increased in size within the confines of the PCL container. Several weeks to over one month after planting, the shoots of the growing plants are able to push the undegraded portion of the PCL container away from the soil surface and the growth of the sugarcane plant continues in a regular fashion thereafter. Ultimately, the undegraded portion of the PCL container falls off or remains adhered to the tip of a growing plant shoot.

Of the 30 artificial seeds planted, 22 (73%) sprouted and survived the six week duration of the experiment. Of the 20 bare plantlets planted, 12 (60%) survived over the same duration. The root masses and heights of the surviving bare plants substantially exceeded those of the surviving plants from artificial seeds. This is a consequence of the delayed release of the roots and shoots of the encapsulated sugarcane plantlets from their PCL containers. No lifting away of the PCL container from the soil by the growing plant—was observed in the time period one week or less after planting. Ultimately, all surviving samples sprouted, although sprouting was delayed in several artificial seeds by greater than one month. Collectively, the results demonstrate the viability of artificial seeds comprised of sugarcane plantlets encapsulated by thin, closed, and rapidly biodegradable containers.

Viability of Artificial Seeds Comprising Rapidly Biodegradable Containers in Field

Artificial seeds comprising sugarcane plantlets encapsulated by PCL film containers were prepared and planted in a field at the DuPont Stine-Haskell Research Center located in Newark, Del. on two separate occasions. In all cases, the artificial seeds were planted in a flat field preparation. The artificial seeds were planted approximately 5-7.5 cm deep in a vertical orientation such that the encapsulated plantlet's shoots were facing upwards. During the first experiment, the field was irrigated immediately after planting and one to two times per week thereafter. During the second experiment, the field was irrigated immediately after planting, but no irrigation was provided thereafter.

Table 22 shows the number of artificial seeds planted for each experiment, as well as the percentage that sprouted over the duration of the experiment (seven and four weeks for the first and second experiments, respectively) and the percentage that survived. The process by which the sugarcane plants established in the surrounding soil from these artificial seeds was similar to that observed in the growth chamber experiment. Over the course of the first one to two weeks after planting, the buried portion of the PCL container rapidly biodegraded, thereby releasing the plantlet roots to the surrounding soil. During the same period, shoot growth occurs within the confinement of the undegraded, above-surface portion of the PCL container. At longer times, continued shoot growth lifts the remainder of the PCL container away from the soil surface and growth continues in a regular fashion thereafter. Survival, as mentioned previously, is defined by visual evidence of a healthy, live plant. A significant number of samples from the first planting survived, but did not sprout. Sprouting was first observed among the planted population after roughly two weeks and the percentage of sprouted samples subsequently increased in a linear fashion with respect to time. In contrast, after the four week duration of the second experiment, no sprouting was observed. This is a consequence of lower temperatures, which significantly reduced the plant growth rate; the average surface soil temperatures measured over the course of the two experiments were 24° C. and 20° C. for the first and second, respectively. Table 22 indicates that moderate to high survival was obtained with these artificial seeds. Accounting for the sub-optimal growing temperatures encountered in the second, the data demonstrate the viability of rapidly biodegradable containers for artificial seeds.

TABLE 22
Sprouting and survival of sugarcane plants from PCL film
containers in field

| | | | Lifting of PCL |
|---|---|---|---|
| Date of Planting | Number Planted | Survival (%) | container (%) |
| First | 39 | 97_{^{†}} | 64_{^{†}} |
| Second | 85 | 66_{^{‡}} | 0_{^{‡}} |

_{^{†}}Recorded 7 weeks after planting
_{^{‡}}Recorded 4 weeks after planting

### Example 30

### Silicate Nutrient Media for Artificial Seeds

The purpose of this study was to examine the use of silicate gels as a nutrient media for the growth of sugarcane plantlets. 45 g potassium silicate solution in water (29.1 wt % solids, Kasil® 1, PQ Corporation, Malvern, Pa.) was added to 255 g deionized water and 300 g Murashige and Skoog (MS) media with 3 wt % sucrose and 0.2 wt % Plant Preservative Mixture (PPM) in a beaker. The mixture was adjusted to pH 7 using nitric acid. This solution was then filter-sterilized using a 1 L, 0.22 um pore size filter assembly (Corning Inc., Corning N.Y.). After sitting for 2 hours, the solution formed a gel. The gel was then submerged in an excess of deionized water and allowed to soak in order to remove the residual salts (potassium nitrate). The gel soaked for 4 days and the deionized water was replaced on the 4^{th} day. On the 5^{th} day, the media was replaced with an excess of MS media with 3 wt % sucrose and 0.2 wt % PPM. After soaking in the MS/sucrose media for 24 h, the excess liquid was drained and the gel was autoclaved prior to testing. Another gel was made using Kasil® 2135.45 g potassium silicate solution in water (35.5 wt % solids, Kasil® 2135, PQ Corporation, Malvern, Pa.) was added to 255 g deionized water and 300 g Murashige and Skoog media with 3 wt % sucrose in a beaker. The mixture was adjusted to pH 7 using nitric acid. This solution was then filter-sterilized using a 1 L 0.22 um pore size filter assembly (Corning Inc., Corning N.Y.). After 2 hours, this formed a gel. The gel was then submerged in an excess of deionized water and allowed to soak in order to remove the residual salts (potassium nitrate). The gel soaked for 4 days and the deionized water was replaced on the 4^{th} day. On the 5^{th} day, the media was replaced with an excess of MS media with 3 wt % sucrose and 0.2 wt % PPM. After soaking in the media for 24 h, the excess liquid was drained and the gel was autoclaved prior to testing. The conductivity of these gels was approximately 5 mS, whereas the conductivity of the media itself was approximately 3 mS. As a control, a gel was prepared using Difco® agar by heating 0.7 wt % Difco® agar in MS media with 3 wt % sucrose and 0.2 wt % PPM at approximately 80° C. until it dissolved, then pouring into Phtyatrays™ (Phytatray™ II, Sigma Aldrich, St. Louis Mo.) and cooling. Under sterile conditions in a laminar flow hood, sugarcane plantlets from tissue culture of meristematic tissue, which had grown for 4 weeks in liquid culture post fragmentation were divided into groups of 12, blotted dry with a paper towel and weighed. These were placed on top of the various gel materials in a 3×4 array pattern in Phytatrays™. The Phytatrays™ were closed with sterile, gas permeable tape (Filter tape, Carolina Biological Supply Company, Burlington, N.C.) and were incubated at 26° C. with 60 microEinsteins/m²/s light from Philips F32T8/ADV841/XEN 25 watt cool white fluorescent tube in the containers for a period of 16 days. After this period of time, the plantlets from each Phytatray™ were removed from the gel, blotted dry and weighed again (fresh weight). The ratio of the weight after 16 days to the initial weight was determined.

In a separate experiment, silicate gels were made in a similar manner as described above, except that the soaking step to remove the residual salts was not performed. Due to the lack of a soaking step, the resultant strength of Murashige and Skoog and sucrose nutrients was 45-50% of the standard MS media strength. A second difference was that the gels were neutralized with acetic acid, instead of nitric acid. A final difference was that the sugarcane plantlets were 15 days in liquid culture at the time of the experiment instead of 4 weeks. For this experiment, low melting agarose at 0.5 wt % in ½ strength Murashige and Skoog nutrient media was used as a control gel instead of Difco® agar. Three replicates of the trays were created in this experiment. The conductivities of the Kasil® based silicate gels without soaking were 13.5 mS for the Kasil® 1 based gel, and beyond the capability of the measuring device (VWR® Traceable® Conducitivity Pen) for the Kasil® 2135 based gel.

TABLE 23
Growth of sugarcane plantlets on silicate gel nutrient media. “A”, “B” and “C”
denote replicates of the same treatment.

| | | Age of | | | | |
|---|---|---|---|---|---|---|
| | | plantlets in | | | | |
| | | liquid | Soaking | | | Ratio of |
| | | culture | duration in | Initial wt/ | Final wt/ | final |
| | | prior to | deionized | 12 | 12 | to |
| | | experiment | water | plantlets | plantlets | initial |
| Gel | Nutrient | (days) | (days) | (g) | (g) | wt |
| Kasil ® 1 | MS media + | 28 | 5 | 5.05 | 14.56 | 2.88 |
| based | 3% sucrose | | | | | |
| silicate | | | | | | |
| gel | | | | | | |
| Kasil ® | MS media + | 28 | 5 | 6.80 | 17.19 | 2.53 |
| 2135 | 3% sucrose | | | | | |
| based | | | | | | |
| silicate | | | | | | |
| gel | | | | | | |
| 0.7 wt % | MS media + | 28 | None | 6.78 | 20.34 | 3.00 |
| Difco ® | 3% sucrose | | | | | |
| agar | | | | | | |
| Kasil ® 1 | ½ Strength | 15 | None | A = 1.42 | A = 1.74 | A = 1.22 |
| based | MS media + | | | B = 1.54 | B = 1.70 | B = 1.10 |
| silicate | 1.5% sucrose | | | C = 1.61 | C = 1.38 | C = 0.86 |
| gel | | | | | | |
| Kasil ® | 45% strength | 15 | None | A = 1.10 | A = 1.17 | A = 1.06 |
| 2135 | MS media + | | | B = 0.95 | B = 0.92 | B = 0.97 |
| based | 1.35% | | | C = 1.37 | C = 1.19 | C = 0.87 |
| silicate | sucrose | | | | | |
| gel | | | | | | |
| Low | ½ strength | 15 | None | A = 1.41 | A = 5.34 | A = 3.78 |
| melting | MS media + | | | B = 1.52 | B = 5.43 | B = 3.57 |
| agarose | 1.5% sucrose | | | C = 1.55 | C = 6.67 | C = 4.30 |

As can be seen in Table 23, the soaking step to remove salts from the silicate gels improved the growth of sugarcane plantlets compared to the gels which had not been soaked. With the soaking step, the silicate gels serve as successful growth media for sugarcane plantlets, whereas without the soaking step, no growth occurred. Furthermore, the plantlets incubated on the non-soaked silicate gels exhibited discoloration and signs of stress.

### Use of Silicate Nutrient Gels in Wax Paper Tube Artificial Seeds

The purpose of this study was to examine the use of silicate gels as a nutrient media in artificial seeds. 15 g potassium silicate solution in water (29.1 wt % solids, Kasil® 1, PQ Corporation) was added to 85 g deionized water and 100 g Murashige and Skoog media with 3 wt % sucrose in a beaker. The mixture was adjusted to pH 7 using nitric acid. This solution was then filter-sterilized using a 1 L 0.22 um filter assembly (Corning Inc., Corning N.Y.). After 2 hours, this formed a gel. The gel was then submerged in an excess of deionized water and allowed to soak in order to remove the residual salts (potassium nitrate). The gel soaked for 5 days and the deionized water was replaced three times during this period. On the 6^{th} day, the media was replaced with an excess of Murashige and Skoog media with 3 wt % sucrose. After soaking in the MS/sucrose media for 24 h, the excess liquid was decanted. The final conductivity of the gel was 3.8 mS. The gel was then autoclaved for sterility prior to testing Wax paper tubes (1.19 cm diameter) were cut to 4 cm lengths with flat openings. The bottom of the tubes was closed using pre-stretched Parafilm® M. Then, a plug of silicate gel nutrient media approximately 2 cm thick was added to the tube. Next, a sugarcane plantlet was placed on top of the nutrient gel. Next the top of the tube was closed with pre-stretched Parafilm® M. In addition, other treatments were studied. This included the testing of agar nutrient media which had been soaked in a Murashige and Skoog nutrient media containing 0.57 ppm ethephon (2-chloroethylphosphonic acid) and 3 wt % sucrose for 24 hours. This was assembled into wax paper tube artificial seeds as described above for the other media. In another treatment, wax paper tube artificial seeds as described above were created containing agar media with Murashige and Skoog nutrients and 3 wt % sucrose, except a thin polyethylene film (produce bag from grocery store) was cut into a rectangle approximately 4×7 cm and wrapped around the end of the top end of the paper tube and held in place using a rubber band, forming an open ended flexible tube structure, instead of covering the tube with pre-stretched Parafilm®. In another treatment, cold-water soluble film (Extra Packaging, Boca Raton, Fla.), was cut into approximately 7.5 cm square pieces. Autoclaved vermiculite was placed in the center of each square, forming a pile occupying an approximately 3 cm diameter circle. Next, pieces of agar media containing Murashige and Skoog nutrients and 3 wt % sucrose amounting to approximately 2-4 g were placed on top of the vermiculite. Next, a sugarcane plantlet was placed amongst and in contact with the agar pieces. Additional vermiculite was added to cover the sugarcane plantlet and the media. Finally, the edges of the cold-water soluble film were gathered forming a packet, and were taped at together at the top, resulting in a semi-spherical shaped artificial seed.

The tube shaped artificial seeds were planted in Metro-Mix® 360 such that the top of the wax paper sections were approximately 0.3-0.5 cm above the soil surface, in 10 cm plastic pots and grown in a (Conviron model BDW-120) at 31° C. during the day, 22° C. during the night, 80% relative humidity and a 13 hr photoperiod, 220 uE/m²). The packet type seeds were buried in Metro-Mix® 360 such that the top of the pouches were in contact with the soil surface, in 10 cm plastic pots and were incubated under the same conditions as the tube shaped artificial seeds.

TABLE 24
Results of growing artificial seeds with various media types and structures.

| | | | Number of | |
|---|---|---|---|---|
| | | | artificial | Number |
| | | | seeds | of seeds |
| Seed | Nutrient | | initially | germinated |
| structure | containing gel | Nutrient | planted | at day 29 |
| 4 cm wax | 0.7 wt % | MS media + 3 | 12 | 6 |
| paper tube | Difco ® agar | wt % sucrose | | |
| 4 cm wax | Kasil ® 1 based | MS media + 3 | 12 | 12 |
| paper tube | silicate gel | wt % sucrose | | |
| 4 cm wax | 0.7 wt % | MS media + 3 | 14 | 7 |
| paper tube | Difco ® agar | wt % sucrose | | |
| | | (soaked in | | |
| | | 0.57 ppm | | |
| | | ethephon | | |
| | | solution) | | |
| 4 cm wax | 0.7 wt % | MS media + 3 | 13 | 4 |
| paper | Difco ® agar | wt % sucrose | | |
| tube-open | | | | |
| polyethylene | | | | |
| bag on top | | | | |
| Cold-water | 0.7 wt % | MS media + 3 | 7 | 1 |
| soluble | Difco ® agar | wt % sucrose | | |
| packet | | | | |

From Table 24, the silicate gel based nutrient media resulted in improved germination of artificial seeds compared to agar-based nutrient media.

### Example 31

### Wax Paper Tube Artificial Seeds with Plantlet Inserted from a Side Opening

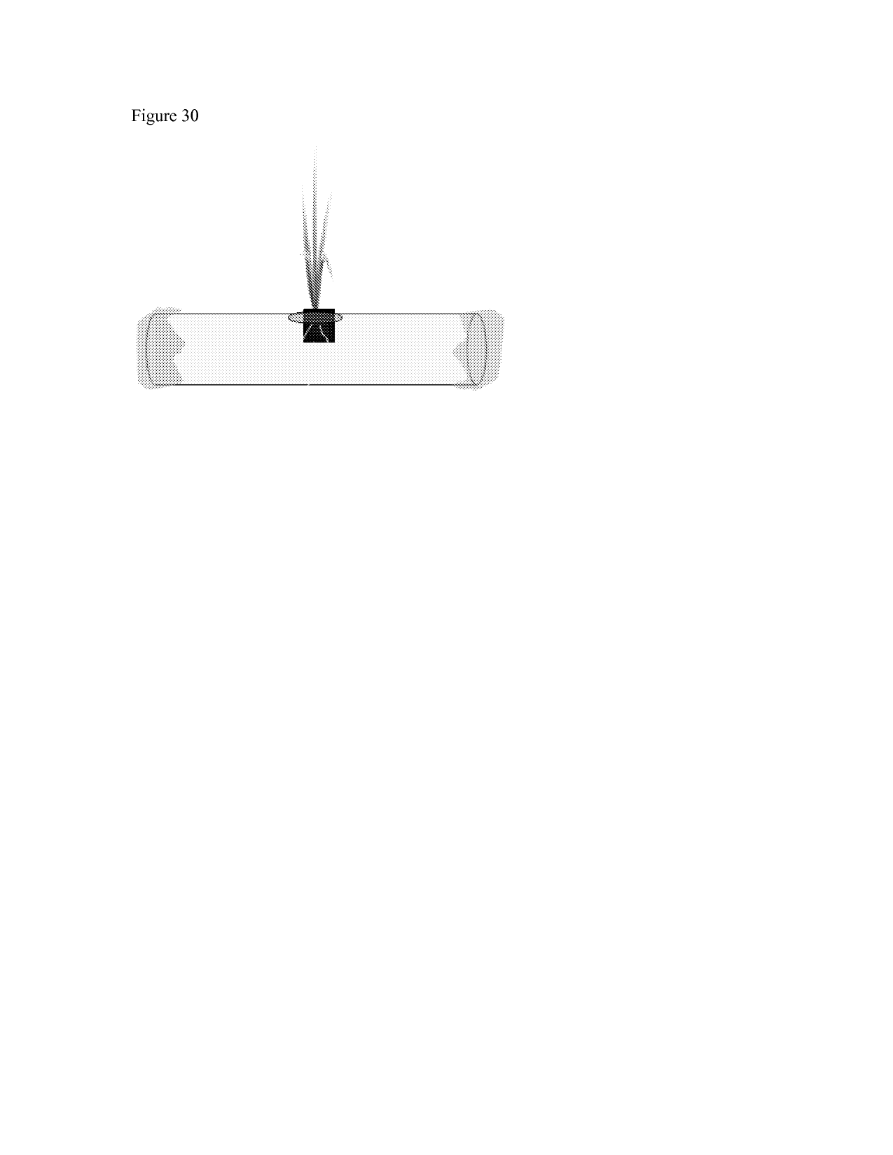

In this example, we studied the insertion of a plantlet from a side opening, in the center of a 5 cm wax paper tube section. 1.19 cm diameter wax paper tubes were cut into 5 cm sections and autoclaved. One end of the wax paper tube was closed with pre-stretched Parafilm® M. Then, nutrient media consisting of 4 wt % low melting agarose with Murashige and Skoog nutrients, 3 wt % sucrose and 0.2 wt % Plant Preservative Mixture with 150 ppm Maxim® 4FS (Syngenta, Wilmington, Del.) and 100 ppm Apron® XL (Syngenta, Wilmington, Del.) were added to fill the paper tube. The second opening of the wax paper tube was closed with pre-stretched Parafilm® M. Next, an approximately 4 mm diameterhole in the center of the 5 cm wax paper tube was made using the sharp end of metal forceps. Then, a sugarcane plantlet, having been previously cultured for 10 days in liquid nutrient media was inserted into the hole, leaving shoots pointing outward (FIG. 30). The final assembly was planted in Metro-Mix® 360 in 10 cm plastic pots with trays in a growth chamber (Conviron model BDW-120) at 31° C. during the day, at 22° C. during the night, 80% relative humidity and a 13 hr photoperiod (220 uE/m²). The tubes were planted horizontally such that the upper tube surface was flush with the soil surface and the plantlet was pointed upward. The survival rate of these tubes was 3 out of 12 planted at day 27. Bare plantlets were also planted, which exhibited a survival rate of 12 out of 24 planted at day 27.

### Example 32

### Effect of Tube Length for Wax Paper Tube Artificial Seeds

The purpose of this example was to study the effect of the length of the wax paper tube on artificial seed survival. Wax paper tubes (1.19 cm diameter) were cut into 4, 8 and 12 cm lengths. The containers were also soaked in Maxim 4FS solution prior to assembly as described in Example 5. The bottom ends of the tubes were crennellated, and covered with pre-stretched Parafilm® M. Metro-Mix® 360 was put inside the tubes as a nutrient source such that an approximately 1 cm thick layer was created. Next, a sugarcane plantlet, which had been in culture for 14 days in liquid proliferation media was placed on top of the soil layer. Additional Metro-Mix® 360 was added so that the tube had a layer approximately 3-4 cm thick of soil. 1 mL deionized water was added to the tube and the top was closed with pre-stretched Parafilm® M. The tubes were planted in Metro-Mix® 360 in 10 cm plastic pots with trays in a growth chamber (Conviron model BDW-120) at 31° C. during the day, at 22° C. during the night, 80% relative humidity and a 13 hr photoperiod (220 uE/m²). The tubes were all planted at approximately 4 cm depth.

TABLE 25
Effect of tube length on germination of wax paper tube artificial seeds at
constant planting depth (4 cm).

| | | Number sprouting |
|---|---|---|
| Tube length (cm) | Number planted | at day 39 |
| 4 | 15 | 4 |
| 8 | 16 | 2 |
| 12 | 14 | 0 |

Table 25 shows that the 4 cm wax paper tubes produced higher levels of sprouting compared to the 8 cm or 12 cm long paper tubes.

In a separate, related experiment, the same three lengths of wax paper tube artificial seed were studied, this time without lids on top and with manually crimped (FIG. 16) bottoms. These were planted so that the tops of the tubes were approximately 0.5-1 cm protruding from the soil surface (deeper planting than in the earlier study that varied with tube length). For the longer tubes, this necessitated the use of deeper 8″ diameter pots.

TABLE 26
Effect of tube length on germination of wax paper tube artificial seeds at
varying planting depth.

| | Approximate | | Number sprouting |
|---|---|---|---|
| Tube length (cm) | planting depth (cm) | Number planted | at day 35 |
| 4 | 3.5 | 15 | 14 |
| 8 | 7 | 15 | 12 |
| 12 | 11 | 16 | 3 |

From Table 26, it can be seen that the long 12 cm tubes had lower sprouting rates compared to the shorter (4 cm, 8 cm) tubes.

A similar experiment was performed in the Brazilian field at DuPont do Brazil, in Paulinia. Similarly, the 4 and 8 cm tubes performed better than the 12 cm long tubes.

### Example 33

### Variation of Paper Type and Diameter for Wax Paper Tube Artificial Seeds

In this experiment, a series of different paper types and diameters were used as wax paper tube artificial seeds. 1.0 cm diameter recycled paper tubes, 2.0 cm diameter recycled paper tubes and 1.2 cm diameter tubes fabricated from water soluble paper (sodium carboxymethylcellulose, ASW 60, Aquasol Corp) were obtained from Precision Products Group, Intl (Westfield, Mass.). These were cut to 5 cm lengths. The paper tubes were assembled by first closing the bottom with pre-stretched Parafilm® M, then adding about a 1 cm layer of Metro-Mix® 360. Next, sugarcane plantlets, which had been cultured for 5 weeks in liquid media prior to the experiment were placed on top of the soil. Next, additional soil was added to create an approximately 4 cm thick layer in the tube. 1 mL deionized water was added. Finally, the top of the tube was closed with pre-stretched Parafilm® M. The tubes were planted in Metro-Mix® 360 in 10 cm plastic pots with trays in a growth chamber (Conviron model BDW-120) at 31° C. during the day, at 22° C. during the night, 80% relative humidity and a 13 hr photoperiod (220 uE/m²). The tubes were planted at approximately 4.5 cm depth.

TABLE 27
Effect of tube composition and diameter on germination of wax paper tube
artificial seeds.

| | | | Number | Percent |
|---|---|---|---|---|
| Tube body | Tube diameter | | sprouting | sprouting at |
| material | (cm) | Number planted | at day 32 | day 32 |
| Standard wax | 1.2 | 24 | 14 | 58% |
| paper (from | | | | |
| Example 2) | | | | |
| Recycled paper | 1.0 | 29 | 8 | 28% |
| Recycled paper | 2.0 | 24 | 11 | 46% |
| Water soluble | 1.2 | 12 | 8 | 67% |
| paper | | | | |
| Bare plantlets | | 36 | 26 | 72% |

From Table 27, it can be seen that the water soluble paper tubes provided a comparable level of sprouting compared to recycled and standard wax paper tubes.

A similar experiment was performed in the Brazilian field environment, at DuPont do Brazil, in Paulinia, comparing the water soluble tubes with non-water soluble wax paper tube artificial seeds, using a stapled bottom, however, poor survival was observed for the water soluble paper tubes, for reasons unclear.

### Example 34

### Viability of Artificial Seeds Comprising Slowly Biodegradable Containers in Field

Artificial seeds comprising sugarcane plantlets encapsulated by polylactide (PLA) film containers were prepared in a similar manner to the PCL film containers described in Example 29. PLA pellets were obtained from NatureWorks (Minnetonka, Minn., grade 4032D) and dissolved in chloroform (EMD Chemicals) at 10% by weight. This solution was cast on a glass substrate using a stainless steel doctor blade with a 5 cm wide and 254 um wide gap. The resultant PLA film was dried, yielding a final thickness of 25.4 um. After removal from the glass substrate, two pieces of film, measuring 5 cm in width, were overlaid and heat-sealed along the two longer edges and one of the shorter edges to create an open pouch. Pouches of both 17.8 and 10 cm 35.6 cm in length were constructed. Each pouch was loaded with 1 g of dry Metro-Mix® 360 growing media. A regenerated sugarcane plantlet was then added to the pouch, followed by an additional 2 g of dry Metro-Mix® 360 and 3 g of deionized water. The plantlets were prepared from cultivar CPO-1372 according to a procedure similar to that described in Example 1. No trimming of the plantlet shoots was necessary for the plantlet to fit entirely within the pouch. Finally, the remaining open edge of the pouch was sealed, forming a closed, air-tight PLA container around the plantlet.

These artificial seeds were planted in a field at the DuPont Stine-Haskell Research Center located in Newark, Del. The field was prepared to give a flat planting surface. In contrast to the PCL film containers described in Examples 29 and 30, the PLA film containers of this example biodegrade in soil over relatively long periods of time—in excess of months. Therefore, the PLA film containers, as constructed, do not provide a mechanism for release of plantlet roots and shoots over a time scale matching the growth characteristics of the plant. Therefore, pathways for escape were created by cutting open the containers at various locations and times. Both the top and bottom seals of the pouches were cut open, thereby creating 5 cm wide slits. In all samples, the bottom seal was removed immediately prior to planting. For half of the samples, the top seal was removed immediately prior to planting, whereas for the remaining half of the samples, the top seal was removed 19 days after planting. The artificial seeds were planted roughly 5-7.6 cm deep in a vertical orientation such that the encapsulated plantlet's shoots were facing upwards. The field was irrigated immediately after planting and generally 3 times per week thereafter.

Table 28 shows the number of artificial seeds planted for each container size and top seal removal time, as well as the percentage of plants that survived, 4 weeks after planting. Little difference in ultimate survival was seen between the four combinations of pouch length and the time at which the top seal of the pouch was removed. However, in comparison to the rapidly biodegradable PCL containers described in Example 29, for which the sugarcane plantlet is fully enclosed by the container in the first several days after planting, the artificial seeds of this example exhibited relatively low viability. This is likely due in part to more favorable growing conditions in the former case; the average temperature and volume fraction of water present in the soil over the duration of Example 29 was 29° C. and 21%, respectively, whereas the corresponding values over the duration of the present experiment were 24° C. and 32%. However, the comparatively slow biodegradation of PLA, which necessitated the removal of container seals during and after planting, is also likely a contributing factor behind the reduced survival rates. Upon planting, the nutritive media of the artificial seed comes in direct contact with the surrounding soil through the opening in the bottom of the container. This surely induces a decrease in the moisture content of the nutritive media at the location of the plantlet roots during the critical, first days after planting. In contrast, the use of a rapidly biodegradable container as described in Example 29 prevents contact between the nutritive media and the surrounding field soil during this initial stage, and its macroscopic degradation enables a gradual establishment of the plantlet roots in the surrounding soil thereafter.

TABLE 28
Survival of sugarcane plants from PLA film containers in field

| Pouch | Time of Top | | Survival of Plants |
|---|---|---|---|
| Length (cm) | Seal Removal | Number Planted | After 4 Weeks (%) |
| 17.8 | 19 days after planting | 25 | 32 |
| 17.8 | at planting | 28 | 39 |
| 35.6 | 19 days after planting | 25 | 28 |
| 35.6 | at planting | 25 | 16 |

### Example 35

### Packet Type Artificial Seeds with Holes

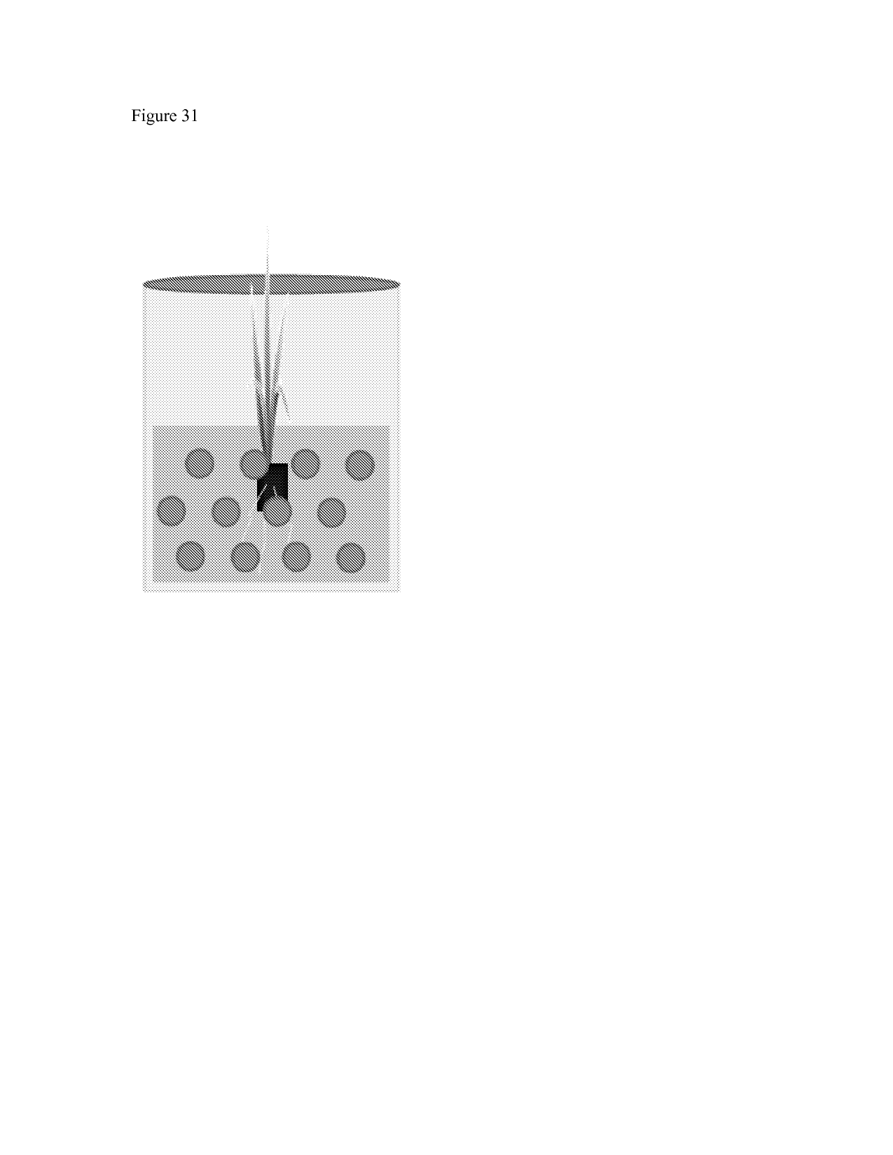

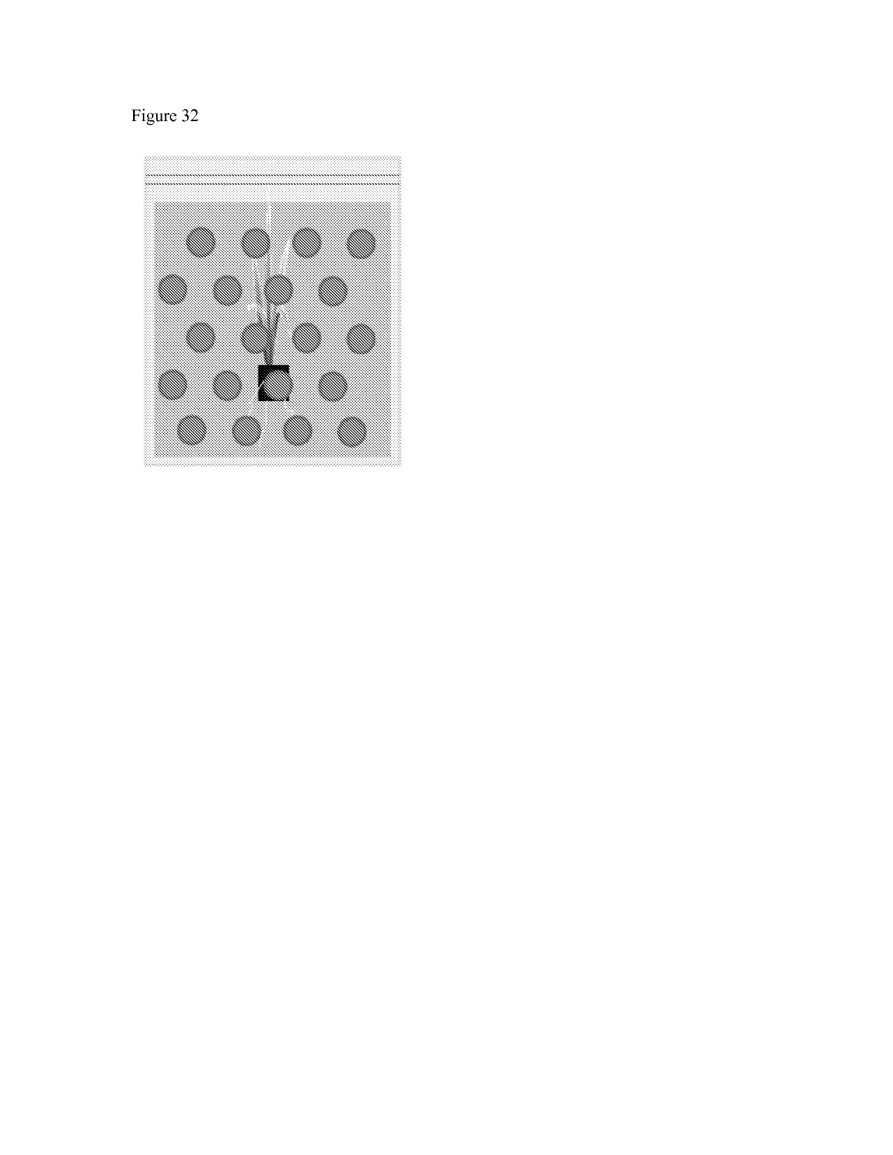

The purpose of this example was to test packet type artificial seeds possessing multiple holes. The packets were fabricated from 6.5 by 10 cm polyethylene sample bags (100 um thick) (Minigrip, Kennesaw, Ga.). In one treatment, a hole punch was used to make approximately 12, 6 mm holes in the bottom half of the sample bag. Next, moist Metro-Mix®-360 growth media and a sugarcane plantlet were added to the sample bag. The growth media approximately half-filled the sample bag. The plantlet shoots were trimmed to approximately 8 cm and the top of the bag was left open with the shoots protruding (FIG. 31). In a second treatment, approximately 20, 6 mm holes were made along the entire length of the sample bag. A sugarcane plantlet was trimmed to about 4 cm and Metro-Mix®-360 growth media was added to fill the sample bag. The top of the sample bag was secured with the built-in seal (FIG. 32). The packets were planted in a vertical orientation in Metro-Mix® 360 growth media with their tops protruding approximately 3 cm in 10 cm plastic pots with trays in a growth chamber (Conviron model BDW-120) at 31° C. during the day, at 22° C. during the night, 80% relative humidity and a 13 hr photoperiod (220 uE/m²).

TABLE 29
Results of packet experiment.

| | | Number initially | Number sprouting |
|---|---|---|---|
| Design | Top open/closed | planted | at day 28 |
| Packet-holes along | Open | 8 | 6 |
| bottom half | | | |
| Packet-holes | Closed | 8 | 0 |
| throughout | | | |
| Bare plantlets | N/a | 8 | 7 |

In Table 29, the packets with holes and the open top provided the best survival, comparable to the bare plantlets. At day 43, the artificial seeds were removed from soil, showing that the roots had grown out of the holes in the packets for the seeds with the open top. No signs of the plantlets remained for the packets with the closed tops.

### Example 36

### Hinged and Expandable Seed Designs

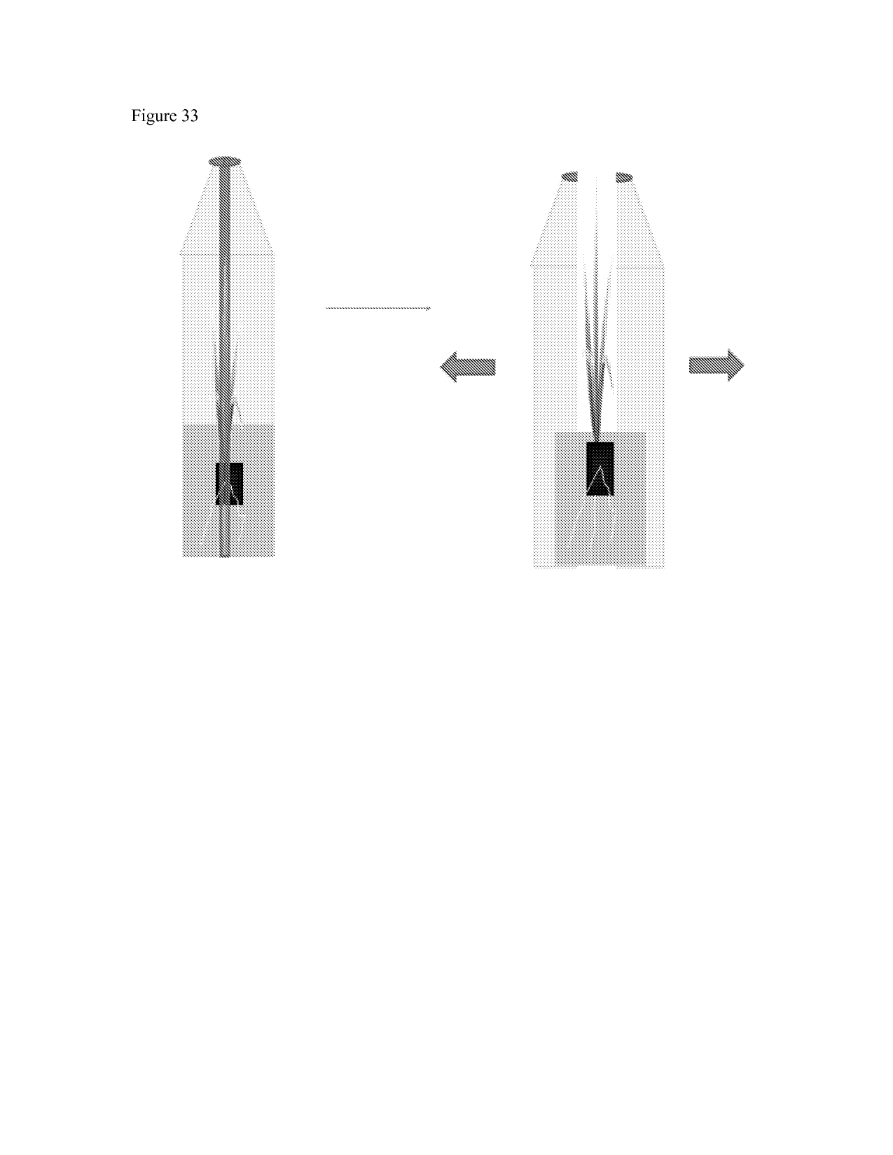

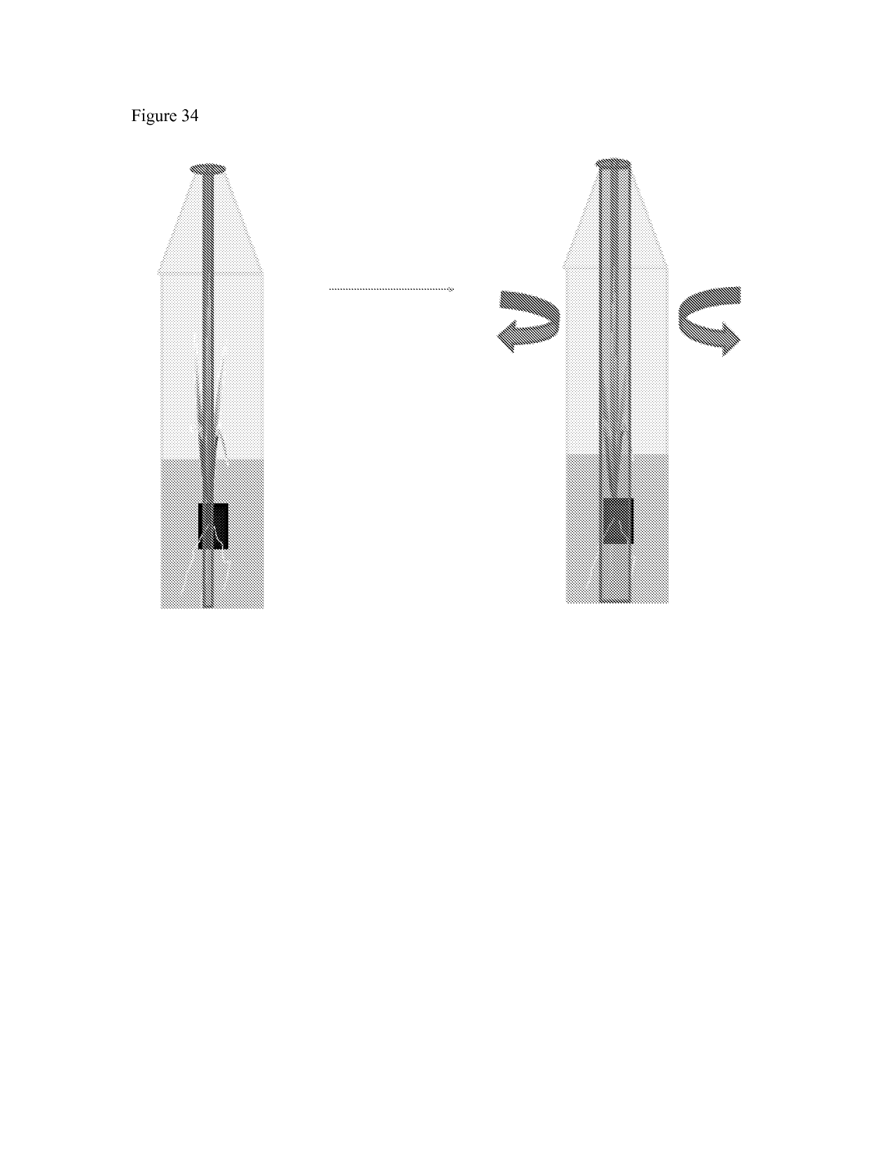

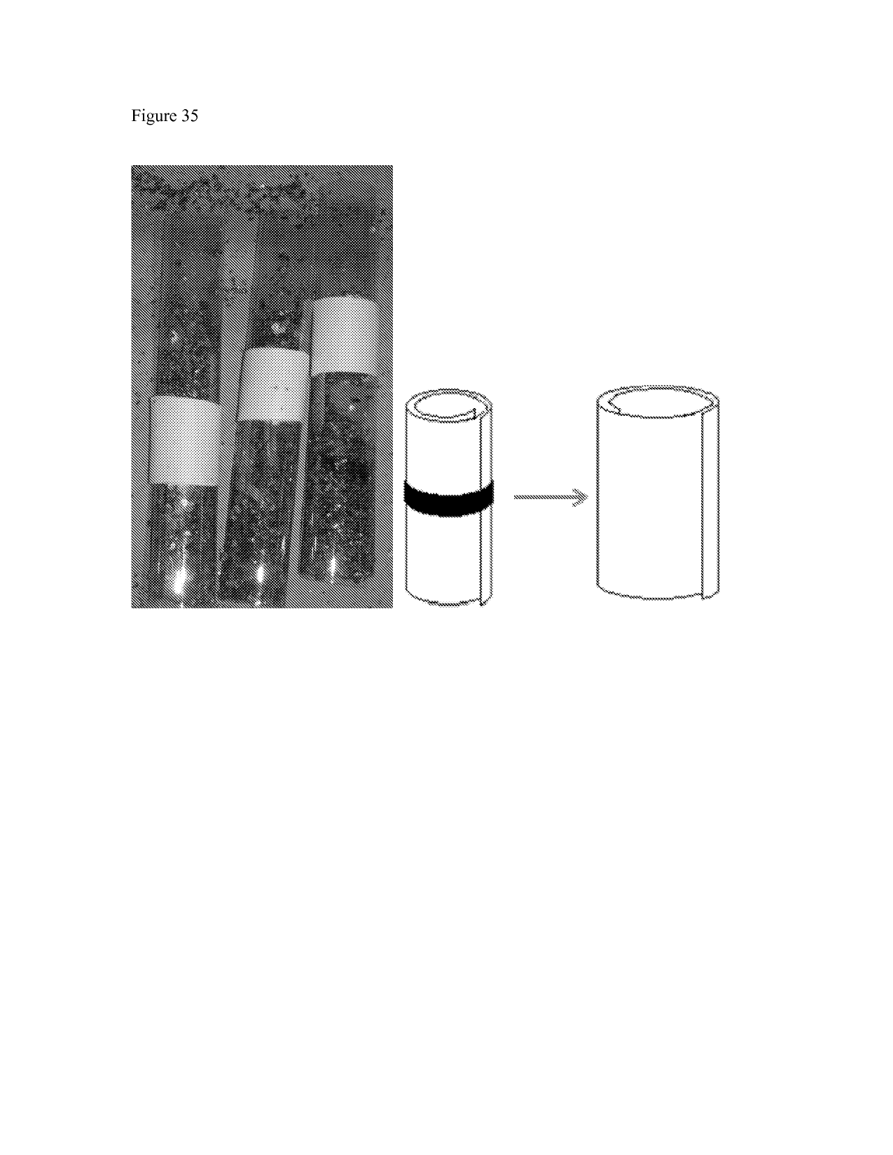

The purpose of this experiment was to study the use of hinged or expandable seed designs. The tips were cut off 50 mL centrifuge tubes (VWR International, LLC, Radnor, Pa.) resulting in 5-8 mm holes in the end. The tubes were then cut lengthwise in half. The two halves of the tubes were then re-connected by hot gluing strips (approximately 2 cm wide by approximately 9 cm long) of cold water soluble plastic film (cut from cold water soluble bags from Extra Packaging Corp, Boca Raton, Fla.). For this design, water would soften the two halves, allowing the plantlet to grow and push apart the two halves of the tube (FIG. 33). In another treatment, the two halves were re-connected by hot gluing one edge together while leaving the other side open, thereby creating a flexible hinge (FIG. 34). These tubes were positioned over the shoots of pre-planted sugarcane plantlets in Metro-Mix® 360 in 2″ pots and then forcibly pressed down with a twisting motion, such that the plant as well as the soil surrounding it were taken up in the tube. In another treatment, 100 um thick Mylar® film was cut into approximately 11 cm×12 cm rectangular pieces. The Mylar® film rectangles were wrapped into an approximately 11 cm long scroll and inserted into 50 mL centrifuge tubes and heated in a convection oven at 100° C. for 18 h in order to form them to the diameter of the 50 mL centrifuge tube (28 mm). Then, the scrolls were removed from the oven and cooled to room temperature. Approximately 2 cm long sections of 2 cm diameter wax paper tube were cut. The scrolls were wrapped more tightly and inserted into the paper bands (FIG. 35). Sugarcane plantlets with moist vermiculite were then inserted into the scrolls to create a plug 4-6 cm thick. The scroll like seeds were planted in a vertical orientation in Metro-Mix® 360 approximately 4 cm deep in 10 cm plastic pots with trays in a growth chamber (Conviron model BDW-120) at 31° C. during the day, at 22° C. during the night, 80% relative humidity and a 13 hr photoperiod (220 uE/m²). The paper bands were cut immediately after planting, allowing the scrolls to expand back to close to their original diameter (28 mm). The hinged tube designs were planted 4-5 cm deep in the same flats with the scroll type seeds.

TABLE 30
Results of hinged and expandable seed design experiment.

| | Number initially | Number sprouting |
|---|---|---|
| Design | planted | at day 28 |
| 50 mL tube with cold water | 15 | 14 |
| soluble film edges | | |
| 50 mL tube with hot glued hinge | 15 | 14 |
| Expandable “scroll”-type | 11 | 10 |
| Bare plantlets | 36 | 34 |

As can be seen in Table 30, all structures performed well in terms of germination and comparably to the bare plantlets. By day 23, there was evidence of some of the hinged seeds expanding to accommodate the growing plantlets.

### Example 37

### Expandable Artificial Seed Structures and Other Variations

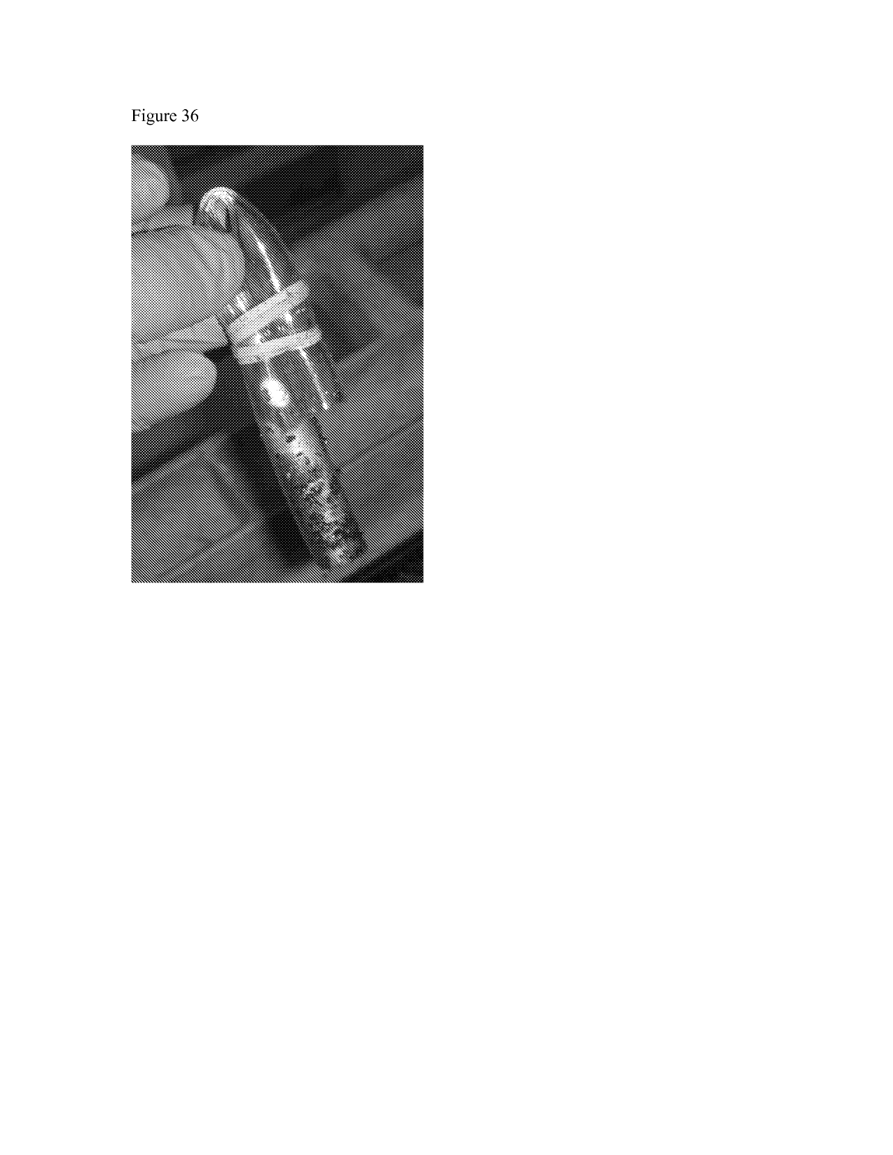

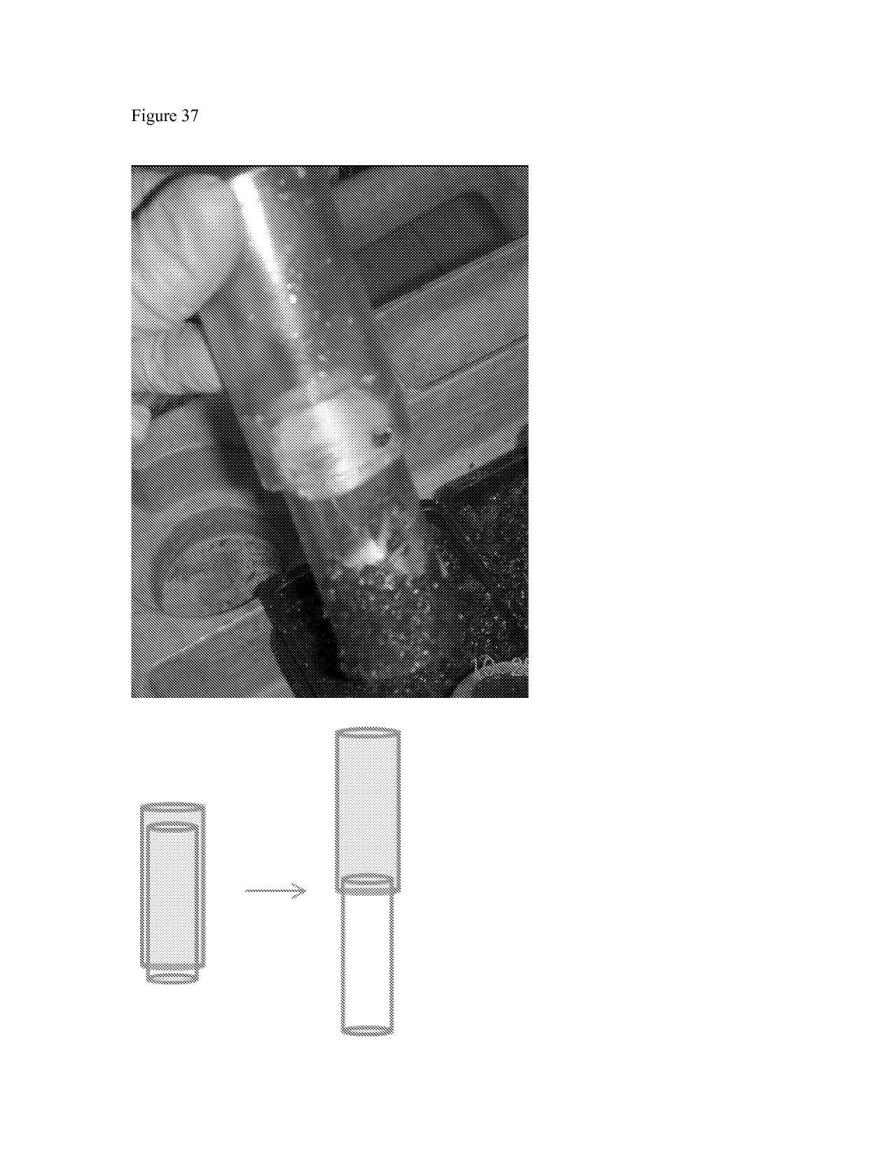

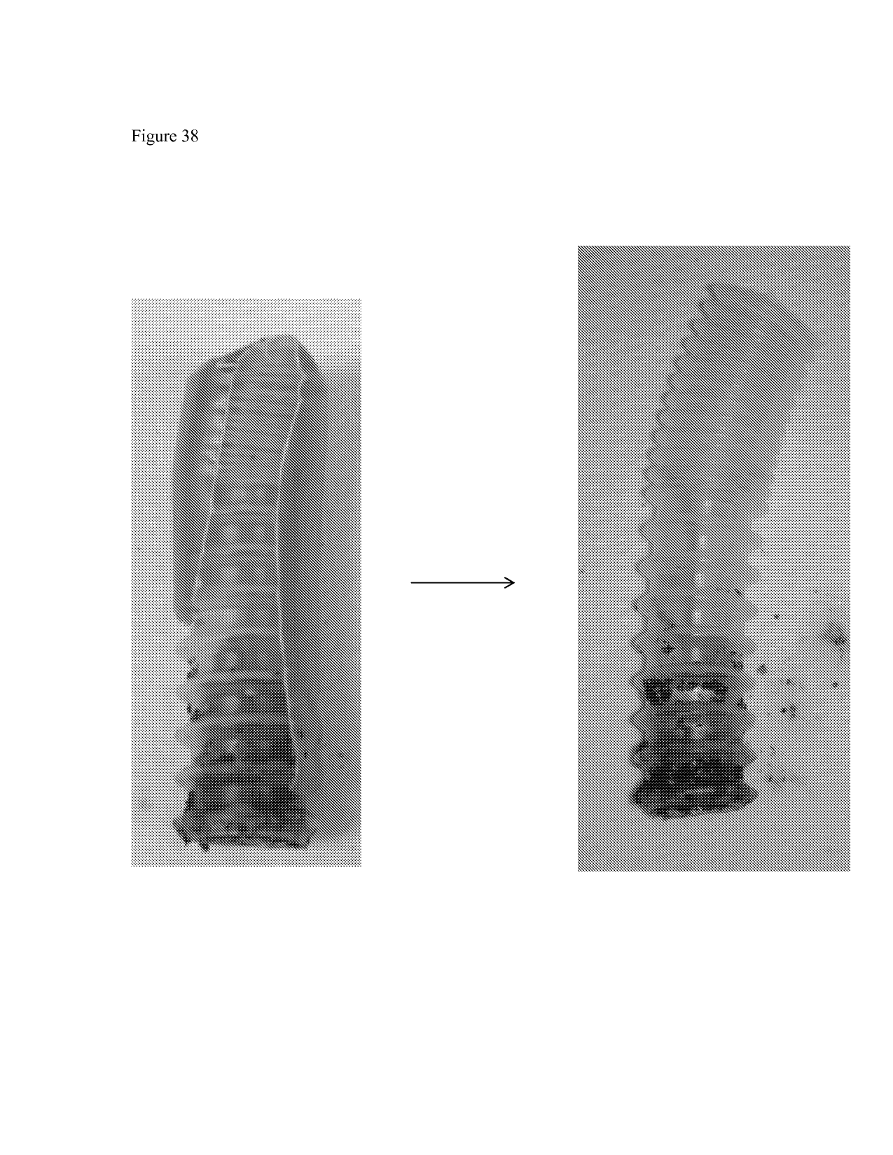

The purpose of this experiment was to test a variety of expandable artificial seed structures. This included foldable, telescoping and accordion-like structures. The purpose of these seed structures was to achieve a smaller size in storage conditions and a larger size in the field after planting. This would be beneficial for increasing storage density on a planter and, as seen in Example 20, increased size of seeds resulted in higher survival rates. 1.25 cm inner diameter Tygon® tubing (1.59 cm outer diameter, MSC Industrial Supply Co., Melville, N.Y.) was cut into 16.5 cm lengths. Next, a sugarcane plantlet and moist Metro-Mix® 360 were inserted into the bottom end of the tube, creating a soil plug approximately 4 cm long. The top 6 cm of the tubing was folded over and secured with a rubber band (FIG. 36). The rubber band was removed at the time of planting, resulting in the unfolding of the tube. In another treatment, a telescoping seed structure was made using transparent plastic pipe. Clear schedule 40 PVC pipe of two different diameters, 3.35 cm outer diameter/2.62 cm inner diameter and 4.22 cm outer diameter/3.45 cm inner diameter (MSC Industrial Supply Co., Melville, N.Y.) were cut into lengths of 7.6 cm. The narrower piece was wrapped with an approximately 2 cm wide band of Parafilm® M and inserted into the wider piece concentrically to create a snug fit. The assembly was positioned over the shoots of sugarcane plantlets which had been planted in moist Metro-Mix® 360 in 10 cm pots and then forcibly pressed down with a twisting motion, such that the plantlet as well as the soil surrounding it were taken up in the tube. The tube was then lifted out, resulting in a soil plug approximately 3 cm thick. Both ends of the tube were left open. The outer section of transparent plastic pipe was slid upward relative to the inner section at the time of planting, leaving an approximately 2 cm overlap, in order to create a taller (˜13 cm) seed structure (FIG. 37). In another treatment, an accordion-like expandable seed was made using the ribbed outlet tube from a plastic hand operated siphon drum pump (MSC Industrial supply co, Melville, N.Y.). The ribbed outlet tube consists of a segment of more compressible, narrowly spaced (every 3 mm) ribs with thinner plastic, adjoining a segment of thicker walled, less compressible more broadly spaced (every 6 mm) ribs and is approximately 1.5 cm in diameter. The ribbed outlet tube was cut such that a 5 cm long segment of the more rigid tubing adjoined a 4 cm long segment of the more flexible tubing. The assembly was positioned over the shoots of sugarcane plantlets which had been planted in moist Metro-Mix® 360 in 2″ pots and then forcibly pressed down with a twisting motion, such that the plantlet as well as the soil surrounding it were taken up in the tube. The tube was then lifted out, resulting in a soil plug approximately 2 cm thick. The more flexible top section was then manually compressed to a length of approximately 2 cm, and taped in position using duct tape. The tape was removed at the time of planting, thereby allowing the tube to expand to a length of 9 cm from a compressed length of 7 cm (FIG. 38). All of the seeds were planted in a vertical orientation in Metro-Mix® 360 approximately 3 cm deep in 10 cm plastic pots with trays in a growth chamber (Conviron model BDW-120) at 31° C. during the day, at 22° C. during the night, 80% relative humidity and a 13 hr photoperiod (220 uE/m²). Bare plantlets were planted as controls.

TABLE 31
Results of expandable artificial seed experiment.

| | Number initially | Number sprouting |
|---|---|---|
| Design | planted | at day 21 |
| Foldable artificial seed | 10 | 10 |
| Telescoping artificial seed | 5 | 5 |
| Accordion-like expandable seed | 10 | 9 |
| Bare plantlet controls | 21 | 18 |

As seen in Table 31, the expandable artificial seeds produced high survival rates.

### Example 38

### Artificial Seeds with Superabsorbents and Other Variations

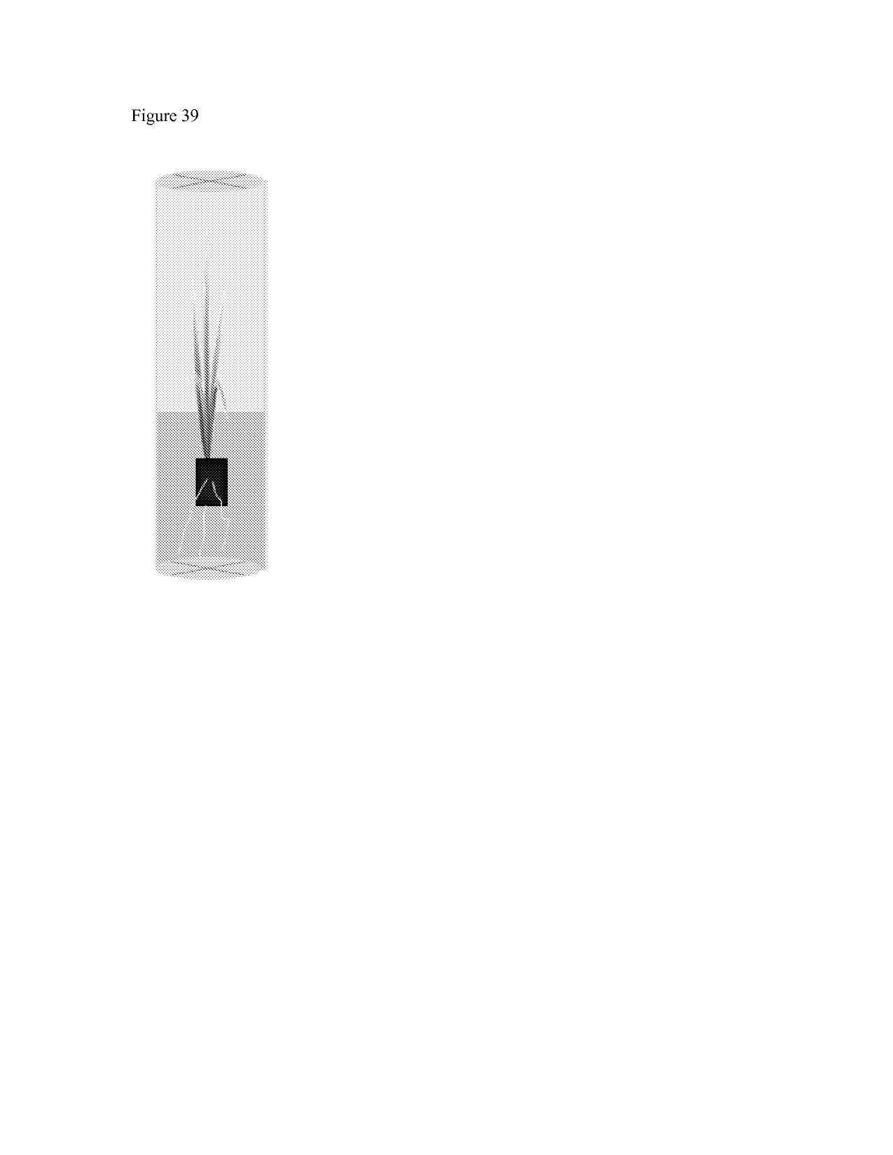

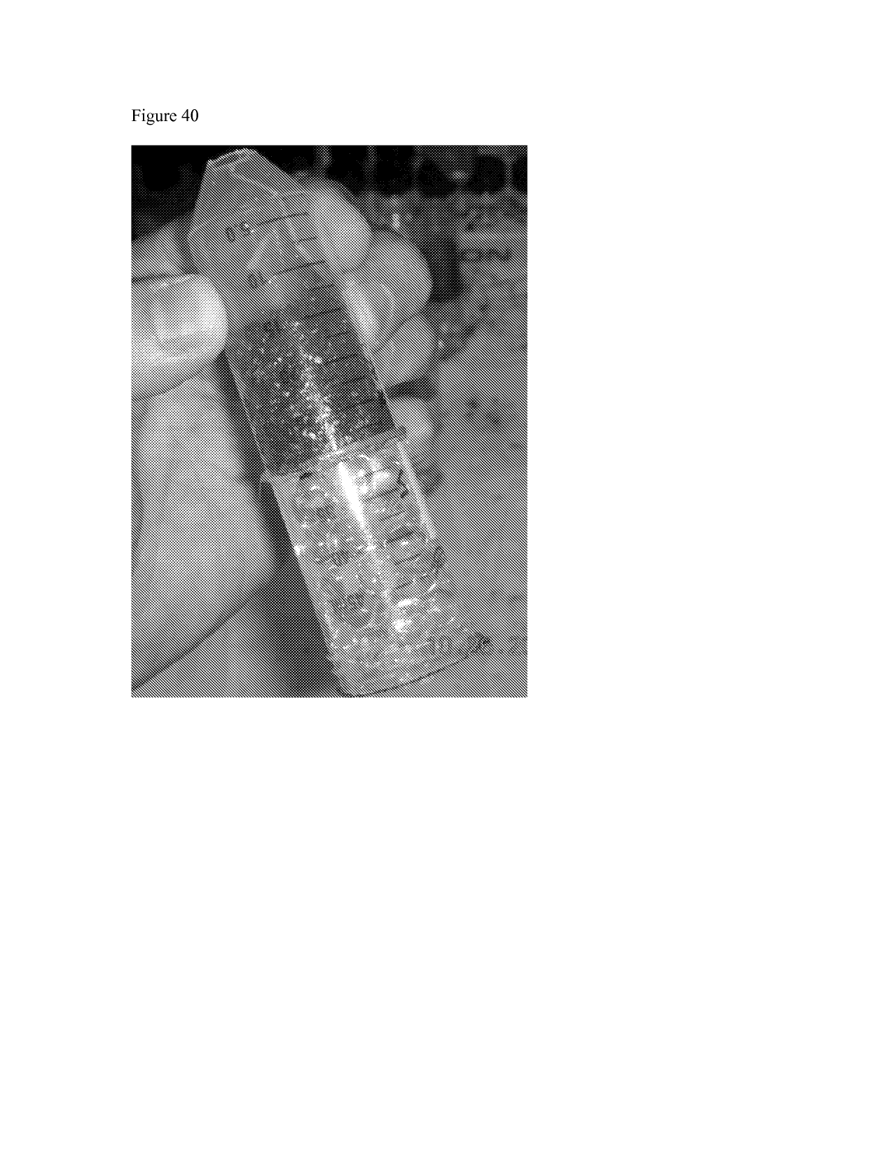

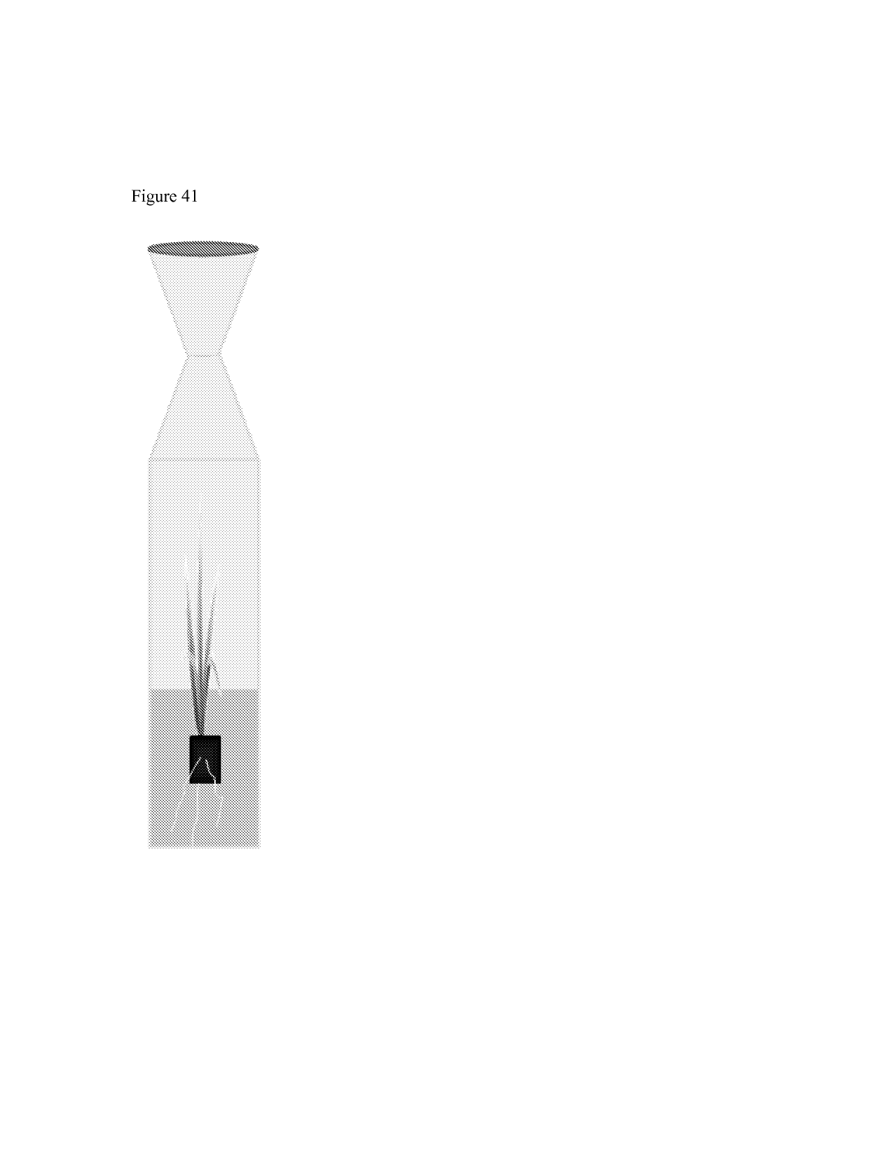

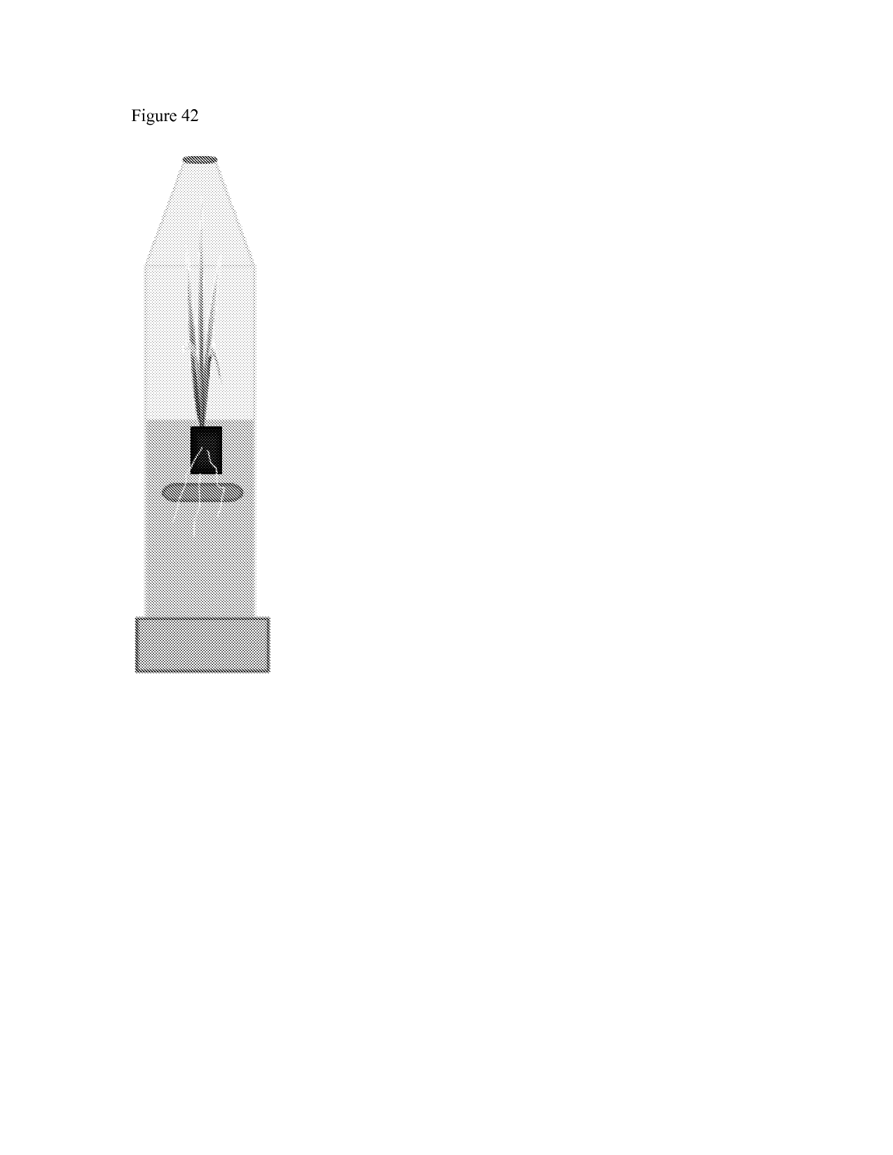

The purpose of this experiment was to study the use of superabsorbents in various configurations in the artificial seeds, as well as other variations including a funnel shaped lid and slotted film lids. In one treatment, the conical tips were entirely cut off 15 mL centrifuge tubes (VWR International, LLC, Radnor, Pa.). The tube was positioned over the shoots of sugarcane plantlets which had been planted in moist Metro-Mix® 360 in 2″ pots and then forcibly pressed down with a twisting motion, such that the plantlet as well as the soil surrounding it were taken up in the tube. Unstretched Parafilm® M was then hot glued to both ends of the tube. A razor blade was used to cut an “X” with the cuts extending to the edges of the tube on both the top and bottom. This created a slotted lid opening on both ends of the tube (FIG. 39). In another treatment, 15 mL centrifuge tubes were used to make artificial seeds as in Example 20, except the bottoms were covered by hot gluing hot water soluble plastic film which had been cut from bags into ˜2 cm squares (Extra Packaging Corp., Boca Raton, Fla.). In another treatment, the tapered tips were cut off 50 mL centrifuge tubes (VWR International, LLC, Radnor, Pa.) revealing a 5-8 mm hole, and the tubes were cut at the 30 mL graduation (4.5 cm from the wide threaded opening). Then the tube with the conical section was positioned over the shoots of sugarcane plantlets which had been planted in moist Metro-Mix® 360 in 2″ pots and then forcibly pressed down with a twisting motion, such that the plantlet as well as the soil surrounding it were taken up in the tube, resulting in a 3 cm soil layer. Then, plastic window screen (Lowe's Home Improvement, Newark, Del.) was hot glued to the bottom below the soil plug. The second piece of the tube was then glued back onto the bottom, and superabsorbent polymer (Magic water beads, magicwaterbeads.com) which had been pre-swollen in deionized water were added to the lower section of the tube. Finally, a second layer of plastic window screen was hot glued to the bottom of the structure (FIG. 40). In another treatment, 50 mL centrifuge tubes were used to fabricate artificial seeds as in Example 20, except superabsorbent beads (Magic water beads) pre-swollen in deionized water were mixed with the soil in an approximate 1:1 volume:volume ratio with the moist Metro-Mix® 360. Also, a thicker segment of soil with beads was used, approximately 5.5 cm thick. In a related treatment, the same procedure was followed, except half of the Magic water beads were pre-swollen in deionized water and half in Miracle-Gro® (The Scotts Company, LLC) fertilizer solution. In another treatment, 50 mL centrifuge tubes (VWR International, LLC, Radnor, Pa.) were used to make artificial seeds as in Example 20, except two 15 mL centrifuge tubes with tapered ends cut off and caps on, containing superabsorbent beads (Magic water beads) pre-swollen in deionized water (for one of the tubes) and pre-swollen in Miracle-Gro® (The Scotts Company, LLC) fertilizer solution (for the other tube), were hot glued to opposite sides of the 50 mL tube, and the bottoms covered with plastic window screen (Lowe's Home Improvement, Newark, Del.) by hot gluing. The 15 mL tubes were positioned parallel to the 50 mL tube and shifted downward such that they extended 2 cm below the open bottom of the 50 mL tube. In another treatment, 50 mL centrifuge tubes were used to fabricate artificial seeds as in Example 20, except that a funnel shaped piece, fabricated by cutting the conical, tapered end off another 50 mL tube was hot glued to the top of the 50 mL tube, with the wide end pointing upward (FIG. 41). In another treatment, 50 mL centrifuge tubes were used to fabricate artificial seeds as in Example 20, except that the bottom plastic cap was put back on the end of the tube and two slots were cut on opposite sides of the tube, 3.5 cm from the capped end, that were perpendicular to the tube axis and were approximately 23 mm in length and 5 mm in width (FIG. 42). This design resulted in a closed cup filled with moist Metro-Mix® 360 at the bottom of the seed, and the slots acted as points through which the roots could grow. All of the seeds were planted in a vertical orientation in Metro-Mix® 360 approximately 3 cm deep in 10 cm plastic pots with trays in a growth chamber (Conviron model BDW-120) at 31° C. during the day, at 22° C. during the night, 60% relative humidity, and a 13 hr photoperiod (220 uE/m²). Bare plantlets were planted as controls. Some artificial seeds and bare plantlets were planted in dry Metro-Mix® 360, while others were planted in moist Metro-Mix® 360 as shown in Table 32. In this experiment, the soil was not watered subsequent to planting.

TABLE 32
Results of testing various superabsorbent containing seeds and other
designs.

| | Initial Metro- | Number | Number |
|---|---|---|---|
| | Mix ® 360 | initially | sprouting |
| Design | moisture condition | planted | at day 15 |
| Slotted unstretched | wet | 15 | 13 |
| Parafilm ® M lidded 15 mL | | | |
| conical tube | | | |
| Hot water soluble | wet | 15 | 12 |
| bottom lidded 15 mL | | | |
| conical tube | | | |
| 50 mL conical tube | wet | 12 | 12 |
| with funnel shaped top | | | |
| Bare plantlets | wet | 18 | 18 |
| Superasborbent in | dry | 11 | 11 |
| screened section 50 mL | | | |
| conical tube | | | |
| Water swollen | dry | 15 | 15 |
| superasborbent beads | | | |
| mixed with soil 50 mL | | | |
| conical tube | | | |
| Fertilizer solution | dry | 14 | 14 |
| swollen superasborbent | | | |
| beads mixed with soil | | | |
| 50 mL conical tube | | | |
| 50 mL conical tube | dry | 11 | 11 |
| with two 15 mL tubes | | | |
| containing | | | |
| superabsorbent beads | | | |
| Side slotted tube with | dry | 18 | 14 |
| closed end containing | | | |
| moist Metro-Mix ® 360 | | | |
| Bare plantlets | dry | 10 | 5 |

In Table 32, it is clear that under wet initial soil conditions, all seed structures performed well, as did the bare plantlets. For seeds planted in dry soil, there was a larger difference in survival, with higher survival for the seed structures than for the bare plantlets. At day 24 after planting, the seeds with the slotted sides with closed ends were exhumed and it was observed that the roots of the plants successfully emerged from through the side slot openings for the seeds that had sprouted.

### Example 39

### Artificial Seeds with Multiple Plantlets

The purpose of this example was to study the use of multiple plantlets in the same artificial seed structure. 2 cm diameter wax paper tubes were cut into 6 cm long sections. Sugarcane plantlets were trimmed to 4 cm length. The bottom ends of the tubes were covered with pre-stretched Parafilm® M. A layer of Metro-Mix® 360 approximately 2 cm thick was added to the bottom. Either 1 or 2 trimmed plantlets were placed on top, and more Metro-Mix® 360 was added until the tube was approximately 75% full. Approximately 3 mL water was added. The top was then covered with pre-stretched Parafilm® M. The artificial seeds were planted in a vertical orientation in Matapeake/sand soil (a mix of a Maryland soil with sand, creating a high sand content soil) such that their tops were approximately 0.5 cm above the soil surface in 10 cm plastic pots with trays in a growth chamber (Conviron model BDW-120) at 31° C. during the day, at 22° C. during the night, 40% relative humidity, and a 13 hr photoperiod (220 uE/m²). The results are summarized in Table 33. The sprouting from the paper tube artificial seeds containing 2 plantlets was comparable under these conditions to that of the artificial seeds containing 1 plantlet.

TABLE 33
Results of multiple plant per seed experiment.

| | Number of | | |
|---|---|---|---|
| | plantlets | Number initially | Number sprouting |
| Design | per seed | planted | at day 26 |
| 2 cm wax paper | 1 | 17 | 10 |
| tube with pre- | | | |
| stretched Parafilm ® | | | |
| on both ends | | | |
| 2 cm wax paper | 2 | 16 | 7 |
| tube with pre- | | | |
| stretched Parafilm ® | | | |
| on both ends | | | |

### Example 40

### Effect of Soil Layer Thickness and Bottom Lid Under Drought Stressed Conditions

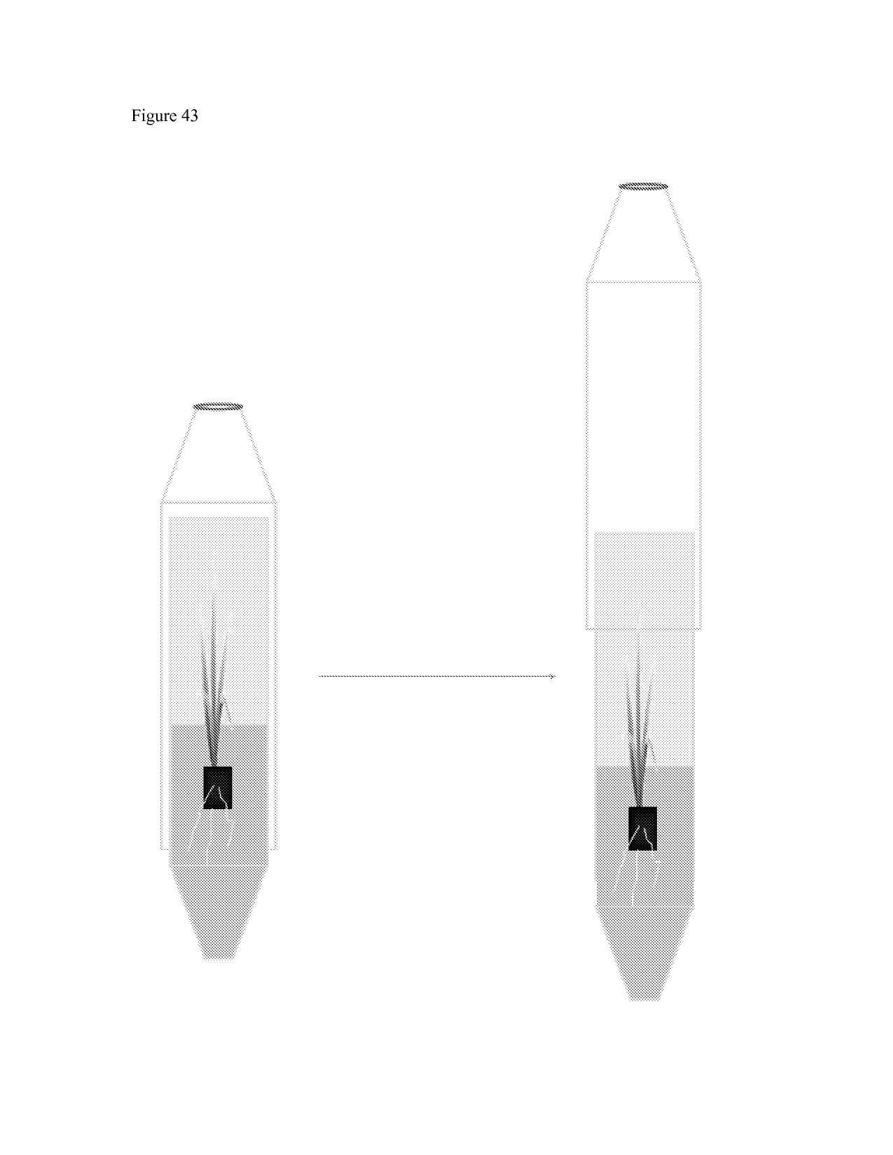

The purpose of this experiment was to study the effect of changing the soil plug thickness in conical tube artificial seeds and using bottom lids under drought stressed conditions. In one treatment, 15 mL conical centrifuge tube artificial seeds were created as in Example 20, with 4 cm thick soil plugs. These were planted about 4 cm deep in 10 CM pots in dry 50:50 Matapeakee/sand soil (a mix of a local Maryland soil with sand, creating a high sand content soil). In another treatment, additional moist Metro-Mix® 360 was added from the bottom end of the tube until the top of the soil layer was 9 cm thick. The bottom of the tube was either left open or covered with pre-stretched Parafilm® M. These were planted about 9 cm deep in 10 cm pots in dry 50:50 Matapeake/sand soil (a mix of a local Maryland soil with sand, creating a high sand content soil). In another treatment, poly(ε-caprolactone) sleeves (75 um thickness) were created by pouring a solution of 8 wt % poly(ε-caprolactone) (Sigma Aldrich, St. Louis, Mo.) in chloroform (EMD Chemicals, a division of Merck KGaA, Darmstadt, Germany) into 50 mL centrifuge tubes, pouring out the excess and allowing the film to dry in a laboratory fume hood at ambient temperature for 2 days. The sleeves spontaneously shrunk away from the walls of the centrifuge tube, and were manually pulled out. They were allowed to dry for an additional 1 week at ambient temperature in the fume hood. The sleeves were then filled to 2 cm from the top with moist Metro-Mix® 360, and a sugarcane plant was then planted in the top. Additional moist Metro-Mix® 360 was added until the soil was about 0.5 cm from the top of the sleeve. The tip of a 50 mL conical polypropylene centrifuge tube (VWR International, LLC, Radnor, Pa.) was cut, revealing a 5-8 mm hole. This tube was then placed over the sleeve and slid down to form two concentric tubes (FIG. 43). Before planting, the centrifuge tube was telescoped upward, leaving about 2 cm overlap with the inner sleeve. The assembly was planted in dry Matapeakee/sand mix such that the top of the sleeve section was nearly flush with the soil surface. The artificial seed pots were placed in a growth chamber (Conviron model BDW-120) at 31° C. during the day, at 22° C. during the night, 40% relative humidity, and a 13 hr photoperiod (220 uE/m²). The artificial seeds were not watered during this experiment.

TABLE 34
Results of experiment studying the effect of soil layer thickness and
bottom lid under drought stressed conditions.

| | | | Number | Number |
|---|---|---|---|---|
| | | | surviving at | surviving at |
| | Soil section | Number | day | day 17 |
| | thickness | initially | 10 without | without |
| Design | (cm) | planted | irrigation | irrigation |
| 15 mL conical | 4 | 30 | 17 | 0 |
| tube open bottom | | | | |
| 15 mL conical | 9 | 30 | 29 | 0 |
| tube open bottom | | | | |
| 15 mL conical | 9 | 30 | 30 | 2 |
| tube with pre- | | | | |
| stretched | | | | |
| Parafilm ® on | | | | |
| bottom | | | | |
| Poly(ε- | 10 | 18 | 18 | 16 |
| caprolactone) | | | | |
| sleeve with | | | | |
| telescoping 50 mL | | | | |
| conical tube | | | | |

As in Table 34, it is clear that the thicker soil layer resulted in higher survival rates under drought stressed conditions. An additional observation was that the artificial seeds with Parafilm® M lids on bottom exhibited better vigor at the end of the 10 day period, compared to the treatment with the open bottom. Also, the telescoping design with the poly(ε-caprolactone) sleeve showed better survival than the 15 mL tube with 4 cm soil layer. After 31 days, the telescoping design with the poly(ε-caprolactone) sleeves were exhumed and it was observed that roots had grown out of the bottoms of the sleeves into the surrounding soil.

### Example 41

### Effect of Conical Lids (15 mL Conical Plastic Tubes) on Top of Wax Paper Tubes in Field Testing for Artificial Seeds in Brazil

Cylindrical wax paper containers (Colossal drinking straw, Aardvark®, Precision Products Group, Ft Wayne, Ind., 1.19 cm outer diameter) were cut into 5 cm and 8 cm lengths. Sugarcane plantlets, cultivar V11 (SP813250) which had been regenerated for 28 days from bud tissue fragments in plantlet regeneration medium were used for this experiment. The plantlet shoots were trimmed to fit in the length of the tubes. The bottoms of the paper tubes were closed by wrapping pre-stretched Parafilm® M across the bottom. A thin approximately 1 cm layer of autoclaved potting soil (Tropstrato® HT) was placed at the bottom of the tubes. The plantlets were placed on the soil layer, and then additional potting soil was added to fill the tube until the plantlet was mostly covered. A volume of approximately 1 mL of water was added into the structure, and then the tops of the tubes were closed with either pre-stretched Parafilm® M or conical lids (15 mL polypropylene centrifuge tubes (Corning®) without holes.

The artificial seeds were planted in a vertical orientation in raised beds at the DuPont do Brasil site in Paulinia (SP), Brazil such that the tops of the tubes were less than 0.5 cm above the soil surface. Bare plantlets without trimming were planted in both the field, as well as a nearby greenhouse onsite (using the same autoclaved potting soil used inside the structures) in 8 cm pots (240 mL volume)). The field soil had been prepared before the experiment using rotary hoes and a bed shaper. After planting, irrigation was performed daily and survival was monitored every two days.

TABLE 35
Results of field experiment with wax paper tube artificial seeds.

| | | | | % |
|---|---|---|---|---|
| | | | | Survival |
| | | | Initial # of | on |
| Seed structure | Top closure | Bottom closure | containers | day 30 |
| 5 cm Wax | Pre-stretched | Pre-stretched | 30 | 20.0 |
| paper tube | Parafilm ® M | Parafilm ® M | | |
| 5 cm Wax | 15 mL centrifuge | Pre-stretched | 30 | 46.7 |
| paper tube | tube | Parafilm ® M | | |
| 8 cm Wax | Pre-stretched | Pre-stretched | 30 | 13.3 |
| paper tube | Parafilm ® M | Parafilm ® M | | |
| 8 cm Wax | 15 mL centrifuge | Pre-stretched | 30 | 10.0 |
| paper tube | tube | Parafilm ® M | | |
| Bare plantlet - | None | None | 35 | 68.6 |
| Field | | | | |
| Bare plantlet - | None | None | 30 | 66.7 |
| Greenhouse | | | | |

As shown in Table 35, the shorter (5 cm) tubes provided higher levels of survival compared to the 8 cm tubes. For the 5 cm tubes, the treatment with a conical plastic lid provided a higher survival rate than the one with pre-stretched Parafilm® M lids. The plantlets were observed to rupture the Parafilm® lids in this experiment.

### Example 42

### Effect of Superabsorbent Polymer Inside Wax Paper Tubes

Cylindrical wax paper containers (Colossal drinking straw, Aardvark®, Precision Products Group, Ft Wayne, Ind., 1.19 cm outer diameter) were cut into 4 cm lengths. Sugarcane plantlets, cultivar V11 (SP813250) which had been regenerated for 28 days from bud tissue fragments in plantlet regeneration medium were used for this experiment. The plantlet shoots were trimmed to approximately 3 cm length before encapsulation. The bottoms of the paper tubes were either stapled along the axis of the tube with half of the staple extending beyond the end of the tube, or closed by wrapping pre-stretched Parafilm® M across the bottom. Approximately 1 cm of the tube was filled either with a superabsorbent polymer (Stockosorb®) solution mixed with Murashige and Skoog nutrient media or with autoclaved potting soil. The tops of the tubes were closed with pre-stretched Parafilm® M.

The artificial seeds were planted in a vertical orientation in 8 cm pots (240 mL volume) filled with a mixture of 1:1 weight to weight ratio Paulinia field soil to sand in the growth chamber. Bare plantlets without trimming were planted in pots filled with the same mixture (field control) and also in pots covered with plastic lids filled with autoclaved potting soil (greenhouse control). The pots were left in a growth chamber. After planting, irrigation was performed daily only for the pots filled with potting soil and survival was monitored every two days. For all the treatments, autopsies were made after 5, 14 and 33 days.

TABLE 36
Results of growth chamber experiment with wax paper tube artificial seeds.

| Seed | Material inside | | Bottom | | Initial # of | % Survival on |
|---|---|---|---|---|---|---|
| structure | the structure | Top closure | closure | Autopsy | containers | day of autopsy |
| Wax paper | 10 g/L of | Pre-stretched | Pre-stretched | 5 days | 15 | 100.0 |
| tube | Polymer mixed | Parafilm ® M | Parafilm ® M | | | |
| | with MS Salt | | | | | |
| Wax paper | 10 g/L of | Pre-stretched | Pre-stretched | 14 days | 15 | 100.0 |
| tube | Polymer mixed | Parafilm ® M | Parafilm ® M | | | |
| | with MS Salt | | | | | |
| Wax paper | 10 g/L of | Pre-stretched | Pre-stretched | 33 days | 15 | 0.0 |
| tube | Polymer mixed | Parafilm ® M | Parafilm ® M | | | |
| | with MS Salt | | | | | |
| Wax paper | Autoclaved | Pre-stretched | Staple | 5 days | 15 | 100.0 |
| tube | potting soil | Parafilm ® M | | | | |
| Wax paper | Autoclaved | Pre-stretched | Staple | 14 days | 15 | 0.0 |
| tube | potting soil | Parafilm ® M | | | | |
| Wax paper | Autoclaved | Pre-stretched | Staple | 33 days | 15 | 0.0 |
| tube | potting soil | Parafilm ® M | | | | |
| Bare plantlet - | — | None | None | 5 days | 15 | 100.0 |
| Field | | | | | | |
| Bare plantlet - | — | None | None | 14 days | 15 | 87.0 |
| Field | | | | | | |
| Bare plantlet - | — | None | None | 33 days | 15 | 93.3 |
| Field | | | | | | |
| Bare plantlet - | — | None | None | 5 days | 15 | 100.0 |
| Greenhouse | | | | | | |
| Bare plantlet - | — | None | None | 14 days | 15 | 33.3 |
| Greenhouse | | | | | | |
| Bare plantlet - | — | None | None | 33 days | 15 | 6.7 |
| Greenhouse | | | | | | |

As shown in Table 36, the final viability (autopsy after 33 days) for the treatments with encapsulation is the same (0%), but the treatment with superabsorbent polymer inside the tubes kept the plantlets alive for a longer period of time. In this experiment, the plantlets ruptured the Parafilm® lids in most cases, although some of the Parafilm® lids exhibited spontaneous rupture in the field environment.

### Example 43

### Effect of Structure Permeability in Artificial Seeds in Brazil Field Testing

Cylindrical wax paper containers (Colossal drinking straw, Aardvark®, Precision Products Group, Ft Wayne, Ind., 1.19 cm outer diameter), 15 mL and 50 mL polypropylene centrifuge tubes (Corning®) tubes were cut into 4 cm lengths. For the centrifuge tubes, the 4 cm section consisted of only the cylindrical (non-conical) portion of the tube. Sugarcane plantlets, cultivar V11 (SP813250) which had been regenerated for 37 days from bud tissue fragments in plantlet regeneration medium were used for this experiment. The plantlet shoots were trimmed to approximately 3 cm length before encapsulation. The bottoms of the tubes were either closed by wrapping pre-stretched Parafilm® M across the bottom or were left opened. In one treatment, the tips of 50 mL centrifuge tubes were cut, creating a 1.5 cm hole and was used as the bottom structure. A thin approximately 1 cm layer of autoclaved potting soil (Tropstrato® HT) was placed at the bottom of the tubes. The plantlets were placed on the soil layer, and then additional potting soil was added to fill the tube until the plantlet was mostly covered. A volume of approximately 1 mL of water was added into the structure. The tops of the tubes were closed with either pre-stretched Parafilm® M, with inverted 15 mL or 50 mL centrifuge tubes. The hole size on the top of the tube was varied from no hole (impermeable to 1.0 cm hole).

The artificial seeds were planted in a vertical orientation in raised beds at the DuPont do Brasil site in Paulinia (SP), Brazil such that the tops of the tubes were less than 0.5 cm above the soil surface. Bare plantlets without trimming were planted in both the field, as well as a nearby greenhouse onsite (using the same autoclaved potting soil used inside the structures) in 8 cm pots (240 mL volume)). The field soil had been prepared before the experiment using rotary hoes and a bed shaper. After planting, no irrigation was performed. Survival was monitored every two days.

TABLE 37
Results of field experiment with wax paper tube artificial seeds.

| | | | | % |
|---|---|---|---|---|
| | | | | Survival |
| Seed | | | Initial # of | on day |
| structure | Top closure | Bottom closure | containers | 30 |
| 4 cm Wax | Pre-stretched | Pre-stretched | 22 | 0.0 |
| paper tube | Parafilm ® M | Parafilm ® M | | |
| 4 cm Wax | 15 mL centrifuge | None | 22 | 4.5 |
| paper tube | tube without hole | | | |
| 4 cm Wax | 15 mL centrifuge | None | 22 | 0.0 |
| paper tube | tube with 0.2 cm | | | |
| | hole | | | |
| 4 cm Wax | 15 mL centrifuge | None | 22 | 0.0 |
| paper tube | tube with 0.5 cm | | | |
| | hole | | | |
| 4 cm 15 mL | 15 mL centrifuge | None | 22 | 4.5 |
| centrifuge | tube with 0.5 cm | | | |
| tube | hole | | | |
| 4 cm 50 mL | 50 mL centrifuge | None | 22 | 9.0 |
| centrifuge | tube with 0.5 cm | | | |
| tube | hole | | | |
| 50 mL | 50 mL centrifuge | None | 22 | 13.6 |
| centrifuge | tube with 1.0 cm | | | |
| tube with | hole | | | |
| 1.5 cm hole | | | | |
| Bare plantlet - | None | None | 22 | 0.0 |
| Field | | | | |
| Bare plantlet - | None | None | 22 | 50.0 |
| Greenhouse | | | | |

As shown in Table 37, in this experiment, all the treatments with encapsulation had a low survival rate (from 0% to 14%). The treatment with the two 50 mL centrifuge tubes on top of each other had the highest survival. The larger hole size provided increased survival for the 15 mL centrifuge tube based artificial seeds. The bare plantlets at the field had a viability of 0%, indicating that the artificial seeds provided improved survival. In this experiment, 91% of the Parafilm® M lids were observed to spontaneously rupture over a period of several days in the field environment, before the plantlets could rupture them. The maximum temperature in this experiment ranged from about 32-35° C. for the first 6 days.

### Example 44

### Synthetic Seeds from Biodegradable Tubes and Cups

Biodegradable tubes and cups were prepared from poly(lactic acid) (4032D grade PLA, NatureWorks, Minnetonka, Minn.), Starch (Sigma Aldrich), α-Cellulose (Sigma Aldrich, St. Louis, Mo.), Chitosan (Sigma Aldrich, St. Louis, Mo.), poly(hydroxyl-butyrate) (PHB, Sigma Aldrich, St. Louis, Mo.), and/or poly(hydroxy-butyrate)-co-poly(hydroxy-valerate) (PHB-PHV, Sigma Aldrich, St. Louis, Mo.). For some blends D-sorbitol (Sigma Aldrich, St. Louis, Mo.) and glycerol (Sigma Aldrich, St. Louis, Mo.) were added as plasticizers. The tubes and cups were formed by pouring a solution of 20% polymer/polymer blend dissolved in chloroform into a 15 mL centrifuge tube or a 100 mL plastic beaker, ensuring the polymer solution coated the entire inner surface of the container. Upon evaporation of the chloroform, the tube or cup delaminated from the surface of the container, and the tube/cup was removed and dried overnight at ambient temperature. Table 38 describes specific polymer blends used to make tubes and cups.

TABLE 38
Polymer compositions used to form biodegradable synthetic seed tube
and cup structure.
(Weight ratio) Composition

Poly(L-lactic acid)
1:1:0.5 Poly(L-lactic acid)/Starch/Sorbitol
1:1:0.5 Poly(L-lactic acid)/Cellulose/Sorbitol
1:1:0.5 Poly(L-lactic acid)/Chitosan/Sorbitol
1:1 PHB/PHB-PHV
1:1:2:0.05 PHB/PHB-PHV/Starch/Sorbitol
1:1:2:0.05 PHB/PHB-PHV/Cellulose/Sorbitol
1:1:2:0.05 PHB/PHB-PHV/Chitosan/Sorbitol
2:1 PLA/Starch/Sorbitol
1:1:0.1 Poly(L-lactic acid)/Starch/Glycerol
1:1:0.1 Poly(L-lactic acid)/Cellulose/Glycerol
1:1:0.1 Poly(L-lactic acid)/Chitosan/Glycerol
10/1/1 PLA/PHB/PHB-PHV
3:2:1:1 PLA/Starch/PHB/PHB-PHV

Sugarcane plantlets (prepared in a similar fashion to Example 1) were planted into potting soil (Metro-Mix® 360). The seed was assembled by placing the tube over the plant and pressing down into the soil. Twenty biodegradable synthetic seeds were planted in a flower box (12 cm deep×60 cm long×20 cm wide) containing 50:50 matapeake/sand soil (a mix of a local Maryland soil with sand, creating a high sand content soil). The plants were grown in a growth chamber Conviron model BDW-120) at 31° C. during the day, at 22° C. during the night, 60% relative humidity, and a 13 hr photoperiod (220 uE/m²) for 4 weeks and given 1 L of water 3 times per week. The sugarcane plants had 95% survival rate for all structures after 8 weeks. Plants in tubes made from more flexible materials, i.e. PLA/chitosan and PLA/PHB/PHB-PHV, were able to break out of the tubes more easily than plants packaged in more rigid tubes, i.e. PLA.

### Example 45

### Biodegradable Ovular Synthetic Seeds

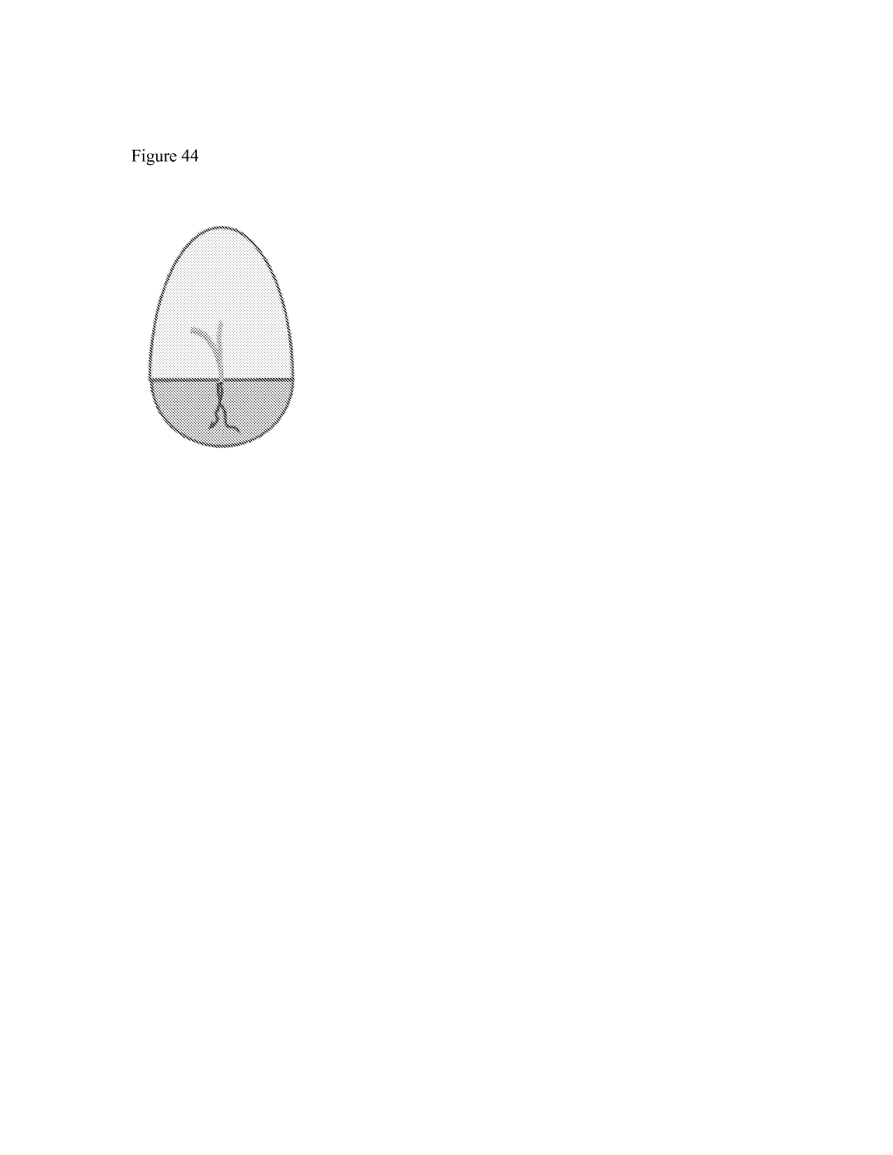

Biodegradable ovoid shaped shells were made from amorphous poly(D,L-lactic acid) (6361D Grade, NatureWorks, Minnetonka, Minn.) and poly(ε-caprolactone) (Sigma Aldrich, St. Louis, Mo.). The eggs were formed by pouring 25 wt % polymer solution in chloroform into a plastic Easter egg (Ovoid shaped, 7.5×3.75 cm). Upon evaporation of the chloroform, the egg shell delaminated from the inner surface of the Easter egg. The ovoid shell was removed from the Easter egg and a 1 cm hole was bored into the top and bottom parts of the egg. The bottom half of the egg shell was filled with moist potting soil (Metro-Mix® 360) and a sugarcane plantlet (see Example 1) was planted inside. The top half of the egg was placed on top (FIG. 44) and secured with Elmer's multipurpose glue or pre-stretched Parafilm® M. The synthetic seed eggs were planted in a flower box (12 cm deep×60 cm long×20 cm wide) with 50:50 matapeake/sand soil (a mix of a local Maryland soil with sand, creating a high sand content soil), such that ⅔ of the ovoid shell was covered with soil. The plants were grown in a growth chamber Conviron model BDW-120) at 31° C. during the day, at 22° C. during the night, 60% relative humidity, and a 13 hr photoperiod (220 uE/m²) and watered with 1 L water 3 times a week. The sugarcane plants in the egg synthetic seed structure had a survival rate of 50% at day 21.

### Example 46

### Synthetic Seeds with Expandable Tube Top

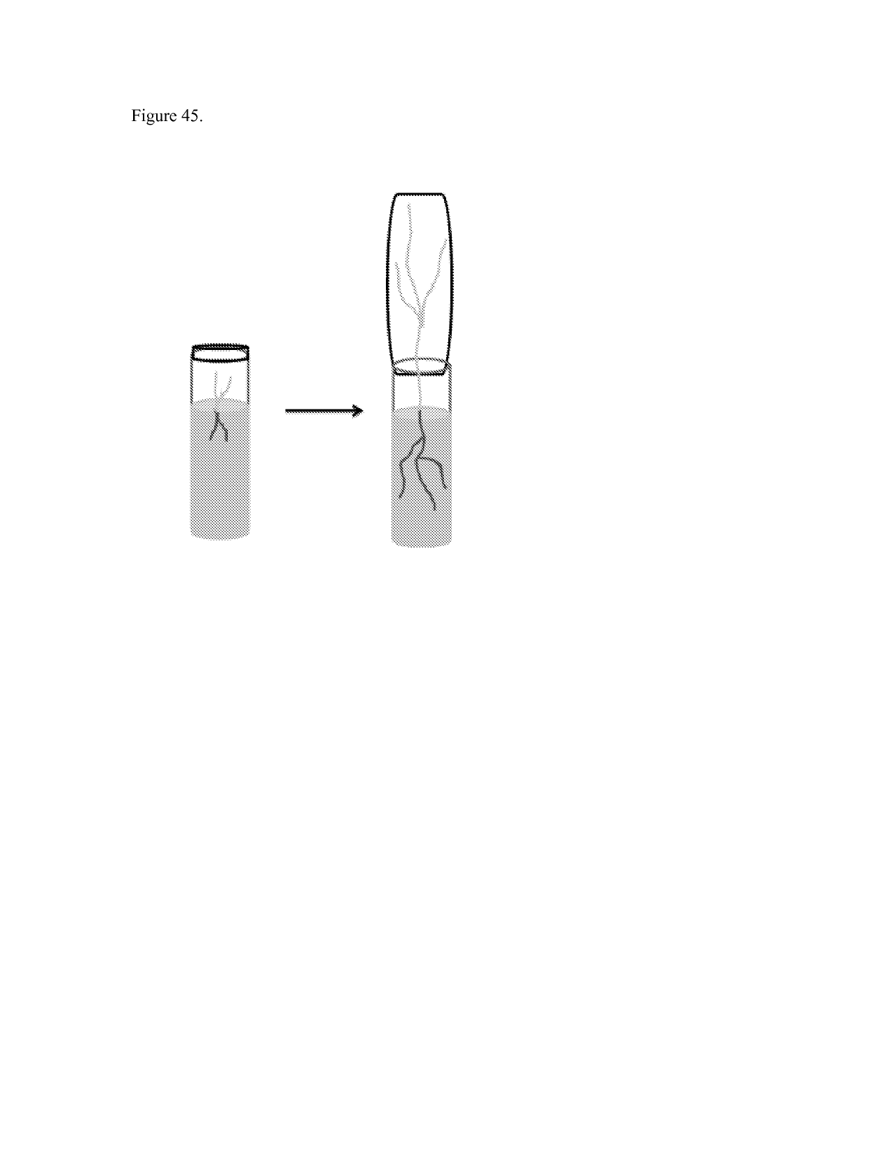

Synthetic seeds with an expandable tube top were prepared from a two-part tube structure, where the bottom half was rigid, and the top half was flexible. The bottom rigid half was made by cutting the conical end off a 50 mL centrifuge tube. The top flexible half was made by thin film casting a dilute polymer solution in chloroform into a 50 mL centrifuge tube. For the flexible material a 1:1 blend of starch (Sigma Aldrich, St. Louis, Mo.) and amorphous poly(D,L-lactic acid) (PLA 6361D Resin, NatureWorks, Minnetonka, Minn.) or a 3:2:1:1 (weight) blend of PLA, starch, poly(hydroxy butyrate) (PHB, Sigma Aldrich, St. Louis, Mo.) and poly(hydroxybutyrate-co-hydroxyvalerate) (PHB-PHV, Sigma Aldrich, St. Louis, Mo.) was used. Once the chloroform was evaporated the thin film in the shape of the tube was removed from the inside of the tube. A small slit (0.5 cm) was cut into the top of the flexible tube and then it was compressed to form a ring. The ring was then glued to the inside top of the rigid tube structure using Scotch® Super Glue (3M, St. Paul, Minn.). Sugarcane plantlets (Example 1) were planted into moist Metro-Mix® 360. The seed structure was placed above the plant and pressed down into the soil to assemble the synthetic seed. Additional moist Metro-Mix® 360 was put into the bottom of the tube so soil filled ⅔ of the rigid portion. The synthetic seeds were planted in a flower box (12 cm deep×60 cm long×20 cm wide) with 50:50 matapeake/sand soil (a mix of a local Maryland soil with sand, creating a high sand content soil) such that ⅔ of the rigid tube was beneath the soil. The plants were grown in the growth chamber Conviron model BDW-120) at 31° C. during the day, at 22° C. during the night, 60% relative humidity, and a 13 hr photoperiod (220 uE/m²) and watered 1 L 3 times a week. As the sugarcane grew the plant pushed the flexible tube up out of the rigid tube, essentially expanding the structure with the growth of the plant (FIG. 45). The expandable tubes had a survival rate of 100% at day 21.

### Example 47

### Storage Testing of Tube and Packet Type Artificial Seeds

Two types of artificial seeds were prepared for a storage study to determine their shelf life: 1) poly(ε-caprolactone) packets (as in Example 29) and 2) 50 mL polypropylene conical tubes with open top and bottom (as in Example 20). 160 replicates of each type were prepared and packaged into a storage bag (VWR Red Line Storage Bag, 32×48 cm, 100 um thickness), with 20 seeds/package. For the conical tube seed structures, tape (VWR general purpose laboratory labeling tape) was placed on the top and the bottom of the structure to cover the openings. An additional 15 of each type of seed structure were prepared to be planted at the onset of the experiment. Four packages of tubes and packets were stored at room temperature (20±1° C.) in the dark for 1 to 4 weeks. Another 4 bags of tubes and packets were stored at subambient temperature (10±2° C.) in the dark for 1 to 4 weeks.

At the start of the storage experiment 15 tubes, 15 packets, and 20 bare plants were planted in a 12 cm deep×60 cm long×20 cm wide flower box with 47.5:47.5:5 matapeake:sand:Metro-Mix® 360 soil. The plants were grown in a growth chamber Conviron model BDW-120) at 31° C. during the day, at 22° C. during the night, 60% relative humidity, and a 13 hr photoperiod (220 uE/m²). Plants were given 1 L of water 3 times a week. After each week, 1 package of tubes and packets were removed from ambient and sub-ambient temperature storage. For the tube seeds the tape was removed from the openings prior to planting. Every week the seeds were planted in flower boxes using with 47.5:47.5:5 matapeake:sand:Metro-Mix® 360 soil, grown in the growth chamber, and given 1 L of water 3 times a week. Plants were grown for 4 weeks. At the end of 4 weeks, the plants were dug out of the flower boxes, the shoot length was measured, and the survival rate was calculated. The results of the four week storage study are shown below in Table 39. These results indicate that storage at sub-ambient temperatures achieves a higher plant survival rate than storage at ambient temperature. Also the length of the shoot decreases with increasing storage time when tube synthetic seed structures are stored at ambient temperature.

TABLE 39
Results of Storage Synthetic Seeds

| | Storage | Storage Length | Survival | Average Length |
|---|---|---|---|---|
| Seed Type | Temperature | (weeks) | Rate (%) | of Shoot (cm) |
| Bare | — | 0 | 55 | 30.2 ± 10.6 |
| Tube | — | 0 | 100 | 37.8 ± 8.0 |
| Packet | — | 0 | 100 | 20.1 ± 3.6 |
| Tube | 20° C. | 1 | 100 | 50.2 ± 13.4 |
| Tube | 20° C. | 2 | 85 | 41.2 ± 12.1 |
| Tube | 20° C. | 3 | 25 | 33.8 ± 3.7 |
| Tube | 20° C. | 4 | 5 | 20.8 |
| Packet | 20° C. | 1 | 100 | 23.0 ± 5.5 |
| Packet | 20° C. | 2 | 90 | 19.8 ± 5.6 |
| Packet | 20° C. | 3 | 40 | 13.8 ± 8.1 |
| Packet | 20° C. | 4 | 0 | — |
| Tube | 10° C. | 1 | 100 | 49.9 ± 5.4 |
| Tube | 10° C. | 2 | 100 | 49.6 ± 8.7 |
| Tube | 10° C. | 3 | 65 | 49.8 ± 12.2 |
| Tube | 10° C. | 4 | 30 | 49.7 ± 10.5 |
| Packet | 10° C. | 1 | 75 | 24.4 ± 3.1 |
| Packet | 10° C. | 2 | 85 | 23.0 ± 3.0 |
| Packet | 10° C. | 3 | 60 | 15.1 ± 4.4 |
| Packet | 10° C. | 4 | 20 | 23.5 ± 3.8 |

### Example 48

### Flexible Foldable Tube Artificial Seeds

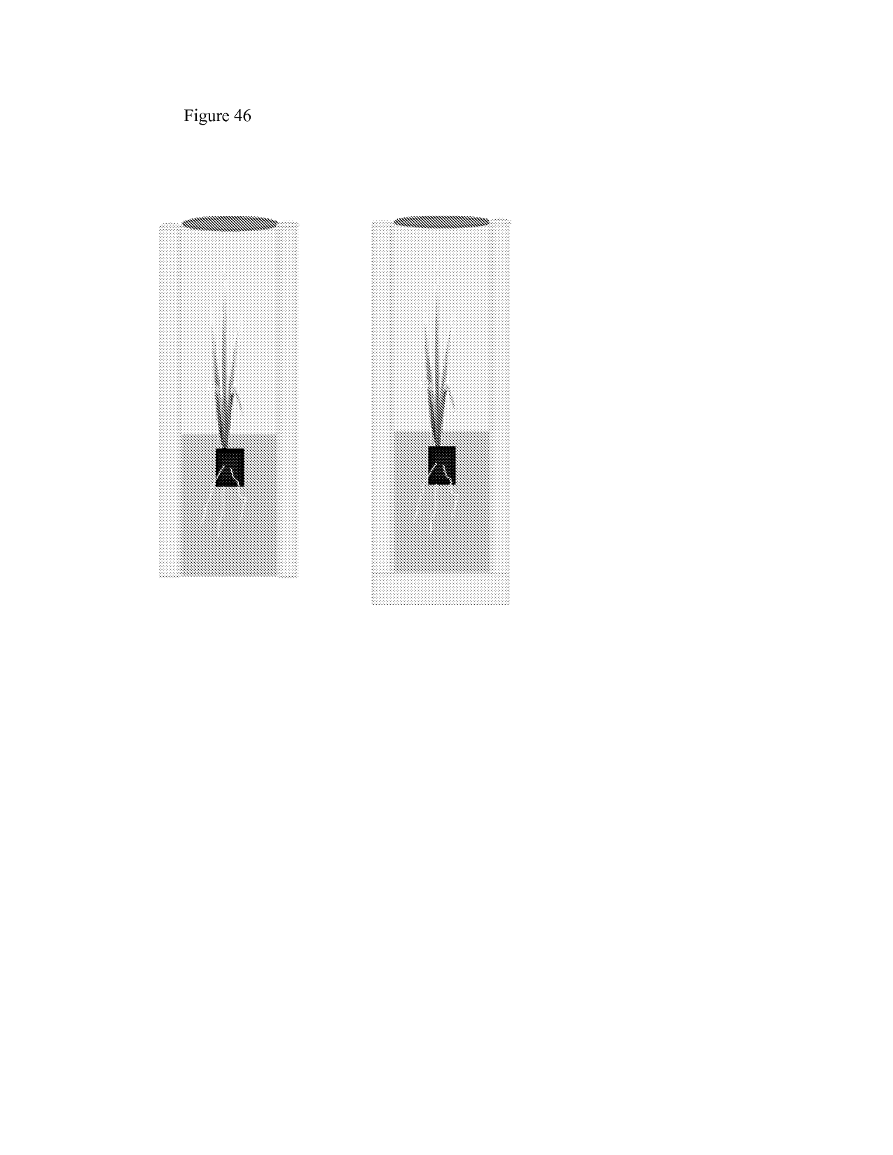

The purpose of this example was to study flexible foldable tube-shaped artificial seeds. Poly(caprolactone) film (50 um thickness) was fabricated from poly(ε-caprolactone) pellets (Capa™ 6800, Perstorp Company, Perstorp, Sweden) using a 28 mm twin screw extruder and a film line. The die temperature was kept at 155° C. and the barrel temperatures ranged from 127-160° C. The film was cut into rectangles approximately 12 cm by 12 cm and heat sealed into a cylindrical shape. Three or four smaller tubular subcompartments were created by hot pressing portions of the tube parallel to the axis of the main tube. The ends of the subcompartments were also heat sealed, while the larger tube was left open at the top end and heat sealed along the bottom in one treatment (FIG. 46). This gave the larger structure increased rigidity. The advantage of using flexible material was that the thickness and therefore amount of polymer were reduced, and also this produced a structure that could be folded to occupy less space prior to planting and would expand back to its larger conformation upon removal of a restraint. A sugarcane plantlet and moist Metro-Mix® 360 were inserted from the bottom of the tube, creating a soil plug approximately 5 cm thick. The structure was planted in a vertical orientation approximately 5 cm deep in 10 cm plastic pots with trays in a growth chamber (Conviron model BDW-120) at 31° C. during the day, at 22° C. during the night, 40% relative humidity, and a 13 hr photoperiod (220 uE/m²). 6 seeds were fabricated and all 6 sprouted at 25 days.

### Example 49

### Production of Poly(Vinyl Alcohol), Starch and Cellulose Fiber Based Composite Films for Artificial Seed Structures

The procedure described below provides an alternative plastic material from renewable resources, with good mechanical and biodegradability properties for use as an artificial seed structure. The material is a composite polymer comprised of poly (vinyl alcohol) (PVOH), a water soluble polymer; corn starch, with approximately 27% amylose and 73% amylopectin, and cellulose fibers as a reinforcement material with high water absorption ability. The polar structure of PVOH enables a good compatibilization with natural polymeric materials, resulting in homogeneous biodegradable films.

In a 1 L beaker approximately 21 g of PVOH was added to 200 mL of distilled water at 90° C. under stirring, until a homogeneous solution was formed. Around 50 mL of water was added to compensate any evaporation loss, followed by addition of 12 g glycerol and 7.5 g urea. This solution was stirred for 10 minutes, 16.5 g corn starch were dissolved in 100 mL of water at room temperature, and this mixture was added to the heated solution. After 30 minutes, 3 g of cellulose fibers and 100 mL of water at 70° C. were added and stirred for extra 40 min, when 10 drops of Hypermaster 602 (Montenegro Quimica, Brazil) antifoaming agent were added. The solution was poured into a wide container with non-stick coating and left to dry overnight in an oven at 40° C. This material was ground in a knife mill and compression molded, resulting in films approximately 350 μm thick.

For cross-linked samples, hexamethoxymethylmelamine (HMMM), a low imino melamine-formaldehyde, and a catalytic amount of citric acid were added after the cellulosic fibers, and the mixture was stirred for an additional 45 minutes at 70° C. before addition of the antifoaming agent. The variation of the compositions is listed in Table 40.

TABLE 40
Composition of the composite films

| | PVOH | Cellulose | Glycerol | | Corn Starch | HMMM | Citric Acid |
|---|---|---|---|---|---|---|---|
| Sample | (%) | Fiber (%) | (%) | Urea (%) | (%) | (%) | (%) |
| Composite 1 | 36.7 | 1.5 | 24.3 | 12.5 | 25 | — | — |
| Composite 2 | 35.0 | 5.0 | 12.5 | 12.5 | 27.5 | — | — |
| Composite 3 | 33.2 | 6.3 | 16.6 | 16.6 | 16.6 | 9.7 | 1.0 |
| Composite 4 | 30.8 | 41.0 | 7.7 | 7.7 | 7.7 | 4.6 | 0.5 |

### Encapsulation of Rice Buds in Biodegradable Packets

Compression molded composite films using composites 2 and 3 from the description above were used to construct rapidly biodegradable containers. A piece of film measuring 7.0 cm in width and 9.5 cm in length was folded overlapping the sides, which were heat-sealed along the two shorter edges to create an open pouch. Both films were split in two treatments: one pouch was loaded with pre-moistened potting soil (Tropstrato® HT) and one rice bud, germinated for two days in germination paper; the other pouch was loaded only with the rice bud.

The structures with and without potting soil were planted side by side in 1.16 L plastic pots with slits in the bottom, filled with Paulinia field soil. All packets were planted 5 cm deep in a vertical orientation such that the packets were completely covered with soil. The pots were maintained in a greenhouse and watered daily. For comparison, bare rice buds were planted in identically prepared and maintained pots.

After two weeks a sampling of the structures indicated partial degradation which was more pronounced for composite 2 than for composite 3, demonstrating the possibility to modulate the degradation by cross-linking the material.

As we can see in Table 41, the appearance rate results for the structures after four weeks were low compared to the control. The sampling showed that the composites completely degraded after this period and some buds were still developing. This experiment demonstrated that these materials are not phytotoxic for rice buds.

TABLE 41
Appearance rates* for composite material pouches and bare bud controls

| | | | Composite 3 | |
|---|---|---|---|---|
| Composite 2 | Composite 2 | Composite 3 | without | |
| with Potting | without Potting | with Potting | Potting | Bare Plants |
| soil (%) | soil (%) | soil (%) | soil (%) | control (%) |
| 23 | 0 | 0 | 0 | 50 |

*appearance rate here is defined as the capability of the plant ruptures the structure (when applicable) and appears above the soil level

### Example 50

### Comparison of Artificial Seeds Comprising Rapidly Biodegradable Lids and Non-Degradable Structures

Wax paper tubes (Colossal drinking straw, Aardvark®, Precision Products Group, Ft Wayne, Ind., 1.19 cm outer diameter), were cut into 4 cm lengths. One open end of each tube was closed with either a 254 μm thick soybean oil gel film, pre-stretched Parafilm® M, 680 μm and 380 μm thick composite 2 cast film (Example 49), or 515 μm and 325 μm thick composite 3 cast film (Example 49).

The soybean oil gel film was prepared as described in Example 26, while the composite materials were prepared according to a procedure similar to that described in Example 49. The composite films were prepared by assembling the paper tubes vertically in the wide pan with non-stick coating before the drying step, such that the solution was kept approximately 1 cm (thicker samples) and 0.5 cm (thinner samples) above the bottom of the tubes, providing a casting film layer at one end of the tubes. The samples were removed using a cork borer after dried. The Parafilm® M paper tubes containers were assembled by first closing the bottom with a 4 cm unidirectionally pre-stretched Parafilm® M. In all structures, a 1 cm layer of autoclaved potting soil Tropstrato® HT was added. After that, sugarcane plantlets, which had been cultured for 5 weeks in plantlet regeneration media were placed on top of the soil, and the leaves were trimmed to fit in the tube. Next, additional soil was added to create an approximately 3 cm thick layer in the tube, and 1 mL water was added. Finally, the top of each tube was covered only with pre-stretched Parafilm® M. For the tubes with Composite 2 or 3 lids, a similar procedure was followed, except starting with tubes with the composite films already cast in the bottom and the top ends sealed with a 1 cm paper section comprised of the same material of the bottom. The tubes were planted in 240 mL plastic pots filled with Paulinia field soil, kept in a growth chamber (Instala Frio) at 28° C. during the day and 18° C. during the night, 70-80% relative humidity, 16 hr photoperiod (190 μE/m²) during the first 16 days. After the day 17, the chamber conditions were changed to 25° C. during the day, with 70% relative humidity and a temperature peak of 30° C. for 2 h, 18° C. during the night, keeping the relative humidity at 75%, and a 14 h photoperiod (same light intensity).

All treatments were irrigated every 2 days using a rain simulator (E.I. DuPont de Nemours, Wilmington Del. 19880) providing 25 mm of rain (flow rate approximately 7.5 L/min). Half of the samples were protected by a plastic conical lid during the rain simulation in order to evaluate the degradation of the material in the absence of direct contact with water.

Immediately after the first rain, 57% of the thinner unprotected samples of composite 2, 29% of the thicker unprotected samples of composite 2 and 14% of the thinner unprotected samples of composite 3 that were exposed to the rain started to rupture, while the other exposed composite samples became opaque. The protected ones remained intact. The plants could only rupture the soybean oil gel and Parafilm® M samples in this period.

After 33 days, and 10 simulated rains the rupture results could be directly related to 2 different causes: the plant ruptured the structure or the material ruptured by itself due to degradation caused by water from the simulated rain. Most of the composite protected samples did not degrade and the plant could not rupture them. The results of the degradation causes are summarized in the Table 42.

TABLE 42
Degradation causes for different top lids of wax paper tube artificial seeds after 33 days
and 10 rain simulations

| Protected | Plant | Material | Not | Unprotected | Plant | Material | Not |
|---|---|---|---|---|---|---|---|
| Samples Lid | Rupture | Degradation | ruptured | Samples Lid | Rupture | Degradation | ruptured |
| Type | (%) | (%) | (%) | type | (%) | (%) | (%) |
| Soybean Oil gel | 57.1 | 14.3 | 28.6 | Soybean oil gel | 57.1 | 0.0 | 42.9 |
| Composite 2 | 0.0 | 0.0 | 100.0 | Composite 2 | 0.0 | 28.6 | 71.4 |
| (680 um thick) | | | | (680 um thick) | | | |
| Composite 2 | 0.0 | 0.0 | 100.0 | Composite 2 | 14.3 | 85.7 | 0.0 |
| (380 um thick) | | | | (380 um thick) | | | |
| Composite 3 | 0.0 | 0.0 | 100.0 | Composite 3 | 0.0 | 14.3 | 85.7 |
| (515 um thick) | | | | (515 um thick) | | | |
| Composite 3 | 14.3 | 0.0 | 85.7 | Composite 3 | 28.6 | 57.1 | 14.3 |
| (325 um thick) | | | | (325 um thick) | | | |
| Parafilm ® M | 42.9 | 0.0 | 57.1 | Parafilm ® M | 42.9 | 0.0 | 57.1 |

From these results one can see that the soybean/Kraton® oil gel samples presents the highest rupture rate caused by the plants, which can be related to the low thickness, weakness of the material, and good moisture barrier properties which keeps water inside the structure.

The survival rate of the plants for all seed structures is shown in Table 43.

TABLE 43
Survival of artificial seeds using different top lid materials.

| | Number | | | | |
|---|---|---|---|---|---|
| | of | | | Number of | |
| | artificial | | | artificial | |
| | seeds | | | seeds | |
| Protected | initially | Survival at | Unprotected | initially | Survival at |
| Samples | planted | day 33, (%) | Samples | planted | day 33, (%) |
| Soybean Oil gel | 7 | 14.3 | Soybean Oil gel | 7 | 28.6 |
| Composite 2 (680 | 7 | 0 | Composite 2 (680 | 7 | 0 |
| um thick) | | | um thick) | | |
| Composite 2 (380 | 7 | 14.3 | Composite 2 (380 | 7 | 14.3 |
| um thick) | | | um thick) | | |
| Composite 3 (515 | 7 | 0 | Composite 3 (515 | 7 | 0 |
| um thick) | | | um thick) | | |
| Composite 3 (325 | 7 | 0 | Composite 3 (325 | 7 | 0 |
| um thick) | | | um thick) | | |
| Pre-stretched | 7 | 14.3 | Pre-stretched | 7 | 28.6 |
| Parafilm ® M | | | Parafilm ® M | | |
| | | | Bare Plantlet | 28 | 85.7 |
| | | | Controls | | |

### Example 51

### Blends of Poly(1,3-Propanediol Succinate) with Poly(Lactic Acid)

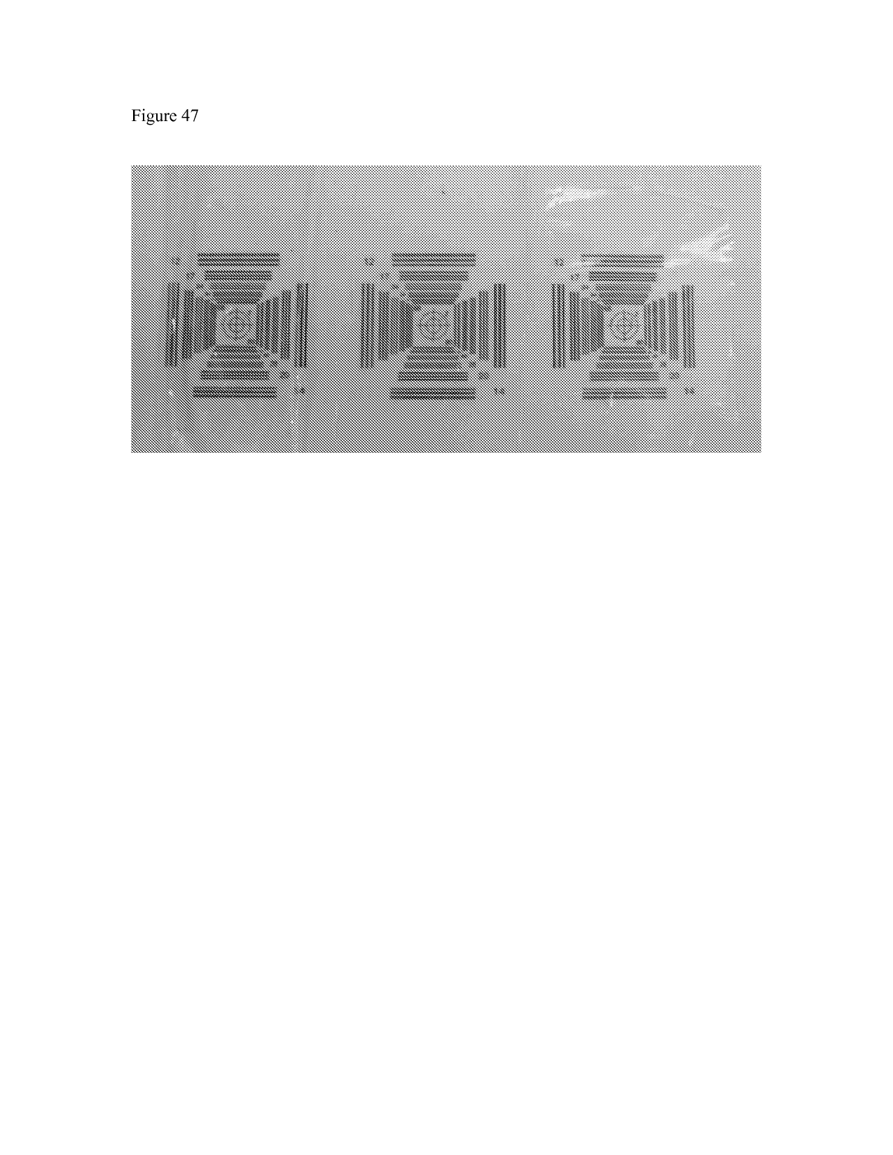

Due to the rigidity and brittleness of poly(lactic acid), blends with other polymers may be desired to improve mechanical properties for artificial seed applications in which seeds may be handled by a mechanical planter. Additionally, poly(lactic acid) is slow to biodegrade at ambient temperature in soil (Shogren, R. L., Doane, W. M., Garlotta, D., Lawton, J. W., Willett, J. L. Polymer Degradation and Stability, 2003, 79, 405-411). Blends with other polymers can help to improve toughness (Afrifah, K. A., Matuana, L. M. Macromolecular Materials and Engineering, 2010, 802-811) and biodegradability (Shogren, R. L., Doane, W. M., Garlotta, D., Lawton, J. W., Willett, J. L. Polymer Degradation and Stability, 2003, 79, 405-411). However, polymer blends, like those discussed in the references cited are opaque due to incompatibility between the two or more polymer phases. For the purposes of artificial seeds, it may be advantageous to allow light to transmit through the materials of seed, to accelerate the growth of certain tissue types. Thus, blends of PLA with biodegradable polyester were pursued. Specifically, blends of poly(lactic acid) (PLA 4032D, NatureWorks, Minnetonka, Minn.) with poly(1,3-propanediol succinate) (Mn=8100 g/mol, Mw=23000 g/mol by size exclusion chromatography) were explored. 1.3 g PLA 4032D and 0.2 g poly(1,3-propanediol succinate) were weighed into a 20 mL glass vial. 8.0 g chloroform was added and the solution stirred for 1 day. The solution was cast onto a poly(tetrafluoroethylene) sheet using a doctor blade with a 40 mil thick gap. This resulted in a film with 13.3 wt % poly(1,3-propanediol succinate) with thickness 75-150 um. A similar procedure was followed to create blends with 22 wt % and 50 wt % poly(1,3-propanediol succinate). All blends were optically translucent to transparent (FIG. 47).

Differential scanning calorimetry (DSC) was used to characterize the blends. This was performed in nitrogen at a heating rate of 10° C./min using a TA Instruments (New Castle, Del.) Model Q100 DSC. The results of the analysis are shown in Table 44. The results indicate that two glass transitions are present in the polymer blends. This indicates that two polymer phases are present, and that the blends are largely immiscible. However, it is noted that the glass transition temperatures change with composition, suggesting some interaction of the phases, possibly plasticization or partial compatibility. An additional observation was a significant crystallization endotherm for the blends, which is absent in pure PLA4032D, as well as a larger melting exotherm. This suggests an influence of the poly(1,3-propylene succinate) to accelerate crystallization, either through the plasticization effect, or through nucleation.

TABLE 44
Thermal transitions for blends of poly(1,3-propanediol succinate) and
semicrystalline poly(lactic acid)

| | Weight | Glass | Melting | Enthalpy | Enthalpy |
|---|---|---|---|---|---|
| Weight | Percent | transition | point | of crystal- | of |
| Percent | Poly(1,3- | temperatures | (2^{nd} | lization | melting |
| Poly(lactic | propanediol | (2^{nd} heat, | heat | (2^{nd} heat, | (2^{nd} heat, |
| acid) | succinate) | ° C.) | ° C.) | J/g) | J/g) |
| 100% | 0% | 61.7 | 166.9 | none | 4.1 |
| 87% | 13% | −36.1, 52.9 | 167.3 | 20.55 | 34.32 |
| 50% | 50% | −33.7, 49.0 | 166.1 | 10.55 | 21.34 |
| 0% | 100% | −33.4 | none | none | none |

Tensile measurements on the blends were performed using a TA-XT2i Texture Analyser (Texture Technologies, Scarsdale, N.Y.). The results are listed below in Table 45. The blends of poly(1,3-propanediol succinate) exhibited higher elongations, lower tensile strengths and less brittleness than pure PLA4032D.

TABLE 45
Tensile measurements on blends of poly(1,3-propanediol succinate) with
poly(lactic acid). The data are listed are 3 replicates.

| | Weight Percent | | |
|---|---|---|---|
| | Poly(1,3- | | |
| Weight Percent | propanediol | Tensile strength | |
| Poly(lactic acid) | succinate) | (MPa) | Elongation (%) |
| 100% | 0% | 40.5, 50.0, 50.7 | 13%, 13%, 12% |
| 78% | 22% | 30.1, 33.3, 33.8 | 25%, 29%, 30% |
| 50% | 50% | 10.3, 10.9, 12.0 | 29%, 57%, >74% |

Artificial seeds were constructed by wrapping the 22 wt % poly(1,3-propanediol succinate) film into a single layer cylindrical tube with diameter of 1.2 cm. The edge of the film along the tube side was hot glued. The tube was cut into approximately 6 cm long sections. A sugarcane plantlet with moist Metro-Mix® 360 was added such that the tube was approximately 75% full and the seeds were planted in 10 cm pots in moist Metro-Mix® 360 in a growth chamber (Conviron model BDW-120) at 31° C. during the day, at 22° C. during the night, 40% relative humidity, and a 13 hr photoperiod (220 uE/m²). 9 artificial seeds were planted and 4 sprouted after 21 days.

### Soil Degradation of Poly(1,3-Propanediol Succinate)

Soil degradation studies of poly(1,3-propanediol succinate) (Mn=22000, Mw=41800 g/mol as measured by size exclusion chromatography) in comparison to other polymers was undertaken at the DuPont Stine Haskell Research Center in Newark, Del. Films of various polyesters were formed through melt pressing at appropriate temperatures above their melting point. The films were of thicknesses ranging from approximately 200 to 400 um in thickness, and were approximately 2 cm wide by 8-12 cm long. Three film samples per composition were tested. The films were taped to the bottoms of aluminum trays using autoclave tape (VWR, Radnor, Pa.) such that the majority of the films were exposed, and the trays were buried horizontally at a depth of approximately 15 cm in the field. The samples were left for a period of 27 days, and then exhumed. The degradation results were judged qualitatively through visual observations. After exhuming, the films were rinsed with water to remove soil and observed a second time. The results in Table 46 indicate that the poly(1,3-propanediol succinate) exhibited similar visual degradation in real soil conditions to other rapidly degradable polymers (poly(3-hydroxy butyrate-co-3-hydroxyvalerate)), but then mechanically disintegrated easily with rinsing, whereas other polymers remained. This suggests and advantage for poly(1,3-propanediol succinate) to disintegrate more readily in a field environment.

TABLE 46
Results of field degradation of polyesters after 27 days.

| | | | | Visual |
|---|---|---|---|---|
| | | | | observation |
| | | Initial Film | Visual observation | after |
| Polymer | Supplier | thickness (um) | before washing | rinsing |
| Poly(lactic acid) | NatureWorks | 250 | All Intact | All Intact |
| PLA 4032D | (Minnetonka, | | | |
| | MN) | | | |
| Poly(D,L-lactide- | Sigma Aldrich | 200 | 2 out of 3 cracked | Cracked |
| co-glycolide) | (St. Louis, | | | pieces |
| | MO) | | | remain |
| Poly(1,3- | Synthesized | 400 | 2 out of 3 cracked | Cracked |
| propanediol | internally at | | | pieces |
| succinate) | E.I. DuPont de | | | washed |
| | Nemours, | | | away |
| | Wilmington, | | | |
| | DE | | | |
| Poly(3-hydroxy | Sigma Aldrich | 250 | 1 out of 3 cracked | Cracked |
| butyrate-co-3- | (St. Louis, | | | pieces |
| hydroxyvalerate) | MO) | | | remain |

An additional experiment was performed to compare the degradation rates of these polymers in Metro-Mix® 360. Similar sized film strips were created in this experiment in triplicate and were buried in a vertical orientation in Metro-Mix® 360 in 10 cm plastic pots and placed in a growth chamber (Conviron model BDW-120) at 31° C. during the day, at 22° C. during the night, 80% relative humidity, and a 13 hr photoperiod (220 uE/m²). The pots were watered periodically, and the films were exhumed after 1 month. The degradation of the samples was judged visually after rinsing (Table 47). It was observed that the poly(1,3-propanediol succinate) degraded significantly in Metro-Mix® 360 after 29 days.

TABLE 47
Results of growth chamber degradation of polyesters after 29 days in
Metro-Mix ® 360.

| | | Initial Film | Visual |
|---|---|---|---|
| | | thickness | observation |
| Polymer | Supplier | (um) | after rinsing |
| Poly(lactic acid) | NatureWorks | 250 | Intact |
| PLA 4032D | (Minnetonka, MN) | | |
| Poly(D,L-lactide- | Sigma Aldrich (St. | 200 | Intact |
| co-glycolide) | Louis, MO) | | |
| Poly(1,3- | Synthesized | 400 | Degraded with |
| propanediol | internally at E.I. | | holes |
| succinate) | DuPont de Nemours, | | |
| | Wilmington, DE | | |
| Poly(3-hydroxy | Sigma Aldrich (St. | 250 | Heavily |
| butyrate-co-3- | Louis, MO) | | degraded with |
| hydroxyvalerate) | | | holes |

Additional blends of poly(1,3-propanediol succinate) with amorphous poly(D,L-lactic acid) (PLA 6361D, NatureWorks, Minnetonka, Minn.) were explored. These were created in the same manner as above and were also uniformly transparent to translucent. Their thermal properties were studied using differential scanning calorimetry, and the results are shown in Table 48.

TABLE 48
Thermal transitions for blends of poly(1,3-propanediol succinate) and
amorphous poly(D,L-lactic acid).

| | Weight | | |
|---|---|---|---|
| Weight | Percent | Glass | |
| Percent | Poly(1,3- | transition | Melting |
| Poly(D,L- | propanediol | temperatures | point (1st |
| lactic acid) | succinate) | (2^{nd} heat, ° C.) | heat ° C.) |
| 100% | 0% | 59.8 | none |
| 90% | 10% | 43.7 | none |
| 70% | 30% | −37.2, 39.5 | none |
| 50% | 50% | −32.2, 43.3 | none |
| 0 | 100% | −33.4 | Not |
| | | | determined |

### Example 52

### Use of Synthetic Seeds with Genetically Engineered Sugarcane Plants

Genetically engineered sugarcane plants are produced through standard technologies (see, e.g., Manickavasagam et al. (2004) Plant Cell Rep 23:134-143; Jain et al. (2007) Plant Cell Rep 26:581-590; Joyce et al. (2010) Plant Cell Rep 29:173-183). Genetically engineered plants contain modified or introduced genes conferring altered agronomic qualities (including but not limited to resistance to herbicides, resistance to insect pests, resistance to diseases, improved yield and improved sugar content). Any synthetic seed design described in this application is useful for facilitating the planting of genetically engineered plants.

Genetically engineered plants regenerated as described in Example 1 or by other equivalent procedures are inserted into seed containers constructed as described in this application from various materials (including but not limited to tubes or containers made from wax paper, poly(lactic) acid, polycaprolactone, poly(3-hydroxy butyrate-co-3-hydroxy valerate) polypropylene, and cellulose composites.) Added to the plants inside the container are materials to support the growth and health of the plants (including but not limited to soil, MetroMix, agar, rock wool, sugar, inorganic salts, MS nutrients, superabsorbent polymers, water, fungicides, insecticides, herbicides, plant growth regulators and plant hormones) leaving an airspace inside the container. The ends of the containers are left open or are sealed with various materials (including, but not limited to Parafilm®, poly(lactic) acid, Alkyd film, Mylar® film, LDL triblock copolymer).

Seeds structures are placed in soil in a growth chamber, greenhouse, screenhouse or field, provided with adequate water, temperature, fertilizer, light, and pest protection and allowed to grow for approximately 4 weeks. Success of these seeds is demonstrated by survival of these plants under these growth conditions.

Herbicide resistance of genetically engineered seeds is demonstrated after 4 weeks of growth in soil. At that time, seed containers and lid materials, if still present around the aerial parts of the plants, are removed from the plants. The plants are then treated with herbicides (including but not limited to glyphosate and sulfonylurea) at typical use rates, suited to the region or environment, that kill or severely injures non-transgenic sugarcane and target weeds. Four weeks after treatment, the plants derived from the synthetic seeds containing genetically engineered herbicide resistant plants are healthy and vigorously growing while non-transgenic sugarcane controls are either dead or seriously injured.

### Example 53

### Use of Synthetic Seeds with Genetically Engineered Sugarcane Plants

Genetically engineered sugarcane plants are produced through standard technologies (see, e.g., Manickavasagam et al. (2004) Plant Cell Rep 23:134-143; Jain et al. (2007) Plant Cell Rep 26:581-590; Joyce et al. (2010) Plant Cell Rep 29:173-183). Genetically engineered plants contain modified or introduced genes conferring altered agronomic qualities (including but not limited to resistance to herbicides, resistance to insect pests, resistance to diseases, improved yield and improved sugar content). Any synthetic seed design described in this application is useful for facilitating the planting of genetically engineered plants.

Genetically engineered plants regenerated as described in Example 1 or by other equivalent procedures are inserted into seed containers. Control plants are of the same variety but not genetically engineered. Both GM and non-GM plants are propagated, regenerated, and handled in the same manner. In addition, GM plants grown from billets are used as another control. All plants, whether derived from micropropagation or from billets are grown in the same conditions.

Bare, unencapsulated plants are used as controls. In addition, plants grown from GM billets are also used as controls.

Wax paper tubes are cut into 4 cm lengths. One open end of each is closed with a film fabricated from a blend of gelatin and starch. The gelatin-starch-glycerol film layer is prepared by evaporating an aqueous solution of gelatin, starch and glycerol. In this solution the concentration of gelatin can be from 0.5 wt % to 5 wt %. The concentration of starch can be from 0.1 wt % to 2 wt %. The concentration of glycerol can be from 2 wt % to 8 wt %. In one embodiment, the solution used to create the film can comprise 2.5 wt % Gelatin (175 Bloom Strength); 1.0 wt % starch and 5.0 wt % glycerol. In another embodiment, the film forming solution can comprise 1.25 wt % gelatin (175 Bloom Strength) and 1.25 wt % gelatin (300 Bloom Strength); 1.0 wt % starch and 5.0 wt % glycerol. A 1.5 cm layer of autoclaved potting soil (Tropstrato® HT) is added. Plants are then placed inside the paper container on the top of the soil with leaves trimmed to fit into the tube. Next, additional soil is added to create an approximately 2 cm thick layer in the tube and enough water to saturate it is added. Finally, the top of each tube is closed using a 15 mL polypropylene centrifuge tube with a 5 mm hole on the top, attached to the paper tube using a piece of Parafilm® M.

The seed structures and bare plants are planted vertically in 470 mL plastic pots filled with a mix of field soil (from Paulinia Experimental Farm), sand and potting soil (Tropstrato® HT), in a volumetric proportion of 1:1:1. Seeds structures are planted with the soil level in the pot flush with the top of the polypropylene tubes and bare plants are planted so that the soil level is at the junction of the roots and shoots. The billets are planted horizontally, in 500 mL pots filled with the same soil mixture, at 2-5 cm below the soil level. The billet plants are transferred to 1 L pots after 3-4 weeks. All plants are irrigated twice a day, and kept inside a greenhouse.

The development and survival of the plants is monitored for up to 8 weeks. The plastic conical lid is removed at this time, when the plants are well-established in the soil.

At this time, GM and non-GM plants produced from the seed constructs and bare plants; and GM plants from billets, all with equivalent vigor and physiological stage, are chosen for herbicide treatments as shown in Table 49. For each treatment shown in the table, 10 plants are tested.

TABLE 49
Herbicide treatments for GM micropropagated plants, non-GM
micropropagated plants, and GM plants from billets.

| | | | |
|---|---|---|---|
| GM | Bare | 1. | Untreated control (no herbicide |
| micropropagated | | | application); |
| plant | | 2. | 850 g.a.i · ha⁻¹ Roundup ® |
| | | | (Glyphosate); |
| | | 3. | 3400 g.a.i · ha⁻¹ Roundup ® |
| | | | (Glyphosate); |
| | | 4. | 50 g.a.i · ha⁻¹ Curavial ® |
| | | | (Sulfumeturon-methyl); |
| | | 5. | 200 g.a.i · ha⁻¹ Curavial ® |
| | | | (Sulfumeturon-methyl). |
| | Encapsulated | 6. | Untreated control (no herbicide |
| | | | application); |
| | | 7. | 850 g.a.i · ha⁻¹ Roundup ® |
| | | | (Glyphosate); |
| | | 8. | 3400 g.a.i · ha⁻¹ Roundup ® |
| | | | (Glyphosate); |
| | | 9. | 50 g.a.i · ha⁻¹ Curavial ® |
| | | | (Sulfumeturon-methyl); |
| | | 10. | 200 g.a.i · ha⁻¹ Curavial ® |
| | | | (Sulfumeturon-methyl). |
| Non-GM | Bare | 11. | Untreated control (no herbicide |
| micropropagated | | | application); |
| plant | | 12. | 850 g.a.i · ha⁻¹ Roundup ® |
| | | | (Glyphosate); |
| | | 13. | 3400 g.a.i · ha⁻¹ Roundup ® |
| | | | (Glyphosate); |
| | | 14. | 50 g.a.i · ha⁻¹ Curavial ® |
| | | | (Sulfumeturon-methyl); |
| | | 15. | 200 g.a.i · ha⁻¹ Curavial ® |
| | | | (Sulfumeturon-methyl). |
| | Encapsulated | 16. | Untreated control (no herbicide |
| | | | application); |
| | | 17. | 850 g.a.i · ha⁻¹ Roundup ® |
| | | | (Glyphosate); |
| | | 18. | 3400 g.a.i · ha⁻¹ Roundup ® |
| | | | (Glyphosate); |
| | | 19. | 50 g.a.i · ha⁻¹ Curavial ® |
| | | | (Sulfumeturon-methyl); |
| | | 20. | 200 g.a.i · ha⁻¹ Curavial ® |
| | | | (Sulfumeturon-methyl). |
| GM plant from | Bare | 21. | Untreated control (no herbicide |
| billet | | | application); |
| | | 22. | 850 g.a.i · ha⁻¹ Roundup ® |
| | | | (Glyphosate); |
| | | 23. | 3400 g.a.i · ha⁻¹ Roundup ® |
| | | | (Glyphosate); |
| | | 24. | 50 g.a.i · ha⁻¹ Curavial ® |
| | | | (Sulfumeturon-methyl); |
| | | 25. | 200 g.a.i · ha⁻¹ Curavial ® |
| | | | (Sulfumeturon-methyl). |

The herbicide spray liquid is prepared in a 2 L PET bottle by dissolving the amount of the herbicide in water to give the correct rate. The plants are placed on a bench in the greenhouse and the application is performed using a bar equipped with 110.02 swing jet nozzles. The spray application parameters are 2.0 bars pressure, 1 m·s⁻¹ velocity, 200 L·ha⁻¹ spray volume, obtaining a spray liquid flow of 0.6 L·min⁻¹ per nozzle. The herbicide spray is applied 50 cm above the top of the plants. For all sulfumeturon-methyl applications, 0.2% Agral® (Nonylphenoxy polyethoxy ethanol) adjuvant is added into the spray liquid.

Assessment of crop response (% phytotoxicity) and plant height is performed at 7, 14, 21, 30 and 45 days after application using a visual evaluation of leaf injury, discolored leaves, overall plant growth and plant vigor. Injury is quantified in 5% increments from no injury (0%) to death (100%). Non-GM plants, both from seed structures and bare plants, show injury by all herbicide treatments within 14 days. For GM plants, from seed structures and bare plants from micropropagation as well as bare plants from billets, no or only minimal injury (less than 10%) is detected for all herbicide treatments.

### Example 54

### Extrusion of Poly(D,L-Lactic Acid) Tubes for Synthetic Seeds

Amorphous poly(D,L-lactic acid) (6361D Resin, Natureworks, LLC., Minnetonka, Minn.) was extruded using a cool screw extruder (1½″ Davis Standard, Davis-Standard LLC, Fulton, N.Y.), into 0.664″ outer diameter tubing with a wall thickness of 0.012″. The tubing was extruded at 13.4 ft/min using a melt temperature of 390° F., screw temperatures of 350-390° F., die temperatures of 390-391° F., and at a screw speed of 14.8 RPM. The extruded tubing was cut into shorter tubes, 6″ in length.

The seed was assembled by heating one end of the tube with a heat gun (Master Heat Gun, Master Appliance, Racine, Wis.) and crimping it closed with clamp forceps. Two grams of potting soil (Metro-Mix® 360) were added to the open end of the tube. One sugarcane plantlet (Example 1) was pressed down into the soil and 2 mL of water was added. The top of the tube was sealed by the same procedure described above. The seeds were stored at room temperature, in light, for 5 days prior to planting.

Before planting, the tops and bottoms of the seeds were cut open using a pair of scissors. The synthetic seeds were planted in a flower box (12 cm deep×60 cm long×20 cm wide) with 50:50 matepeake/sand soil (a mix of a local Delaware soil with sand, creating a high sand content soil), such that soil level inside the tube was aligned with the soil level outside. The plants were grown in a growth chamber (Conviron model BDW-120) at 31° C. during the day, at 22° C. during the night, 40% relative humidity, and a 13 hr photoperiod (220 uE/m²) and watered 1 L 3 times a week. The sugarcane plants in the PLA tube synthetic seed structure had a survival rate of 70% at day 14.

What is claimed is:

1. An artificial seed comprising one or more regenerable plant tissues, a container comprising a degradable portion, an unobstructed airspace, and a nutrient source, and further comprising one or more features selected from the group consisting of:
a) a penetrable or degradable region through which the regenerable plant tissue grows,
b) a monolayer water soluble portion of the container,
c) a region of the container that flows or creeps between about 1° C. and 50° C.,
d) a separable closure which is physically displaced during regenerable plant tissue growth,
e) one or more openings in sides or bottom of the container,
f) a conical or tapered region leading to an opening less than 2 cm wide at the apex and wherein the angle of the conical or tapered region is less than 135 degrees measured from opposite sides, and
g) a plurality of flexible flaps through which the regenerable tissue grows.

2. The artificial seed of claim 1, where the container or a region of the container or a closure further comprises one or more of the following: polyesters, polyamides, polyolefins, cellulose, cellulose derivatives, polysaccharides, polyethers, polyurethanes, polycarbonates, poly(alkyl methacrylate)s, poly(alkyl acrylate)s, poly(acrylic acids), poly(meth)acrylic acids, polyphosphazenes, polyimides, polyanhydrides, polyamines, polydienes, polyacrylamides, poly(siloxanes), poly(vinyl alcohol), poly(vinyl esters), poly(vinyl ethers), natural polymers, block copolymers, crosslinked polymers, proteins, waxes, oils, plasticizers, antioxidants, nucleating agents, impact modifiers, processing aids, tougheners, colorants, fillers, stabilizers, flame retardants, natural rubber, polysulfones, or polysulfides; or blends thereof; or crosslinked versions thereof.

3. The artificial seed of claim 1, wherein the container further comprises a component selected from the group consisting of:
a) amorphous poly(D,L-lactic acid), poly(lactic acid), poly(L-lactic acid), poly(D-lactic acid), poly(meso-lactic acid), poly(rac-lactic acid), or poly(D,L-lactic acid), (poly(hydroxyalkanoate), poly(hydroxybutyrate), poly(hydroxybutyrate-co-valerate), poly(caprolactone), poly(butylene succinate), poly(ethylene succinate), poly(ethylene carbonate), poly(propylene carbonate), starch, gelatin, thermoplastic starch, poly(butylene terephthalate adipate), poly(propylene terephthalate succinate), poly(propylene terephthalate adipate), poly(vinyl alcohol), poly(ethylene glycol), cellulose, chitosan, cellulose acetate, or cellulose butyrate acetate,
b) a polyester with greater than 5 mol percent aliphatic monomer content,
c) a crosslinked version of amorphous poly(D,L-lactic acid), poly(lactic acid), poly(L-lactic acid), poly(D-lactic acid), poly(meso-lactic acid), poly(rac-lactic acid), or poly(D,L-lactic acid), (poly(hydroxyalkanoate), poly(hydroxybutyrate), poly(hydroxybutyrate-co-valerate), poly(caprolactone), poly(butylene succinate), poly(ethylene succinate), poly(ethylene carbonate), poly(propylene carbonate), starch, gelatin, thermoplastic starch, poly(butylene terephthalate adipate), poly(propylene terephthalate succinate), poly(propylene terephthalate adipate), poly(vinyl alcohol), poly(ethylene glycol), cellulose, chitosan, cellulose acetate, cellulose butyrate acetate, or a polyester with greater than 5 mol percent aliphatic monomer content,
d) a plasticizer, wherein the plasticizer is present at less than 30 wt % of the total composition,
e) acetyl tributyl citrate, tributyl citrate, di-n-octyl sebacate, di-2-ethylhexylsebacate, di-2-ethylhexylsuccinate, diisooctyl adipate, di-2-ethylhexyl adipate, diisooctyl glutarate, di-2-ethylhexyl glutarate, poly(ethylene glycol), poly(ethylene glycol) monolaurate, sorbitol, glycerol, poly(propylene glycol), or water,
f) copolymers of two or more of caprolactone, lactic acid, D-lactide, L-lactide, meso-lactide, D,L-lactide, sebacic acid, succinic acid, adipic acid, glycolic acid, oxalic acid, ethylene glycol, 1,2-propanediol, 1,3-propanediol, 1,3-butanediol, 1,4-butanediol, 1,5-pentanediol, 2,2,4,4-tetramethyl-1,3-cyclobutanediol, 1,6-hexanediol, terephthalic acid, isophthalic acid, dimethyl siloxane, succinic anhydride, a diisocyanate, a crosslinker, or phthalic anhydride,
g) an antioxidant, a nucleating agent, an impact modifier, a processing aid, a toughener, a colorant, a filler, a stabilizer, or a flame retardant,
h) paper, water soluble paper, recycled paper, bond paper, kraft paper, waxed paper, or coated paper;
i) a combination of two or more of components a) through h) above, and
j) a blend comprising two or more of components a) through i) above.

4. The artificial seed of claim 1, wherein a region of the container or closure further comprises a component selected from the group consisting of:
a) random, block or gradient copolymers of lactic acid with caprolactone,
b) random, block or gradient copolymers of lactic acid with dimethylsiloxane,
c) an alkyd resin,
d) poly(vinyl alcohol), starch, cellulose, poly(ethylene glycol), agar, xanthan gum, alginate, hydroxypropylcellulose, methylcellulose, a water soluble protein, a water soluble carbohydrate, a water soluble synthetic polymer, or carboxymethylcellulose,
e) blends of two or more of the following: poly(vinyl alcohol), starch, cellulose, glycerol, poly(ethylene glycol), citric acid, urea, water, sodium acetate, potassium nitrate, ammonium nitrate, fertilizers, agar, xanthan gum, alginate, hydroxypropylcellulose, methylcellulose, a water soluble protein, a water soluble carbohydrate, a water soluble synthetic polymer, a crosslinker, or carboxymethylcellulose,
f) a gel comprising a block copolymer and an oil,
g) sodium carboxymethylcellulose,
h) wax-impregnated water soluble paper,
i) amorphous poly(D,L-lactic acid), poly(lactic acid), poly(L-lactic acid), poly(D-lactic acid), poly(meso-lactic acid), poly(rac-lactic acid), or poly(D,L-lactic acid), (poly(hydroxyalkanoate), poly(hydroxybutyrate), poly(hydroxybutyrate-co-valerate), poly(caprolactone), poly(butylene succinate), poly(ethylene succinate), poly(ethylene carbonate), poly(propylene carbonate), starch, thermoplastic starch, gelatin, poly(butylene terephthalate adipate), poly(propylene terephthalate succinate), poly(propylene terephthalate adipate), poly(vinyl alcohol), poly(ethylene glycol), cellulose, chitosan, cellulose acetate, cellulose butyrate acetate; or a crosslinked version thereof,
j) a polyester with greater than 5 mol percent aliphatic monomer content,
k) a crosslinked version of amorphous poly(D,L-lactic acid), poly(lactic acid), poly(L-lactic acid), poly(D-lactic acid), poly(meso-lactic acid), poly(rac-lactic acid), or poly(D,L-lactic acid), poly(hydroxyalkanoate), poly(hydroxybutyrate), poly(hydroxybutyrate-co-valerate), poly(caprolactone), poly(butylene succinate), poly(ethylene succinate), poly(ethylene carbonate), poly(propylene carbonate), starch, gelatin, thermoplastic starch, poly(butylene terephthalate adipate), poly(propylene terephthalate succinate), poly(propylene terephthalate adipate), poly(vinyl alcohol), poly(ethylene glycol), cellulose, chitosan, cellulose acetate, cellulose butyrate acetate, or a polyester with greater than 5 mol percent aliphatic monomer content,
l) a plasticizer, wherein the plasticizer is present at less than 30 wt % of the total composition,
m) acetyl tributyl citrate, tributyl citrate, di-n-octyl sebacate, di-2-ethylhexylsebacate, di-2-ethylhexylsuccinate, diisooctyl adipate, di-2-ethylhexyl adipate, diisooctyl glutarate, di-2-ethylhexyl glutarate, poly(ethylene glycol), poly(ethylene glycol) monolaurate, sorbitol, glycerol, poly(propylene glycol), or water,
n) copolymers of two or more of caprolactone, lactic acid, D-lactide, L-lactide, meso-lactide, D,L-lactide, sebacic acid, succinic acid, adipic acid, glycolic acid, oxalic acid, ethylene glycol, 1,2-propanediol, 1,3-propanediol, 1,3-butanediol, 1,4-butanediol, 1,5-pentanediol, 2,2,4,4-tetramethyl-1,3-cyclobutanediol, 1,6-hexanediol, terephthalic acid, isophthalic acid, succinic anhydride, a diisocyanate, a crosslinker, or phthalic anhydride,
o) an antioxidant, a nucleating agent, an impact modifier, a processing aid, a toughener, a colorant, a filler, a stabilizer, or a flame retardant,
p) a wax, Parafilm® or Nescofilm®,
q) paper, water soluble paper, recycled paper, bond paper, kraft paper, waxed paper, or coated paper; or
r) a combination of two or more of components a) through q) above, and
s) a blend comprising two or more of components a) through r) above.

5. The artificial seed of claim 1, wherein the container is expandable.

6. The artificial seed of claim 5, wherein said artificial seed is expandable through a method selected from the group consisting of:
a) telescoping of two or more tubular members,
b) unfolding,
c) inflation,
d) unraveling; and
e) stretching.

7. The artificial seed of claim 1, wherein the nutrient source further comprises a component selected from the group consisting of:
a) soil,
b) coconut coir,
c) vermiculite,
d) an artificial growth medium,
e) agar,
f) a superabsorbent polymer,
g) a plant growth regulator,
h) a plant hormone,
i) micronutrients,
j) macronutrients,
k) water,
l) a fertilizer,
m) peat,
n) a combination of two or more of components a) through m) above, and
o) a blend comprising two or more of components a) through n) above.

8. The artificial seed of claim 1, wherein the regenerable plant tissue is a regenerable tissue selected from the group consisting of:
a) sugar cane, a graminaceous plant, saccharum spp, saccharum spp hybrids, miscanthus, switchgrass, energycane, sterile grasses, bamboo, cassava, corn, rice, banana, potato, sweet potato, yam, pineapple, trees, willow, poplar, mulberry, ficus spp, oil palm, date palm, poaceae, verbena, vanilla, tea, hops, Erianthus spp, intergeneric hybrids of Saccharum, Erianthus and Sorghum spp, African violet, apple, date, fig, guava, mango, maple, plum, pomegranate, papaya, avocado, blackberries, garden strawberry, grapes, canna, cannabis, citrus, lemon, orange, grapefruit, tangerine, or dayap,
b) a genetically modified plant of a) above,
c) a micropropagated version of a) above, and
d) a genetically modified, micropropagated version of a) above.

9. The artificial seed of claim 1, wherein the container further comprises a component selected from the group consisting of:
a) a cylindrical tube with a conical top,
b) a two part tube with a porous bottom section and a nonporous top section,
c) a flexible packet,
d) a semi-flexible packet,
e) a rolled tube structure, capable of unraveling,
f) an anchoring device,
g) a multi-part tube with a hinged edge,
h) a multi-part tube held together with adhesive,
i) a tubular shape,
j) a container portion in contact with soil that degrades faster than the portion above soil,
k) an airspace comprising multiple compartments,
l) a closed bottom end that retains moisture,
m) a cap attached by an adhesive joint,
n) a cap attached by insertion into the container, and
o) a weak region.

10. The artificial seed of claim 1, wherein the container or closure further comprises a material selected from the group consisting of:
a) a transparent, translucent or semi-translucent material,
b) an opaque material,
c) a porous material,
d) a nonporous material,
e) a permeable material,
f) an impermeable material; and
g) any one of materials a) through f) above, wherein the material is biodegradable, hydrolytically degradable, or compostable.

11. The artificial seed of claim 1, where one or more of the openings are secured using a component selected from the group consisting of:
a) a crimp,
b) a fold,
c) a porous material,
d) mesh,
e) screen,
f) cotton,
g) gauze; and
h) a staple.

12. The artificial seed of claim 1, wherein the artificial seed further comprises an agent selected from the group consisting of:
a) a fungicide,
b) a nematicide,
c) an insecticide,
d) an antimicrobial compound,
e) an antibiotic,
f) a biocide,
g) an herbicide,
h) plant growth regulator or stimulator,
i) microbes,
j) a molluscicide,
k) a miticide,
l) an acaricide,
m) a bird repellant,
n) an insect repellant,
o) a plant hormone; and
p) a rodent repellant.

13. A method of storing the artificial seed of claim 1, comprising obtaining the artificial seed and storing said artificial seed before planting in one or more of the following conditions:
a) ambient conditions,
b) sub-ambient temperature,
c) sub-ambient oxygen levels, or
d) under sub-ambient illumination, and
wherein the regenerable plant tissue remains viable.

14. A method of planting the artificial seed of claim 1, comprising obtaining the artificial seed and performing a step from the group consisting of:
a) introducing one or more breaches in said artificial seed wherein the breaches facilitate the growth of the regenerable plant tissues,
b) expanding the artificial seed, and
c) the combination of a) and b) above.
